SEQUENCE LISTING (18 sequences)

SEQ ID NO: 1 - DNA, 3412 bp, Homo sapiens
agccgagcagcctggcaacggcggtggcgcccggagcccgagagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaggggggaccctggcaccgcattagagctcgaaataaaggcgatagctgactttcatttggggcatttgtaaaaagtagattaaaatatttgcctccatgtagaacttgaactaacataatcttaaactcttgaatatgtgccttctagaatacatattacaagaaaactacagggtccacacggcaatcagaagaaaggagctgagatgaggttttggaaaaccctgacacctttaaaaagcagtttttgaaagacaaaatttagatttaatttacgtcttgagaaatactatatatacaatatatattttgtgggcttaattgaaacaacattattttaaaatcaaaggggatatatgtttgtggaatggattttcctgaagctgcttaacagttgctttggattctctaagatgaatccaaatgtgaaagatgcatgttactgccaaaaccaaattgagctcagcttcctaggcattacccaaaagcaaggtgtttaagtaattgccagcttttataccatcatgagtggtgacttaaggagaaatagctgtatagatgagtttttcattatttggaaatttaggggtagaaaatgttttcccctaattttccagagaagcctatttttatatttttaaaaaactgacagggcccagttaaatatgatttgcattttttaaatttgccagttttattttctaaattctttcatgagcttgcctaaaattcggaatggttttcgggttgtggcaaaccccaaagagagcactgtccaaggatgtcgggagcatcctgctgcttaggggaatgttttcgcaaatgttgctctagtcagtccagctcatctgccaaaatgtagggctaccgtcttggatgcatgagctattgctagagcatcatccttagaaatcagtgccccagatgtacatgtgttgagcgtattcttgaaagtattgtgtttatgcatttcaatttcaatggtgttggcttcccctccccaccccacgcgtgcataaaaactggttctacaaatttttacttgaagtaccaggccgtttgctttttcaggttgttttgttttatagtattaagtgaaattttaaatgcacagttctatttgctatctgaactaattcatttattaagtatatttgtaaaagctaaggctcgagttaaaacaatgaagtgttttacaatgatttgtaaaggactatttataactaatatggttttgttttcaatgaattaagaaagattaaatatatctttgtaaattattttatgtcatagtttaattggtctaccaagtaagacatctcaaatacagtagtataatgtatgaattttgtaagtataagaaattttattagacattctcttactttttgtaaatgctgtaaatatttcataaattaacaaagtgtcactccataaaaagaaagctaatactaatagcctaaaagattttgtgaaatttcatgaaaactttttaatggcaataatgactaaagacctgctgtaataaatgtattaactgaaacctaa

SEQ ID NO: 2 - protein, 603 aa, Homo sapiens
MASASTSKYNSHSLENESIKRTSRDGVNRDLTEAVPRLPGETLITDKEVIYICPFNGPIKGRVYITNYRLYLRSLETDSSLILDVPLGVISRIEKMGGATSRGENSYGLDITCKDMRNLRFALKQEGHSRRDMFEILTRYAFPLAHSLPLFAFLNEEKFNVDGWTVYNPVEEYRRQGLPNHHWRITFINKCYELCDTYPALLVVPYRASDDDLRRVATFRSRNRIPVLSWIHPENKTVIVRCSQPLVGMSGKRNKDDEKYLDVIRETNKQISKLTIYDARPSVNAVANKATGGGYESDDAYHNAELFFLDIHNIHVMRESLKKVKDIVYPNVEESHWLSSLESTHWLEHIKLVLTGAIQVADKVSSGKSSVLVHCSDGWDRTAQLTSLAMLMLDSFYRSIEGFEILVQKEWISFGHKFASRIGHGDKNHTDADRSPIFLQFIDCVWQMSKQFPTAFEFNEQFLIIILDHLYSCRFGTFLFNCESARERQKVTERTVSLWSLINSNKEKFKNPFYTKEINRVLYPVASMRHLELWVNYYIRWNPRIKQQQPNPVEQRYMELLALRDEYIKRLEELQLANSAKLSDPPTSPSSPSQMMPHVQTHF

SEQ ID NO: 3 - DNA, 1060 bp, Artificial Sequence
  Synthetic Construct
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 4 - DNA, 1822 bp, Artificial Sequence
  Synthetic Construct
agtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctga

SEQ ID NO: 5 - DNA, 12471 bp, Artificial Sequence
  Synthetic Construct
tcgcgcgtttcggtgatgacggtgaaaacctctgacacatgcagctcccggacgtcattgtcgatcctgcaggcgtacggtaaaaaaaggcatagctaacaaggtgtggaaaaagaattagtggttagagagtgagctattcgttgaaacaattgcgttcttgaaacaattcttgctggtaaaatgtcacattttatgtgactacaggtggaggattggcacataacctaaccagtgggggaaacaattgacctctggatttgtccaagtgtatagtagcatttgcccaatcgaatggtcctggtaaggtgttaatgttgactagaaccaaaggtggaagttgcagggaaactggtttagtacaagggtggacaccaggcagtcatccagaggcccattaaaggccttggaatgtttttccgaaggagaatcactccctcttctctcgcttaaagttttaggggattcatgaacagctgctgtgggatagtttcatgtccctagcaattgtaaagcaactgagggtggcttaaaccagttttagctttagggttagggttactggactaaaatttgagaaattcataaatcttaaggaaatccattgtgagttttcattatgagtgcatccaatgtataatttccatgaccctcccatgcaagtgagcatgtgaatcaggaaacgttacaagaacccaacaaactcaaccactactagacaggcgatcacttccagttagtatgcaactttctgtgtaattttagttaccattaaaatctggatgaccttagtgtaaggaaaaaataccttgaatagtgttaaagatgtacacttggtgtcaggcattgtaacattgataaatctgtgtaaggtgctttttgaaaacttcaaagctgcatcaagtcaagtacaagaaaggccatggctgctaaagctgttgaagatgtgggatggaactgggtcacattggtgttaacagcgttgtgcagagccggcaggatcttggtgtgagcgaacattagtctatttaataaagctgtgtgaatgttgtagaggtgaggatgctcacttgaaaactcactgaagaacacttggccccttgaactaaagtgcttctatcaagttcagtgagaaattccgaattacaagcataggtactagaaaagttttgaaaagcagtatagagcaacataagcacattcataaaattagtgatgtagaaagtgaaatttccacgtatggtcactcccagagaaaaaaaatacgtttatttaccttttttaaaaataggggatttcaggccgggtgaggtggctcacgcctgtaatcccagcactttgggaggcccaggtgggcggatcacctgaggtcaggagttggagggatggcaaatcccatctctacaaaatatacaaaaaaatagctgggtgtgttggcaggcgcctgtaatcccagctactcggaaggctgaggcaggagaatccctggaaccagggatgtggaggttgcagtgagccgagattgtgtaactgcattccagcctgggcaacaagagcaagactccgtatcaggaaaaaaaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacaagatttctattttaattggattcttagggctaataaaataatcagccttagcacttatttatttattttttttgagagggagtctcgctctgttgtccatgctggagtgcagtggcgtgatctcggctcactgcaagctccacctcatgagttcacaccattctcctgcctcagtctcccgagtagctgggactccaggcgccctctacaaagcccgtctaattttttttgtatttttagtagagacagggtttcactgtgttagccaggatggtcttgatctcctgaccttgtgatctgcccgcctcggcctcccaaagtgctgggattataggcttgagccactgctcccggccagcacttatttttataattcttcatgattactgtgttactgtcccatgggccgccagggccagctaggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctagcacgcgctaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggactctagaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgcaagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaccggtccgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgaccggccgctctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctagaggccgccagggccatatttctcaatttttaaatttttcaaaaaaattaatccttaatgtgcatatttttgaattgttaatataactttttgaggtgatgtcttcatgtgtttcaactacttaaaaacttttaaacagtatataataaaaaatcttccaggccactcacacctgtaatcccagcactttgggaggctgaggtgggcagatcacctgagggcaggagttcgagaccagcctggccaatatatatatattcatatattcatatatatatatatattcatatattcatatatatatattcatatattcatatatatatatatatatatatatagcaaaacctcatctctaataaaatacaaaaattagctgagcgtggtgatggatgcctgtagtcccagctactcgggaggctgaggcaggagaatctcttgaacctgggaggtggaggttgcagtgagctgagatggtgccactgccctccagcctgagtgacagagcgagactcggtctccaaaaaaaaacaacaaaaaaatcttccatccttgtctcccatccaccccttccccccagcatgtacttgcagactttatgcatatacagtgagtactgtatatacacaaataataaaaaaatcatatatataatatatgtaattcccctttacatgaaaggtagcacactggtctgtacagtctgtctgcactgtgctatttcactttatatttttatagtttgacagagttctaacatttctttttttttttttttaacagagtcttgttcctgattgttaaattttaaagcatcctaaagtttggtttcacacttgaatgaataccatgtaaggattcacttacatagatgtggttgcctgaatcttaagaataaaataacattgtttgtatttatttaaattagtgttccttttatggtttgcctgaaagcacaacaaaatcctcaccaagatattacaattatgactcccatacaggtaaactgtttagagattggcaagcaccttttaatgaaaggagtcagccagcttagtgtgcagtatttatttctgccggaagagggagcttcagggacagactttggtttagtcatgaagcctccagcactcccaagcggttgtggttgaccaagcaatttatgcttttacctttctacttccagaggcttgtttacttatcagtaagcattaatttagtgtcccctcagatgccttttactttcttcttttctgcctagaataagctgctcttccaattttgcagctacatgtttccaccccagttggaatttctccataacatccattgtagctatccttcaatctacagcctctatttcctgttatagctggtcaggtctaatccctcaaaatactctgtcccctgcttcccttatctgctggccacctttttcccccacatacacactgccatgtcccacccttcactcaagttgttccctgccacctcaacaaatttaagtccataaaatagagtaagtgttcctgactgttaaattttaaagcatcccaaagtctgatttcacactcgaatgaatactatgtacggattcatttacatagatgcggttgcatgagtcttaacaaaaaaataacattatttgtatttattcaaagtactgtcaagatataatgtcaagacctaattcaaaggttccacaaagccttccttgactgcccccaacgaagattatccattttccctgaaatcccattgacttttctattttgtaaggaggctcgtgagactctgtctaaaaacaaaacaaaacaaaaagaaacaatcaaacggcttgcttctgttctttgatctgctagtaagcaaaaattacacatggtgacaggagctatgtgaggctgtcaggttgaatgggaggagtttgggatcctgcttgtggatggttggaagaggctttcgggaaagacagtatttatgtgagacctggaagatgggccttagctttgcagaaggtggagaggcaggaaatagcacgggggccctggggctggaagacttgggcatatttgaggaacagaaaggagaccagcataactgaggtgggaaaagcatgtgaagagatggggctggaggaggccgggagtggtggctcacgcctgtaatcccagcactttgggaggccaaggcaggcggatcatgagctcaggagattgagaccatcctggctaacacggtgaaaccccctctctactaaaaatacaaaaaaaaaaaaaaaaaaaattagctgggcgtggtggcaggagcctgtagtcccagctacctgggaggctgaggcaggagaatggcgtgaacctggaaggctgagcttgcagtgagccgagattgcaccactgcactccagcctgggagacagagagagactccctctcaaaaaaacaaacaaacgaaacaaaacaaaacaaaaattagccaggcgtggtggtatgcacctgtaatcccagctactcgggaggttgaggcaggagaaacgcttgaactcaggaggcggaggttgcagtgagccgagactgcgccactgcactccagcctgggtgacagagggagactccatctcaaaaaaaaaaatttttttttttttacaaacggtgtctccctctgtcgcccaggctggagtgcagtggtgtgatcacagctcactccagcctcaacctccccagctgaagccatcctcttgcctcagcctcctaagtagctgggactacaggcgcgcacctccaggcttggctcttattctttttattgtttttgaaactatagaacctatttttaaaaaatgttttggttgtttttattgctgcttttccttttggggttagaacacaagttttgatgggaaacaggttagaacacattcatctcttcccatagcgatggtcatagaaaaacggggcatatttataaactctcagttgatcttaaaatgtgcaaaagctgccgaactcctgggagtgagctcgagccctgcaggatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 6 - DNA, 1896 bp, Homo sapiens
agacgggtggggcggggcccaactgtccccagctccttcagccctttctgtccctcccagtgaggccagctgcggtgaagagggtgctctcttgcctggagttccctctgctacggctgccccctcccagccctggcccactaagccagacccagctgtcgccattcccacttctggtcctgccacctcctgagctgccttcccgcctggtctgggtagagtcatggcctcgagcacaggtgaccggagccaggcggtgaggcatggactgagggcgaaggtgctgacgctggacggcatgaacccgcgtgtgcggagagtggagtacgcagtgcgtggccccatagtgcagcgagccttggagctggagcaggagctgcgccagggtgtgaagaagcctttcaccgaggtcatccgtgccaacatcggggacgcacaggctatggggcagaggcccatcaccttcctgcgccaggtcttggccctctgtgttaaccctgatcttctgagcagccccaacttccctgacgatgccaagaaaagggcggagcgcatcttgcaggcgtgtgggggccacagtctgggggcctacagcgtcagctccggcatccagctgatccgggaggacgtggcgcggtacattgagaggcgtgacggaggcatccctgcggaccccaacaacgtcttcctgtccacaggggccagcgatgccatcgtgacggtgctgaagctgctggtggccggcgagggccacacacgcacgggtgtgctcatccccatcccccagtacccactctactcggccacgctggcagagctgggcgcagtgcaggtggattactacctggacgaggagcgtgcctgggcgctggacgtggccgagcttcaccgtgcactgggccaggcgcgtgaccactgccgccctcgtgcgctctgtgtcatcaaccctggcaaccccaccgggcaggtgcagacccgcgagtgcatcgaggccgtgatccgcttcgccttcgaagagcggctctttctgctggcggacgaggtgtaccaggacaacgtgtacgccgcgggttcgcagttccactcattcaagaaggtgctcatggagatggggccgccctacgccgggcagcaggagcttgcctccttccactccacctccaagggctacatgggcgagtgcgggttccgcggcggctatgtggaggtggtgaacatggacgctgcagtgcagcagcagatgctgaagctgatgagtgtgcggctgtgcccgccggtgccaggacaggccctgctggacctggtggtcagcccgcccgcgcccaccgacccctcctttgcgcagttccaggctgagaagcaggcagtgctggcagagctggcggccaaggccaagctcaccgagcaggtcttcaatgaggctcctggcatcagctgcaacccagtgcagggcgccatgtactccttcccgcgcgtgcagctgcccccgcgggcggtggagcgcgctcaggagctgggcctggcccccgatatgttcttctgcctgcgcctcctggaggagaccggcatctgcgtggtgccagggagcggctttgggcagcgggaaggcacctaccacttccggatgaccattctgccccccttggagaaactgcggctgctgctggagaagctgagcaggttccatgccaagttcaccctcgagtactcctgagcaccccagctggggccaggctgggtcgccctggactgtgtgctcaggagccctgggaggctctggagcccactgtacttgctcttgatgcctggcggggtggggtggggggggtgctgggcccctgcctctctgcaggtccctaataaagctgtgtggcagtctgactccaaaaaaaaaaa

SEQ ID NO: 7 - protein, 496 aa, Homo sapiens
MASSTGDRSQAVRHGLRAKVLTLDGMNPRVRRVEYAVRGPIVQRALELEQELRQGVKKPFTEVIRANIGDAQAMGQRPITFLRQVLALCVNPDLLSSPNFPDDAKKRAERILQACGGHSLGAYSVSSGIQLIREDVARYIERRDGGIPADPNNVFLSTGASDAIVTVLKLLVAGEGHTRTGVLIPIPQYPLYSATLAELGAVQVDYYLDEERAWALDVAELHRALGQARDHCRPRALCVINPGNPTGQVQTRECIEAVIRFAFEERLFLLADEVYQDNVYAAGSQFHSFKKVLMEMGPPYAGQQELASFHSTSKGYMGECGFRGGYVEVVNMDAAVQQQMLKLMSVRLCPPVPGQALLDLVVSPPAPTDPSFAQFQAEKQAVLAELAAKAKLTEQVFNEAPGISCNPVQGAMYSFPRVQLPPRAVERAQELGLAPDMFFCLRLLEETGICVVPGSGFGQREGTYHFRMTILPPLEKLRLLLEKLSRFHAKFTLEYS

SEQ ID NO: 8 - DNA, 3958 bp, Homo sapiens
gttcacttactggtgcccagcaccgggcactcagtgaggccttgcaatacggttcacttgttgaattgaattcgtcaattgttgagcgggccgtgctgccaggcaccgcacgttgtgtgtctgccctactctcgtggaaccaaggtccagtggagagaggaacataagcgaatgaagaaagaagcaatagagtctcagtgataagttcttggaaaaaaggtctttgccaatcctgattcccggaagagagtcggcagaattccttcctttaggcccctctggagccttggccgaccagccacttctgtggtctggggacagtcaccgagggtatcaaaaagccattcacagaggtcatccgagccaacatcggggacgcccaggctatggggcagcagccaatcaccttcctccggcaggtgatggcactatgcacctacccaaacctgctggacagccccagcttcccagaagatgctaagaaacgtgcccggcggatcctgcaggcttgtggcgggaacagcctggggtcctacagtgctagccagggtgtcaactgcatccgtgaagatgtggctgcctacatcaccaggagggatggcggtgtgcctgcggaccccgacaacatctacctgaccacgggagctagtgacggcatttctacgatcctgaagatcctcgtctccgggggcggcaagtcacggacaggtgtgatgatccccatcccacaatatcccctctattcagctgtcatctctgagctcgacgccatccaggtgaattactacctggacgaggagaactgctgggcgctgaatgtgaatgagctccggcgggcggtgcaggaggccaaagaccactgtgatcctaaggtgctctgcataatcaaccctgggaaccccacaggccaggtacaaagcagaaagtgcatagaagatgtgatccactttgcctgggaagagaagctctttctcctggctgatgaggtgtaccaggacaacgtgtactctccagattgcagattccactccttcaagaaggtgctgtacgagatggggcccgagtactccagcaacgtggagctcgcctccttccactccacctccaagggctacatgggcgagtgtggttacagaggaggctacatggaggtgatcaacctgcaccctgagatcaagggccagctggtgaagctgctgtcggtgcgcctgtgccccccagtgtctgggcaggccgccatggacattgtcgtgaaccccccggtggcaggagaggagtcctttgagcaattcagccgagagaaggagtcggtcctgggtaatctggccaaaaaagcaaagctgacggaagacctgtttaaccaagtcccaggaattcactgcaaccccttgcagggggccatgtacgccttccctcggatcttcattcctgccaaagctgtggaggctgctcaggcccatcaaatggctccagacatgttctactgcatgaagctcctggaggagactggcatctgtgtcgtgcccggcagtggctttgggcagagggaaggcacttaccacttcaggatgactatcctccctccagtggagaagctgaaaacggtgctgcagaaggtgaaagacttccacatcaacttcctggagaagtacgcgtgaggacgcctgagccccagcgggagacctgtccttggctcttcctcccaatgcccgtcaggctgaactcgcctcccccgtgactctgcctcgggcctcgcagaggccgctggtcacttcgtcatcattttgcccctggagacgtctttctttgtgccttgatgttgagagcgcctctcttttgagcaaacaagcattctatatgcaaccagagtagaggggacctgctcagcaggtgtgaccagggttctctgaatctgttattgtttttgcttctggaaagttcatttggggtttacaacaactaggatgtgttgggtgagatgtttcagatctggagaaatgagcaggtgtcgggaaatgtgtgacttaaccgtggtgagggctggaaatccaaactcaccaccatgatctgtgaaataaagcccttagcggtgtgaagcatccggtcctttgaacagaagggcctggaaggcccctggggctgagaaagggtccgcccggtggcctggaggcaggcgccgggagcgcagtagcacgtggactgggcaggatgttgcactagcttggggtagatgctgggggctgcggccacggtcagagggccccactgtgaggcgtgggtgtgagccaggctgcaggaggaactgggcctccgcttcccagcaacgcagccaggcctgagaattctgtgcgcccggcgggctttgggaatgaggggttcccttgaacatgcgtaggctggaaccccgtctgagaggtctccctgaatttcagtgacacatagtgcagcccggcagtgtcccacttccgtggagagagccgctggaatggtgtggacccatcccgcgggtgaccggtgcctgttctcccctgaccgagcctgtgagcacatcgccccctgctggcgacagcggggaaatgagggctgaaaatatcctccccacaagggcaatccccgggacctgccgagcagccaaggccctgtcctttcttgaatggtggcgagctgaatctggtcggtttcctagcttttaggtggtaaaagtgcctggcagcttggctgccgtggaggagtcagtcgtggttggaggttcattgccgtgctttcatgcagagtgttttgccttcatgttagcttccggctcccctcccaggctgcagactctgacctgtggcatcaggcttctcccagtacaggagggtgccatcccccagcatgcggcttctctgccattagcagccctgggcgggccgaccacactcgaggctgcggtgctacgggcttagccctcgcctccctcactgggagcttccccatcctccctgccttccccagtgggaagttagggaagctcaggagcctgggaccccgcatgtcccaaaatgggattggagaagctggagagaaagcagaagaggccgaggagtgaggcagcagcctctatgctgtgatttccacaccgggtccgtgcagaggaaacagaaactcccaactgtccttacccaccgacatcacagcccctatgaagaaagtagccacaatctcaaataacaaaagggaatgttctaaaactttttcttccttaaaaaatggagaaaattgcacttgtgcttgctgtgtggtatataaaccaggattagtcccagggtcgtgaggtttctggtgaaaaggttaaatcgtagaagctagtatattttttatatttttgtaacaattgcttttttcatgggggaggcggggttagtatttatagtcctaacaagtccagtaattttttataaatcttcagattataaacagcccctaaaaactttacaacgtttacacagttttttaaaaagagactgtatacacttgatttgctttcaaaataaataaggtcagctagtctaggaggttaacgtcgggtaggaatgctgatcatgataggtttggttttctacagattctgttccggtgcctttcctatccaggcaccacctgagaaagttgtcatttgaggtcgcacttggaagttacatctgtgaagtttctgtcattcgtccagatctgtgtgtgtagcatgtgctgaggaagcacgtgctgggctgtgcctcagacagtgcatcaccgggcacccagaggcttgcctggctattcctgttctggtgtgtgtggagtgttggggaggaacagatgcagatcaacctgtggctgttttcccgtctaggttctcacaggtatctcctgacagaggtacttaacaatggctctgctggaaatttctataaataaaatgtccaaaatggtgactgcgttt

SEQ ID NO: 9 - protein, 423 aa, Homo sapiens
MGQQPITFLRQVMALCTYPNLLDSPSFPEDAKKRARRILQACGGNSLGSYSASQGVNCIREDVAAYITRRDGGVPADPDNIYLTTGASDGISTILKILVSGGGKSRTGVMIPIPQYPLYSAVISELDAIQVNYYLDEENCWALNVNELRRAVQEAKDHCDPKVLCIINPGNPTGQVQSRKCIEDVIHFAWEEKLFLLADEVYQDNVYSPDCRFHSFKKVLYEMGPEYSSNVELASFHSTSKGYMGECGYRGGYMEVINLHPEIKGQLVKLLSVRLCPPVSGQAAMDIVVNPPVAGEESFEQFSREKESVLGNLAKKAKLTEDLFNQVPGIHCNPLQGAMYAFPRIFIPAKAVEAAQAHQMAPDMFYCMKLLEETGICVVPGSGFGQREGTYHFRMTILPPVEKLKTVLQKVKDFHINFLEKYA

SEQ ID NO: 10 - DNA, 2613 bp, Homo sapiens
gggctgcccgggcctcactcgggccccgcggccgcctttataaggcggcgggggtggtggcccgggccgcgttgcgctcccgccactccgcgcccgctatcctggctccgtgctcccacgcgcttgtgcctggacggaccctcgccagtgctctgcgcaggattggaacatcagttaacatctgaccactgccagcccaccccctcccacccacgtcgattgcatctctgggctccagggataaagcaggtcttggggtgcaccatgatttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggcagccttgctgcaggccccctgctgctcgcgctggccctctaccccctgagcgtcctgttctgagggcccagggcccgggcacccacaagcccgtgacagatgccaacttcccacacggcagcccccccctcaaggggcagggaggtgggggcctcctcagcctctgcaactgcaagaaaggggacccaagaaaccaaagtctgccgcccacctcgctcccctctggaatcttccccaagggccaaacccacttctggcctccagcctttgctccctccccgctgccctttggccaacagggtagatttctcttgggcaggcagagagtacagactgcagacattctcaaagcctcttatttttctagcgaacgtatttctccagacccagaggccctgaagcctccgtggaacattctggatctgaccctcccagtctcatctcctgaccctcccactcccatctccttacctctggaaccccccaggccctacaatgctcatgtccctgtccccaggcccagccctccttcaggggagttgaggtctttctcctcaggacaaggccttgctcactcactcactccaagaccaccagggtcccaggaagccggtgcctgggtggccatcctacccagcgtggcccaggccgggaagagccacctggcagggctcacactcctgggctctgaacacacacgccagctcctctctgaagcgactctcctgtttggaacggcaaaaaaaaatttttttttctctttttggtggtggttaaaagggaacacaaaacatttaaataaaactttccaaatatttccgaggacaaaaaaaaaaa

SEQ ID NO: 11 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 12 - DNA, 2140 bp, Homo sapiens
gcgttccttctcccctgtgccttcgtcgtcagaagctggcgattggttaatcgcgttgccaagctttggacgcggctcgaccattggaggccgcgggcccgcccccgccggctaggtgaaggtgagtgtctcctccagtcgcaacggccagacctgacctgccagctccgggcgtggggtgaaatctcttgattcctagtctctcgatatggcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttggggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacaatgagagagtcgggaatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccagtgaagaaacaccacccgtccagtaccaccaaagtagttctctgtcatgtgtgttccctgcctgcacaaacctacatgtacataccatggattagagacacttgcaggactgaaaggctgctctggtgaggcagcctctgtttaaaccggccccacatgaagagaacatcccttgagacgaatttggagactgggattagagcctttggaggtcaaagcaaattaagatttttatttaagaataaaagagtactttgatcatgagacataggtatcttgtccctctcactaaaaaggagtgttgtgtgtggcggccacgtgcttctatgtggtgtttgactctgtacaaattctagtcccaaagatcaagttgtctgaaggagccaaagtgtgaatgtgggtgtcggctgcggcattaaattcatcatctcaacccagagtgtctggtctccctgctctttctgcatggttgtgtccctagtcctaagctttggttctttagggtgactgtggtaagaaggatatttaatcatgacatgcacggacacgtacatatttaactgaaacaagttttaccaaacagtatttactcgtgatgtgcgtagtgcattctgatatttttgagccattctattgtgttctacttcacctaaaaaaataaaataaaaatgttgatcaagaaaaaaaaaaaaaa

SEQ ID NO: 13 - protein, 413 aa, Homo sapiens
MAPPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYNERVGNLTVVGKEPESILQVLSQMEKIVRITWSNPPAQGARIVASTLSNPELFEEWTGNVKTMADRILTMRSELRARLEALKTPGTWNHITDQIGMFSFTGLNPKQVEYLVNEKHIYLLPSGRINVSGLTTKNLDYVATSIHEAVTKIQ

SEQ ID NO: 14 - DNA, 2693 bp, Homo sapiens
aggaggagctgggtcacagcagggaatcttagcttggttttggtgtgctgctggatgaccagaccgggcgtcgggtgagcccagaagtgagagcagttggctgtgccccagtgctgtgtgacccagaggcgccgctcaccctctctgagctggtggacatcataggtggggaagctcaggtcagggcactcccatgagtgtctggaggcctgagtcccattctcagctctgccatatgcttgctgcgctctagaggagttcctcttcctctccaagcctcggtttatgtacccgtgcagtgggagtgagttgcacttcggggtgaagggggcaagacttgtgtgggcgcatcctgcagaaggatcccacagcgggtgcagcccagaactgtcttctgaggaagaggtgctctcctgggcccccactgtccccaggcctcagcaaggcaagtgaggtgctgccgtcatccaggctggacagttcagtgatttgcctgaggccccacagcagagttcaactggagacagagaaaccagctagaggcagagggaggtaacacggagtcccccagaaaggtctgggctgcgcgtgcttcaggtaacctcccttgaccttcaggagaacgagaaggctgcctgatcagagagtccctgaagaagattctgtggctacaggcttcagcagagtgtgagggagaccccggttatttcctcagctatttccaccaaatcctcctgtctttcgtggccaacaccccaggcaaggcttggggcccccgtctgctgctggacgcagagccatgaagaagaagttagtggtgctgggcctgctggccgtggtcctggtgctggtcattgtcggcctctgtctctggctgccctcagcctccaaggaacctgacaaccatgtgtacaccagggctgccgtggccgcggatgccaagcagtgctcgaagattgggagggatgcactgcgggacggtggctctgcggtggatgcagccattgcagccctgttgtgtgtggggctcatgaatgcccacagcatgggcatcgggggtggcctcttcctcaccatctacaacagcaccacacgaaaagctgaggtcatcaacgcccgcgaggtggcccccaggctggcctttgccaccatgttcaacagctcggagcagtcccagaagggggggctgtcggtggcggtgcctggggagatccgaggctatgagctggcacaccagcggcatgggcggctgccctgggctcgcctcttccagcccagcatccagctggcccgccagggcttccccgtgggcaagggcttggcggcagccctggaaaacaagcggaccgtcatcgagcagcagcctgtcttgtgtgaggtgttctgccgggatagaaaggtgcttcgggagggggagagactgaccctgccgcagctggctgacacctacgagacgctggccatcgagggtgcccaggccttctacaacggcagcctcacggcccagattgtgaaggacatccaggcggccgggggcattgtgacagctgaggacctgaacaactaccgtgctgagctgatcgagcacccgctgaacatcagcctgggagacgtggtgctgtacatgcccagtgcgccgctcagcgggcccgtgctggccctcatcctcaacatcctcaaagggtacaacttctcccgggagagcgtggagagccccgagcagaagggcctgacgtaccaccgcatcgtagaggctttccggtttgcctacgccaagaggaccctgcttggggaccccaagtttgtggatgtgactgaggtggtccgcaacatgacctccgagttcttcgctgcccagctccgggcccagatctctgacgacaccactcacccgatctcctactacaagcccgagttctacacgccggatgacgggggcactgctcacctgtctgtcgtcgcagaggacggcagtgctgtgtccgccaccagcaccatcaacctctactttggctccaaggtccgctccccggtcagcgggatcctgttcaataatgaaatggacgacttcagctctcccagcatcaccaacgagtttggggtacccccctcacctgccaatttcatccagccagggaagcagccgctctcgtccatgtgcccgacgatcatggtgggccaggacggccaggtccggatggtggtgggagctgctgggggcacacagatcaccacggccactgcactggccatcatctacaacctctggttcggctatgacgtgaagcgggccgtggaggagccccggctgcacaaccagcttctgcccaacgtcacgacagtggagagaaacattgaccaggcagtgactgcagccctggagacccggcaccatcacacccagatcgcgtccaccttcatcgctgtggtgcaagccatcgtccgcacggctggtggctgggcagctgcctcggactccaggaaaggcggggagcctgccggctactgagtgctccaggaggacaaggctgacaagcaatccagggacaagatactcaccaggaccaggaaggggactctgggggaccggcttcccctgtgagcagcagagcagcacaataaatgaggccactgtgccaggctccaggtggcctccctggcctgtctccccactcaaaaaaaaaaaaaaaaaa

SEQ ID NO: 15 - protein, 569 aa, Homo sapiens
MKKKLVVLGLLAVVLVLVIVGLCLWLPSASKEPDNHVYTRAAVAADAKQCSKIGRDALRDGGSAVDAAIAALLCVGLMNAHSMGIGGGLFLTIYNSTTRKAEVINAREVAPRLAFATMFNSSEQSQKGGLSVAVPGEIRGYELAHQRHGRLPWARLFQPSIQLARQGFPVGKGLAAALENKRTVIEQQPVLCEVFCRDRKVLREGERLTLPQLADTYETLAIEGAQAFYNGSLTAQIVKDIQAAGGIVTAEDLNNYRAELIEHPLNISLGDVVLYMPSAPLSGPVLALILNILKGYNFSRESVESPEQKGLTYHRIVEAFRFAYAKRTLLGDPKFVDVTEVVRNMTSEFFAAQLRAQISDDTTHPISYYKPEFYTPDDGGTAHLSVVAEDGSAVSATSTINLYFGSKVRSPVSGILFNNEMDDFSSPSITNEFGVPPSPANFIQPGKQPLSSMCPTIMVGQDGQVRMVVGAAGGTQITTATALAIIYNLWFGYDVKRAVEEPRLHNQLLPNVTTVERNIDQAVTAALETRHHHTQIASTFIAVVQAIVRTAGGWAAASDSRKGGEPAGY

SEQ ID NO: 16 - DNA, 19 bp, Artificial Sequence
  Synthetic Construct
ccccaacttcaccttccag

SEQ ID NO: 17 - DNA, 18 bp, Artificial Sequence
  Synthetic Construct
attagccacaccagccac

SEQ ID NO: 18 - DNA, 23 bp, Artificial Sequence
  Synthetic Construct
tgccccatgtgcaaactcacttc

(19) United States
(12) Patent Application Publication
(10) Pub. No.: US 2024/0366785 A1
(43) Pub. Date: Nov. 7, 2024

(54) COMPOSITIONS AND METHODS FOR IMPROVED TREATMENT OF X-LINKED MYOTUBULAR MYOPATHY

(71) Applicant: AUDENTES THERAPEUTICS, INC., San Francisco, CA (US)

(72) Inventors: Weston MILLER, San Francisco, CA (US); Nathan BACHTELL, San Francisco, CA (US)

(21) Appl. No.: 18/563,633

(22) PCT Filed: May 24, 2022
(86) PCT No.: PCT/US2022/030716

§ 371 Date: Nov. 22, 2023

Related U.S. Application Data
(60) Provisional application No. 63/245,611, filed on Sep. 17, 2021.
(60) Provisional application No. 63/192,279, filed on May 24, 2021.

(51) Int. Cl. A61K 48/00; A61K 31/575; A61K 38/17; A61P 21/00; C12N 15/86

(52) U.S. Cl. CPC A61K 48/0033; A61K 31/575; A61K 38/1709; A61K 48/0075; A61K 48/0083; A61P 21/00; C12N 15/86; C12N 2750/14143; C12N 2750/14171

(57) ABSTRACT

The present invention provides methods for treating co-morbid cholestatic liver dysfunction (e.g., cholestasis and hyperbilirubinemia) associated with a neuromuscular disorder. In certain embodiments, the invention provides methods for assessing readiness of a subject with X-linked myotubular myopathy (XLMTM) for combination therapy with an anti-cholestatic agent.

### SEQUENCE LISTING

The instant application contains a Sequence Listing which has been submitted electronically in ASCII format and is hereby incorporated by reference in its entirety. Said ASCII copy, created on May 24, 2022 is named “51037-057WO3_Sequence_Listing_5_24_22_ST25” and is 69,743 bytes in size.

### FIELD OF THE INVENTION

The present invention relates to a method for the treatment of cholestatic liver dysfunction associated with current treatments of neuromuscular disorders in patients, such as human patients.

### BACKGROUND OF THE INVENTION

X-linked myotubular myopathy (XLMTM) is a fatal monogenic disease of skeletal muscle, resulting from loss-of-function mutations in Myotubularin 1 (MTM1). Approximately one in every 50,000 newborn boys has XLMTM, which typically displays as marked hypotonia and respiratory failure. In extremely rare cases, females can develop a severe form of XLMTM. Survival beyond the postnatal period requires intensive support, including respiratory support (i.e., mechanical ventilation) at birth in 85-90% of patients, ongoing 24-hour ventilator dependence in nearly 50% of patients, and tracheostomy in ˜60% of patients. Until recently, only supportive treatment options, such as ventilator use or a feeding tube, were available. Recently, gene therapy approaches involving the delivery of MTM1 have been developed for the treatment of XLMTM. However, there is a need in the art for improved methods of administering gene therapy to patients having XLMTM.

### SUMMARY OF THE INVENTION

The disclosure provides methods for treating X-linked myotubular myopathy (XLMTM) in a human patient in need thereof. In some embodiments, the patient is administered a therapeutically effective amount of a viral vector containing a transgene encoding myotubularin 1 (MTM1) and an anti-cholestatic agent.

In one aspect, the disclosure provides a method of treating XLMTM in a human patient in need thereof, the method including administering to the patient (i) a therapeutically effective amount of a transgene encoding MTM1 and (ii) an anti-cholestatic agent, wherein the anti-cholestatic agent is administered to the patient in one or more doses that commence within about six weeks (e.g., about six weeks before or about six weeks after) of administration of the transgene to the patient.

In a second aspect, the disclosure provides a method of reducing stiffness and/or joint contractures in a human patient diagnosed as having XLMTM, the method comprising administering to the patient (i) a therapeutically effective amount of a viral vector comprising a transgene encoding MTM1 and (ii) an anti-cholestatic agent, wherein the anti-cholestatic agent is administered to the patient in one or more doses that commence within about six weeks (e.g., about six weeks before or about six weeks after) of administration of the viral vector to the patient.

In another aspect, the disclosure provides a method of increasing diaphragm and/or respiratory muscle progression in a human patient diagnosed as having XLMTM, the method comprising administering to the patient (i) a therapeutically effective amount of a viral vector comprising a transgene encoding MTM1 and (ii) an anti-cholestatic agent, wherein the anti-cholestatic agent is administered to the patient in one or more doses that commence within about six weeks (e.g., about six weeks before or about six weeks after) of administration of the viral vector to the patient.

In some embodiments, the anti-cholestatic agent is administered to the patient in one or more doses that commence within about five weeks (e.g., about five weeks before or about five weeks after) of administration of the transgene to the patient, optionally wherein the anti-cholestatic agent is administered to the patient in one or more doses that commence within about four weeks (e.g., about four weeks before or about four weeks after), within about three weeks (e.g., about three weeks before or about three weeks after), within about two weeks (e.g., about two weeks before or about two weeks after), or within about one week (e.g., about one week before or about one week after, about six days before or about six days after, about five days before or about five days after, about four days before or about four days after, about three days before or about three days after, about two days before or about two days after, or about one day before or about one day after) of administration of the transgene to the patient.

In some embodiments, the anti-cholestatic agent is administered to the patient in one or more doses that commence on the same day as administration of the transgene to the patient.

In another aspect, the disclosure provides a method of treating XLMTM in a human patient in need thereof and who has been previously administered an anti-cholestatic agent, the method including administering to the patient a therapeutically effective amount of a transgene encoding MTM1.

In another aspect, the disclosure provides a method of reducing stiffness and/or joint contractures in a human patient diagnosed as having XLMTM and who has been previously administered an anti-cholestatic agent, the method comprising administering to the patient a therapeutically effective amount of a viral vector comprising a transgene encoding MTM1.

In another aspect, the disclosure provides a method of increasing diaphragm and/or respiratory muscle progression in a human patient diagnosed as having XLMTM and who has been previously administered an anti-cholestatic agent, the method comprising administering to the patient a therapeutically effective amount of a viral vector comprising a transgene encoding MTM1.

In some embodiments of either of the foregoing aspects, the method further includes monitoring the patient for development of cholestasis or hyperbilirubinemia.

In some embodiments of either of the foregoing aspects, the patient is monitored for the development of cholestasis, hyperbilirubinemia, or one or more symptoms thereof by evaluating a parameter in a blood sample obtained from the patient, wherein a finding that the parameter is above a reference level identifies the patient as having cholestasis, hyperbilirubinemia, or one or more symptoms thereof.

In some embodiments, the parameter includes the level of a serum bile acid in the blood sample. In some embodiments, the serum bile acid is cholic acid, chenodeoxycholic acid, deoxycholic acid, or ursodeoxycholic acid.

In some embodiments, the parameter includes one or more results of a liver function test.

In some embodiments of any of the foregoing aspects, the parameter includes the level of aspartate aminotransferase or alanine aminotransferase in the blood sample.

In another aspect, the disclosure provides a method of treating XLMTM in a human patient in need thereof, the method including: (a) administering to the patient a transgene encoding MTM1, (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of treating XLMTM in a human patient in need thereof, the method including: (a) administering to the patient a viral vector including a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of reducing stiffness and/or joint contractures in a human patient diagnosed as having XLMTM, the method comprising: (a) administering to the patient a viral vector comprising a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of increasing diaphragm and/or respiratory muscle progression in a human patient diagnosed as having XLMTM, the method comprising: a) administering to the patient a viral vector comprising a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of treating XLMTM in a human patient in need thereof, the method including: (a) administering to the patient a transgene encoding MTM1, (b) determining that the patient exhibits cholestasis, hyperbilirubinemia, or one or more symptoms thereof, and (c) administering to the patient an anti-cholestatic agent.

In another aspect, the disclosure provides a method of treating XLMTM in a human patient in need thereof, the method including: (a) administering to the patient a viral vector including a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), (b) determining that the patient exhibits cholestasis, hyperbilirubinemia, or one or more symptoms thereof, and (c) administering to the patient an anti-cholestatic agent.

In another aspect, the disclosure provides a method of reducing stiffness and/or joint contractures in a human patient diagnosed as having XLMTM, the method comprising: (a) administering to the patient a viral vector comprising a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), (b) determining that the patient exhibits cholestasis, hyperbilirubinemia, or one or more symptoms thereof, and (c) administering to the patient an anti-cholestatic agent.

In another aspect, the disclosure provides a method of increasing diaphragm and/or respiratory muscle progression in a human patient diagnosed as having XLMTM, the method comprising: (a) administering to the patient a viral vector comprising a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), (b) determining that the patient exhibits cholestasis, hyperbilirubinemia, or one or more symptoms thereof, and (c) administering to the patient an anti-cholestatic agent.

In another aspect, the disclosure provides a method of treating XLMTM in a human patient in need thereof that is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), the method including: (a) administering to the patient a therapeutically effective amount of a transgene encoding MTM1, (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of reducing stiffness and/or joint contractures in a human patient diagnosed as having XLMTM, the method comprising: (a) administering to the patient a therapeutically effective amount of a viral vector comprising a transgene encoding MTM1, (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of increasing diaphragm and/or respiratory muscle progression in a human patient diagnosed as having XLMTM, the method comprising: (a) administering to the patient a therapeutically effective amount of a viral vector comprising a transgene encoding MTM1, (b) monitoring the patient for development of cholestasis, hyperbilirubinemia, or one or more symptoms thereof, and, if the patient exhibits cholestasis, hyperbilirubinemia, or one or more symptoms thereof, (c) administering to the patient an anti-cholestatic agent.

In another aspect, the disclosure provides a method of treating XLMTM in a human patient in need thereof that is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), the method including: (a) administering to the patient a therapeutically effective amount of a transgene encoding MTM1, (b) determining that the patient exhibits cholestasis, hyperbilirubinemia, or one or more symptoms thereof, and (c) administering to the patient an anti-cholestatic agent.

In another aspect, the disclosure provides a method of treating XLMTM in a human patient in need thereof that is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), the method including: (a) administering to the patient a therapeutically effective amount of a transgene encoding MTM1, (b) determining that the patient exhibits cholestasis, hyperbilirubinemia, or one or more symptoms thereof, and (c) administering to the patient an anti-cholestatic agent.

In another aspect, the disclosure provides a method of treating or preventing cholestasis or hyperbilirubinemia in a human patient that has XLMTM and who has been previously administered a transgene encoding MTM1, the method including administering to the patient an anti-cholestatic agent.

In another aspect, the disclosure provides a method of treating or preventing cholestasis or hyperbilirubinemia in a human patient that has XLMTM and who has been previously administered a viral vector including a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less), the method including administering to the patient an anti-cholestatic agent.

In another aspect, the disclosure provides a method of treating or preventing cholestasis or hyperbilirubinemia in a human patient that has XLMTM, has been previously administered a transgene encoding MTM1, and that was five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene, the method including administering to the patient an anti-cholestatic agent.

In some embodiments of any of the foregoing aspects, the transgene encoding MTM1 was administered to the patient by transduction with a viral vector containing a transgene encoding MTM1.

In some embodiments of any of the foregoing aspects, the patient is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

In some embodiments of any of the foregoing aspects, the patient is four years old or younger (e.g., 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector, optionally wherein the patient is three years old or younger (e.g., 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), two years old or younger (e.g., 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), one year old or younger (e.g., 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), or six months old or younger (e.g., 6 months old or younger, 5 months old or younger, 4 months old or younger, 3 months old or younger, 2 months old or younger, or 1 month old or younger).

In some embodiments of any of the foregoing aspects, the patient was from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

In some embodiments of any of the foregoing aspects, the viral vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

In some embodiments of any of the foregoing aspects, the viral vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), optionally wherein the viral vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), less than about 1.5×10¹⁴ vg/kg (e.g., less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), or less than about 1.4×10¹⁴ vg/kg (e.g., less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

In some embodiments of any of the foregoing aspects, the viral vector is administered to the patient in an amount of from about 3×10¹³ vg/kg to about 2.3×10¹⁴ vg/kg, optionally wherein the viral vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg. For example, the viral vector may be administered to the patient in an amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments of any of the foregoing aspects, the viral vector is administered to the patient in an amount of about 1.3×10¹⁴ vg/kg.

In some embodiments of any of the foregoing aspects, the transgene or viral vector is administered to the patient in a single dose including the amount.

In some embodiments of any of the foregoing aspects, the transgene or viral vector is administered to the patient in two or more (e.g., two or more, three or more, four or more, five or more, six or more, seven or more, eight or more, nine or more, or ten or more) doses that, together, comprise the amount.

In some embodiments of any of the foregoing aspects, the transgene or viral vector is administered to the patient in two or more (e.g., two or more, three or more, four or more, five or more, six or more, seven or more, eight or more, nine or more, or ten or more) doses that each, individually, comprise the amount.

In some embodiments of any of the foregoing aspects, the two or more (e.g., two or more, three or more, four or more, five or more, six or more, seven or more, eight or more, nine or more, or ten or more) doses are separated from one another by one year or more (e.g., one year or more, one year and six months or more, two years or more, three years or more, four years or more, or five years or more).

In some embodiments of any of the foregoing aspects, the two or more doses are administered to the patient within about 12 months (e.g., about 12 months, about 11 months, about 10 months, about 9 months, about 8 months, about 7 months, about 6 months, about 5 months, about 4 months, about 3 months, about 2 months, or about 1 month) of one another.

In some embodiments of any of the foregoing aspects, the viral vector is selected from the group consisting of adeno-associated virus (AAV), adenovirus, lentivirus, retrovirus, poxvirus, baculovirus, herpes simplex virus, vaccinia virus, and a synthetic virus.

In some embodiments, the viral vector is an AAV. In some embodiments, the AAV is an AAV1, AAV2, AAV3, AAV4, AAV5, AAV6, AAV7, AAV8, AAV9, AAVrh10, or AAVrh74 serotype.

In some embodiments, the viral vector is a pseudotyped AAV. In some embodiments, the pseudotyped AAV is AAV2/8 or AAV2/9, optionally wherein the pseudotyped AAV is AAV2/8.

In some embodiments, the transgene encoding MTM1 is operably linked to a muscle specific promoter. In some embodiments, the muscle specific promotor is a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3.

In some embodiments, the muscle specific promoter is a desmin promoter.

In some embodiments of any of the foregoing aspects, the viral vector is resamirigene bilparvovec.

In some embodiments of any of the foregoing aspects, the viral vector is administered to the patient by way of intravenous, intramuscular, intradermal, or subcutaneous administration.

In some embodiments of any of the foregoing aspects, the anti-cholestatic agent is selected from the group consisting of a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor.

In some embodiments, (i) the FXR ligand is obeticholic acid, cilofexor, tropifexor, tretinoin, or EDP-305; (ii) the FGF-19 mimetic is aldafermin; (iii) the TGR5 agonist is INT-777 or INT-767; (iv) the PPAR agonist is bezafibrate, seladelpar, or elafibrinor; (v) the PPAR-alpha agonist is fenofibrate; (vi) the PPAR-delta agonist is seladelpar; (vii) the dual PPAR-alpha and PPAR-delta agonist is elafibranor; (viii) the ASBT inhibitor is odevixibat, maralixibat, or linerixibat; (ix) the immunomodulatory drug is rituximab, abatacept, ustekinumab, infliximab, baricitinib, or FFP-104; (x) the antifibrotic therapy is a vitamin D receptor agonist or simtuzumab; and/or (xi) the NOX inhibitor is setanaxib.

In some embodiments, the bile acid is ursodeoxycholic acid (e.g., ursodiol), nor-ursodeoxycholic acid, or a pharmaceutically acceptable salt thereof. In some embodiments, the bile acid is ursodiol.

In some embodiments of any of the foregoing aspects, the bile acid is administered to the patient in a single dose. In some embodiments, the bile acid is administered to the patient in a plurality of doses.

In some embodiments, the bile acid is administered to the patient in an amount of from about 5 mg/kg/dose to about 20 mg/kg/dose, optionally wherein the bile acid is administered to the patient in an amount of from about 6 mg/kg/dose to about 19 mg/kg/dose, from about 7 mg/kg/dose to about 18 mg/kg/dose, from about 8 mg/kg/dose to about 17 mg/kg/dose, from about 10 mg/kg/dose to about 15 mg/kg/dose, or from about 12 mg/kg/dose to about 13 mg/kg/dose. For example, the bile acid is administered to the patient in an amount of about 5 mg/kg/dose, 6 mg/kg/dose, 7 mg/kg/dose, 8 mg/kg/dose, 9 mg/kg/dose, 10 mg/kg/dose, 11 mg/kg/dose, 12 mg/kg/dose, 13 mg/kg/dose, 14 mg/kg/dose, 15 mg/kg/dose, 16 mg/kg/dose, 17 mg/kg/dose, 18 mg/kg/dose, 19 mg/kg/dose, or 20 mg/kg/dose.

In some embodiments, the bile acid is administered to the patient in an amount of from about 5 mg/kg/dose to about 11 mg/kg/dose, optionally wherein the bile acid is administered to the patient in an amount of from about 6 mg/kg/dose to about 10 mg/kg/dose, or from about 7 mg/kg/dose to about 9 mg/kg/dose. For example, the bile acid is administered to the patient in an amount of about 5 mg/kg/dose, 6 mg/kg/dose, 7 mg/kg/dose, 8 mg/kg/dose, 9 mg/kg/dose, 10 mg/kg/dose, or 11 mg/kg/dose.

In some embodiments, the bile acid is administered to the patient in one or more (e.g., one or more, two or more, three or more, four or more, five or more, six or more, seven or more, eight or more, nine or more, or ten or more) doses per day, week, or month.

In some embodiments, the bile acid is administered to the patient in one or more e.g., one or more, two or more, three or more, four or more, five or more, six or more, seven or more, eight or more, nine or more, or ten or more) doses per day, optionally wherein the bile acid is administered to the patient in one dose per day, in two doses per day, three doses per day, four doses per day, or five doses per day.

In some embodiments, the bile acid is administered to the patient in one dose per day.

In some embodiments, the bile acid is administered to the patient in an amount of from about 5 mg/kg/day to about 40 mg/kg/day, optionally wherein (i) the bile acid is administered to the patient in an amount of from about 6 mg/kg/day to about 39 mg/kg/day, from about 8 mg/kg/day to about 37 mg/kg/day, from about 13 mg/kg/day to about 32 mg/kg/day, or from about 20 mg/kg/day to about 25 mg/kg/day, or (ii) the bile acid is administered to the patient in an amount of from about 17 mg/kg/day to about 23 mg/kg/day, from about 18 mg/kg/day to about 22 mg/kg/day, or from about 19 mg/kg/day to about 21 mg/kg/day. For example, the bile acid is administered to the patient in an amount of about 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 25 mg/kg/day, 30 mg/kg/day, 35 mg/kg/day, or 40 mg/kg/day. In some embodiments, the bile acid is administered to the patient in an amount of 20 mg/kg/day.

In some embodiments, the bile acid is administered to the patient by way of a unit dosage form including 250 mg of the bile acid. In some embodiments of any of the foregoing aspects, the bile acid is administered to the patient by way of a unit dosage form including 500 mg of the bile acid.

In some embodiments of any of the foregoing aspects, the bile is administered to the patient by way of enteral administration.

In some embodiments of any of the foregoing aspects, the patient does not have a history of cholestasis or hyperbilirubinemia. In some embodiments, the patient does not have a history of any underlying liver disease.

In some embodiments of any of the foregoing aspects, the patient was born at greater than or equal to 35 weeks of gestational age and is or was from term age (e.g., adjusted term age) to about 5 years old (e.g., 1 day old to about 5 years old, 2 days old to about 5 years old, 3 days old to about 5 years old, 4 days old to about 5 years old, 5 days old to about 5 years old, 6 days old to about 5 years old, 7 days old to about 5 years old, 8 days old to about 5 years old, 9 days old to about 5 years old, 10 days old to about 5 years old, 11 days old to about 5 years old, 12 days old to about 5 years old, 13 days old to about 5 years old, 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the transgene or viral vector.

In some embodiments of any of the foregoing aspects, the patient is male.

In some embodiments of any of the foregoing aspects, the patient requires mechanical ventilatory support, optionally wherein mechanical ventilatory support includes invasive mechanical ventilatory support and noninvasive mechanical ventilatory support.

In some embodiments of any of the foregoing aspects, upon administering the transgene or viral vector to the patient, the patient exhibits a change from baseline in hours of mechanical ventilation support over time, optionally wherein the patient exhibits the change from baseline in hours of mechanical ventilation support over time by about 24 weeks after administration of the transgene or viral vector to the patient, optionally wherein the patient displays the change from baseline in hours of mechanical ventilation support over time by about 20 weeks, 16 weeks, 12 weeks, 8 weeks, or 4 weeks after administration of the viral vector to the patient.

In some embodiments of any of the foregoing aspects, upon administering the transgene or viral vector to the patient, the patient achieves functionally independent sitting for at least 30 seconds, optionally wherein the patient achieves the functionally independent sitting by about 24 weeks after administration of the transgene or viral vector to the patient, optionally wherein the patient displays the functionally independent sitting for at least 30 seconds by about 20 weeks, 16 weeks, 12 weeks, 8 weeks, or 4 weeks after administration of the viral vector to the patient.

In some embodiments of any of the foregoing aspects, upon administering the transgene or viral vector to the patient, the patient displays a reduction in required mechanical ventilator support to about 16 hours or less per day, optionally wherein the patient displays the reduction in required mechanical ventilator support by about 24 weeks after administration of the viral vector to the patient, optionally wherein the patient displays the reduction in required mechanical ventilator support by about 20 weeks, 16 weeks, 12 weeks, 8 weeks, or 4 weeks after administration of the viral vector to the patient.

In some embodiments of any of the foregoing aspects, upon administering the transgene or viral vector to the patient, the patient displays a change from baseline on the Children's Hospital of Philadelphia Infant Test of Neuromuscular Disorders (CHOP INTEND), optionally wherein the patient displays the change from baseline on the CHOP INTEND by about 24 weeks after administration of the transgene or viral vector to the patient, optionally wherein the patient displays the change from baseline on the CHOP INTEND by about 20 weeks, 16 weeks, 12 weeks, 8 weeks, or 4 weeks after administration of the viral vector to the patient.

In some embodiments of any of the foregoing aspects, upon administering the transgene or viral vector to the patient, the patient displays a change from baseline in (MIP), optionally wherein the patient displays the change from baseline in MIP by about 24 weeks after administration of the viral vector to the patient, optionally wherein the patient displays the change from baseline in MIP by about 20 weeks, 16 weeks, 12 weeks, 8 weeks, or 4 weeks after administration of the viral vector to the patient.

In some embodiments of any of the foregoing aspects, upon administering the transgene or viral vector to the patient, the patient displays a change from baseline in quantitative analysis of myotubularin expression in a muscle biopsy, optionally wherein the patient displays the change from baseline in quantitative analysis of myotubularin expression in a muscle biopsy by about 24 weeks after administration of the transgene or viral vector to the patient, optionally wherein the patient displays the change from baseline in quantitative analysis of myotubularin expression in a muscle biopsy by about 20 weeks, 16 weeks, 12 weeks, 8 weeks, or 4 weeks after administration of the viral vector to the patient. In some embodiments, the change from baseline in quantitative analysis of myotubularin expression in a muscle biopsy persists for at least 48 weeks (e.g., 49 weeks, 50 weeks, 51 weeks, 52, weeks, 1 year, or 2 years) after administration of the viral vector to the patient.

In some embodiments of any of the foregoing aspects, upon administering the viral vector to the patient, the patient displays a reduction of stiffness and/or joint contractures, optionally wherein the patient displays the reduction of stiffness and/or joint contractures by about 24 weeks after administration of the viral vector to the patient, optionally wherein the patient displays the reduction of stiffness and/or joint contractures by about 20 weeks, 16 weeks, 12 weeks, 8 weeks, or 4 weeks after administration of the viral vector to the patient.

In some embodiments of any of the foregoing aspects, upon administering the viral vector to the patient, the patient displays diaphragm and/or respiratory muscle progression, optionally wherein the patient displays the diaphragm and/or respiratory muscle progression by about 24 weeks after administration of the viral vector to the patient, optionally wherein the patient displays the diaphragm and/or respiratory muscle progression by about 20 weeks, 16 weeks, 12 weeks, 8 weeks, or 4 weeks after administration of the viral vector to the patient.

In some embodiments of any of the foregoing aspects, the patient is determined to exhibit cholestasis or one or more symptoms thereof by a finding that the patient exhibits a serum total bile acids level that is greater than 14 μmol/L (e.g., greater than 14 μmol/L, 15 μmol/L, 16 μmol/L, 17 μmol/L, 18 μmol/L, 19 μmol/L, 20 μmol/L, 21 μmol/L, 22 μmol/L, 23 μmol/L, 24 μmol/L, 25 μmol/L, 26 μmol/L, 27 μmol/L, 28 μmol/L, 29 μmol/L, 30 μmol/L, 31 μmol/L, 32 μmol/L, 33 μmol/L, 34 μmol/L, 35 μmol/L, 36 μmol/L, 37 μmol/L, 38 μmol/L, 39 μmol/L, 40 μmol/L, 41 μmol/L, 42 μmol/L, 43 μmol/L, 44 μmol/L, 45 μmol/L, 46 μmol/L, 47 μmol/L, 48 μmol/L, 49 μmol/L, 50 μmol/L, 51 μmol/L, 52 μmol/L, 53 μmol/L, 54 μmol/L, 55 μmol/L, 56 μmol/L, 57 μmol/L, 58 μmol/L, 59 μmol/L, 60 μmol/L, 61 μmol/L, 62 μmol/L, 63 μmol/L, 64 μmol/L, 65 μmol/L, 66 μmol/L, 67 μmol/L, 68 μmol/L, 69 μmol/L, 70 μmol/L, 71 μmol/L, 72 μmol/L, 73 μmol/L, 74 μmol/L, 75 μmol/L, 76 μmol/L, 77 μmol/L, 78 μmol/L, 79 μmol/L, 80 μmol/L, 81 μmol/L, 82 μmol/L, 83 μmol/L, 84 μmol/L, 85 μmol/L, 86 μmol/L, 87 μmol/L, 88 μmol/L, 89 μmol/L, 90 μmol/L, 91 μmol/L, 92 μmol/L, 93 μmol/L, 94 μmol/L, 95 μmol/L, 96 μmol/L, 97 μmol/L, 98 μmol/L, 99 μmol/L, or 100 μmol/L).

In some embodiments of any of the foregoing aspects, the patient is determined to exhibit cholestasis or one or more symptoms thereof by a finding that the patient exhibits one or more parameters in a blood test that is increased or decreased relative to a reference level.

In some embodiments of any of the foregoing aspects, the blood test is a liver function test.

In some embodiments of any of the foregoing aspects, the one or more parameters includes the level of gamma-glutamyl transferase, alkaline phosphatase, aspartate aminotransferase, and/or alanine aminotransferase.

In some embodiments of any of the foregoing aspects, the patient is determined to exhibit hyperbilirubinemia or one or more symptoms thereof by a finding that the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

In some embodiments of any of the foregoing aspects, upon administering the viral vector to the patient, the patient displays a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test by about 3 weeks after administration of the viral vector to the patient.

In some embodiments of any of the foregoing aspects, the bilirubin level comprises a direct bilirubin level or a total bilirubin level.

In some embodiments of any of the foregoing aspects, the patient is determined to exhibit cholestasis, hyperbilirubinemia, or one or more symptoms thereof by a finding that the patient exhibits a parameter in blood test that is increased relative to a reference level.

In some embodiments of any of the foregoing aspects, the parameter includes the level of a serum bile acid.

In some embodiments of any of the foregoing aspects, the serum bile acid is cholic acid, chenodeoxycholic acid, deoxycholic acid, or ursodeoxycholic acid.

In some embodiments of any of the foregoing aspects, the blood test is a liver function test.

In some embodiments of any of the foregoing aspects, the parameter includes the level of aspartate aminotransferase or alanine aminotransferase.

In one aspect, the disclosure provides a kit including a transgene encoding MTM1 and a package insert, wherein the package insert instructs a user of the kit to administer the transgene to a patient having XLMTM in accordance with the method of any one of the foregoing aspects.

In one aspect, the disclosure provides a kit including a viral vector including a transgene encoding MTM1 and a package insert, wherein the package insert instructs a user of the kit to administer the viral vector to a patient having XLMTM in accordance with the method of any one of the foregoing aspects.

In one aspect, the disclosure provides a kit including an anti-cholestatic agent and a package insert, wherein the package insert instructs a user of the kit to administer the anti-cholestatic agent to a patient to treat or prevent cholestasis or hyperbilirubinemia in accordance with the method of any one of the foregoing aspects.

### BRIEF DESCRIPTION OF THE DRAWINGS

FIG. 1. is a schematic drawing of an exemplary pseudotyped adeno-associated virus (AAV) 2/8 (AAV2/8) viral vector for the expression of the human myotubularin 1 (hMTM1) gene (e.g., resamirigene bilparvovec). From left to right, the shaded arrows and rectangles represents the nucleic acid sequences encoding a human desmin (hDes) promotor (SEQ ID NO: 3) operatively linked to a Beta-globin Intron, a hMTM1 gene (SEQ ID NO: 4), a Beta-globin poly-adenylation signal (Beta-globin_pA), and flanking AAV2 inverted terminal repeat sequences (ITR). Abbreviations: AAV2_ITR, adeno associated virus 2 inverted terminal repeat; Beta-globin_pA, human Beta-globin polyadenylation signal; hDes, human desmin promotor; hMTM1, human myotubularin complementary DNA.

FIG. 2 is a graph showing changes in total and/or direct bilirubin (fold over the upper limit of normal (ULN)) in human patients treated with 3.0×10¹⁴ vg/kg (3e14; light gray) or 1.0×10¹⁴ vg/kg (1e14; dark gray) of resamirigene bilparvovec, as described in Example 2, below. Solid rectangles define the 25th to 75th percentiles; crosses define the mean; lower whiskers define the minimum observations above the lower fence (excluding outliers); upper whiskers define the maximum observation below the upper fence (excluding outliers); circles denote outliers, defined as any value above the upper or below the lower fence [greater than 75th percentile+1.5*IQR; or less than 25th percentile−1.5*] where IQR is interquartile range (75th-25th percentiles).

FIG. 3 is a regression plot showing the change from baseline in total bilirubin (mg/dL) in human patients treated with 3.0×10¹⁴ vg/kg (light gray solid line) or 1.0×10¹⁴ vg/kg (dark gray solid line) of resamirigene bilparvovec, as described in Example 2, below. The regression curve for each group is fitted to all individual subjects dosed at the respective dose (3.0×10¹⁴ vg/kg or 1.0×10¹⁴ vg/kg of resamirigene bilparvovec; light gray dashed lines and dark gray dashed lines, respectively).

FIGS. 4A-4C are graphs showing respiratory and motor function outcomes after therapy with resamirigene bilparvovec for individual patients. Shown is the time course of ventilator dependence over 24 hours among treated ASPIRO patients on invasive (FIG. 4A), MIP (FIG. 4B), and CHOP INTEND scores (FIG. 4C) for treated ASPIRO patients with a locally estimated scatterplot smoothing regression curve fitted to lower-dose patients, higher-dose patients, and the control group, respectively.

FIGS. 5A-B are heatmaps showing the T-cell and B-cell responses to administration of MTM1 after resamirigene bilparvovec from peripheral blood mononuclear cells (PBMC)/serum samples. FIG. 5A shows an ELISpot assay measuring interferon-γ (IFN-γ) release in response to stimulation of participants PBMCs with MTM1 peptide pool over time in samples from treated participants by mutation type. Shown are IFN-γ cytokine secretion data measured by T cell ELISpot assay. Results are expressed as the number of SFC (spot forming cells) per 10⁶ PBMCs. A result was deemed significant if the mean SFC/10⁶ cells minus two standard errors is greater than 2× the respective negative control wells, and where p 50.05. Additionally, a 50 SFC/10⁶ cut-off was used in determining positive response to AAV8 or MTM1 peptide pool stimulation. Common causes for a result ‘not determined’ include the insufficiency of PBMCs available and failure of samples to respond to stimulation with positive controls. Although a definitive conclusion cannot be made based on the ELISpot data, a number of clinical and histopathological observations do not support a T-cell-mediated immune response as a causative factor in the occurrence of the hepatobiliary severe adverse effects (SAEs). Among the participants that had negative ELISpot results at baseline, participants 12 and 01 later reported cholestatic SAEs. Participant 23 eventually reported SAEs of thrombocytopenia and myocarditis. The baseline sample for participant 09 was positive; over the course of the study he developed severe cholestatic liver dysfunction and fatal sepsis, immune system disorder, and liver disorder. Subjects with only negative (or negative and not determined) results upon dosing included 25, 12 (who eventually developed severe cholestatic liver dysfunction and fatal gastrointestinal bleed), and 06 (who eventually developed severe cholestatic liver dysfunction and fatal sepsis). Patients 11, 33 and 38 did not have ELISpot data. FIG. 5B shows the anti-MTM1 antibody titer overtime in samples from treated participants by mutation type. Shown are anti-MTM1 antibody titer data measured before and after resamirigene bilparvovec dosing of ASPIRO subjects. ‘Not detected’ results are also shown, while presence of antibody titers are shown by heat gradient. MTM1 gene mutations type present in ASPIRO subjects are shown as loss of function (LOF), partial loss of function (PLOF), and inframe exonic deletions (IFED).

FIG. 6 is a diagram of the experimental design for a patient enrollment and dosing trial in the clinical trial ASPIRO.

FIGS. 7A-7D are graphs showing the respiratory and ventilation outcomes after resamirigene bilparvovec. FIGS. 7A and 7B are a graph and quantification, respectively, showing the percent changes from baseline for treated participants as compared with pooled control participants in least-squares mean ventilator hours per 24 hours, while FIGS. 7C and 7D are a graph and quantification, respectively, showing maximal inspiratory pressure (MIP). Participants from the higher-dose cohort (3.0×10¹⁴ vg/kg resamirigene bilparvovec) were weaned from ventilation more gradually using a more conservative algorithm with more frequent timepoints compared with the lower-dose cohort (1.0×10¹⁴ vg/kg resamirigene bilparvovec). Error bars indicate standard errors. F-tests and associated error bar values are from a mixed effect ANOVA model, indicating a highly significant percent reduction in change from baseline in hours of ventilation per day in treated vs. the control arm over time. Ventilator dependence, data were collected by e-diary (i.e., very frequent reporting) for controls and higher dose participants and collected at discreet site visits for lower-dose participants.

FIGS. 8A and 8B are a graph and quantification, respectively showing motor function after resamirigene bilparvovec. Shown are the changes from baseline in least-squares mean motor function score on the Children's Hospital of Philadelphia Infant Test of Neuromuscular Disorders (CHOP INTEND) scale among treated participants as compared with the pooled control participants. Scores on the CHOP INTEND scale range from 0 to 64, with higher scores indicating better function; a 4-point increment is deemed to be clinically significant. Error bars indicate standard errors.

FIGS. 9A and 9B are a graph and quantification, respectively, showing the achievement of major motor milestones in individual resamirigene bilparvovec-treated and control patients. White boxes start at the age at dosing in the Gene Transfer Clinical Study in X-Linked Myotubular Myopathy (ASPIRO) or age of enrollment (INCEPTUS). The length of the box indicates the patient's time on study. Icons indicate age at motor milestone achievement.

FIGS. 10A-B are a graph and set of photomicrographs, respectively, showing myotubularin (MTM1) protein expression and histopathological changes after resamirigene bilparvovec treatment. FIG. 10A is a graph showing the quantification of an immunoblot of MTM1 protein expression after administration of 1×10¹⁴ vg/kg or 3×10¹⁴ vg/kg resamirigene bilparvovec, respectively, in muscle biopsy samples from individual patients. FIG. 10B is a set of images showing hematoxylin and eosin (H&E) and nicotinamide adenine dinucleotide (NADH) staining in a muscle biopsy sample taken from a patient administered 1×10¹⁴ vg/kg (patient 08) or 3×10¹⁴ vg/kg (patient 25) resamirigene bilparvovec, or an age-matched control respectively; at baseline, week 24, and week 48, respectively.

FIG. 11 is a heatmap showing the inflammatory response (e.g., none, very mild, mold, mild to moderate, moderate, moderate to severe, or severe) in patients receiving resamirigene bilparvovec, as assessed by the expression level of CD3 in patients administered a lower (1×10¹⁴ vg/kg) or a higher dose (3×10¹⁴ vg/kg) of resamirigene bilparvovec.

FIG. 12 is a set of images showing the inflammatory response in patients receiving resamirigene bilparvovec, as assessed with CD3 expression in patients administered 1×10¹⁴ vg/kg (patients 17 and 8), at baseline, week 24, and week 48, respectively.

FIG. 13 is a graph showing the Kaplan-Meier analysis of overall survival with the time-to-event analysis based upon the event being death since study enrollment. As of the date cut for the analysis, subjects without event were not included.

FIGS. 14 and 15 are the histopathology of liver biopsies taken from participant 12 day 85 post dose (FIG. 14) and participant 06 during autopsy (FIG. 15). Hemotoxylin and eosin (H&E) stains are shown alongside staining for BSEP, a bile transport protein.

FIG. 14 shows hepatocyte degeneration and giant cell formation, intracellular and extracellular bile collections, bile ductular proliferation, and minimal inflammation.

FIG. 15 shows hepatocyte degeneration, necrosis, and giant cell formation, intracellular and extracellular bile collections, bile ductular proliferation, severe fibrosis, and no significant inflammation.

### DEFINITIONS

As used herein, the term “about” refers to a value that is within 10% above or below the value being described. For example, “100 pounds” as used in the context of weight described herein includes quantities that are within 10% above or below 100 lbs. Additionally, when used in the context of a list of numerical quantities, it is to be understood that the term “about,” when preceding a list of numerical quantities, applies to each individual quantity recited in the list.

As used herein, the terms “administering,” “administration,” and the like refer to directly giving a patient a therapeutic agent (e.g., a pharmaceutical composition including a viral vector including a nucleic acid sequence encoding an Myotubularin 1 (MTM1) gene operably linked to a muscle specific promoter) by any effective route. Exemplary routes of administration are described herein and include systemic administration routes, such as intravenous injection, as well as routes of administration directly to the central nervous system of the patient, such as by way of intrathecal injection or intracerebroventricular injection, among others.

As used herein, the term “age-adjusted norms” refers to the process of a normalization of data by age, which is a technique that is used to allow populations of subjects to be compared when the age profiles of the populations are different. As used herein, the term “norm” refers to data that does not undergo a normalization by age, as populations of subjects across age profiles are similar.

As used herein, the terms “alanine aminotransferase” and “ALT” refer to a protein whose amino acid sequence comprises or consists of an amino acid sequence of a naturally occurring wild-type ALT protein (e.g., ALT1 and ALT2) as well as proteins whose amino acid sequence comprises or consists of an amino acid sequence of a naturally occurring allelic variants of ALT (GPT or GPT2 e.g., splice variants or allelic variants). Human GPT nucleic acid sequence is provided in NCBI RefSeq Acc. No. NM_005309.2 (SEQ ID NO: 6), and an exemplary wild-type ALT1 amino acid sequence is provided in NCBI RefSeq Acc. No. NP_005300.1 (SEQ ID NO: 7). Human GPT2 nucleic acid sequence is provided in NCBI RefSeq Acc. No. NM_001142466.2 (SEQ ID NO: 8), and an exemplary wild-type ALT2 amino acid sequence is provided in NCBI RefSeq Acc. No. NP_001135938.1 (SEQ ID NO: 9).

As used herein, the terms “alkaline phosphatase” and “ASP” refer to a protein whose amino acid sequence comprises or consists of an amino acid sequence of a naturally occurring wild-type ASP protein as well as proteins whose amino acid sequence comprises or consists of an amino acid sequence of a naturally occurring allelic variants of ASP (e.g., splice variants or allelic variants). Human ASP nucleic acid sequence is provided in NCBI RefSeq Acc. No. NM_000478.5 (SEQ ID NO: 10), and an exemplary wild-type ASP amino acid sequence is provided in NCBI RefSeq Acc. No. NP_000469.3 (SEQ ID NO: 11).

As used herein, the term “anti-cholestatic agent” refers to a substance, such as a small molecule that acts to increase bile formation and/or antagonize the effect of hydrophobic bile acids on biological membranes. The term “antagonize,” as used herein with regard to a protein, refers to a molecule that decreases signal transduction resulting from the interaction of the protein with one or more of its binding partners. The antagonist may result in a decrease in the binding of the protein to one or more of its binding partners relative to binding of the two proteins in the absence of the antagonist.

As used herein, the terms “aspartate aminotransferase” and “AST” refer to a protein whose amino acid sequence comprises or consists of an amino acid sequence of a naturally occurring wild-type AST protein as well as proteins whose amino acid sequence comprises or consists of an amino acid sequence of a naturally occurring allelic variants of AST (e.g., splice variants or allelic variants). Human AST nucleic acid sequence is provided in NCBI RefSeq Acc. No NM_002079.2 (SEQ ID NO: 12), and an exemplary wild-type ASP amino acid sequence is provided in NCBI RefSeq Acc. No. NP_002070.1 (SEQ ID NO: 13).

As used herein, the terms “bile acid test” and “serum bile acid test” refer to the procedure in which a pre-prandial (i.e., before eating) blood sample is collected for a baseline, followed by a meal and followed about two hours later by the collection of a postprandial (i.e., after eating) blood sample. Both blood samples are tested for bile acid levels and the pre-prandial sample is used as a reference. As used herein, “bile acid” refers to the steroid acids found predominantly in the bile of mammals and other vertebrates.

As used herein, the terms “Children's Hospital of Philadelphia Infant Test of Neuromuscular Disorders” and “CHOP INTEND” refer to a validated motor outcome measure developed for the evaluation of weak infants, such as those with a disease of skeletal muscle (e.g., X-linked myotubular myopathy (XLMTM)). CHOP INTEND uses a 0-64-point scale where higher scores indicate better motor function. As used herein, the term “motor function score” refers to a score on the 0-64-point scale of the CHOP INTEND (e.g., a scale of >45 on the CHOP INTEND).

As used herein, the term “cholestasis” refers to a condition where bile cannot flow from the liver to the duodenum. The two clinical distinctions are the “obstructive” type of cholestasis where there is a mechanical blockage in the duct system that can occur from a gallstone or malignancy, and “metabolic” types of cholestasis which are disturbances in bile formation that can occur because of genetic defects or acquired as a side effect of many medications. As used herein, the term “bile” refers to the digestive fluid that is secreted by the liver to aid in the digestion of fats.

As used herein, a “combination therapy” means that two (or more) different agents or treatments are administered to a subject as part of a defined treatment regimen for a particular disease or condition (e.g., a neuromuscular disorder). In some embodiments, a “combination therapy” may include a procedure. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues.

Therapeutic agents can be administered by the same route or by different routes. For example, a first therapeutic agent of the combination may be administered by intravenous injection while a second therapeutic agent of the combination may be administered enterally. In another example, an agent of the therapeutic combination may be administered by intravenous injection and a procedure (e.g., nasobiliary drainage (NBD)) of the therapeutic combination may be performed.

As used herein, the term “dose” refers to the quantity of a therapeutic agent, such as a viral vector described herein, that is administered to a subject at a particular instant for the treatment of a disorder, such as to treat or ameliorate one or more symptoms of a neuromuscular disorder described herein (e.g., XLMTM). A therapeutic agent as described herein may be administered in a single dose or in multiple doses over the course of a treatment period, as defined herein. In each case, therapeutic agent may be administered using one or more unit dosage forms of therapeutic agent, a term that refers to a one or more discrete compositions containing a therapeutic agent that collectively constitute a single dose of the agent.

As used herein, the terms “effective amount,” “therapeutically effective amount,” and the like, when used in reference to a therapeutic composition, such as a vector construct described herein, refer to a quantity sufficient to, when administered to the subject, including a mammal, for example a human, effect beneficial or desired results, such as clinical results. For example, in the context of treating neuromuscular disorders, such as XLMTM, these terms refer to an amount of the composition sufficient to achieve a treatment response as compared to the response obtained without administration of the composition of interest. An “effective amount,” “therapeutically effective amount,” and the like, of a composition, such as a vector construct of the present disclosure, also include an amount that results in a beneficial or desired result in a subject as compared to a control.

As used herein, the terms “gamma-glutamyl transferase” and “GGT” refers to a protein whose amino acid sequence comprises or consists of an amino acid sequence of a naturally occurring wild-type GGT protein as well as proteins whose amino acid sequence comprises or consists of an amino acid sequence of a naturally occurring allelic variants of GGT (GGT1, GGT2, and GGT3 e.g., splice variants or allelic variants). Human GGT1 nucleic acid sequence is provided in NCBI RefSeq Acc. No NM_001288833.1 (SEQ ID NO: 14), and an exemplary wild-type GGT1 amino acid sequence is provided in NCBI RefSeq Acc. No. NP_001275762.1 (SEQ ID NO: 15).

As used herein, the term “gestational age” describes how far along a particular pregnancy is and is measured from the first day of a pregnant female subject's last menstrual cycle to the current date. As used herein, the term “labor” (which may also be termed birth) relates to the expulsion of the fetus and placenta from the uterus of a pregnant female subject. For a normal pregnancy, labor may occur at a gestational age of about 40 weeks.

As used herein, the term “hyperbilirubinemia” refers to a condition in which there is a higher-than-normal level of bilirubin in the blood. As used herein, the term “bilirubin” refers to a compound that occurs in the normal catabolic pathway that breaks down heme in vertebrates. This catabolism is a necessary process in the body's clearance of waste products that arise from the destruction of aged or abnormal red blood cells. As used herein, a “bilirubin test” refers to a measurement of the amount of bilirubin in a patient's blood.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein in a sample.

As used herein, the terms “liver function test” and “LFT” refers to a hepatic panel (e.g., a group of blood tests that provide information about the state of a patient's liver). A hepatic panel may include measurement of the level of gamma-glutamyl transferase, the level of alkaline phosphatase, the level of aspartate aminotransferase, the level of alanine aminotransferase, the level of albumin, the level of bilirubin, the prothrombin time, the activated partial thromboplastin time, or a combination thereof.

As used herein, the terms “maximal inspiratory pressure” and “MIP” refer to a variable in mechanical ventilation including the total airway pressure delivered, generally used to overcome both respiratory system compliance as well as airway resistance. In as pressure-controlled mode, the MIP includes the sum of the positive-end expiratory pressure and the “delta pressure.” As used herein, the term “delta pressure” refers to a variable in mechanical ventilation including the difference between the MIP and the positive-end expiratory pressure.

As used herein, the term “mechanical ventilatory support” refers to the medical term for artificial ventilation where mechanical means are used to assist or replace spontaneous breathing. As used herein, the term “invasive mechanical ventilatory support” refers to the medical term for artificial ventilation where air is delivered via a tube that is inserted into a patient's windpipe through the mouth or nose and mechanical means are used to assist or replace spontaneous breathing. As used herein, the term “noninvasive mechanical ventillatory support” refers to mechanical ventilatory support in which air is delivered to a patient through a sealed mask that can be placed over the mouth, nose, or the whole face.

As used herein, the term “operably linked” refers to a first molecule joined to a second molecule, wherein the molecules are so arranged that the first molecule affects the function of the second molecule.

The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell.

Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, a therapeutic agent is considered to be “provided” to a patient if the patient is directly administered therapeutic agent or if the patient is administered a substance that is processed or metabolized in vivo so as to yield therapeutic agent endogenously. For example, a patient, such as a patient having a neuromuscular disorder described herein, may be provided a nucleic acid molecule encoding a therapeutic protein (e.g., MTM1) by direct administration of the nucleic acid molecule or by administration of a substance (e.g., viral vector or cell) that is processed in vivo so as to yield the desired nucleic acid molecule.

As used herein, the terms “patient” and “subject” refer to an organism that receives treatment for a particular disease or condition as described herein (such as a neuromuscular disorder, e.g., XLMTM).

Examples of subjects and patients include mammals, such as humans, receiving treatment for a disease or condition described herein.

By a “reference” is meant any useful reference used to compare protein levels related to cholestatsis, hyperbilirubinemia, or one or more symptoms thereof. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having cholestatsis, hyperbilirubinemia, or one or more symptoms thereof; a sample from a subject that is diagnosed with cholestatsis, hyperbilirubinemia, or one or more symptoms thereof; a sample from a subject that has been treated for cholestatsis, hyperbilirubinemia, or one or more symptoms thereof; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having cholestatsis, hyperbilirubinemia, or one or more symptoms thereof. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “term age” refers to the age of a patient (e.g., a newborn) born between 37 weeks of gestational age and 42 weeks of gestational age. For example, if the patient was born at 35 weeks of gestational age, the patient is at term age at 14 days old.

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “treat” and “treatment” refer to therapeutic treatment, in which the object is to prevent or slow down (lessen) an undesired physiological change or disorder, such as the progression of a neuromuscular disorder, such as XLMTM, among others. Beneficial or desired clinical results include, but are not limited to, alleviation of symptoms (e.g., stiffness and/or joint contractures), diminishment of extent of disease, stabilized (i.e., not worsening) state of disease, delay or slowing of disease progression, amelioration or palliation of the disease state, and remission (whether partial or total), whether detectable or undetectable. In the context of neuromuscular disorders, such as XLMTM, treatment of a patient may manifest in one or more detectable changes, such as an increase in the concentration of MTM1 protein or nucleic acids (e.g., DNA or RNA, such as mRNA) encoding MTM1, or an increase in MTM1 activity (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or more. The concentration of MTM1 protein may be determined using protein detection assays known in the art, including ELISA assays described herein.

The concentration of MTM1-encoding nucleic acids may be determined using nucleic acid detection assays (e.g., RNA Seq assays) described herein. Additionally, treatment of a patient suffering from a neuromuscular disorder, such as XLMTM, may manifest in improvements in a patient's muscle function (e.g., skeletal muscle function) as well as improvements in muscle coordination. For example, manifestation of an improvement may include increasing diaphragm and/or respiratory muscle progression.

As used herein, the terms “X-linked myotubular myopathy” and “XLMTM” refer to the genetically inherited neuromuscular disorder that is caused by mutations of the MTM1 gene and is characterized by symptoms including mild to profound muscle weakness, hypotonia (diminished muscle tone), feeding difficulties, and/or severe breathing complications. Human MTM1 has NCBI Gene ID NO 4534. An exemplary wild-type human MTM1 nucleic acid sequence is provided in NCBI RefSeq Acc. No. NM_000252.3 (SEQ ID NO: 1), and an exemplary wild-type myotubularin 1 amino acid sequence is provided in NCBI RefSeq Acc. No. NP_000243.1 (SEQ ID NO: 2).

As used herein, the term “vector” refers to a nucleic acid, e.g., DNA or RNA, that may function as a vehicle for the delivery of a gene of interest into a cell (e.g., a mammalian cell, such as a human cell), such as for purposes of replication and/or expression. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Chemical Terms

The chemical terminology used herein is for the purpose of describing various aspects and embodiments of the disclosure and is not intended to be limiting.

In the following chemical definitions, a notation in which an integral number immediately follows an atomic symbol indicates the quantity of atoms of that element that are present in a particular chemical moiety. As will be understood, other atoms, such as hydrogen atoms, or substituent groups described herein, may be present, as necessary, to satisfy the valence of a particular atom. For example, an unsubstituted “C₂ alkyl group” has the formula —CH₂CH₃. When used in conjunction with the groups defined herein, a reference to a number of carbon atoms includes the divalent carbon in acetal and ketal groups but does not include the carbonyl carbon in acyl, ester, carbonate, amide, or carbamate groups.

A reference to a number of oxygen, nitrogen, or sulfur atoms in a heteroaryl group only includes those atoms that form a part of a heterocyclic ring.

As used herein, a phrase of the form “optionally substituted X” (e.g., optionally substituted alkyl) is intended to be equivalent to “X, wherein X is optionally substituted” (e.g., “alkyl, wherein the alkyl is optionally substituted”). It is not intended to mean that the feature “X” (e.g., alkyl) per se is optional. As described herein, certain compounds may contain one or more “optionally substituted” moieties. In general, the term “substituted”, whether preceded by the term “optionally” or not, means that one or more hydrogens of the designated moiety are replaced with a suitable substituent, such as any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents that may be used in conjunction with the compounds of the disclosure are preferably those that result in the formation of stable or chemically feasible compounds. The term “stable,” as used herein, refers to compounds that are not substantially altered when subjected to conditions that allow for their production, detection, and, in certain embodiments, recovery, purification, and use for one or more of the purposes disclosed herein.

As used herein, the term “aliphatic” refers to a saturated or unsaturated, straight, branched, or cyclic hydrocarbon. The term “aliphatic” includes, but is not limited to, alkyl, alkenyl, alkynyl, cycloalkyl, cycloalkenyl, and cycloalkynyl moieties, and thus incorporates each of these definitions. In some embodiments, “aliphatic” is used to indicate those aliphatic groups having from 1 to 20 carbon atoms.

The aliphatic chain may be, for example, mono-unsaturated, di-unsaturated, tri-unsaturated, or polyunsaturated, or alkynyl. Unsaturated aliphatic groups can be in a cis or trans configuration. In some embodiments, the aliphatic group contains from 1 to about 12 carbon atoms, such as from 1 to about 6 carbon atoms or from 1 to about 4 carbon atoms. In some embodiments, the aliphatic group contains from 1 to about 8 carbon atoms. In some embodiments, the aliphatic group is C₁-C₂, C₁-C₃, C₁-C₄, C₁-C₅, or C₁-C₆. The specified ranges used herein indicate an aliphatic group having each member of the range described as an independent species. For example, the term “C₁-C₆ aliphatic” as used herein indicates a straight or branched alkyl, alkenyl, or alkynyl group having from 1, 2, 3, 4, 5, or 6 carbon atoms and is intended to mean that each of these is described as an independent species. For example, the term “C₁-C₄ aliphatic” as used herein indicates a straight or branched alkyl, alkenyl, or alkynyl group having from 1, 2, 3, or 4 carbon atoms and is intended to mean that each of these is described as an independent species. In some embodiments, the aliphatic group is substituted with one or more functional groups that results in the formation of a stable moiety.

As used herein, the term “heteroaliphatic” refers to an aliphatic moiety that contains at least one heteroatom in its chain, such as an amine, carbonyl, carboxy, oxo, thio, phosphate, phosphonate, nitrogen, phosphorus, silicon, or boron atom in place of a carbon atom. In some embodiments, the heteroatom present is nitrogen. In some embodiments, the heteroatom present is oxygen. In some embodiments, the heteroatom present is sulfur. The term “heteroaliphatic” includes, but is not limited to, heteroalkyl, heteroalkenyl, heteroalkynyl, heterocycloalkyl, heterocycloalkenyl, and heterocycloalkynyl moieties. In some embodiments, “heteroaliphatic” is used to indicate a heteroaliphatic group (cyclic, acyclic, substituted, unsubstituted, branched or unbranched) having from 1 to 20 carbon atoms. In some embodiments, the heteroaliphatic group is optionally substituted in a manner that results in the formation of a stable moiety. Nonlimiting examples of heteroaliphatic moieties are polyethylene glycol, polyalkylene glycol, amide, polyamide, glycolide, polylactide, polyglycolide, thioether, ether, alkyl-heterocycle-alkyl, —O-alkyl-O-alkyl, and alkyl-O-haloalkyl.

As used herein, the term “acyl” refers to a carbonyl substituent, such as a carbonyl substituent in which the carbonyl carbon is bound to an alkyl group, an alkenyl group, an alkynyl group, an optionally substituted oxygen moiety, an optionally substituted nitrogen moiety, and the like. Exemplary acyl groups include, without limitation, formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

As used herein, the term “acyloxy” refers to the chemical moiety —OC(O)R in which R is C₁-C₆ alkyl, aryl, heteroaryl, C₁-C₆ alkyl aryl, or C₁-C₆ alkyl heteroaryl.

As used herein, the term “alkyl” refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms). As used herein, the term “alkylene” refers to a divalent alkyl group.

As used herein, the term “alkenyl,” whether recited alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms). As used herein, the term “alkenylene” refers to a divalent alkenyl group.

As used herein, the term “alkynyl,” whether recited alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms). As used herein, the term “alkynylene” refers to a divalent alkynyl group.

As used herein, the term “amino” represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

As used herein, the term “aryl” refers to an aromatic mono- or polycarbocyclic radical of, e.g., 6 to 12, carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

As used herein, the term “arylalkyl” represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

As used herein, the term “bridged cyclyl” refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

As used herein, the term “carbocyclyl” refers to a non-aromatic C₃-C₁₂, monocyclic or polycyclic (e.g., bicyclic or tricyclic) structure in which the rings are formed by carbon atoms. Carbocyclyl structures include cycloalkyl groups (e.g., cyclohexyl) and unsaturated carbocyclyl radicals (e.g., cyclohexenyl).

Polycyclic carbocyclyl includes spirocyclic carbocyclyl, bridged carbocyclyl, and fused carbocyclyl. As used herein, the term “carbocyclylene” refers to a divalent carbocyclyl group.

As used herein, the term “cycloalkyl” refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

As used herein, the terms “halo” and “halogen” mean a fluorine (fluoro), chlorine (chloro), bromine (bromo), or iodine (iodo) radical.

As used herein, the term “heteroalkyl” refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers to alkyl-O— (e.g., methoxy and ethoxy), and an “alkylamino” which, as used herein, refers to —N(alkyl)R^{Na}, where R^{Na} is H or alkyl (e.g., methylamino). As used herein, the term “heteroalkylene” refers to a divalent heteroalkyl group.

As used herein, the term “heteroalkenyl” refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers to alkenyl-O—. As used herein, the term “heteroalkenylene” refers to a divalent heteroalkenyl group.

As used herein, the term “heteroalkynyl” refers to an alkynyl group, as defined herein, in which one or more of the constituent carbon atoms have been replaced by nitrogen, oxygen, or sulfur. In some embodiments, the heteroalkynyl group is further substituted with 1, 2, 3, or 4 substituent groups as described herein for alkynyl groups. Examples of heteroalkynyl groups are an “alkynoxy” which, as used herein, refers to alkynyl-O—. As used herein, the term “heteroalkynylene” refers to a divalent heteroalkynyl group.

As used herein, the term “heteroaryl” refers to an aromatic monocyclic or polycyclic structure of 5 to 12 atoms having at least one aromatic ring containing 1, 2, or 3 ring atoms selected from nitrogen, oxygen, and sulfur, with the remaining ring atoms being carbon. In some embodiments, one or two ring carbon atoms of the heteroaryl group are replaced with a carbonyl group. Examples of heteroaryl groups are pyridyl, pyrazoyl, benzooxazolyl, benzoimidazolyl, benzothiazolyl, imidazolyl, oxaxolyl, and thiazolyl. As used herein, the term “heteroarylene” refers to a divalent heteroaryl group.

As used herein, the term “heteroarylalkyl” represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

As used herein, the term “heterocyclyl” refers a monocyclic or polycyclic radical (e.g., bicyclic or tricyclic) having 3 to 12 atoms having at least one non-aromatic ring containing 1, 2, 3, or 4 ring atoms selected from N, O, or S, and no aromatic ring containing any N, O, or S atoms. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. As used herein, the term “heterocyclylene” refers to a divalent heterocyclyl group.

As used herein, the term “heterocyclylalkyl” represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

As used herein, the term “hydroxyalkyl” refers to an alkyl group substituted with an —OH group.

As used herein, the term “hydroxyl” refers to an —OH group.

As used herein, the term “imine” refers to a ═NR^{N} group, where R^{N} is, e.g., H or alkyl.

As used herein, the term “N-protecting group” refers to those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

As used herein, the term “nitro” refers to an —NO₂ group.

As used herein, the term “oxo” refers to an ═O group.

As used herein, the term “sulfonyl” refers to chemical moiety —SO₂—R in which R is hydrogen, aryl, heteroaryl, C₁-C₆ alkyl, C₁-C₆ alkyl substituted with one or more halogens, such as a —SO₂—CF₃ substituent, C₁-C₆ alkyl aryl, or C₁-C₆ alkyl heteroaryl.

As used herein, the term “sulfonylamino” refers to the chemical moiety —NRSO₂—R′ in which each of R and R′ is independently hydrogen, C₁-C₆ alkyl, aryl, heteroaryl, C₁-C₆ alkyl aryl, or C₁-C₆ alkyl heteroaryl.

As used herein, the term “sulfonyloxy” refers to the chemical moiety —OSO₂—R in which R is hydrogen, C₁-C₆ alkyl, C₁-C₆ alkyl substituted with one or more halogens, such as a —OSO₂—CF₃ substituent, aryl, heteroaryl, C₁-C₆ alkyl aryl, or C₁-C₆ alkyl heteroaryl.

As used herein, the term “thiol” refers to an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups described herein may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, oxo, sulfonyl, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

### Depictions of Chemical Structures

Compounds of the disclosure may have one or more asymmetric carbon atoms and may exist in the form of optically pure enantiomers, mixtures of enantiomers (e.g., racemates), optically pure diastereoisomers, mixtures of diastereoisomers, diastereoisomeric racemates, or mixtures of diastereoisomeric racemates. The optically active forms can be obtained, for example, by resolution of the racemates, by asymmetric synthesis or asymmetric chromatography (chromatography with a chiral adsorbent or eluant). Accordingly, the compounds disclosed herein may exist in various stereoisomeric forms.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. The term “enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms.

Enantiomers of a compound can be prepared, for example, by separating an enantiomer from a racemate using one or more well-known techniques and methods, such as, for example, chiral chromatography and separation methods based thereon. The terms “racemate” and “racemic mixture” refer to a compound containing two enantiomers, wherein such mixtures exhibit no optical activity; i.e., they do not rotate the plane of polarized light. The term “geometric isomer” refers to isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side of the carbon-carbon double bond) configuration. “R,” “S,” “S*,” “R*,” “E,” “Z,” “cis,” and “trans,” indicate configurations relative to the core molecule.

When the stereochemistry of a compound disclosed herein is named or depicted by structure, the named or depicted stereoisomer is greater than 50% by weight (e.g., at least 60%, 70%, 80%, 90%, 99%, or 99.9% by weight) relative to its other stereoisomers. For example, when a single enantiomer is named or depicted by structure, the depicted or named enantiomer is greater than 50% by weight (e.g., at least 60%, 70%, 80%, 90%, 99%, or 99.9% by weight) optically pure. Similarly, when a single diastereomer is named or depicted by structure, the depicted or named diastereomer is greater than 50% by weight (e.g., at least 60%, 70%, 80%, 90%, 99%, or 99.9% by weight) pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers.

Additionally, when the stereochemistry of a compound disclosed herein is named or depicted by structure, the named or depicted stereoisomer is greater than 50% by mole fraction (e.g., at least 60%, 70%, 80%, 90%, 99%, or 99.9% by mole fraction) relative to its other stereoisomers. For example, when a single enantiomer is named or depicted by structure, the depicted or named enantiomer is greater than 50% by mole fraction (e.g., at least 60%, 70%, 80%, 90%, 99%, or 99.9% by mole fraction) relative to the other enantiomer. When a single diastereomer is named or depicted by structure, the depicted or named diastereomer is greater than 50% by mole fraction (e.g., at least 60%, 70%, 80%, 90%, 99%, or 99.9% by mole fraction) relative to the other diastereomer(s) of the indicated compound. For enantiomeric compounds, percent purity by mole fraction is calculated as the ratio of the molar quantity of the enantiomer of interest relative to the sum of the molar quantities of (i) the enantiomer of interest and (ii) the optical isomer. Similarly, for diastereomeric compounds, percent purity by moles fraction is calculated as the ratio of the molar quantity of the diastereomer of interest relative to the total molar quantities of all diastereomers present for the indicated compound.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer.

When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The present disclosure embraces all of these forms.

### Polymorphic Compounds

As will be appreciated by one of skill in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

### DETAILED DESCRIPTION

The present disclosure provides compositions and methods that can be used for treating neuromuscular disorders, particularly X-linked myotubular myopathy (XLMTM). In accordance with the compositions and methods described herein, a patient (e.g., a human patient) having XLMTM may be administered a viral vector, such as an adeno-associated viral (AAV) vector, that contains a transgene encoding Myotubularin 1 (MTM1). The AAV vector may be, for example, a pseudotyped AAV vector, such as an AAV vector containing AAV2 inverted terminal repeats packaged within capsid proteins from AAV8 (AAV2/8). In some embodiments, the transgene is operably linked to a transcription regulatory element, such as a promoter that induces gene expression in a muscle cell. An exemplary promoter that may be used in conjunction with the compositions and methods of the disclosure is a desmin promoter. In some embodiments, the AAV2/8 viral vector comprising a transgene encoding MTM1 is resamirigene bilparvovec.

The present disclosure is based, at least in part, on the discovery of methods of therapeutic and prophylactic treatment that address a significant medical need associated with the existing gene therapy approaches involving the delivery of MTM1 to patients in need thereof (e.g., patients with XLMTM). The present disclosure is also based, in part, on the discovery that the existing gene therapy approaches involving the delivery of MTM1 to patients in need thereof (e.g., patients with XLMTM) are associated with risks, including cholestatic syndromes, such as cholestasis, hyperbilirubinemia, or one or more symptoms thereof. More particularly, the present invention relates to the discovery of a method comprising administration of a viral vector comprising a transgene encoding MTM1 (e.g., resamirigene bilparvovec) and an anti-cholestatic agent (e.g., a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor) as a prophylactic treatment for cholestatic syndromes associated with the existing gene therapy approaches involving the delivery of MTM1 to patients in need thereof (e.g., patients with XLMTM). In some embodiments, the anti-cholestatic agent is a bile acid. In some embodiments, the bile acid is ursodeoxycholic acid (e.g., ursodiol).

In some embodiments, the disclosure describes a method of treating or preventing cholestasis or hyperbilirubinemia in a human patient that has XLMTM and who has been administered a therapeutically effective amount of a viral vector comprising a transgene encoding MTM1 includes administering to the patient an anti-cholestatic agent.

In some embodiments, the disclosure describes a method of treating XLMTM in a human patient in need thereof and who has been previously administered an anti-cholestatic agent, the method comprising administering to the patient a therapeutically effective amount of a viral vector comprising a transgene encoding MTM1.

In some embodiments, the disclosure describes a method of reducing stiffness and/or joint contractures in a human patient diagnosed as having XLMTM, the method comprising administering to the patient a viral vector comprising a transgene encoding MTM1 and (an anti-cholestatic agent.

In some embodiments, the disclosure describes a method of increasing diaphragm and/or respiratory muscle progression in a human patient diagnosed as having XLMTM, the method comprising administering to the patient a viral vector comprising a transgene encoding MTM1 and (an anti-cholestatic agent.

In some embodiments, the disclosure describes a method of reducing stiffness and/or joint contractures in a human patient diagnosed as having XLMTM and who has been previously administered an anti-cholestatic agent, the method comprising administering to the patient a therapeutically effective amount of a viral vector comprising a transgene encoding MTM1.

In some embodiments, the disclosure describes a method of increasing diaphragm and/or respiratory muscle progression in a human patient diagnosed as having XLMTM and who has been previously administered an anti-cholestatic agent, the method comprising administering to the patient a therapeutically effective amount of a viral vector comprising a transgene encoding MTM1.

The sections that follow provide a description of therapeutic agents and parameters for assessing cholestasis, hyperbilirubinemia, or one or more symptoms thereof that result in the administration of an anti-cholestatic agent described herein. The following sections also describe various transduction agents that may be used in conjunction with the compositions and methods of the disclosure.

### Methods of Treatment

In some embodiments, the patient is a newborn (e.g., 0-4 months old), an infant (e.g., 0-5 months old), a toddler (e.g., 6-12 months old), a child aged 1-3 years old, or a child aged 3-5 years old at the time of administration of the viral vector.

In some embodiments, the patient is a newborn (e.g., 0-4 months old) at the time of administration of the viral vector. For example, in some embodiments, the patient is a newborn that is about 0 to about 4 months old (e.g., 0 months old to about 4 months old, 1 month old to about 4 months old, 2 months old to about 4 months old, or 3 months old to about 4 months old). In some embodiments, the patient is 0 months old. In some embodiments, the patient is 1 month old. In some embodiments, the patient is 2 months old. In some embodiments, the patient is 3 months old. In some embodiments, the patient is 4 months old.

In some embodiments, the patient is a newborn (e.g., less than about 4 months old) at the time of administration of the viral vector. For example, in some embodiments, the patient is a newborn that is less than about 4 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is an infant (e.g., 0-5 months old) at the time of administration of the viral vector. For example, in some embodiments, the patient is an infant that is about 0 months old to about 5 months old (e.g., 0 months old to about 5 months old, 1 month old to about 5 months old, 2 months old to about 5 months old, 3 months old to about 5 months old, or 4 months old to about 5 months old). In some embodiments, the patient is 0 months old. In some embodiments, the patient is 1 month old. In some embodiments, the patient is 2 months old. In some embodiments, the patient is 3 months old. In some embodiments, the patient is 4 months old. In some embodiments, the patient is 3 months old. In some embodiments, the patient is 5 months old.

In some embodiments, the patient is an infant (e.g., less than about 5 months old) at the time of administration of the viral vector. For example, in some embodiments, the patient is an infant that is less than about 5 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a toddler (e.g., 6-12 months old) at the time of administration of the viral vector. For example, in some embodiments, the patient is an infant that is about 6 months old to about 12 months old (e.g., 6 months old to about 12 months old, 7 months old to about 12 months old, 8 months old to about 12 months old, 9 months old to about 12 months old, 10 months old to about 12 months old, or 11 months old to about 12 months old). In some embodiments, the patient is 6 months old. In some embodiments, the patient is 7 months old. In some embodiments, the patient is 8 months old. In some embodiments, the patient is 9 months old. In some embodiments, the patient is 10 months old. In some embodiments, the patient is 11 months old. In some embodiments, the patient is 12 months old.

In some embodiments, the patient is a toddler (e.g., less than about 12 months old) at the time of administration of the viral vector. For example, in some embodiments, the patient is a toddler that is less than about 12 months old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a child aged 1-3 years old at the time of administration of the viral vector. For example, in some embodiments, the patient is a child that is about 1 year old to about 3 years old (e.g., 1 year old to about 3 years old or 2 years old to about 3 years old). In some embodiments, the patient is 1 year old. In some embodiments, the patient is 2 years old. In some embodiments, the patient is 3 years old.

In some embodiments, the patient is a child (e.g., less than about 3 years old) at the time of administration of the viral vector. For example, in some embodiments, the patient is a child that is less than about 3 years old. In some embodiments, the patient is less than about 3 years old. In some embodiments, the patient is less than about 2 years old. In some embodiments, the patient is less than about 1 year old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a child aged 3-5 years old at the time of administration of the viral vector. For example, in some embodiments, the patient is a child that is about 3 years old to about 5 years old (e.g., 3 years old to about 5 years old or 4 years old to about 5 years old). In some embodiments, the patient is 3 years old. In some embodiments, the patient is 4 years old. In some embodiments, the patient is 5 years old.

In some embodiments, the patient is a child (e.g., less than about 5 years old) at the time of administration of the viral vector. For example, in some embodiments, the patient is a child that is less than about 5 years old. In some embodiments, the patient is less than about 5 years old. In some embodiments, the patient is less than about 4 years old. In some embodiments, the patient is less than about 3 years old. In some embodiments, the patient is less than about 2 years old. In some embodiments, the patient is less than about 1 year old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the viral vector.

In some embodiments, the patient was born at greater than or equal to 35 weeks of gestational age (e.g., 35 weeks of gestational age, 36 weeks of gestational age, 37 weeks of gestational age, 38 weeks of gestational age, 39 weeks of gestational age, 40 weeks of gestational age, 41 weeks of gestational age, and 42 weeks of gestational age) and is between adjusted term age (e.g., 37 weeks of gestational age or greater) to about 5 years old at the time of administration of the viral vector. For example, if the patient was born at 35 weeks of gestational age, the patient is at term age at 14 days old.

In some embodiments, the patient was born at 35 weeks of gestational age and is between adjusted term age to about 5 years old (e.g., 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the viral vector.

In some embodiments, the patient was born at 36 weeks of gestational age and is between adjusted term age to about 5 years old (e.g., 7 days old to about 5 years old, 8 days old to about 5 years old, 9 days old to about 5 years old, 10 days old to about 5 years old, 11 days old to about 5 years old, 12 days old to about 5 years old, 13 days old to about 5 years old, 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the viral vector.

In some embodiments, the patient was born at 37 weeks of gestational age and is between adjusted term age to about 5 years old (e.g., 1 day old to about 5 years old, 2 days old to about 5 years old, 3 days old to about 5 years old, 4 days old to about 5 years old, 5 days old to about 5 years old, 6 days old to about 5 years old, 7 days old to about 5 years old, 8 days old to about 5 years old, 9 days old to about 5 years old, 10 days old to about 5 years old, 11 days old to about 5 years old, 12 days old to about 5 years old, 13 days old to about 5 years old, 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the viral vector.

In some embodiments, the patient is male.

In some embodiments, the patient is female.

### X-Linked Myotubular Myopathy

XLMTM is a rare, life-threatening, congenital myopathy caused by a loss-of-function mutation in the MTM1 gene and is characterized in most patients by profound muscle weakness and hypotonia at birth, which results in severe respiratory insufficiency, inability to sit up, stand or walk, and early mortality.

The myopathy associated with XLMTM impairs the development of motor skills such as sitting, standing, and walking. Affected infants may also have difficulties with feeding due to muscle weakness.

Individuals with this condition often do not have the muscle strength to breathe on their own and must be supported with mechanical ventilation. Some affected individuals require mechanical ventilation only periodically, such as during sleep, while others require mechanical ventilation continuously. Patients having XLMTM may also have weakness in the muscles that control eye movement (ophthalmoplegia), weakness in other muscles of the face, and absent reflexes (areflexia).

In XLMTM, muscle weakness often disrupts normal bone development and can lead to fragile bones, an abnormal curvature of the spine (scoliosis), and joint deformities (contractures) of the hips and knees. Patients having XLMTM may have a large head with a narrow and elongated face and a high, arched roof of the mouth (palate). Patients may also have liver disease, recurrent ear and respiratory infections, or seizures.

As a consequence of their severe breathing difficulties, patients having XLMTM usually survive only into early childhood; however, some patients with this condition have lived into adulthood. The compositions and methods of the disclosure provide the important medical benefit of being able to prolong the lifetimes of such patients by restoring functional MTM1 expression. Moreover, the compositions and methods described herein can be used to improve patients' quality of life post-treatment (e.g., reducing stiffness and/or joint contractures or increasing diaphragm and/or respiratory muscle progression), as the disclosure provides a series of guidelines that can be used to determine a patient's eligibility for being weaned off of mechanical ventilation.

### Cholestasis and Hyperbilirubinemia

Cholestasis is any condition in which the flow of bile acid from the liver is slowed or blocked, while hyperbilirubinemia is a condition in which there is an accumulation of bilirubin in the blood and serum bile acids appear to remain normal. By contrast, cholestatic syndromes are characterized by marked bile acidemia with normal to slightly elevated bilirubin levels.

In some embodiments, the patient is monitored for the development of cholestasis. In some embodiments, the patient is monitored for the development of hyperbilirubinemia. In some embodiments, the patient is monitored for the development of cholestasis, hyperbilirubinemia, or one or more symptoms thereof. In some embodiments, the patient is monitored for the development of cholestasis, hyperbilirubinemia, or one or more symptoms thereof by evaluating a parameter in a blood sample obtained from the patient, wherein a finding that the parameter is above a reference level identifies the patient as having cholestasis, hyperbilirubinemia, or one or more symptoms thereof

In some embodiments, the patient is monitored for the development of hyperbilirubinemia and if the patient exhibits cholestasis, hyperbilirubinemia, or one or more symptoms thereof, the patient is administered an anti-cholestatic agent.

In some embodiments, it is determined that the patient exhibits cholestasis, hyperbilirubinemia, or one or more symptoms thereof and the patient is administered an anti-cholestatic agent.

In some embodiments, a patient is determined to exhibit cholestasis or one or more symptoms thereof when the patient exhibits one or more parameters (e.g., total bile acids level, gamma-glutamyl transferase (GGT) level, alkaline phosphatase (ASP) level, aspartate aminotransferase (AST) level, and/or alanine aminotransferase (ALT) level), as measured in a serum bile acid test and/or blood test (e.g., a liver function test (LFT)), that is greater than or less than the age-adjusted norm.

In some embodiments, a patient is determined to exhibit hyperbilirubinemia or one or more symptoms thereof when the patient exhibits a bilirubin level, as measured in a blood test (e.g., a bilirubin test), that is greater than the norm.

In some embodiments, the disclosure provides a method of treating cholestasis in a human patient that has XLMTM and who has been previously administered a viral vector comprising a transgene encoding MTM1 (e.g., resamirigene bilparvovec), the method including administering to the patient an anti-cholestatic agent.

In some embodiments, the disclosure provides a method of treating hyperbilirubinemia in a human patient that has XLMTM and who has been previously administered a viral vector comprising a transgene encoding MTM1 (e.g., resamirigene bilparvovec), the method including administering to the patient an anti-cholestatic agent.

In some embodiments, the disclosure provides a method of preventing cholestasis in a human patient that has XLMTM and who has been previously administered a viral vector comprising a transgene encoding MTM1 (e.g., resamirigene bilparvovec), the method including administering to the patient an anti-cholestatic agent.

In some embodiments, the disclosure provides a method of preventing hyperbilirubinemia in a human patient that has XLMTM and who has been previously administered a viral vector comprising a transgene encoding MTM1 (e.g., resamirigene bilparvovec), the method including administering to the patient an anti-cholestatic agent.

In some embodiments, the patient does not have a history of cholestasis or hyperbilirubinemia.

In some embodiments, the patient does not have a history of any underlying liver disease.

### Vectors for Delivery of Exogenous Nucleic Acids to Target Cells

### Viral Vectors for Nucleic Acid Delivery

Viral genomes provide a rich source of vectors that can be used for the efficient delivery of a gene of interest (e.g., a transgene encoding MTM1) into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox).

Other viruses useful for delivering polynucleotides encoding antibody light and heavy chains or antibody fragments of the invention include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the invention are recombinant nucleic acid constructs that include (1) a transgene to be expressed (e.g., a polynucleotide encoding a MTM1 protein) and (2) viral nucleic acids that facilitate integration and expression of the heterologous genes. The viral nucleic acids may include those sequences of AAV that are required in cis for replication and packaging (e.g., functional inverted terminal repeats (ITRs)) of the DNA into a virion. In typical applications, the transgene encodes MTM1, which is useful for correcting a MTM1 mutation in patients suffering from neuromuscular disorders, such as XLMTM. Such rAAV vectors may also contain marker or reporter genes. Useful rAAV vectors have one or more of the AAV wild type genes deleted in whole or in part but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery. rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of MTM1 in muscle cells. rAAV serotype 9 has also been found to be an efficient transducer of muscle cells. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J.

Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 vector encoding a therapeutic protein pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants are described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Resamirigene Bilparvovec

As described herein, a pseudotyped AAV vector including a nucleic acid sequence encoding a MTM1 gene (SEQ ID NO: 4) operably linked to a desmin promotor (SEQ ID NO:3) flanked by AAV2 ITR and packaged within capsid proteins from AAV8 (AAV2/8) as well as the other genetic components listed in Table 1, refers to the compound known by the international proprietary name (INN) of resamirigene bilparvovec.

Resamirigene bilparvovec is a non-replicating recombinant AAV8 vector expressing a non-codon-optimized human MTM1 cDNA under the control of the muscle-specific human desmin promoter. The MTM1 expression cassette was built by cloning a synthetic DNA sequence complementary to the coding portion (nucleotides 43-1864) of the wild-type human MTM1 transcript (NCBI Ref. Seq NM_000252.3) downstream of the 1.05-kb human desmin enhancer/promoter region. The second intron and polyadenylation sequence of the human p-globin gene (HBB) were inserted upstream and downstream respectively of the MTM1 synthetic cDNA to mediate RNA processing. The expression cassette was flanked by AAV serotype-2 (AAV2) inverted terminal repeats (ITRs). The vector was produced in an AAV8 capsid by two-plasmid transfection in HEK293 cells in suspension culture in bioreactors a full GMP process.

In some embodiments, a method of treating a disorder (e.g., XLMTM) or alleviating one or more symptoms (e.g., stiffness and/or join contractures or need for diaphragm and/or respiratory muscle progression) of a disorder (e.g., XLMTM) in a human patient in need thereof, includes administering to the patient a therapeutically effective amount of resamirigene bilparvovec during a treatment period.

In some embodiments, a method of weaning a human patient off of mechanical ventilation, includes a patient that has previously been administered a therapeutically effective amount of resamirigene bilparvovec. The components of resamirigene bilparvovec are shown in Table 1, below:

TABLE 1
Resamirigene Bilparvovec Nucleic Acid Sequence (SEQ ID NO: 5)

| Range | | |
|---|---|---|
| (nucleotides, | | |
| relative to | Length | |
| SEQ ID NO: 5) | (nucleotides) | Genetic Component |
| 3080-3198 | 119 | AAV2 ITR |
| 3199-3256 | 58 | Linker sequence |
| 3257-4316 | 1,060 | Human desmin promoter (SEQ ID NO: 3) |
| 4317-4354 | 38 | Linker sequence |
| 4355-4460 | 106 | Human Beta-globin intron |
| 4373-4848 | 476 | Human Beta-globin intron |
| 4458-4902 | 445 | Human Beta-globin intron |
| 4917-6738 | 1,822 | Human MTM1 coding sequence (SEQ ID |
| | | NO: 4) |
| 6739-6759 | 21 | Linker sequence |
| 6760-7519 | 760 | Human Beta-globin poly-adenylation |
| | | sequence |
| 7520-7551 | 32 | Linker sequence |
| 7552 | 7,696 | AAV2 ITR |

As described herein, resamirigene bilparvovec refers to the AAV vector having the nucleic acid sequence of SEQ ID NO: 5, shown below:

| | |
|---|---|
| TCGCGCGTTT CGGTGATGAC GGTGAAAACC TCTGACACAT GCAGCTCCCG | 50 |
| GACGTCATTG TCGATCCTGC AGGCGTACGG TAAAAAAAGG CATAGCTAAC | 100 |
| AAGGTGTGGA AAAAGAATTA GTGGTTAGAG AGTGAGCTAT TCGTTGAAAC | 150 |
| AATTGCGTTC TTGAAACAAT TCTTGCTGGT AAAATGTCAC ATTTTATGTG | 200 |
| ACTACAGGTG GAGGATTGGC ACATAACCTA ACCAGTGGGG GAAACAATTG | 250 |
| ACCTCTGGAT TTGTCCAAGT GTATAGTAGC ATTTGCCCAA TCGAATGGTC | 300 |
| CTGGTAAGGT GTTAATGTTG ACTAGAACCA AAGGTGGAAG TTGCAGGGAA | 350 |
| ACTGGTTTAG TACAAGGGTG GACACCAGGC AGTCATCCAG AGGCCCATTA | 400 |
| AAGGCCTTGG AATGTTTTTC CGAAGGAGAA TCACTCCCTC TTCTCTCGCT | 450 |
| TAAAGTTTTA GGGGATTCAT GAACAGCTGC TGTGGGATAG TTTCATGTCC | 500 |
| CTAGCAATTG TAAAGCAACT GAGGGTGGCT TAAACCAGTT TTAGCTTTAG | 550 |
| GGTTAGGGTT ACTGGACTAA AATTTGAGAA ATTCATAAAT CTTAAGGAAA | 600 |
| TCCATTGTGA GTTTTCATTA TGAGTGCATC CAATGTATAA TTTCCATGAC | 650 |
| CCTCCCATGC AAGTGAGCAT GTGAATCAGG AAACGTTACA AGAACCCAAC | 700 |
| AAACTCAACC ACTACTAGAC AGGCGATCAC TTCCAGTTAG TATGCAACTT | 750 |
| TCTGTGTAAT TTTAGTTACC ATTAAAATCT GGATGACCTT AGTGTAAGGA | 800 |
| AAAAATACCT TGAATAGTGT TAAAGATGTA CACTTGGTGT CAGGCATTGT | 850 |
| AACATTGATA AATCTGTGTA AGGTGCTTTT TGAAAACTTC AAAGCTGCAT | 900 |
| CAAGTCAAGT ACAAGAAAGG CCATGGCTGC TAAAGCTGTT GAAGATGTGG | 950 |
| GATGGAACTG GGTCACATTG GTGTTAACAG CGTTGTGCAG AGCCGGCAGG | 1000 |
| ATCTTGGTGT GAGCGAACAT TAGTCTATTT AATAAAGCTG TGTGAATGTT | 1050 |
| GTAGAGGTGA GGATGCTCAC TTGAAAACTC ACTGAAGAAC ACTTGGCCCC | 1100 |
| TTGAACTAAA GTGCTTCTAT CAAGTTCAGT GAGAAATTCC GAATTACAAG | 1150 |
| CATAGGTACT AGAAAAGTTT TGAAAAGCAG TATAGAGCAA CATAAGCACA | 1200 |
| TTCATAAAAT TAGTGATGTA GAAAGTGAAA TTTCCACGTA TGGTCACTCC | 1250 |
| CAGAGAAAAA AAATACGTTT ATTTACCTTT TTTAAAAATA GGGGATTTCA | 1300 |
| GGCCGGGTGA GGTGGCTCAC GCCTGTAATC CCAGCACTTT GGGAGGCCCA | 1350 |
| GGTGGGCGGA TCACCTGAGG TCAGGAGTTG GAGGGATGGC AAATCCCATC | 1400 |
| TCTACAAAAT ATACAAAAAA ATAGCTGGGT GTGTTGGCAG GCGCCTGTAA | 1450 |
| TCCCAGCTAC TCGGAAGGCT GAGGCAGGAG AATCCCTGGA ACCAGGGATG | 1500 |
| TGGAGGTTGC AGTGAGCCGA GATTGTGTAA CTGCATTCCA GCCTGGGCAA | 1550 |
| CAAGAGCAAG ACTCCGTATC AGGAAAAAAA AAAGGGGGGG TTGGATTTCG | 1600 |
| CTTGTTGCAT AGGTTGGTCT CAAACTCCTG GCCTCAAGTG ATTCTCCTGC | 1650 |
| CTCTGCCTCC CAAAGTGCTG AGATTACAGG TGTGAGGCAC CATGCCAGGT | 1700 |
| CTCTTACTGT TTGTAATTAA ATACATACAC ATTTTGTGTG TTTGTGTGCA | 1750 |
| CCTTTATAAA GTCAAAGGTG ATAGTAACCC ATTTAAGTTC CTACTCAATT | 1800 |
| TTACTTTCCA GGGATAACTA ACTACTTTTT CTTTTTGAGA TGGAGTCTCG | 1850 |
| CTGTGTAGCC CAGGCTGGAG TGCAGTGGCA CCATCTCGGC TCACTGCAAG | 1900 |
| CTCCTCCTCC CTGGTTCACG CTATTCTCCT GCCTCAGCCT CCCCAACAAC | 1950 |
| TAGGACTACA GGCTCACCTC GCCATACCTG GCTAATTTTT TGTATTTTTA | 2000 |
| GTAGAGACAG GGTTTCACTG TGTTAGCCAG GATGGTCTCG ATCTCCTGAC | 2050 |
| CTTGTGATCC GCCTGCCTCT GCCTCCCAAA GTGCTGGGAT TACAGGCATG | 2100 |
| AGCAACCTCA CCCAGCTGGG ATAACTACTT TTTACAGGTT GATATTCTTT | 2150 |
| TGGACTTTTC CCCTGTGTAA AAATATACTA TATTTGTTAT GTACATATTA | 2200 |
| TGTACATACA GACACAAATT GGACCATTCT CAGTATAATG ATTCTCAGGT | 2250 |
| TTTTTTTTTT TTTTTGAGGT GGGGAACTAG ATAATTATGG ACATCTTTCC | 2300 |
| ATACTAGCAT ATCAATATCT ACCTCATTCT TTTTAATATT TTTGCTAGTA | 2350 |
| TTCCATTGTA TGAATGTCCT ATGATTTACT TAACCTGTCC ATCAATATTT | 2400 |
| GTTTCCAGGT TTTTGCTATT ATAATGCTGC TGCAAAGTAC ATCCTCACAC | 2450 |
| ATCTTTATTT TGTCTATTCA TATTTCTGTA AGATAGGTTA CTAAAGTTGG | 2500 |
| AACTGCCAAA TTAACACTAT CATACTATTT TGTTTTTTAA TTTTAATTTT | 2550 |
| TTAAAAAATG TAAAATGTGC AATTTCAAGA GGAGAAACTT GAACACAAGG | 2600 |
| AGCAAAATCT ATTTTTATAA CATCCTATTA AAAGCTTGCT TTACATAAAG | 2650 |
| ATTTTGAAAG AATAGCATAA ATACAAGATT TCTATTTTAA TTGGATTCTT | 2700 |
| AGGGCTAATA AAATAATCAG CCTTAGCACT TATTTATTTA TTTTTTTTGA | 2750 |
| GAGGGAGTCT CGCTCTGTTG TCCATGCTGG AGTGCAGTGG CGTGATCTCG | 2800 |
| GCTCACTGCA AGCTCCACCT CATGAGTTCA CACCATTCTC CTGCCTCAGT | 2850 |
| CTCCCGAGTA GCTGGGACTC CAGGCGCCCT CTACAAAGCC CGTCTAATTT | 2900 |
| TTTTTGTATT TTTAGTAGAG ACAGGGTTTC ACTGTGTTAG CCAGGATGGT | 2950 |
| CTTGATCTCC TGACCTTGTG ATCTGCCCGC CTCGGCCTCC CAAAGTGCTG | 3000 |
| GGATTATAGG CTTGAGCCAC TGCTCCCGGC CAGCACTTAT TTTTATAATT | 3050 |
| CTTCATGATT ACTGTGTTAC TGTCCCATGG GCCGCCAGGG CCAGCTAGGT | 3100 |
| TGGCCACTCC CTCTCTGCGC GCTCGCTCGC TCACTGAGGC CGGGCGACCA | 3150 |
| AAGGTCGCCC GACGCCCGGG CTTTGCCCGG GCGGCCTCAG TGAGCGAGCG | 3200 |
| AGCGCGCAGA GAGGGAGTGG CCAACTCCAT CACTAGGGGT TCCTCCTAGC | 3250 |
| ACGCGCTACC CCCTGCCCCC CACAGCTCCT CTCCTGTGCC TTGTTTCCCA | 3300 |
| GCCATGCGTT CTCCTCTATA AATACCCGCT CTGGTATTTG GGGTTGGCAG | 3350 |
| CTGTTGCTGC CAGGGAGATG GTTGGGTTGA CATGCGGCTC CTGACAAAAC | 3400 |
| ACAAACCCCT GGTGTGTGTG GGCGTGGGTG GTGTGAGTAG GGGGATGAAT | 3450 |
| CAGGGAGGGG GCGGGGGACC CAGGGGGCAG GAGCCACACA AAGTCTGTGC | 3500 |
| GGGGGTGGGA GCGCACATAG CAATTGGAAA CTGAAAGCTT ATCAGACCCT | 3550 |
| TTCTGGAAAT CAGCCCACTG TTTATAAACT TGAGGCCCCA CCCTCGACAG | 3600 |
| TACCGGGGAG GAAGAGGGCC TGCACTAGTC CAGAGGGAAA CTGAGGCTCA | 3650 |
| GGGCCAGCTC GCCCATAGAC ATACATGGCA GGCAGGCTTT GGCCAGGATC | 3700 |
| CCTCCGCCTG CCAGGCGTCT CCCTGCCCTC CCTTCCTGCC TAGAGACCCC | 3750 |
| CACCCTCAAG CCTGGCTGGT CTTTGCCTGA GACCCAAACC TCTTCGACTT | 3800 |
| CAAGAGAATA TTTAGGAACA AGGTGGTTTA GGGCCTTTCC TGGGAACAGG | 3850 |
| CCTTGACCCT TTAAGAAATG ACCCAAAGTC TCTCCTTGAC CAAAAAGGGG | 3900 |
| ACCCTCAAAC TAAAGGGAAG CCTCTCTTCT GCTGTCTCCC CTGACCCCAC | 3950 |
| TCCCCCCCAC CCCAGGACGA GGAGATAACC AGGGCTGAAA GAGGCCCGCC | 4000 |
| TGGGGGCTGC AGACATGCTT GCTGCCTGCC CTGGCGAAGG ATTGGTAGGC | 4050 |
| TTGCCCGTCA CAGGACCCCC GCTGGCTGAC TCAGGGGCGC AGGCCTCTTG | 4100 |
| CGGGGGAGCT GGCCTCCCCG CCCCCACGGC CACGGGCCGC CCTTTCCTGG | 4150 |
| CAGGACAGCG GGATCTTGCA GCTGTCAGGG GAGGGGAGGC GGGGGCTGAT | 4200 |
| GTCAGGAGGG ATACAAATAG TGCCGACGGC TGGGGGCCCT GTCTCCCCTC | 4250 |
| GCCGCATCCA CTCTCCGGCC GGCCGCCTGC CCGCCGCCTC CTCCGTGCGC | 4300 |
| CCGCCAGCCT CGCCCGGACT CTAGAGGATC CAGATCTAAG CTTCTCTGGT | 4350 |
| CACCGATCCT GAGAACTTCA GGGTGAGTCT ATGGGACCCT TGATGTTTTC | 4400 |
| TTTCCCCTTC TTTTCTATGG TTAAGTTCAT GTCATAGGAA GGGGAGAAGT | 4450 |
| AACAGGGTAC ACATATTGAC CAAATCAGGG TAATTTTGCA TTTGTAATTT | 4500 |
| TAAAAAATGC TTTCTTCTTT TAATATACTT TTTTGTTTAT CTTATTTCTA | 4550 |
| ATACTTTCCC TAATCTCTTT CTTTCAGGGC AATAATGATA CAATGTATCA | 4600 |
| TGCCTCTTTG CACCATTCTA AAGAATAACA GTGATAATTT CTGGGTTAAG | 4650 |
| GCAATAGCAA TATTTCTGCA TATAAATATT TCTGCATATA AATTGTAACT | 4700 |
| GATGTAAGAG GTTTCATATT GCTAATAGCA GCTACAATCC AGCTACCATT | 4750 |
| CTGCTTTTAT TTTATGGTTG GGATAAGGCT GGATTATTCT GAGTCCAAGC | 4800 |
| TAGGCCCTTT TGCTAATCAT GTTCATACCT CTTATCTTCC TCCCACAGCT | 4850 |
| CCTGGGCAAC GTGCTGGTCT GTGTGCTGGC CCATCACTTT GGCAAAGAAT | 4900 |
| TCCGCGGGCG GCCGCAAGTT TCCAGGATGG CTTCTGCATC AACTTCTAAA | 4950 |
| TATAATTCAC ACTCCTTGGA GAATGAGTCT ATTAAGAGGA CGTCTCGAGA | 5000 |
| TGGAGTCAAT CGAGATCTCA CTGAGGCTGT TCCTCGACTT CCAGGAGAAA | 5050 |
| CACTAATCAC TGACAAAGAA GTTATTTACA TATGTCCTTT CAATGGCCCC | 5100 |
| ATTAAGGGAA GAGTTTACAT CACAAATTAT CGTCTTTATT TAAGAAGTTT | 5150 |
| GGAAACGGAT TCTTCTCTAA TACTTGATGT TCCTCTGGGT GTGATCTCGA | 5200 |
| GAATTGAAAA AATGGGAGGC GCGACAAGTA GAGGAGAAAA TTCCTATGGT | 5250 |
| CTAGATATTA CTTGTAAAGA CATGAGAAAC CTGAGGTTCG CTTTGAAACA | 5300 |
| GGAAGGCCAC AGCAGAAGAG ATATGTTTGA GATCCTCACG AGATACGCGT | 5350 |
| TTCCCCTGGC TCACAGTCTG CCATTATTTG CATTTTTAAA TGAAGAAAAG | 5400 |
| TTTAACGTGG ATGGATGGAC AGTTTACAAT CCAGTGGAAG AATACAGGAG | 5450 |
| GCAGGGCTTG CCCAATCACC ATTGGAGAAT AACTTTTATT AATAAGTGCT | 5500 |
| ATGAGCTCTG TGACACTTAC CCTGCTCTTT TGGTGGTTCC GTATCGTGCC | 5550 |
| TCAGATGATG ACCTCCGGAG AGTTGCAACT TTTAGGTCCC GAAATCGAAT | 5600 |
| TCCAGTGCTG TCATGGATTC ATCCAGAAAA TAAGACGGTC ATTGTGCGTT | 5650 |
| GCAGTCAGCC TCTTGTCGGT ATGAGTGGGA AACGAAATAA AGATGATGAG | 5700 |
| AAATATCTCG ATGTTATCAG GGAGACTAAT AAACAAATTT CTAAACTCAC | 5750 |
| CATTTATGAT GCAAGACCCA GCGTAAATGC AGTGGCCAAC AAGGCAACAG | 5800 |
| GAGGAGGATA TGAAAGTGAT GATGCATATC ATAACGCCGA ACTTTTCTTC | 5850 |
| TTAGACATTC ATAATATTCA TGTTATGCGG GAATCTTTAA AAAAAGTGAA | 5900 |
| GGACATTGTT TATCCTAATG TAGAAGAATC TCATTGGTTG TCCAGTTTGG | 5950 |
| AGTCTACTCA TTGGTTAGAA CATATCAAGC TCGTTTTGAC AGGAGCCATT | 6000 |
| CAAGTAGCAG ACAAAGTTTC TTCAGGGAAG AGTTCAGTGC TTGTGCATTG | 6050 |
| CAGTGACGGA TGGGACAGGA CTGCTCAGCT GACATCCTTG GCCATGCTGA | 6100 |
| TGTTGGATAG CTTCTATAGG AGCATTGAAG GGTTCGAAAT ACTGGTACAA | 6150 |
| AAAGAATGGA TAAGTTTTGG ACATAAATTT GCATCTCGAA TAGGTCATGG | 6200 |
| TGATAAAAAC CACACCGATG CTGACCGTTC TCCTATTTTT CTCCAGTTTA | 6250 |
| TTGATTGTGT GTGGCAAATG TCAAAACAGT TCCCTACAGC TTTTGAATTC | 6300 |
| AATGAACAAT TTTTGATTAT AATTTTGGAT CATCTGTATA GTTGCCGATT | 6350 |
| TGGTACTTTC TTATTCAACT GTGAATCTGC TCGAGAAAGA CAGAAGGTTA | 6400 |
| CAGAAAGGAC TGTTTCTTTA TGGTCACTGA TAAACAGTAA TAAAGAAAAA | 6450 |
| TTCAAAAACC CCTTCTATAC TAAAGAAATC AATCGAGTTT TATATCCAGT | 6500 |
| TGCCAGTATG CGTCACTTGG AACTCTGGGT GAATTACTAC ATTAGATGGA | 6550 |
| ACCCCAGGAT CAAGCAACAA CAGCCGAATC CAGTGGAGCA GCGTTACATG | 6600 |
| GAGCTCTTAG CCTTACGCGA CGAATACATA AAGCGGCTTG AGGAACTGCA | 6650 |
| GCTCGCCAAC TCTGCCAAGC TTTCTGATCC CCCAACTTCA CCTTCCAGTC | 6700 |
| CTTCGCAAAT GATGCCCCAT GTGCAAACTC ACTTCTGACC GGTCCGAGGG | 6750 |
| CCCAGATCTA ATTCACCCCA CCAGTGCAGG CTGCCTATCA GAAAGTGGTG | 6800 |
| GCTGGTGTGG CTAATGCCCT GGCCCACAAG TATCACTAAG CTCGCTTTCT | 6850 |
| TGCTGTCCAA TTTCTATTAA AGGTTCCTTT GTTCCCTAAG TCCAACTACT | 6900 |
| AAACTGGGGG ATATTATGAA GGGCCTTGAG CATCTGGATT CTGCCTAATA | 6950 |
| AAAAACATTT ATTTTCATTG CAATGATGTA TTTAAATTAT TTCTGAATAT | 7000 |
| TTTACTAAAA AGGGAATGTG GGAGGTCAGT GCATTTAAAA CATAAAGAAA | 7050 |
| TGAAGAGCTA GTTCAAACCT TGGGAAAATA CACTATATCT TAAACTCCAT | 7100 |
| GAAAGAAGGT GAGGCTGCAA ACAGCTAATG CACATTGGCA ACAGCCCCTG | 7150 |
| ATGCCTATGC CTTATTCATC CCTCAGAAAA GGATTCAAGT AGAGGCTTGA | 7200 |
| TTTGGAGGTT AAAGTTTTGC TATGCTGTAT TTTACATTAC TTATTGTTTT | 7250 |
| AGCTGTCCTC ATGAATGTCT TTTCACTACC CATTTGCTTA TCCTGCATCT | 7300 |
| CTCAGCCTTG ACTCCACTCA GTTCTCTTGC TTAGAGATAC CACCTTTCCC | 7350 |
| CTGAAGTGTT CCTTCCATGT TTTACGGCGA GATGGTTTCT CCTCGCCTGG | 7400 |
| CCACTCAGCC TTAGTTGTCT CTGTTGTCTT ATAGAGGTCT ACTTGAAGAA | 7450 |
| GGAAAAACAG GGGGCATGGT TTGACTGTCC TGTGAGCCCT TCTTCCCTGC | 7500 |
| CTCCCCCACT CACAGTGACC GGCCGCTCTA GGAGGAACCC CTAGTGATGG | 7550 |
| AGTTGGCCAC TCCCTCTCTG CGCGCTCGCT CGCTCACTGA GGCCGGGCGA | 7600 |
| CCAAAGGTCG CCCGACGCCC GGGCTTTGCC CGGGCGGCCT CAGTGAGCGA | 7650 |
| GCGAGCGCGC AGAGAGGGAG TGGCCAACCT AGAGGCCGCC AGGGCCATAT | 7700 |
| TTCTCAATTT TTAAATTTTT CAAAAAAATT AATCCTTAAT GTGCATATTT | 7750 |
| TTGAATTGTT AATATAACTT TTTGAGGTGA TGTCTTCATG TGTTTCAACT | 7800 |
| ACTTAAAAAC TTTTAAACAG TATATAATAA AAAATCTTCC AGGCCACTCA | 7850 |
| CACCTGTAAT CCCAGCACTT TGGGAGGCTG AGGTGGGCAG ATCACCTGAG | 7900 |
| GGCAGGAGTT CGAGACCAGC CTGGCCAATA TATATATATT CATATATTCA | 7950 |
| TATATATATA TATATTCATA TATTCATATA TATATATTCA TATATTCATA | 8000 |
| TATATATATA TATATATATA TAGCAAAACC TCATCTCTAA TAAAATACAA | 8050 |
| AAATTAGCTG AGCGTGGTGA TGGATGCCTG TAGTCCCAGC TACTCGGGAG | 8100 |
| GCTGAGGCAG GAGAATCTCT TGAACCTGGG AGGTGGAGGT TGCAGTGAGC | 8150 |
| TGAGATGGTG CCACTGCCCT CCAGCCTGAG TGACAGAGCG AGACTCGGTC | 8200 |
| TCCAAAAAAA AACAACAAAA AAATCTTCCA TCCTTGTCTC CCATCCACCC | 8250 |
| CTTCCCCCCA GCATGTACTT GCAGACTTTA TGCATATACA GTGAGTACTG | 8300 |
| TATATACACA AATAATAAAA AAATCATATA TATAATATAT GTAATTCCCC | 8350 |
| TTTACATGAA AGGTAGCACA CTGGTCTGTA CAGTCTGTCT GCACTGTGCT | 8400 |
| ATTTCACTTT ATATTTTTAT AGTTTGACAG AGTTCTAACA TTTCTTTTTT | 8450 |
| TTTTTTTTTA ACAGAGTCTT GTTCCTGATT GTTAAATTTT AAAGCATCCT | 8500 |
| AAAGTTTGGT TTCACACTTG AATGAATACC ATGTAAGGAT TCACTTACAT | 8550 |
| AGATGTGGTT GCCTGAATCT TAAGAATAAA ATAACATTGT TTGTATTTAT | 8600 |
| TTAAATTAGT GTTCCTTTTA TGGTTTGCCT GAAAGCACAA CAAAATCCTC | 8650 |
| ACCAAGATAT TACAATTATG ACTCCCATAC AGGTAAACTG TTTAGAGATT | 8700 |
| GGCAAGCACC TTTTAATGAA AGGAGTCAGC CAGCTTAGTG TGCAGTATTT | 8750 |
| ATTTCTGCCG GAAGAGGGAG CTTCAGGGAC AGACTTTGGT TTAGTCATGA | 8800 |
| AGCCTCCAGC ACTCCCAAGC GGTTGTGGTT GACCAAGCAA TTTATGCTTT | 8850 |
| TACCTTTCTA CTTCCAGAGG CTTGTTTACT TATCAGTAAG CATTAATTTA | 8900 |
| GTGTCCCCTC AGATGCCTTT TACTTTCTTC TTTTCTGCCT AGAATAAGCT | 8950 |
| GCTCTTCCAA TTTTGCAGCT ACATGTTTCC ACCCCAGTTG GAATTTCTCC | 9000 |
| ATAACATCCA TTGTAGCTAT CCTTCAATCT ACAGCCTCTA TTTCCTGTTA | 9050 |
| TAGCTGGTCA GGTCTAATCC CTCAAAATAC TCTGTCCCCT GCTTCCCTTA | 9100 |
| TCTGCTGGCC ACCTTTTTCC CCCACATACA CACTGCCATG TCCCACCCTT | 9150 |
| CACTCAAGTT GTTCCCTGCC ACCTCAACAA ATTTAAGTCC ATAAAATAGA | 9200 |
| GTAAGTGTTC CTGACTGTTA AATTTTAAAG CATCCCAAAG TCTGATTTCA | 9250 |
| CACTCGAATG AATACTATGT ACGGATTCAT TTACATAGAT GCGGTTGCAT | 9300 |
| GAGTCTTAAC AAAAAAATAA CATTATTTGT ATTTATTCAA AGTACTGTCA | 9350 |
| AGATATAATG TCAAGACCTA ATTCAAAGGT TCCACAAAGC CTTCCTTGAC | 9400 |
| TGCCCCCAAC GAAGATTATC CATTTTCCCT GAAATCCCAT TGACTTTTCT | 9450 |
| ATTTTGTAAG GAGGCTCGTG AGACTCTGTC TAAAAACAAA ACAAAACAAA | 9500 |
| AAGAAACAAT CAAACGGCTT GCTTCTGTTC TTTGATCTGC TAGTAAGCAA | 9550 |
| AAATTACACA TGGTGACAGG AGCTATGTGA GGCTGTCAGG TTGAATGGGA | 9600 |
| GGAGTTTGGG ATCCTGCTTG TGGATGGTTG GAAGAGGCTT TCGGGAAAGA | 9650 |
| CAGTATTTAT GTGAGACCTG GAAGATGGGC CTTAGCTTTG CAGAAGGTGG | 9700 |
| AGAGGCAGGA AATAGCACGG GGGCCCTGGG GCTGGAAGAC TTGGGCATAT | 9750 |
| TTGAGGAACA GAAAGGAGAC CAGCATAACT GAGGTGGGAA AAGCATGTGA | 9800 |
| AGAGATGGGG CTGGAGGAGG CCGGGAGTGG TGGCTCACGC CTGTAATCCC | 9850 |
| AGCACTTTGG GAGGCCAAGG CAGGCGGATC ATGAGCTCAG GAGATTGAGA | 9900 |
| CCATCCTGGC TAACACGGTG AAACCCCCTC TCTACTAAAA ATACAAAAAA | 9950 |
| AAAAAAAAAA AAAATTAGCT GGGCGTGGTG GCAGGAGCCT GTAGTCCCAG | 10000 |
| CTACCTGGGA GGCTGAGGCA GGAGAATGGC GTGAACCTGG AAGGCTGAGC | 10050 |
| TTGCAGTGAG CCGAGATTGC ACCACTGCAC TCCAGCCTGG GAGACAGAGA | 10100 |
| GAGACTCCCT CTCAAAAAAA CAAACAAACG AAACAAAACA AAACAAAAAT | 10150 |
| TAGCCAGGCG TGGTGGTATG CACCTGTAAT CCCAGCTACT CGGGAGGTTG | 10200 |
| AGGCAGGAGA AACGCTTGAA CTCAGGAGGC GGAGGTTGCA GTGAGCCGAG | 10250 |
| ACTGCGCCAC TGCACTCCAG CCTGGGTGAC AGAGGGAGAC TCCATCTCAA | 10300 |
| AAAAAAAAAT TTTTTTTTTT TTACAAACGG TGTCTCCCTC TGTCGCCCAG | 10350 |
| GCTGGAGTGC AGTGGTGTGA TCACAGCTCA CTCCAGCCTC AACCTCCCCA | 10400 |
| GCTGAAGCCA TCCTCTTGCC TCAGCCTCCT AAGTAGCTGG GACTACAGGC | 10450 |
| GCGCACCTCC AGGCTTGGCT CTTATTCTTT TTATTGTTTT TGAAACTATA | 10500 |
| GAACCTATTT TTAAAAAATG TTTTGGTTGT TTTTATTGCT GCTTTTCCTT | 10550 |
| TTGGGGTTAG AACACAAGTT TTGATGGGAA ACAGGTTAGA ACACATTCAT | 10600 |
| CTCTTCCCAT AGCGATGGTC ATAGAAAAAC GGGGCATATT TATAAACTCT | 10650 |
| CAGTTGATCT TAAAATGTGC AAAAGCTGCC GAACTCCTGG GAGTGAGCTC | 10700 |
| GAGCCCTGCA GGATCATTGT CACATGTGAG CAAAAGGCCA GCAAAAGGCC | 10750 |
| AGGAACCGTA AAAAGGCCGC GTTGCTGGCG TTTTTCCATA GGCTCCGCCC | 10800 |
| CCCTGACGAG CATCACAAAA ATCGACGCTC AAGTCAGAGG TGGCGAAACC | 10850 |
| CGACAGGACT ATAAAGATAC CAGGCGTTTC CCCCTGGAAG CTCCCTCGTG | 10900 |
| CGCTCTCCTG TTCCGACCCT GCCGCTTACC GGATACCTGT CCGCCTTTCT | 10950 |
| CCCTTCGGGA AGCGTGGCGC TTTCTCATAG CTCACGCTGT AGGTATCTCA | 11000 |
| GTTCGGTGTA GGTCGTTCGC TCCAAGCTGG GCTGTGTGCA CGAACCCCCC | 11050 |
| GTTCAGCCCG ACCGCTGCGC CTTATCCGGT AACTATCGTC TTGAGTCCAA | 11100 |
| CCCGGTAAGA CACGACTTAT CGCCACTGGC AGCAGCCACT GGTAACAGGA | 11150 |
| TTAGCAGAGC GAGGTATGTA GGCGGTGCTA CAGAGTTCTT GAAGTGGTGG | 11200 |
| CCTAACTACG GCTACACTAG AAGAACAGTA TTTGGTATCT GCGCTCTGCT | 11250 |
| GAAGCCAGTT ACCTTCGGAA AAAGAGTTGG TAGCTCTTGA TCCGGCAAAC | 11300 |
| AAACCACCGC TGGTAGCGGT GGTTTTTTTG TTTGCAAGCA GCAGATTACG | 11350 |
| CGCAGAAAAA AAGGATCTCA AGAAGATCCT TTGATCTTTT CTACGGGGTC | 11400 |
| TGACGCTCAG TGGAACGAAA ACTCACGTTA AGGGATTTTG GTCATGAGAT | 11450 |
| TATCAAAAAG GATCTTCACC TAGATCCTTT TAAATTAAAA ATGAAGTTTT | 11500 |
| AAATCAAGCC CAATCTGAAT AATGTTACAA CCAATTAACC AATTCTGATT | 11550 |
| AGAAAAACTC ATCGAGCATC AAATGAAACT GCAATTTATT CATATCAGGA | 11600 |
| TTATCAATAC CATATTTTTG AAAAAGCCGT TTCTGTAATG AAGGAGAAAA | 11650 |
| CTCACCGAGG CAGTTCCATA GGATGGCAAG ATCCTGGTAT CGGTCTGCGA | 11700 |
| TTCCGACTCG TCCAACATCA ATACAACCTA TTAATTTCCC CTCGTCAAAA | 11750 |
| ATAAGGTTAT CAAGTGAGAA ATCACCATGA GTGACGACTG AATCCGGTGA | 11800 |
| GAATGGCAAA AGTTTATGCA TTTCTTTCCA GACTTGTTCA ACAGGCCAGC | 11850 |
| CATTACGCTC GTCATCAAAA TCACTCGCAT CAACCAAACC GTTATTCATT | 11900 |
| CGTGATTGCG CCTGAGCGAG ACGAAATACG CGATCGCTGT TAAAAGGACA | 11950 |
| ATTACAAACA GGAATCGAAT GCAACCGGCG CAGGAACACT GCCAGCGCAT | 12000 |
| CAACAATATT TTCACCTGAA TCAGGATATT CTTCTAATAC CTGGAATGCT | 12050 |
| GTTTTTCCGG GGATCGCAGT GGTGAGTAAC CATGCATCAT CAGGAGTACG | 12100 |
| GATAAAATGC TTGATGGTCG GAAGAGGCAT AAATTCCGTC AGCCAGTTTA | 12150 |
| GTCTGACCAT CTCATCTGTA ACATCATTGG CAACGCTACC TTTGCCATGT | 12200 |
| TTCAGAAACA ACTCTGGCGC ATCGGGCTTC CCATACAAGC GATAGATTGT | 12250 |
| CGCACCTGAT TGCCCGACAT TATCGCGAGC CCATTTATAC CCATATAAAT | 12300 |
| CAGCATCCAT GTTGGAATTT AATCGCGGCC TCGACGTTTC CCGTTGAATA | 12350 |
| TGGCTCATAA CACCCCTTGT ATTACTGTTT ATGTAAGCAG ACAGTTTTAT | 12400 |
| TGTTCATGAT GATATATTTT TATCTTGTGC AATGTAACAT CAGAGATTTT | 12450 |
| GAGACACGGG CCAGAGCTGC A | 12500 |

### Methods for the Delivery of Exogenous Nucleic Acids to Target Cells

### Transfection Techniques

Techniques that can be used to introduce a transgene, such as a MTM1 transgene described herein, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids (e.g., nucleic acids capable of expression in e.g., neurons, glial cells, or non-neural cells, such as colon and kidney cells). Electroporation of mammalian cells is described in detail, e.g., in Chu et al., Nucleic Acids Research 15:1311 (1987), the disclosure of which is incorporated herein by reference. A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., J. Vis. Exp. 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Top Curr Chem. 228:227 (2003), the disclosure of which is incorporated herein by reference) polyethylenimine, and DEAE-dextran, the use of which as a transfection agent is described in detail, for example, in Gulick et al., Curr Protoc Mol Biol. 40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Ther. 9:102 (2002), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods Cell Biol. 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nat. Rev. Genet. 11:636 (2010); and in Joung et al., Nat. Rev. Mol. Cell Biol. 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Pharmaceutical Compositions and Routes of Administration

The gene therapy agents described herein may contain a transgene, such as a transgene encoding MTM1 and may be incorporated into a vehicle for administration into a patient, such as a human patient suffering from a neuromuscular disorder (for example, XLMTM). Pharmaceutical compositions containing vectors, such as viral vectors, that contain the transcription regulatory elements (e.g., a desmin promoter) described herein operably linked to a therapeutic transgene can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Viral vectors, such as AAV vectors and others described herein, containing the transcription regulatory element operably linked to a therapeutic transgene may be administered to a patient (e.g., a human patient) by a variety of routes of administration. The route of administration may vary, for example, with the onset and severity of disease, and may include, e.g., intradermal, transdermal, parenteral, intravenous, intramuscular, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### Kits

The compositions described herein can be provided in a kit for use in treating a neuromuscular disorder (e.g., XLMTM). In some embodiments, the kit may include one or more viral vectors as described herein. The kit can include a package insert that instructs a user of the kit, such as a physician of skill in the art, to perform any one of the methods described herein. The kit may optionally include a syringe or other device for administering the composition. In some embodiments, the kit may include one or more additional therapeutic agents.

In some embodiments, the kit may include one or more anti-cholestatic agents as described herein. The kit can include a package insert that instructs a user of the kit, such as a physician of skill in the art, to perform any one of the methods described herein. The kit may optionally include a syringe or other device for administering the composition. In some embodiments, the kit may include one or more additional therapeutic agents.

### Dosing Regimens

### Dosing Regimens Involving AAV-MTM1 Vectors

Using the compositions and methods of the disclosure, a patient having a neuromuscular disorder (e.g., XLMTM) may be administered an AAV vector containing a transgene encoding MTM1 (e.g., resamirigene bilparvovec) in an amount of about 1.3×10¹⁴ vg/kg. Administration of the vector to the patient in such a quantity can achieve the beneficial effect of augmenting MTM1 expression in the patient, e.g., to within 50% or 200% of wild-type levels, without inducing toxic side effects.

In some embodiments, the AAV vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 1.5×10¹⁴ vg/kg (e.g., less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of less than about 1×10¹⁴ vg/kg (e.g., less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less). For example, the AAV vector may be administered to the patient in an amount of about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg.

In some embodiments, the AAV vector is administered to the patient in an amount of from about 3×10¹³ vg/kg to about 2.3×10¹⁴ vg/kg (e.g., in an amount of from about 3×10¹³ vg/kg to about 2.3×10¹⁴ vg/kg). For example, the AAV vector may be administered to the patient in an amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 4×10¹³ vg/kg to about 2.3×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 5×10¹³ vg/kg to about 2.3×10¹⁴ vg/kg, such as an in amount of about 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 6×10¹³ vg/kg to about 2.3×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 7×10¹³ vg/kg to about 2.3×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 8×10¹³ vg/kg to about 2.3×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 9×10¹³ vg/kg to about 2.3×10¹⁴ vg/kg, such as an in amount of 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of from about 1×10¹⁴ vg/kg to about 2.3×10¹⁴ vg/kg, such as an in amount 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, or 2.3×10¹⁴ vg/kg.

In some embodiments, the AAV vector is administered to the patient in an amount of about 3×10¹³ vg/kg. In some embodiments, the AAV vector is administered to the patient in an amount of about 4×10¹³ vg/kg. In some embodiments, the AAV vector is administered to the patient in an amount of about 5×10¹³ vg/kg. In some embodiments, the AAV vector is administered to the patient in an amount of about 6×10¹³ vg/kg. In some embodiments, the AAV vector is administered to the patient in an amount of about 7×10¹³ vg/kg. In some embodiments, the AAV vector is administered to the patient in an amount of about 8×10¹³ vg/kg. In some embodiments, the AAV vector is administered to the patient in an amount of about 9×10¹³ vg/kg. In some embodiments, the AAV vector is administered to the patient in an amount of about 1×10¹⁴ vg/kg. In some embodiments, the AAV vector is administered to the patient in an amount of about 1.1×10¹⁴ vg/kg. In some embodiments, the AAV vector is administered to the patient in an amount of about 1.2×10¹⁴ vg/kg. In some embodiments, the AAV vector is administered to the patient in an amount of about 1.3×10¹⁴ vg/kg. In some embodiments, the AAV vector is administered to the patient in an amount of about 1.4×10¹⁴ vg/kg. In some embodiments, the AAV vector is administered to the patient in an amount of about 1.5×10¹⁴ vg/kg. In some embodiments, the AAV vector is administered to the patient in an amount of about 1.6×10¹⁴ vg/kg. In some embodiments, the AAV vector is administered to the patient in an amount of about 1.7×10¹⁴ vg/kg. In some embodiments, the AAV vector is administered to the patient in an amount of about 1.8×10¹⁴ vg/kg. In some embodiments, the AAV vector is administered to the patient in an amount of about 1.9×10¹⁴ vg/kg. In some embodiments, the AAV vector is administered to the patient in an amount of about 2×10¹⁴ vg/kg. In some embodiments, the AAV vector is administered to the patient in an amount of about 2.1×10¹⁴ vg/kg. In some embodiments, the AAV vector is administered to the patient in an amount of about 2.2×10¹⁴ vg/kg. In some embodiments, the AAV vector is administered to the patient in an amount of about 2.3×10¹⁴ vg/kg. In some embodiments, the AAV vector is administered to the patient in an amount of about 2.4×10¹⁴ vg/kg. In some embodiments, the AAV vector is administered to the patient in an amount of about 2.5×10¹⁴ vg/kg. In some embodiments, the AAV vector is administered to the patient in an amount of about 2.6×10¹⁴ vg/kg. In some embodiments, the AAV vector is administered to the patient in an amount of about 2.7×10¹⁴ vg/kg. In some embodiments, the AAV vector is administered to the patient in an amount of about 2.8×10¹⁴ vg/kg. In some embodiments, the AAV vector is administered to the patient in an amount of about 2.9×10¹⁴ vg/kg. In some embodiments, the AAV vector is administered to the patient in an amount of about 3×10¹⁴ vg/kg.

In some embodiments, the AAV vector is administered to the patient in a single dose comprising the amount (e.g., less than about 3×10¹⁴ vg/kg).

In some embodiments, the AAV vector is administered to the patient in two or more (e.g., two, three, four, five, six, seven, eight, nine, or ten) doses that, together, comprise the amount (e.g., less than about 3×10¹⁴ vg/kg).

In some embodiments, the AAV vector is administered to the patient in two or more (e.g., two, three, four, five, six, seven, eight, nine, or ten) doses that each, individually, comprise the amount (e.g., less than about 3×10¹⁴ vg/kg).

In some embodiments, the two or more (e.g., two, three, four, five, six, seven, eight, nine, or ten) doses are separated from one another by one year or more (e.g., one year, one year and one day, one year and one month, one year and six months, two years, three years, four years, or five years).

In some embodiments, the two or more (e.g., two, three, four, five, six, seven, eight, nine, or ten) doses are administered to the patient within about 12 months (e.g., about 12 months, about 11 months, about 10 months, about 9 months, about 8 months, about 7 months, about 6 months, about 5 months, about 4 months, about 3 months, about 2 months, or about 1 month) of one another

### Combination Therapies

An AAV vector containing a transgene encoding MTM1 (e.g., resamirigene bilparvovec) described herein can be administered in combination with a one or more additional therapeutic procedures (e.g., nasobiliary drainage (NBD)) and/or agents (e.g., an anti-cholestatic agent) for the treatment of a neuromuscular disorder (e.g., XLMTM).

### Therapeutic Procedure

In some embodiments, the one or more additional therapeutic procedures is NBD. NBD is a therapeutic procedure that is performed to help drain bile (e.g., when the bile duct is blocked, a biliary drain may help bile to flow from the liver into the intestine). In some embodiments, NBD is performed with a biliary drain (also known as a biliary stent), which is a thin, hollow, flexible tube with several small holes along the sides. A biliary drain may be inserted in a patient's bile duct to allow it to drain.

### Therapeutic Agent

In some embodiments, the one or more additional therapeutic agents is an anti-cholestatic agent (e.g., a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor) or a combination thereof.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence within about six weeks before or after (e.g., about six weeks before or after, about five weeks before or after, about four weeks before or after, about three weeks before or after, about two weeks before or after, or about one week before or after) administration of the viral vector to the patient.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence within about five weeks before or after (e.g., about five weeks before or after, about four weeks before or after, about three weeks before or after, about two weeks before or after, or about one week before or after) administration of the viral vector to the patient.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence within about one week before or after (e.g., about one week before or after, about six days before or after, about five days before or after, about four days before or after, about three days before or after, about two days before or after, or about one day before or after) administration of the viral vector to the patient.

In some embodiments, the anti-cholestatic agent is administered to the patient in one or more (e.g., one, two, three, four, five, six, seven, eight, nine, ten, fifteen, twenty, thirty, forty, fifty, sixty, and seventy) doses that commence on the same day (e.g., 24^{th} hour, on the 23^{rd} hour, on the 22^{nd} hour, on the 21^{st} hour, on the 20^{th} hour, on the 19^{th} hour, on the 18^{th} hour, on the 17^{th} hour, on the 16^{th} hour, on the 15^{th} hour, on the 14^{th} hour, on the 13^{th} hour, on the 12^{th} hour, on the 11^{th} hour, on the 10^{th} hour, on the 9^{th} hour, on the 8^{th} hour, on the 7^{th} hour, on the 6^{th} hour, on the 5^{th} hour, on the 4^{th} hour, on the 3^{rd} hour, on the 2^{nd} hour, on the 1^{st} hour, on the 60^{th} minute, on the 59^{th} minute, on the 58^{th} minute, on the 57^{th} minute, on the 56^{th} minute, on the 55^{th} minute, on the 50^{th} minute, on the 40^{th} minute, on the 30^{th} minute, on the 20^{th} minute, on the 10^{th} minute, or on the same minute) of administration of the viral vector to the patient.

In some embodiments, the anti-cholestatic agent is a bile acid. In some embodiments, the bile acid is ursodeoxycholic acid or a derivative thereof or nor-ursodeoxycholic acid. In some embodiments, the bile acid is ursodiol.

In some embodiments, the anti-cholestatic agent is an FXR ligand. In some embodiments, the FXR ligand is obeticholic acid, cilofexor, tropifexor, tretinoin, or EDP-305.

In some embodiments, the one or more anti-cholestatic agent is an FGF-19 mimetic. In some embodiment, the FGF-19 mimetic is aldafermin.

In some embodiments, the anti-cholestatic agent is a TGR5 agonist. In some embodiments, the TGR5 agonist is INT-777 or INT-767.

In some embodiments, the anti-cholestatic agent is a PPAR agonist. In some embodiments, the PPAR agonist is bezafibrate, seladelpar, or elafibrinor.

In some embodiments, the anti-cholestatic agent is a PPAR-alpha agonist. In some embodiments, the PPAR-alpha agonist is fenofibrate.

In some embodiments, the anti-cholestatic agent is a PPAR-delta agonist. In some embodiments, the PPAR-delta agonist is seladelpar.

In some embodiments, the anti-cholestatic agent is a dual PPAR-alpha and PPAR-delta agonist.

In some embodiments, the dual PPAR-alpha-delta agonist is elafibranor.

In some embodiments, the one or more anti-cholestatic agent is an ASBT inhibitor. In some embodiments, the ASBT inhibitor is odevixibat, maralixibat, or linerixibat.

In some embodiments, the anti-cholestatic agent is an immunomodulatory drug. In some embodiments, the immunomodulatory drug is rituximab, abatacept, ustekinumab, infliximab, baricitinib, or FFP104.

In some embodiments, the anti-cholestatic agent is an antifibrotic therapy. In some embodiments, the antifibrotic therapy is a vitamin D receptor (VDR) agonist or simtuzumab.

In some embodiments, the anti-cholestatic agent is a NOX inhibitor. In some embodiments, the NOX inhibitor is setanaxib.

In some embodiments, a therapeutically effective amount of a viral vector comprising a transgene encoding MTM1 (e.g., resamirigene bilparvovec) and an anti-cholestatic agent are administered to a patient in need thereof. In some embodiments, a therapeutically effective amount of resamirigene bilparvovec and an anti-cholestatic agent are administered to a patient in need thereof. In some embodiments, a therapeutically effective amount of resamirigene bilparvovec and an anti-cholestatic agent are administered to a patient in need thereof, wherein the anti-cholestatic agent is a bile acid. In some embodiments, a therapeutically effective amount of resamirigene bilparvovec and an anti-cholestatic agent are administered to a patient in need thereof, wherein the anti-cholestatic agent is ursodiol. In some embodiments, a therapeutically effective amount of resamirigene bilparvovec and ursodiol are administered to a patient in need thereof.

### Anti-Cholestatic Agent

Using the compositions and methods of the disclosure, a patient having a neuromuscular disorder (e.g., XLMTM) may be administered an AAV vector containing a transgene encoding MTM1 and an anti-cholestatic agent.

In some embodiments, a patient is administered an anti-cholestatic agent.

In some embodiments, a patient is administered an anti-cholestatic when a patient is monitored for cholestasis, hyperbilirubinemia, or one or more symptoms thereof and it is determined that the patient exhibits cholestasis or hyperbilirubinemia or one or more symptoms thereof.

In some embodiments, a patient is administered an anti-cholestatic when it is determined that the patient exhibits cholestasis or hyperbilirubinemia or one or more symptoms thereof.

In some embodiments, the anti-cholestatic agent is selected from the list comprising a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor.

In some embodiments, the anti-cholestatic agent is a bile acid. In some embodiments, the bile acid is ursodeoxycholic acid or a derivative thereof or nor-ursodeoxycholic acid. In some embodiments, the bile acid is ursodiol.

In some embodiments, the anti-cholestatic agent is an FXR ligand. In some embodiments, the FXR ligand is obeticholic acid, cilofexor, tropifexor, tretinoin, or EDP-305.

In some embodiments, the one or more anti-cholestatic agent is an FGF-19 mimetic. In some embodiment, the FGF-19 mimetic is aldafermin.

In some embodiments, the anti-cholestatic agent is a TGR5 agonist. In some embodiments, the TGR5 agonist is INT-777 or INT-767.

In some embodiments, the anti-cholestatic agent is a PPAR agonist. In some embodiments, the PPAR agonist is bezafibrate, seladelpar, or elafibrinor.

In some embodiments, the anti-cholestatic agent is a PPAR-alpha agonist. In some embodiments, the PPAR-alpha agonist is fenofibrate.

In some embodiments, the anti-cholestatic agent is a PPAR-delta agonist. In some embodiments, the PPAR-delta agonist is seladelpar.

In some embodiments, the anti-cholestatic agent is a dual PPAR-alpha and PPAR-delta agonist.

In some embodiments, the dual PPAR-alpha-delta agonist is elafibranor.

In some embodiments, the one or more anti-cholestatic agent is an ASBT inhibitor. In some embodiments, the ASBT inhibitor is odevixibat, maralixibat, or linerixibat.

In some embodiments, the anti-cholestatic agent is an immunomodulatory drug. In some embodiments, the immunomodulatory drug is rituximab, abatacept, ustekinumab, infliximab, baricitinib, or FFP104.

In some embodiments, the anti-cholestatic agent is an antifibrotic therapy. In some embodiments, the antifibrotic therapy is a vitamin D receptor (VDR) agonist or simtuzumab.

In some embodiments, the anti-cholestatic agent is a NOX inhibitor. In some embodiments, the NOX inhibitor is setanaxib.

### I. Bile Acid

Using the methods described herein, a bile acid can be administered to the subject. In some embodiments, the bile acid is ursodeoxycholic acid or a derivative thereof or nor-ursodeoxycholic acid. In some embodiments, the bile acid is ursodiol.

Ursodiol and other known variants have the genus structure depicted below:

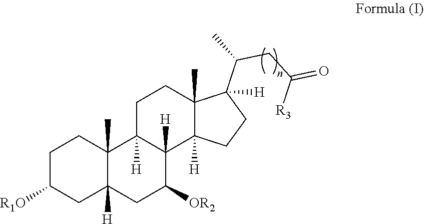

wherein each of R₁ and R₂ is, independently, hydrogen, optionally substituted alkyl, optionally substituted alkenyl, optionally substituted alkynyl, optionally substituted cycloalkyl, optionally substituted heterocyclyl, optionally substituted aryl, or optionally substituted heteroaryl;
R₃ is OR₄, NHR₄, or SR₄;
R₄ is hydrogen, optionally substituted alkyl, optionally substituted alkenyl, optionally substituted alkynyl, optionally substituted cycloalkyl, optionally substituted heterocyclyl, optionally substituted aryl, or optionally substituted heteroaryl; and
n is an integer from 0 to 4,
or a pharmaceutically acceptable salt thereof.

Such compounds are described in, e.g., U.S. Pat. No. 4,828,763, the disclosure of which is incorporated herein by reference.

### Dosing Regimens Involving a Bile Acid

The bile acid described herein may be administered in an amount of from about 5 mg/kg/dose to about 20 mg/kg/dose (e.g., in an amount of from about 5 mg/kg/dose to about 20 mg/kg/dose). For example, the bile acid may be administered to the patient in an amount of about 5 mg/kg/dose, 5.1 mg/kg/dose, 5 mg/kg/dose, 5.1 mg/kg/dose, 5.2 mg/kg/dose, 5.3 mg/kg/dose, 5.4 mg/kg/dose, 5.5 mg/kg/dose, 6 mg/kg/dose, 6.5 mg/kg/dose, 7 mg/kg/dose, 8 mg/kg/dose, 9 mg/kg/dose, 10 mg/kg/dose, 11 mg/kg/dose, 12 mg/kg/dose, 13 mg/kg/dose, 14 mg/kg/dose, 15 mg/kg/dose, 16 mg/kg/dose, 17 mg/kg/dose, 18 mg/kg/dose, 19 mg/kg/dose, or 20 mg/kg/dose.

For example, in some embodiments, the bile acid is administered to the patient in an amount of from about 5 mg/kg/dose to about 11 mg/kg/dose, such as in an amount of about 5 mg/kg/dose, 5.1 mg/kg/dose, 5.2 mg/kg/dose, 5.3 mg/kg/dose, 5.4 mg/kg/dose, 5.5 mg/kg/dose, 5.6 mg/kg/dose, 5.7 mg/kg/dose, 5.8 mg/kg/dose, 5.9 mg/kg/dose, 6 mg/kg/dose, 6.1 mg/kg/dose, 6.2 mg/kg/dose, 6.3 mg/kg/dose, 6.4 mg/kg/dose, 6.5 mg/kg/dose, 6.6 mg/kg/dose, 6.7 mg/kg/dose, 6.8 mg/kg/dose, 6.9 mg/kg/dose, 7 mg/kg/dose, 7.1 mg/kg/dose, 7.2 mg/kg/dose, 7.3 mg/kg/dose, 7.4 mg/kg/dose, 7.5 mg/kg/dose, 7.6 mg/kg/dose, 7.7 mg/kg/dose, 7.8 mg/kg/dose, 7.9 mg/kg/dose, 8 mg/kg/dose, 8.1 mg/kg/dose, 8.2 mg/kg/dose, 8.3 mg/kg/dose, 8.4 mg/kg/dose, 8.5 mg/kg/dose, 8.6 mg/kg/dose, 8.7 mg/kg/dose, 8.8 mg/kg/dose, 8.9 mg/kg/dose, 9 mg/kg/dose, 9.1 mg/kg/dose, 9.2 mg/kg/dose, 9.3 mg/kg/dose, 9.4 mg/kg/dose, 9.5 mg/kg/dose, 9.6 mg/kg/dose, 9.7 mg/kg/dose, 9.8 mg/kg/dose, 9.9 mg/kg/dose, 10 mg/kg/dose, 10.1 mg/kg/dose, 10.2 mg/kg/dose, 10.3 mg/kg/dose, 10.4 mg/kg/dose, 10.5 mg/kg/dose, 10.6 mg/kg/dose, 10.7 mg/kg/dose, 10.8 mg/kg/dose, 10.9 mg/kg/dose, or 11 mg/kg/dose.

In some embodiments, the bile acid is administered to the patient in a single dose.

In some embodiments, the bile acid is administered to the patient in a plurality of doses.

In some embodiments, the bile acid is administered to the patient in one or more doses per day (one dose per day, two doses per day, three doses per day, four doses per day, five doses per day, six doses per day, seven doses per day, eight doses per day, nine doses per day, and ten doses per day), week (one dose per week, two doses per week, three doses per week, four doses per week, five doses per week, six doses per week, seven doses per week, eight doses per week, nine doses per week, and ten doses per week, eleven doses per week, twelve doses per week, thirteen doses per week, and fourteen doses per week), or month (one dose per month, two doses per month, three doses per month, four doses per month, five doses per month, six doses per month, seven doses per month, eight doses per month, nine doses per month, and ten doses per month, eleven doses per month, twelve doses per month, thirteen doses per month, fourteen doses per month, fifteen doses per month, sixteen doses per month, seventeen doses per month, eighteen doses per month, nineteen doses per month, twenty doses per month, twenty one doses per month, twenty two doses per month, twenty three doses per month, twenty four doses per month, twenty five doses per month, twenty six doses per month, twenty seven doses per month, twenty eight doses per month, twenty nine doses per month, and thirty doses per month).

For example, in some embodiments, the bile acid is administered to the patient in one or more doses per day, such as in one dose per day, two doses per day, three doses per day, four doses per day, five doses per day, six doses per day, seven doses per day, eight doses per day, nine doses per day, or ten doses per day.

In some embodiments, the two or more doses of the bile acid that, together, total the specified amount are separated from one another, for example, by an hour or more. In some embodiments, the two or more doses are administered to the patient within about 24 hours of one another (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours of one another).

In some embodiments, the bile acid is administered to the patient in one dose per day, two doses per day, three doses per day, four doses per day, or five doses per day.

In some embodiments, the bile acid is administered to the patient in one dose per day.

In some embodiments, ursodiol is administered in an amount of from about 5 mg/kg/day to about 40 mg/kg/day (e.g., in an amount of from about 5 mg/kg/day to about 40 mg/kg/day). For example, ursodiol may be administered to the patient in an amount of about 5 mg/kg/day, 5.1 mg/kg/day, 5 mg/kg/day, 5.1 mg/kg/day, 5.2 mg/kg/day, 5.3 mg/kg/day, 5.4 mg/kg/day, 5.5 mg/kg/day, 6 mg/kg/day, 6.5 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 25 mg/kg/day, 30 mg/kg/day, 35 mg/kg/day, or 40 mg/kg/day.

For example, in some embodiments, ursodiol is administered to the patient in an amount of from about 15 mg/kg/day to about 25 mg/kg/day, such as in an amount of about 15 mg/kg/day, 15.1 mg/kg/day, 15.2 mg/kg/day, 15.3 mg/kg/day, 15.4 mg/kg/day, 15.5 mg/kg/day, 15.6 mg/kg/day, 15.7 mg/kg/day, 15.8 mg/kg/day, 15.9 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, or 25 mg/kg/day.

For example, in some embodiments, ursodiol is administered to the patient in an amount of from about 16 mg/kg/day to about 24 mg/kg/day, such as in an amount of about 16 mg/kg/day, 16.1 mg/kg/day, 16.2 mg/kg/day, 16.3 mg/kg/day, 16.4 mg/kg/day, 16.5 mg/kg/day, 16.6 mg/kg/day, 16.7 mg/kg/day, 16.8 mg/kg/day, 16.9 mg/kg/day, 17 mg/kg/day, 18 mg/kg/day, 19 mg/kg/day, 20 mg/kg/day, 21 mg/kg/day, 22 mg/kg/day, 23 mg/kg/day, or 24 mg/kg/day.

For example, in some embodiments, ursodiol is administered to the patient in an amount of from about 17 mg/kg/day to about 23 mg/kg/day, such as in an amount of about 17 mg/kg/day, 17.1 mg/kg/day, 17.2 mg/kg/day, 17.3 mg/kg/day, 17.4 mg/kg/day, 17.5 mg/kg/day, 17.6 mg/kg/day, 17.7 mg/kg/day, 17.8 mg/kg/day, 17.9 mg/kg/day, 18 mg/kg/day, 19 mg/kg/day, 20 mg/kg/day, 21 mg/kg/day, 22 mg/kg/day, or 23 mg/kg/day.

For example, in some embodiments, ursodiol is administered to the patient in an amount of from about 18 mg/kg/day to about 22 mg/kg/day, such as in an amount of about 18 mg/kg/day, 18.1 mg/kg/day, 18.2 mg/kg/day, 18.3 mg/kg/day, 18.4 mg/kg/day, 18.5 mg/kg/day, 18.6 mg/kg/day, 18.7 mg/kg/day, 18.8 mg/kg/day, 18.9 mg/kg/day, 19 mg/kg/day, 20 mg/kg/day, 21 mg/kg/day, or 22 mg/kg/day.

For example, in some embodiments, ursodiol is administered to the patient in an amount of from about 19 mg/kg/day to about 21 mg/kg/day, such as in an amount of about 19 mg/kg/day, 19.1 mg/kg/day, 19.2 mg/kg/day, 19.3 mg/kg/day, 19.4 mg/kg/day, 19.5 mg/kg/day, 19.6 mg/kg/day, 19.7 mg/kg/day, 19.8 mg/kg/day, 19.9 mg/kg/day, 20 mg/kg/day, 20.1 mg/kg/day, 20.2 mg/kg/day, 20.3 mg/kg/day, 20.4 mg/kg/day, 20.5 mg/kg/day, 20.6 mg/kg/day, 20.7 mg/kg/day, 20.8 mg/kg/day, 20.9 mg/kg/day, or 21 mg/kg/day.

In some embodiments, ursodiol is administered to the patient in an amount of 20 mg/kg/day.

In some embodiments, ursodiol is administered to the patient in one or more doses per week, such as in one dose per week, two doses per week, three doses per week, four doses per week, five doses per week, ten doses per week, fifteen doses per week, twenty doses per week, thirty doses per week, fifty doses per week, sixty doses per week, and seventy doses per week.

In some embodiments, ursodiol is administered to the patient in one or more doses per month, such as in one dose per month, two doses per month, three doses per month, four doses per month, five doses per month, ten doses per month, fifteen doses per month, twenty doses per month, thirty doses per month, fifty doses per month, sixty doses per month, seventy doses per month, eighty doses per month, ninety doses per month, one hundred doses per month, two hundred doses per month, and three hundred doses per month.

In some embodiments, ursodiol is administered to the patient by way of a unit dosage form comprising 250 mg of the ursodiol.

In some embodiments, ursodiol is administered to the patient by way of a unit dosage form comprising 500 mg of the ursodiol.

Ia. Nor-Ursodeoxycholic Acid

Using the methods described herein, nor-Ursodeoxycholic acid can be administered to the subject.

Nor-Ursodeoxycholic acid is the INN for the compound with the chemical structure depicted below.

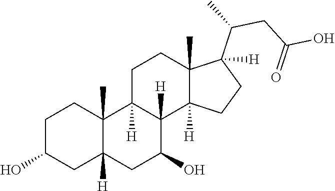

Ib. Ursodeoxycholic Acid and Derivatives

Using the methods described herein, ursodeoxycholic acid or a derivative thereof can be administered to the subject.

Ursodeoxycholic acid is the INN for the compound also known as 128-13-2 and the drug name ursodiol.

The chemical structure of ursodiol is depicted below.

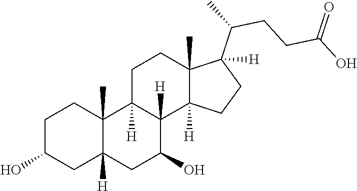

Ibi. Dosing Regimens Involving Ursodiol

Ursodiol described herein may be administered in an amount of from about 5 mg/kg/dose to about 20 mg/kg/dose (e.g., in an amount of from about 5 mg/kg/dose to about 20 mg/kg/dose). For example, ursodiol may be administered to the patient in an amount of about 5 mg/kg/dose, 5.1 mg/kg/dose, 5 mg/kg/dose, 5.1 mg/kg/dose, 5.2 mg/kg/dose, 5.3 mg/kg/dose, 5.4 mg/kg/dose, 5.5 mg/kg/dose, 6 mg/kg/dose, 6.5 mg/kg/dose, 7 mg/kg/dose, 8 mg/kg/dose, 9 mg/kg/dose, 10 mg/kg/dose, 11 mg/kg/dose, 12 mg/kg/dose, 13 mg/kg/dose, 14 mg/kg/dose, 15 mg/kg/dose, 16 mg/kg/dose, 17 mg/kg/dose, 18 mg/kg/dose, 19 mg/kg/dose, or 20 mg/kg/dose.

For example, in some embodiments, ursodiol is administered to the patient in an amount of from about 5 mg/kg/dose to about 11 mg/kg/dose, such as in an amount of about 5 mg/kg/dose, 5.1 mg/kg/dose, 5.2 mg/kg/dose, 5.3 mg/kg/dose, 5.4 mg/kg/dose, 5.5 mg/kg/dose, 5.6 mg/kg/dose, 5.7 mg/kg/dose, 5.8 mg/kg/dose, 5.9 mg/kg/dose, 6 mg/kg/dose, 6.1 mg/kg/dose, 6.2 mg/kg/dose, 6.3 mg/kg/dose, 6.4 mg/kg/dose, 6.5 mg/kg/dose, 6.6 mg/kg/dose, 6.7 mg/kg/dose, 6.8 mg/kg/dose, 6.9 mg/kg/dose, 7 mg/kg/dose, 7.1 mg/kg/dose, 7.2 mg/kg/dose, 7.3 mg/kg/dose, 7.4 mg/kg/dose, 7.5 mg/kg/dose, 7.6 mg/kg/dose, 7.7 mg/kg/dose, 7.8 mg/kg/dose, 7.9 mg/kg/dose, 8 mg/kg/dose, 8.1 mg/kg/dose, 8.2 mg/kg/dose, 8.3 mg/kg/dose, 8.4 mg/kg/dose, 8.5 mg/kg/dose, 8.6 mg/kg/dose, 8.7 mg/kg/dose, 8.8 mg/kg/dose, 8.9 mg/kg/dose, 9 mg/kg/dose, 9.1 mg/kg/dose, 9.2 mg/kg/dose, 9.3 mg/kg/dose, 9.4 mg/kg/dose, 9.5 mg/kg/dose, 9.6 mg/kg/dose, 9.7 mg/kg/dose, 9.8 mg/kg/dose, 9.9 mg/kg/dose, 10 mg/kg/dose, 10.1 mg/kg/dose, 10.2 mg/kg/dose, 10.3 mg/kg/dose, 10.4 mg/kg/dose, 10.5 mg/kg/dose, 10.6 mg/kg/dose, 10.7 mg/kg/dose, 10.8 mg/kg/dose, 10.9 mg/kg/dose, or 11 mg/kg/dose.

In some embodiments, ursodiol is administered to the patient in a single dose.

In some embodiments, ursodiol is administered to the patient in a plurality of doses.

In some embodiments, the ursodiol is administered to the patient in one or more doses per day (one dose per day, two doses per day, three doses per day, four doses per day, five doses per day, six doses per day, seven doses per day, eight doses per day, nine doses per day, and ten doses per day), week (one dose per week, two doses per week, three doses per week, four doses per week, five doses per week, six doses per week, seven doses per week, eight doses per week, nine doses per week, and ten doses per week, eleven doses per week, twelve doses per week, thirteen doses per week, and fourteen doses per week), or month (one dose per month, two doses per month, three doses per month, four doses per month, five doses per month, six doses per month, seven doses per month, eight doses per month, nine doses per month, and ten doses per month, eleven doses per month, twelve doses per month, thirteen doses per month, fourteen doses per month, fifteen doses per month, sixteen doses per month, seventeen doses per month, eighteen doses per month, nineteen doses per month, twenty doses per month, twenty one doses per month, twenty two doses per month, twenty three doses per month, twenty four doses per month, twenty five doses per month, twenty six doses per month, twenty seven doses per month, twenty eight doses per month, twenty nine doses per month, and thirty doses per month).

For example, in some embodiments, ursodiol is administered to the patient in one or more doses per day, such as in one dose per day, two doses per day, three doses per day, four doses per day, five doses per day, six doses per day, seven doses per day, eight doses per day, nine doses per day, or ten doses per day.

In some embodiments, the two or more doses of ursodiol that, together, total the specified amount are separated from one another, for example, by an hour or more. In some embodiments, the two or more doses are administered to the patient within about 24 hours of one another (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours of one another).

In some embodiments, ursodiol is administered to the patient in one dose per day, two doses per day, three doses per day, four doses per day, or five doses per day.

In some embodiments, ursodiol is administered to the patient in one dose per day.

In some embodiments, ursodiol is administered in an amount of from about 5 mg/kg/day to about 40 mg/kg/day (e.g., in an amount of from about 5 mg/kg/day to about 40 mg/kg/day). For example, ursodiol may be administered to the patient in an amount of about 5 mg/kg/day, 5.1 mg/kg/day, 5 mg/kg/day, 5.1 mg/kg/day, 5.2 mg/kg/day, 5.3 mg/kg/day, 5.4 mg/kg/day, 5.5 mg/kg/day, 6 mg/kg/day, 6.5 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 25 mg/kg/day, 30 mg/kg/day, 35 mg/kg/day, or 40 mg/kg/day.

For example, in some embodiments, ursodiol is administered to the patient in an amount of from about 15 mg/kg/day to about 25 mg/kg/day, such as in an amount of about 15 mg/kg/day, 15.1 mg/kg/day, 15.2 mg/kg/day, 15.3 mg/kg/day, 15.4 mg/kg/day, 15.5 mg/kg/day, 15.6 mg/kg/day, 15.7 mg/kg/day, 15.8 mg/kg/day, 15.9 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, or 25 mg/kg/day.

For example, in some embodiments, ursodiol is administered to the patient in an amount of from about 16 mg/kg/day to about 24 mg/kg/day, such as in an amount of about 16 mg/kg/day, 16.1 mg/kg/day, 16.2 mg/kg/day, 16.3 mg/kg/day, 16.4 mg/kg/day, 16.5 mg/kg/day, 16.6 mg/kg/day, 16.7 mg/kg/day, 16.8 mg/kg/day, 16.9 mg/kg/day, 17 mg/kg/day, 18 mg/kg/day, 19 mg/kg/day, 20 mg/kg/day, 21 mg/kg/day, 22 mg/kg/day, 23 mg/kg/day, or 24 mg/kg/day.

For example, in some embodiments, ursodiol is administered to the patient in an amount of from about 17 mg/kg/day to about 23 mg/kg/day, such as in an amount of about 17 mg/kg/day, 17.1 mg/kg/day, 17.2 mg/kg/day, 17.3 mg/kg/day, 17.4 mg/kg/day, 17.5 mg/kg/day, 17.6 mg/kg/day, 17.7 mg/kg/day, 17.8 mg/kg/day, 17.9 mg/kg/day, 18 mg/kg/day, 19 mg/kg/day, 20 mg/kg/day, 21 mg/kg/day, 22 mg/kg/day, or 23 mg/kg/day.

For example, in some embodiments, ursodiol is administered to the patient in an amount of from about 18 mg/kg/day to about 22 mg/kg/day, such as in an amount of about 18 mg/kg/day, 18.1 mg/kg/day, 18.2 mg/kg/day, 18.3 mg/kg/day, 18.4 mg/kg/day, 18.5 mg/kg/day, 18.6 mg/kg/day, 18.7 mg/kg/day, 18.8 mg/kg/day, 18.9 mg/kg/day, 19 mg/kg/day, 20 mg/kg/day, 21 mg/kg/day, or 22 mg/kg/day.

For example, in some embodiments, ursodiol is administered to the patient in an amount of from about 19 mg/kg/day to about 21 mg/kg/day, such as in an amount of about 19 mg/kg/day, 19.1 mg/kg/day, 19.2 mg/kg/day, 19.3 mg/kg/day, 19.4 mg/kg/day, 19.5 mg/kg/day, 19.6 mg/kg/day, 19.7 mg/kg/day, 19.8 mg/kg/day, 19.9 mg/kg/day, 20 mg/kg/day, 20.1 mg/kg/day, 20.2 mg/kg/day, 20.3 mg/kg/day, 20.4 mg/kg/day, 20.5 mg/kg/day, 20.6 mg/kg/day, 20.7 mg/kg/day, 20.8 mg/kg/day, 20.9 mg/kg/day, or 21 mg/kg/day.

In some embodiments, ursodiol is administered to the patient in an amount of 20 mg/kg/day.

In some embodiments, ursodiol is administered to the patient in one or more doses per week, such as in one dose per week, two doses per week, three doses per week, four doses per week, five doses per week, ten doses per week, fifteen doses per week, twenty doses per week, thirty doses per week, fifty doses per week, sixty doses per week, and seventy doses per week.

In some embodiments, ursodiol is administered to the patient in one or more doses per month, such as in one dose per month, two doses per month, three doses per month, four doses per month, five doses per month, ten doses per month, fifteen doses per month, twenty doses per month, thirty doses per month, fifty doses per month, sixty doses per month, seventy doses per month, eighty doses per month, ninety doses per month, one hundred doses per month, two hundred doses per month, and three hundred doses per month.

In some embodiments, ursodiol is administered to the patient by way of a unit dosage form comprising 250 mg of the ursodiol.

In some embodiments, ursodiol is administered to the patient by way of a unit dosage form comprising 500 mg of the ursodiol.

### II. FXR Ligand

Using the methods described herein, an FXR ligand can be administered to the subject. In some embodiments, the FXR ligand is obeticholic acid, cilofexor, tropifexor, tretinoin, or EDP-305.

IIa. Obeticholic Acid

Using the methods described herein, obeticholic acid can be administered to the subject. Obeticholic acid is the INN for the compound also known by the code name of INT-747. Obeticholic acid has the chemical structure depicted below.

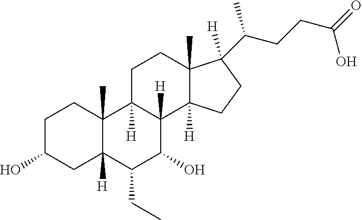

IIb. Cilofexor

Using the methods described herein, cilofexor can be administered to the subject. Cilofexor is the INN for the compound also known by the code name of GS-9674. Cilofexor has the chemical structure depicted below.

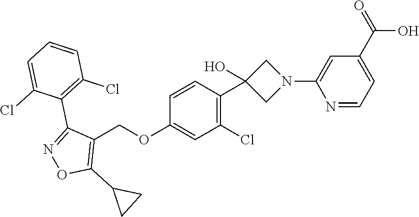 [SMILES: O=C(O)c1ccnc(N2CC(O)(c3ccc(OCc4c(-c5c(Cl)cccc5Cl)noc4C4CC4)cc3Cl)C2)c1]

IIc. Tropifexor

Using the methods described herein, tropifexor can be administered to the subject. Tropifexor is the INN for the compound also known by the code name of LJN452. Tropifexor has the chemical structure depicted below.

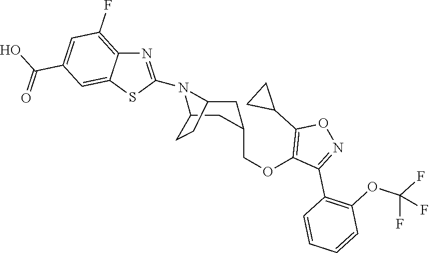 [SMILES: O=C(O)c1cc(F)c2nc(N3C4CCC3CC(COc3c(-c5ccccc5OC(F)(F)F)noc3C3CC3)C4)sc2c1]

IId. Tretinoin

Using the methods described herein, tretinoin can be administered to the subject. Tretinoin is the INN for the compound also known by the code name of 302-79-4. Retinoin has the chemical structure depicted below.

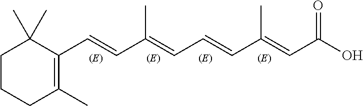 [SMILES: CC1=C(/C=C/C(C)=C/C=C/C(C)=C/C(=O)O)C(C)(C)CCC1]

IIe. EDP-305 Using the methods described herein, EDP-305 can be administered to the subject. EDP-305 is the code name of compound with the chemical structure depicted below.

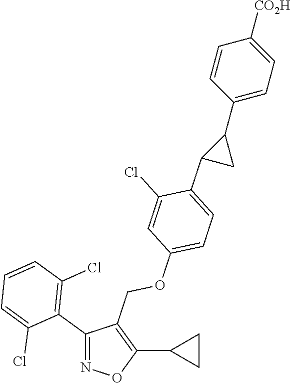 [SMILES: O=C(O)c1ccc(C2CC2c2ccc(OCc3c(-c4c(Cl)cccc4Cl)noc3C3CC3)cc2Cl)cc1]

iii. Fibroblast Growth Factor 19 (FGF-19) Mimetic

Using the methods described herein, an FGF-19 mimetic can be administered to the subject. In some embodiment, the FGF-19 mimetic is aldafermin.

IIIa. Aldafermin

Using the methods described herein, aldafermin can be administered to the subject. Aldafermin is the INN for the compound also known by the code name of NGM282 and the chemical formula of C₉₄₀—H₁₄₇₂N₂₆₆O₂₇₉S₁₁.

### IV. Takeda-G-Protein-Receptor-5 (TGR5) Agonist

Using the methods described herein, a TGR5 agonist can be administered to the subject. In some embodiments, the TGR5 agonist is INT-777 or INT-767.

IVa. INT-777

Using the methods described herein, INT-777 can be administered to the subject. INT-777 is the code name of compound also known by the name of S-EMCA.

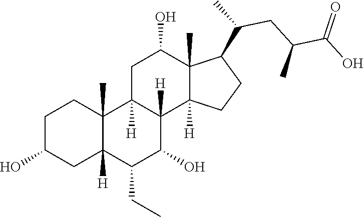

IVb. INT-767

Using the methods described herein, INT-767 can be administered to the subject. INT-767 is the code name of compound with the chemical structure depicted below.

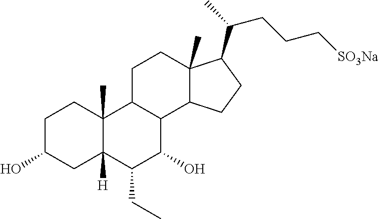

### V. Peroxisome Proliferator-Activated Receptor (PPAR) Agonist

Using the methods described herein, a PPAR agonist can be administered to the subject. In some embodiments, the PPAR agonist is bezafibrate, seladelpar, or elafibrinor.

Va. Bezafibrate

Using the methods described herein, Bezafibrate can be administered to the subject. Bezafibrate is the INN for the compound also known by the code name of C10AB02. Bezafibrate has the chemical structure depicted below.

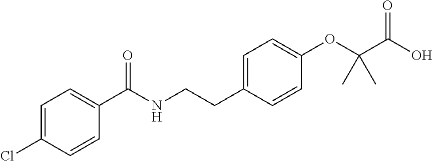 [SMILES: CC(C)(Oc1ccc(CCNC(=O)c2ccc(Cl)cc2)cc1)C(=O)O]

Vb. Seladelpar

Using the methods described herein, seladelpar can be administered to the subject. Seladelpar is the INN for the compound also known by the code name of MBX-8025. Seladelpar has the chemical structure depicted below.

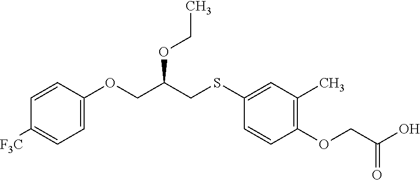 [SMILES: CCO[C@H](COc1ccc(C(F)(F)F)cc1)CSc1ccc(OCC(=O)O)c(C)c1]

Vc. Elafibrinor Using the methods described herein, elafibrinor can be administered to the subject. Elafibrinor is the INN for the compound also known by the code name of GFT505. Elafibrinor has the chemical structure depicted below.

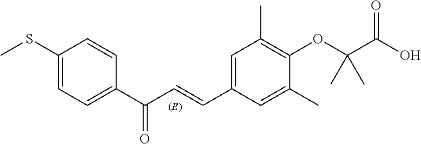 [SMILES: CSc1ccc(C(=O)/C=C/c2cc(C)c(OC(C)(C)C(=O)O)c(C)c2)cc1]

### VI. PPAR-Alpha Agonist

Using the methods described herein, a PPAR-alpha agonist can be administered to the subject.

In some embodiments, the PPAR-alpha agonist is fenofibrate.

VIa. Fenofibrate

Using the methods described herein, fenofibrate can be administered to the subject. Fenofibrate is the INN for the compound with the chemical structure depicted below.

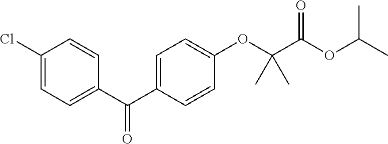 [SMILES: CC(C)OC(=O)C(C)(C)Oc1ccc(C(=O)c2ccc(Cl)cc2)cc1]

### VII. PPAR-Delta Agonist

Using the methods described herein, a PPAR-delta agonist can be administered to the subject.

In some embodiments, the PPAR-delta agonist is seladelpar.

VIIa. Seladelpar

Using the methods described herein, seladelpar can be administered to the subject. Seladelpar is the INN for the compound also known by the code name of MBX-8025. Seladelpar has the chemical structure depicted below.

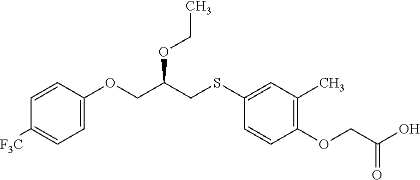 [SMILES: CCO[C@H](COc1ccc(C(F)(F)F)cc1)CSc1ccc(OCC(=O)O)c(C)c1]

### VIII. Dual PPAR-Alpha and PPAR-Delta Agonist

Using the methods described herein, a dual PPAR-alpha and PPAR-delta agonist can be administered to the subject. In some embodiments, the dual PPAR-alpha-delta agonist is elafibranor.

VIIIa. Elafibranor

Using the methods described herein, elafibrinor can be administered to the subject. Elafibrinor is the INN for the compound also known by the code name of GFT505. Elafibrinor has the chemical structure depicted below.

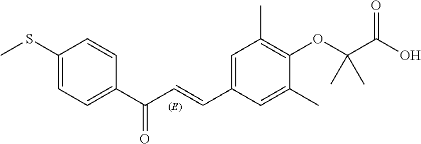 [SMILES: CSc1ccc(C(=O)/C=C/c2cc(C)c(OC(C)(C)C(=O)O)c(C)c2)cc1]

### IX. Apical Sodium-Dependent Bile Acid Transporter (ASBT) Inhibitor

Using the methods described herein, an ASBT inhibitor can be administered to the subject. In some embodiments, the ASBT inhibitor is odevixibat, maralixibat, or linerixibat.

IXa. Odevixibat

Using the methods described herein, odevixibat can be administered to the subject. Odevixibat is the INN for the compound also known by the code name of A4250. Odevixibat has the chemical structure depicted below.

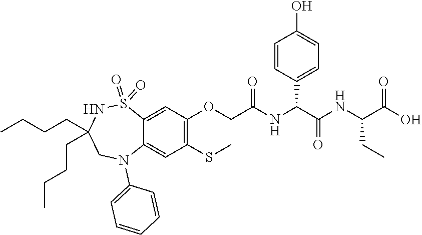 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

IXb. Maralixibat

Using the methods described herein, maralixibat can be administered to the subject. Maralixibat is the INN for the compound with the chemical structure depicted below.

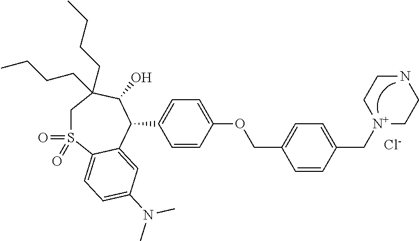 [SMILES: CCCCC1(CCCC)CS(=O)(=O)c2ccc(N(C)C)cc2[C@@H](c2ccc(OCc3ccc(C[NH+]4CCNCC4)cc3)cc2)[C@H]1O ; [Cl-]]

IXc. Linerixibat

Using the methods described herein, linerixibat can be administered to the subject. Linerixibat is the INN for the compound with the chemical structure depicted below.

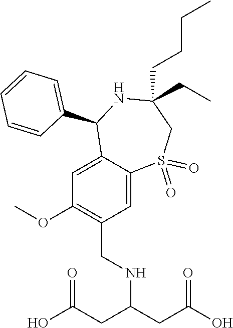 [SMILES: CCCC[C@]1(CC)CS(=O)(=O)c2cc(CNC(CC(=O)O)CC(=O)O)c(OC)cc2[C@@H](c2ccccc2)N1]

### X. Immunomodulatory Drug

Using the methods described herein, an immunomodulatory drug can be administered to the subject. In some embodiments, the immunomodulatory drug is rituximab, abatacept, ustekinumab, infliximab, baricitinib, or FFP104.

Xa. Rituximab

Using the methods described herein, rituximab can be administered to the subject. Rituximab is the INN for the antibody with the chemical formula C₆₄₁₆-H₉₈₇₄-N₁₆₈₈-O₁₉₈₇-S₄₄.

Xb. Abatacept

Using the methods described herein, abatacept can be administered to the subject. Abatacept is the INN for the antibody with the chemical formula C₃₄₉₈H₅₄₅₈N₉₂₂O₁₀₉₀S₃₂.

Xc. Ustekinumab

Using the methods described herein, ustekinumab can be administered to the subject. Ustekinumab is the INN for the antibody with the chemical formula C₆₄₈₂H₁₀₀₀₄N₁₇₁₂O₂₀₁₆S₄₆.

Xd. Infliximab

Using the methods described herein, infliximab can be administered to the subject. Infliximab is the INN for the antibody with the chemical formula C₆₄₂₈H₉₉₁₂N₁₆₉₄O₁₉₈₇S₄₆.

Xe. Baricitinib

Using the methods described herein, baricitinib can be administered to the subject. Baricitinib is the INN for the compound with the chemical structure depicted below.

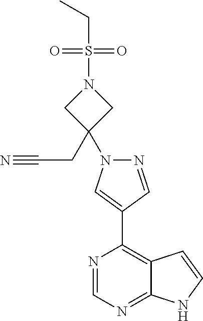 [SMILES: CCS(=O)(=O)N1CC(CC#N)(n2cc(-c3ncnc4[nH]ccc34)cn2)C1]

Xf. FFP104

Using the methods described herein, FFP104 can be administered to the subject. FFP104 is an anti-CD40 monoclonal antibody.

### XI. Antifibrotic Therapy

Using the methods described herein, an antifibrotic therapy can be administered to the subject. In some embodiments, the antifibrotic therapy is a vitamin D receptor (VDR) agonist or simtuzumab.

XIa. VDR Agonist

Using the methods described herein, a VDR agonist can be administered to the subject. Exemplary VDR agonists include but are not limited to the compounds known by the INN names of seocalcitol, elocalcitol, and calcipotriol.

XIai. Seocalcitol

Using the methods described herein, seocalcitol can be administered to the subject. Seocalcitol is the INN for the compound with the chemical structure depicted below.

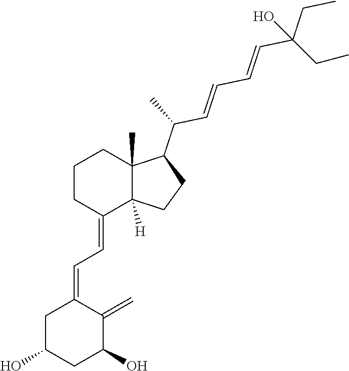 [SMILES: C=C1/C(=C\C=C2/CCC[C@]3(C)[C@@H]([C@H](C)/C=C/C=C/C(O)(CC)CC)CC[C@@H]23)C[C@@H](O)C[C@@H]1O]

XIaii. Elocalcitol

Using the methods described herein, elocalcitol can be administered to the subject. Elocalcitol is INN for the compound with the chemical structure depicted below.

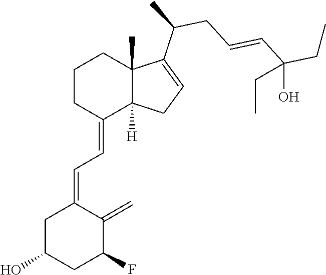 [SMILES: C=C1/C(=C\C=C2/CCC[C@]3(C)C([C@@H](C)C/C=C/C(O)(CC)CC)=CC[C@@H]23)C[C@@H](O)C[C@@H]1F]

XIaiii. Calcipotriol

Using the methods described herein, calcipotriol can be administered to the subject. Calcipotriol is INN for the compound with the chemical structure depicted below.

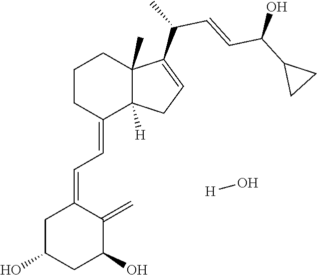

XIb. Simtuzumab

Using the methods described herein, simtuzumab can be administered to the subject.

Simtuzumab is the INN for the antibody also known by the code name of GS-6624 and with the chemical formula of C₆₅₅₈H₁₀₁₃₄N₁₇₃₆O₂₀₃₇S₅₀.

### XII. Nicotinamide Adenine Dinucleotide Phosphate Oxidase (NOX) Inhibitor

Using the methods described herein, a NOX inhibitor can be administered to the subject. In some embodiments, the NOX inhibitor is setanaxib.

XIIa. Setanaxib

Using the methods described herein, setanaxib can be administered to the subject. Setanaxib is the INN for the compound known by the code name of GKT831. Setanaxib has the chemical structure depicted below.

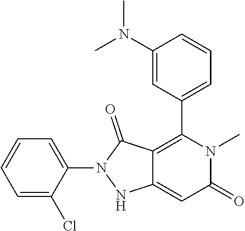 [SMILES: CN(C)c1cccc(-c2c3c(=O)n(-c4ccccc4Cl)[nH]c3cc(=O)n2C)c1]

### Recommended Clinical Parameters for Monitoring a Patient for Development of Cholestasis or Hyperbilirubinemia

In some embodiments, a patient is monitored for the development of cholestasis by a serum bile acid test and/or blood test (e.g., an LFT), as descried herein.

In some embodiments, a patient is monitored for the development of hyperbilirubinemia by a blood test (e.g., bilirubin test), as descried herein.

In some embodiments, a patient is monitored for the development of cholestasis, and if the patient exhibits cholestasis or one or more symptoms thereof, the patient is administered an anti-cholestatic agent.

In some embodiments, a patient is monitored for the development of hyperbilirubinemia, and if the patient exhibits hyperbilirubinemia or one or more symptoms thereof, the patient is administered an anti-cholestatic agent.

In some embodiments, a patient is monitored for the development of cholestasis or hyperbilirubinemia by a blood test (e.g., a serum acid bile test or a liver function test). In some embodiments, a patient is monitored for the development of cholestasis or hyperbilirubinemia by a blood test (e.g., a serum acid bile test or a liver function test), and if the patient exhibits cholestasis or hyperbilirubinemia or one or more symptoms thereof, the patient is administered an anti-cholestatic agent.

In some embodiments, the patient is determined to exhibit cholestasis, hyperbilirubinemia, or one or more symptoms thereof by a finding that the patient exhibits a parameter (e.g., a serum bile acid level) in blood test (e.g., a serum acid bile test) that is increased relative to a reference level.

In some embodiments, the patient is determined to exhibit cholestasis, hyperbilirubinemia, or one or more symptoms thereof by a finding that the patient exhibits a serum bile acid (e.g., cholic acid, chenodeoxycholic acid, deoxycholic acid, or ursodeoxycholic acid) level in blood test (e.g., a serum acid bile test) that is increased relative to a reference level.

In some embodiments, the blood test is a liver function test.

In some embodiments, a patient is monitored for the development of cholestasis or hyperbilirubinemia by a liver function test, and if the patient exhibits cholestasis or hyperbilirubinemia or one or more symptoms thereof, the patient is administered an anti-cholestatic agent.

In some embodiments, the patient is determined to exhibit cholestasis, hyperbilirubinemia, or one or more symptoms thereof by a finding that the patient exhibits a parameter (e.g., aspartate aminotransferase level or alanine aminotransferase level) in liver function test that is increased or decreased relative to a reference level.

### I. Serum Bile Acid Test

In some embodiments, a patient is monitored for the development of cholestasis or hyperbilirubinemia. In some embodiments, a patient is monitored for the development of cholestasis or hyperbilirubinemia with a serum bile acid test. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia with a serum bile acid test, and if the patient exhibits cholestasis or hyperbilirubinemia or one or more symptoms thereof, the patient is administered an anti-cholestatic agent.

In some embodiments, a patient is monitored for cholestasis or hyperbilirubinemia by the patient's bile acid (e.g., cholic acid, chenodeoxycholic acid, deoxycholic acid, or ursodeoxycholic acid) levels, as measured with a serum bile acid test.

In some embodiments, it is determined that a patient exhibits cholestasis, hyperbilirubinemia, or one or more symptoms thereof and is administered an anti-cholestatic agent when one or more of the patient's bile acid (e.g., cholic acid, chenodeoxycholic acid, deoxycholic acid, or ursodeoxycholic acid) levels, as measured with a serum bile acid test, is greater than the norm.

In some embodiments, it is determined that a patient exhibits cholestasis, hyperbilirubinemia, or one or more symptoms thereof when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's ursodeoxycholic acid level, as measured with a serum bile acid test, is greater than 2 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's ursodeoxycholic acid level, as measured with a serum bile acid test, is greater than 5 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

### II. Liver Function Test

In some embodiments, a patient is monitored for the development of cholestasis or hyperbilirubinemia with an LFT. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia with an LFT, and if the patient exhibits cholestasis or hyperbilirubinemia or one or more symptoms thereof, the patient is administered an anti-cholestatic agent.

In some embodiments, it is determined that a patient exhibits cholestasis, hyperbilirubinemia, or one or more symptoms thereof and is administered an anti-cholestatic agent when a parameter (e.g., ASP level or AST level) of the patient's LFT is greater than the age-adjusted norm, as descried herein.

IIa. Aspartate Aminotransferase

In some embodiments, a patient is monitored for the development of cholestasis or hyperbilirubinemia by measuring the patient's AST level in an LFT. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia by measuring the patient's AST level in an LFT, and if the patient exhibits cholestasis or hyperbilirubinemia or one or more symptoms thereof, the patient is administered an anti-cholestatic agent.

In some embodiments, a patient is monitored for the development of cholestasis or hyperbilirubinemia by measuring the patient's AST level in an LFT and it is determined that a patient exhibits cholestasis, hyperbilirubinemia, or one or more symptoms thereof and is administered an anti-cholestatic agent when the patient's AST level is greater than the norm.

In some embodiments, it is determined that a patient exhibits cholestasis, hyperbilirubinemia, or one or more symptoms thereof when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis, hyperbilirubinemia, or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIb. Alanine Aminotransferase

In some embodiments, a patient is monitored for the development of cholestasis or hyperbilirubinemia by measuring the patient's ALT level in an LFT. In some embodiments, a patient is monitored for the development of cholestasis, hyperbilirubinemia, or one or more symptoms thereof, and if the patient exhibits cholestasis or hyperbilirubinemia or one or more symptoms thereof, the patient is administered an anti-cholestatic agent. In some embodiments, a patient is monitored for the development of cholestasis or hyperbilirubinemia by measuring the patient's ALT level in an LFT, and if the patient exhibits cholestasis or hyperbilirubinemia or one or more symptoms thereof, the patient is administered an anti-cholestatic agent.

In some embodiments, a patient is monitored for the development of cholestasis or hyperbilirubinemia by measuring the patient's ALT level in an LFT and it is determined that a patient exhibits cholestasis, hyperbilirubinemia, or one or more symptoms thereof and is administered an anti-cholestatic agent when the patient's ALT level is greater than the norm.

In some embodiments, it is determined that a patient exhibits cholestasis or one or more symptoms thereof when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Recommended Clinical Parameters for Monitoring a Patient for Development of Cholestasis

### I. Serum Bile Acid Test

In some embodiments, a patient is monitored for the development of cholestasis. In some embodiments, a patient is monitored for the development of cholestasis with a serum bile acid test. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis with a serum bile acid test, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when the patient's total bile acids level, as measured with a serum bile acid test, is greater than the norm.

In some embodiments, it is determined that a patient exhibits cholestasis or one or more symptoms thereof and is administered an anti-cholestatic agent when the patient's total bile acids level, as measured with a serum bile acid test, is greater than 14 μmol/L (e.g., 15 μmol/L, 16 μmol/L, 17 μmol/L, 18 μmol/L, 19 μmol/L, 20 μmol/L, 21 μmol/L, 22 μmol/L, 23 μmol/L, 24 μmol/L, 25 μmol/L, 26 μmol/L, 27 μmol/L, 28 μmol/L, 29 μmol/L, 30 μmol/L, 31 μmol/L, 32 μmol/L, 33 μmol/L, 34 μmol/L, 35 μmol/L, 36 μmol/L, 37 μmol/L, 38 μmol/L, 39 μmol/L, 40 μmol/L, 41 μmol/L, 42 μmol/L, 43 μmol/L, 44 μmol/L, 45 μmol/L, 46 μmol/L, 47 μmol/L, 48 μmol/L, 49 μmol/L, 50 μmol/L, 51 μmol/L, 52 μmol/L, 53 μmol/L, 54 μmol/L, 55 μmol/L, 56 μmol/L, 57 μmol/L, 58 μmol/L, 59 μmol/L, 60 μmol/L, 61 μmol/L, 62 μmol/L, 63 μmol/L, 64 μmol/L, 65 μmol/L, 66 μmol/L, 67 μmol/L, 68 μmol/L, 69 μmol/L, 70 μmol/L, 71 μmol/L, 72 μmol/L, 73 μmol/L, 74 μmol/L, 75 μmol/L, 76 μmol/L, 77 μmol/L, 78 μmol/L, 79 μmol/L, 80 μmol/L, 81 μmol/L, 82 μmol/L, 83 μmol/L, 84 μmol/L, 85 μmol/L, 86 μmol/L, 87 μmol/L, 88 μmol/L, 89 μmol/L, 90 μmol/L, 91 μmol/L, 92 μmol/L, 93 μmol/L, 94 μmol/L, 95 μmol/L, 96 μmol/L, 97 μmol/L, 98 μmol/L, 99 μmol/L, and 100 μmol/L).

### II. Blood Test

In some embodiments, a patient is monitored for the development of cholestasis with a blood test (e.g., LFT or a bilirubin test). In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis with an LFT, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when one or more parameters (e.g., GGT level, ASP level, AST level, ALT level, and bilirubin level) of the patient's blood test (e.g., a LFT or a bilirubin test) is greater than the age-adjusted norm, as descried herein.

IIa. Liver Function Test

In some embodiments, a patient is monitored for the development of cholestasis with an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis with an LFT, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when one or more parameters (e.g., GGT level, ASP level, AST level, and ALT level) of the patient's LFT is greater than the age-adjusted norm, as descried herein.

IIai. Gamma-Glutamyl Transferase

In some embodiments, a patient is monitored for the development of cholestasis by measuring the patient's GGT level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's GGT level in an LFT, and if the patient exhibits cholestasis or one or more symptoms thereof, the patient is administered an anti-cholestatic agent.

In some embodiments, it is determined that a patient exhibits cholestasis or one or more symptoms thereof and is administered an anti-cholestatic agent when the patient exhibits a GGT level that is greater than the age-adjusted norm.

In some embodiments, the patient is a newborn (e.g., 0-6 months old), a toddler (e.g., 6-12 months old), or a child aged 1-5 years old. In some embodiments, the patient is a newborn of the age from 0-6 months old. In some embodiments, the patient is a toddler of the age from 6-12 months old. In some embodiments, the patient is a child of the age from 1-5 years old.

In some embodiments, a patient is a newborn (e.g., 0-6 months old) and it is determined that a patient exhibits cholestasis or one or more symptoms thereof and is administered an anti-cholestatic agent when the patient's GGT level is outside of the normal range of about 12-122 U/L (e.g., 12-122 U/L, 13-122 U/L, 14-122 U/L, 15-122 U/L, 16-122 U/L, 17-122 U/L, 18-122 U/L, 19-122 U/L, 20-122 U/L, 25-122 U/L, 30-122 U/L, 40-122 U/L, 50-122 U/L, 60-122 U/L, 70-122 U/L, 80-122 U/L, 90-122 U/L, 100-122 U/L, 110-122 U/L, 120-122 U/L, or 121-122 U/L).

In some embodiments, the patient is a male newborn (e.g., 0-6 months old) and it is determined that a patient exhibits cholestasis or one or more symptoms thereof and is administered an anti-cholestatic agent when the patient's GGT level is less than 12 U/L (e.g., 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a male newborn (e.g., 0-6 months old) and it is determined that a patient exhibits cholestasis or one or more symptoms thereof and is administered an anti-cholestatic agent when the patient's GGT level is greater than 122 U/L (e.g., 123 U/L, 124 U/L, 125 U/L, 126 U/L, 127 U/L, 128 U/L, 129 U/L, 130 U/L, 135 U/L, 140 U/L, 150 U/L, 160 U/L, 170 U/L, 180 U/L, 190 U/L, and 200 U/L).

In some embodiments, the patient is a male toddler (e.g., 6-12 months old) and it is determined that a patient exhibits cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a male toddler (e.g., 6-12 months old) and it is determined that a patient exhibits cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a male child aged 1-5 years old and it is determined that a patient exhibits cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a male child aged 1-5 years old and it is determined that a patient exhibits cholestasis or one or more symptoms thereof and is administered an anti-cholestatic agent when the patient is GGT level is less than 3 U/L (e.g., 2 U/L and 1 U/L).

In some embodiments, the patient is a male child aged 1-5 years old and it is determined that a patient exhibits cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female newborn (e.g., 0-6 months old) and it is determined that a patient exhibits cholestasis or one or more symptoms thereof and is administered an anti-cholestatic agent when the patient's GGT level is outside of the normal range of about 15-132 U/L (e.g., 15-132 U/L, 16-132 U/L, 17-132 U/L, 18-132 U/L, 19-132 U/L, 20-132 U/L, 25-132 U/L, 30-132 U/L, 40-132 U/L, 50-132 U/L, 60-132 U/L, 70-132 U/L, 80-132 U/L, 90-132 U/L, 100-132 U/L, 110-132 U/L, 120-132 U/L, 130-132 U/L, and 131-132 U/L).

In some embodiments, the patient is a female newborn (e.g., 0-6 months old) and it is determined that a patient exhibits cholestasis or one or more symptoms thereof and is administered an anti-cholestatic agent when the patient's GGT level is less than 15 U/L (e.g., 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a female newborn (e.g., 0-6 months old) and it is determined that a patient exhibits cholestasis or one or more symptoms thereof and is administered an anti-cholestatic agent when the patient's GGT level is greater than 132 U/L (e.g., 133 U/L, 134 U/L, 135 U/L, 136 U/L, 137 U/L, 138 U/L, 139 U/L, 140 U/L, 145 U/L, 150 U/L, 160 U/L, 170 U/L, 180 U/L, 190 U/L, and 200 U/L).

In some embodiments, the patient is a female toddler (e.g., 6-12 months old) it is determined that a patient exhibits cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a female toddler (e.g., 6-12 months old) and it is determined that a patient exhibits cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female child aged 1-5 years old it is determined that a patient exhibits cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a female child aged 1-5 years old and it is determined that a patient exhibits cholestasis or one or more symptoms thereof and is administered an anti-cholestatic agent when the patient's GGT level is less than 3 U/L (e.g., 2 U/L and 1 U/L).

In some embodiments, the patient is a female child aged 1-5 years old and it is determined that a patient exhibits cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

IIaii. Alkaline Phosphatase

In some embodiments, a patient is monitored for the development of cholestasis by measuring the patient's ASP level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's ASP level in an LFT, and if the patient exhibits cholestasis or one or more symptoms thereof, the patient is administered an anti-cholestatic agent.

In some embodiments, a patient is monitored for the development of cholestasis by measuring the patient's ASP level in an LFT and it is determined that a patient exhibits cholestasis or one or more symptoms thereof and is administered an anti-cholestatic agent when the patient's ASP level that is greater than the norm.

In some embodiments, it is determined that a patient exhibits cholestasis or one or more symptoms thereof and is administered an anti-cholestatic agent when the patient's ASP level is outside of the normal range of about 50 to 300 U/L e.g., about 51 to 300 U/L, about 52 to U/L, about 53 to 300 U/L, about 54 to 300 U/L, about 55 to 300 U/L, about 56 to 300 U/L, about 57 to 300 U/L, about 58 to 300 U/L, about 59 to 300 U/L, about 60 to 300 U/L, about 65 to 300 U/L, about 70 to 300 U/L, about 80 to 300 U/L, about 90 to 300 U/L, about 100 to 300 U/L, about 125 to 300 U/L, about 150 to 300 U/L, about 175 to 300 U/L, about 200 to 300 U/L, about 225 to 300 U/L, about 250 to 300 U/L, or about 275 to 300 U/L).

In some embodiments, it is determined that a patient exhibits cholestasis or one or more symptoms thereof and is administered an anti-cholestatic agent when the patient's ASP level is less than 50 U/L (e.g., 50 U/L, 49 U/L, 48 U/L, 47 U/L, 46 U/L, 45 U/L, 44 U/L, 43 U/L, 42 U/L, 41 U/L, 40 U/L, 39 U/L, 38 U/L, 37 U/L, 36 U/L, 35 U/L, 34 U/L, 33 U/L, 32 U/L, 31 U/L, 30 U/L, 29 U/L, 28 U/L, 27 U/L, 26 U/L, 25 U/L, 24 U/L, 23 U/L, 22 U/L, 21 U/L, 20 U/L, 19 U/L, 18 U/L, 17 U/L, 16 U/L, 15 U/L, 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, and 1 U/L).

In some embodiments, it is determined that a patient exhibits cholestasis or one or more symptoms thereof and is administered an anti-cholestatic agent when the patient's ASP level is greater than 300 U/L (e.g., 300 U/L, 301 U/L, 302 U/L, 303 U/L, 304 U/L, 305 U/L, 306 U/L, 307 U/L, 308 U/L, 309 U/L, 310 U/L, 311 U/L, 312 U/L, 313 U/L, 314 U/L, 315 U/L, 316 U/L, 317 U/L, 318 U/L, 319 U/L, 320 U/L, 321 U/L, 322 U/L, 323 U/L, 324 U/L, 325 U/L, 330 U/L, 340 U/L, 350 U/L, 400 U/L, and 500 U/L).

IIaiii. Aspartate Aminotransferase

In some embodiments, a patient is monitored for the development of cholestasis by measuring the patient's AST level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's AST level in an LFT, and if the patient exhibits cholestasis or one or more symptoms thereof, the patient is administered an anti-cholestatic agent.

In some embodiments, a patient is monitored for the development of cholestasis by measuring the patient's AST level in an LFT and it is determined that a patient exhibits cholestasis or one or more symptoms thereof and is administered an anti-cholestatic agent when the patient's AST level is greater than the norm.

In some embodiments, it is determined that a patient exhibits cholestasis or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIaiv. Alanine Aminotransferase

In some embodiments, a patient is monitored for the development of cholestasis by measuring the patient's ALT level in an LFT. In some embodiments, a patient is monitored for the development of cholestasis, and if the patient exhibits cholestasis or one or more symptoms thereof, the patient is administered an anti-cholestatic agent. In some embodiments, a patient is monitored for the development of cholestasis by measuring the patient's ALT level in an LFT, and if the patient exhibits cholestasis or one or more symptoms thereof, the patient is administered an anti-cholestatic agent.

In some embodiments, a patient is monitored for the development of cholestasis by measuring the patient's ALT level in an LFT and it is determined that a patient exhibits cholestasis or one or more symptoms thereof and is administered an anti-cholestatic agent when the patient's ALT level is greater than the norm.

In some embodiments, it is determined that a patient exhibits cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Recommended Clinical Parameters for Monitoring a Patient for Development of Hyperbilirubinemia

### Bilirubin Test

In some embodiments, a patient is monitored for the development of hyperbilirubinemia. In some embodiments, a patient is monitored for the development of hyperbilirubinemia with a bilirubin test. In some embodiments, a patient is monitored for the development of hyperbilirubinemia, and if the patient exhibits hyperbilirubinemia or one or more symptoms thereof, the patient is administered an anti-cholestatic agent. In some embodiments, a patient is monitored for the development of hyperbilirubinemia with a bilirubin test, and if the patient exhibits hyperbilirubinemia or one or more symptoms thereof, the patient is administered an anti-cholestatic agent.

In some embodiments, it is determined that a patient exhibits hyperbilirubinemia or one or more symptoms thereof and is administered an anti-cholestatic agent when the patient exhibits a bilirubin level that is greater than the norm.

In some embodiments, it is determined that a patient exhibits hyperbilirubinemia or one or more symptoms thereof and is administered an anti-cholestatic agent when the patient's total bilirubin level is greater than 1.2 mg/dL (e.g., 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, it is determined that a patient exhibits hyperbilirubinemia or one or more symptoms thereof and is administered an anti-cholestatic agent when the patient's direct bilirubin level is greater than 0.2 mg/dL (e.g., 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, the patient is determined to exhibit hyperbilirubinemia or one or more symptoms thereof and is administered an anti-cholestatic agent when the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

Recommended Clinical Parameters for Determining that a Patient Exhibits Cholestasis or Hyperbilirubinemia or a Symptom Thereof

In some embodiments, it is determined that the patient exhibits cholestasis, hyperbilirubinemia, or one or more symptoms thereof by determining that one or more parameters (e.g., total bile acids level, GGT level, ASP level, AST level, and ALT level) of the patient's serum bile acid test and/or blood test (e.g., an LFT) is greater than or less than the age-adjusted norm, as descried herein, and the patient is administered an anti-cholestatic agent.

In some embodiments, it is determined that the patient exhibits cholestasis, hyperbilirubinemia, or one or more symptoms thereof by determining that one or more parameters (e.g., bilirubin level) of the patient's blood test (e.g., bilirubin test) is greater than the norm, as descried herein, and the patient is administered an anti-cholestatic agent.

In some embodiments, it is determined that a patient exhibits cholestasis, hyperbilirubinemia, or one or more symptoms thereof and is administered an anti-cholestatic agent when one or more of the patient's bile acid (e.g., cholic acid, chenodeoxycholic acid, deoxycholic acid, or ursodeoxycholic acid) levels, as measured with a serum bile acid test, is greater than the norm.

In some embodiments, it is determined that a patient exhibits cholestasis, hyperbilirubinemia, or one or more symptoms thereof and is administered an anti-cholestatic agent when a parameter (e.g., ASP level or AST level) of the patient's LFT is greater than the age-adjusted norm, as descried herein.

### I. Serum Bile Acid Test

In some embodiments, it is determined that a patient exhibits cholestasis, hyperbilirubinemia, or one or more symptoms thereof and is administered an anti-cholestatic agent when one or more of the patient's bile acid (e.g., cholic acid, chenodeoxycholic acid, deoxycholic acid, or ursodeoxycholic acid) levels, as measured with a serum bile acid test, is greater than the norm.

In some embodiments, it is determined that a patient exhibits cholestasis, hyperbilirubinemia, or one or more symptoms thereof when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's cholic acid level, as measured with a serum bile acid test, is greater than 5 nmol/mL (e.g., 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof and is administered an anti-cholestatic agent when the patient's chenodeoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL).

In some embodiments, it is determined that a patient exhibits cholestasis, hyperbilirubinemia, or one or more symptoms thereof when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's deoxycholic acid level, as measured with a serum bile acid test, is greater than 6 nmol/mL (e.g., 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof when the patient's ursodeoxycholic acid level, as measured with a serum bile acid test, is greater than 2 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

In some embodiments, it is determined that a patient exhibits cholestasis, hyperbilirubinemia, or one or more symptoms thereof and is administered an anti-cholestatic agent when the patient's ursodeoxycholic acid level, as measured with a serum bile acid test, is greater than 5 nmol/mL (e.g., 2 nmol/mL, 3 nmol/mL, 4 nmol/mL, 5 nmol/mL, 6 nmol/mL, 7 nmol/mL, 8 mol/mL, 9 nmol/mL, 10 nmol/mL, 15 nmol/mL, 20 nmol/mL, 30 nmol/mL, 40 nmol/mL, 50 nmol/mL, 60 nmol/mL, 70 nmol/mL, 80 nmol/mL, 90 nmol/mL, and 100 nmol/mL.

### II. Liver Function Test

In some embodiments, it is determined that a patient exhibits cholestasis, hyperbilirubinemia, or one or more symptoms thereof and is administered an anti-cholestatic agent when a parameter (e.g., ASP level or AST level) of the patient's LFT is greater than the age-adjusted norm, as descried herein.

IIa. Aspartate Aminotransferase

In some embodiments, a patient is monitored for the development of cholestasis or hyperbilirubinemia by measuring the patient's AST level in an LFT and it is determined that a patient exhibits cholestasis, hyperbilirubinemia, or one or more symptoms thereof and is administered an anti-cholestatic agent when the patient's AST level is greater than the norm.

In some embodiments, it is determined that a patient exhibits cholestasis, hyperbilirubinemia, or one or more symptoms thereof when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis, hyperbilirubinemia, or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIb. Alanine Aminotransferase

In some embodiments, a patient is monitored for the development of cholestasis or hyperbilirubinemia by measuring the patient's ALT level in an LFT and it is determined that a patient exhibits cholestasis, hyperbilirubinemia, or one or more symptoms thereof and is administered an anti-cholestatic agent when the patient's ALT level is greater than the norm.

In some embodiments, it is determined that a patient exhibits cholestasis or one or more symptoms thereof when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

Recommended Clinical Parameters for Determining that a Patient Exhibits Cholestasis or a Symptom Thereof

### I. Serum Bile Acid Test

In some embodiments, a patient is determined to exhibit cholestasis or one or more symptoms thereof and is administered an anti-cholestatic agent when the patient exhibits an acid bile level, as measured with a serum bile acid test, that is greater than the norm.

In some embodiments, a patient is determined to exhibit cholestasis or one or more symptoms thereof and is administered an anti-cholestatic agent when the patient's total bile acids level, as measured determined with a serum bile acid test, is greater than 14 μmol/L (e.g., 15 μmol/L, 16 μmol/L, 17 μmol/L, 18 μmol/L, 19 μmol/L, 20 μmol/L, 21 μmol/L, 22 μmol/L, 23 μmol/L, 24 μmol/L, 25 μmol/L, 26 μmol/L, 27 μmol/L, 28 μmol/L, 29 μmol/L, 30 μmol/L, 31 μmol/L, 32 μmol/L, 33 μmol/L, 34 μmol/L, 35 μmol/L, 36 μmol/L, 37 μmol/L, 38 μmol/L, 39 μmol/L, 40 μmol/L, 41 μmol/L, 42 μmol/L, 43 μmol/L, 44 μmol/L, 45 μmol/L, 46 μmol/L, 47 μmol/L, 48 μmol/L, 49 μmol/L, 50 μmol/L, 51 μmol/L, 52 μmol/L, 53 μmol/L, 54 μmol/L, 55 μmol/L, 56 μmol/L, 57 μmol/L, 58 μmol/L, 59 μmol/L, 60 μmol/L, 61 μmol/L, 62 μmol/L, 63 μmol/L, 64 μmol/L, 65 μmol/L, 66 μmol/L, 67 μmol/L, 68 μmol/L, 69 μmol/L, 70 μmol/L, 71 μmol/L, 72 μmol/L, 73 μmol/L, 74 μmol/L, 75 μmol/L, 76 μmol/L, 77 μmol/L, 78 μmol/L, 79 μmol/L, 80 μmol/L, 81 μmol/L, 82 μmol/L, 83 μmol/L, 84 μmol/L, 85 μmol/L, 86 μmol/L, 87 μmol/L, 88 μmol/L, 89 μmol/L, 90 μmol/L, 91 μmol/L, 92 μmol/L, 93 μmol/L, 94 μmol/L, 95 μmol/L, 96 μmol/L, 97 μmol/L, 98 μmol/L, 99 μmol/L, and 100 μmol/L).

### II. Blood Test

In some embodiments, a patient is determined to exhibit cholestasis or one or more symptoms thereof and is administered an anti-cholestatic agent when one or more parameters (e.g., GGT level, ASP level, AST level, ALT level, and bilirubin level) of the patient's blood test (e.g., a LFT or a bilirubin test) is greater than the age-adjusted norm, as descried herein.

IIa. Liver Function Test

In some embodiments, a patient is determined to exhibit cholestasis or one or more symptoms thereof and is administered an anti-cholestatic agent when one or more parameters (e.g., GGT level, ASP level, AST level, and ALT level) of the patient's LFT is greater than the age-adjusted norm, as descried herein.

IIai. Gamma-Glutamyl Transferase

In some embodiments, a patient is determined to exhibit cholestasis or one or more symptoms thereof and is administered an anti-cholestatic agent when the patient exhibits a GGT level, as measured in a LFT, that is greater than the age-adjusted norm.

In some embodiments, the patient is a newborn (e.g., 0-6 months old), a toddler (e.g., 6-12 months old), or a child aged 1-5 years old. In some embodiments, the patient is a newborn of the age from 0-6 months old. In some embodiments, the patient is a toddler of the age from 6-12 months old. In some embodiments, the patient is a child of the age from 1-5 years old.

In some embodiments, a patient is a newborn (e.g., 0-6 months old) and is determined to exhibit cholestasis or one or more symptoms thereof and is administered an anti-cholestatic agent when the patient's GGT level is outside of the normal range of about 12-122 U/L (e.g., 12-122 U/L, 13-122 U/L, 14-122 U/L, 15-122 U/L, 16-122 U/L, 17-122 U/L, 18-122 U/L, 19-122 U/L, 20-122 U/L, 25-122 U/L, 30-122 U/L, 40-122 U/L, 50-122 U/L, 60-122 U/L, 70-122 U/L, 80-122 U/L, 90-122 U/L, 100-122 U/L, 110-122 U/L, 120-122 U/L, or 121-122 U/L).

In some embodiments, the patient is a male newborn (e.g., 0-6 months old) and is determined to exhibit cholestasis or one or more symptoms thereof and is administered an anti-cholestatic agent when the patient's GGT level is less than 12 U/L (e.g., 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a male newborn (e.g., 0-6 months old) and is determined to exhibit cholestasis or one or more symptoms thereof and is administered an anti-cholestatic agent when the patient's GGT level is greater than 122 U/L (e.g., 123 U/L, 124 U/L, 125 U/L, 126 U/L, 127 U/L, 128 U/L, 129 U/L, 130 U/L, 135 U/L, 140 U/L, 150 U/L, 160 U/L, 170 U/L, 180 U/L, 190 U/L, and 200 U/L).

In some embodiments, the patient is a male toddler (e.g., 6-12 months old) and is determined to exhibit cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a male toddler (e.g., 6-12 months old) and is determined to exhibit cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a male child aged 1-5 years old and is determined to exhibit cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a male child aged 1-5 years old and is determined to exhibit cholestasis or one or more symptoms thereof and is administered an anti-cholestatic agent when the patient's GGT level is less than about 3 U/L (e.g., 2 U/L and 1 U/L).

In some embodiments, the patient is a male child aged 1-5 years old and is determined to exhibit cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female newborn (e.g., 0-6 months old) and is determined to exhibit cholestasis or one or more symptoms thereof and is administered an anti-cholestatic agent when the patient's GGT level is outside of the normal range of about 15-132 U/L (e.g., 15-132 U/L, 16-132 U/L, 17-132 U/L, 18-132 U/L, 19-132 U/L, 20-132 U/L, 25-132 U/L, 30-132 U/L, 40-132 U/L, 50-132 U/L, 60-132 U/L, 70-132 U/L, 80-132 U/L, 90-132 U/L, 100-132 U/L, 110-132 U/L, 120-132 U/L, 130-132 U/L, and 131-132 U/L).

In some embodiments, the patient is a female newborn (e.g., 0-6 months old) and is determined to exhibit cholestasis or one or more symptoms thereof and is administered an anti-cholestatic agent when the patient's GGT level is less than about 15 U/L (e.g., 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, or 1 U/L).

In some embodiments, the patient is a female newborn (e.g., 0-6 months old) and is determined to exhibit cholestasis or one or more symptoms thereof and is administered an anti-cholestatic agent when the patient's GGT level is greater than 132 U/L (e.g., 133 U/L, 134 U/L, 135 U/L, 136 U/L, 137 U/L, 138 U/L, 139 U/L, 140 U/L, 145 U/L, 150 U/L, 160 U/L, 170 U/L, 180 U/L, 190 U/L, and 200 U/L).

In some embodiments, the patient is a female toddler (e.g., 6-12 months old) and is determined to exhibit cholestasis or one or more symptoms thereof and is administered an anti-cholestatic agent when the patient's GGT level is outside of the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

In some embodiments, the patient is a female toddler (e.g., 6-12 months old) and is determined to exhibit cholestasis or one or more symptoms thereof and is administered an anti-cholestatic agent when the patient's GGT level is greater than 39 U/L (e.g., 40 U/L, 41 U/L, 42 U/L, 43 U/L, 44 U/L, 45 U/L, 46 U/L, 47 U/L, 48 U/L, 49 U/L, 50 U/L, 55 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

In some embodiments, the patient is a female child aged 1-5 years old and is determined to exhibit cholestasis or one or more symptoms thereof and is administered an anti-cholestatic agent when the patient's GGT level is outside of the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

In some embodiments, the patient is a female child aged 1-5 years old and is determined to exhibit cholestasis or one or more symptoms thereof and is administered an anti-cholestatic agent when the patient's GGT level is less than about 3 U/L (e.g., 2 U/L and 1 U/L).

In some embodiments, the patient is a female child aged 1-5 years old and is determined to exhibit cholestasis or one or more symptoms thereof and is administered an anti-cholestatic agent when the patient's GGT level is greater than 22 U/L (e.g., 23 U/L, 24 U/L, 25 U/L, 26 U/L, 27 U/L, 28 U/L, 29 U/L, 30 U/L, 35 U/L, 40 U/L, 50 U/L, 60 U/L, 70 U/L, 80 U/L, 90 U/L, 100 U/L, 110 U/L, 1120 U/L, 130 U/L, 140 U/L, 150 U/L, 160 U/L, 170 U/L, 180 U/L, 190 U/L, and 200 U/L).

IIaii. Alkaline Phosphatase

In some embodiments, a patient is determined to exhibit cholestasis or one or more symptoms thereof and is administered an anti-cholestatic agent when the patient exhibits an ASP level, as measured in a LFT, that is greater than the norm.

In some embodiments, a patient is determined to exhibit cholestasis or one or more symptoms thereof and is administered an anti-cholestatic agent when the patient's ASP level is outside of the normal range of about 50 to 300 U/L (e.g., about 51 to 300 U/L, about 52 to U/L, about 53 to 300 U/L, about 54 to 300 U/L, about 55 to 300 U/L, about 56 to 300 U/L, about 57 to 300 U/L, about 58 to 300 U/L, about 59 to 300 U/L, about 60 to 300 U/L, about 65 to 300 U/L, about 70 to 300 U/L, about 80 to 300 U/L, about 90 to 300 U/L, about 100 to 300 U/L, about 125 to 300 U/L, about 150 to 300 U/L, about 175 to 300 U/L, about 200 to 300 U/L, about 225 to 300 U/L, about 250 to 300 U/L, or about 275 to 300 U/L).

In some embodiments, a patient is determined to exhibit cholestasis or one or more symptoms thereof and is administered an anti-cholestatic agent when the patient's ASP level is less than about 50 U/L (e.g., 50 U/L, 49 U/L, 48 U/L, 47 U/L, 46 U/L, 45 U/L, 44 U/L, 43 U/L, 42 U/L, 41 U/L, 40 U/L, 39 U/L, 38 U/L, 37 U/L, 36 U/L, 35 U/L, 34 U/L, 33 U/L, 32 U/L, 31 U/L, 30 U/L, 29 U/L, 28 U/L, 27 U/L, 26 U/L, 25 U/L, 24 U/L, 23 U/L, 22 U/L, 21 U/L, 20 U/L, 19 U/L, 18 U/L, 17 U/L, 16 U/L, 15 U/L, 14 U/L, 13 U/L, 12 U/L, 11 U/L, 10 U/L, 9 U/L, 8 U/L, 7 U/L, 6 U/L, 5 U/L, 4 U/L, 3 U/L, 2 U/L, and 1 U/L).

In some embodiments, a is determined to exhibit cholestasis or one or more symptoms thereof and is administered an anti-cholestatic agent when the patient's ASP level is greater than 300 U/L (e.g., 300 U/L, 301 U/L, 302 U/L, 303 U/L, 304 U/L, 305 U/L, 306 U/L, 307 U/L, 308 U/L, 309 U/L, 310 U/L, 311 U/L, 312 U/L, 313 U/L, 314 U/L, 315 U/L, 316 U/L, 317 U/L, 318 U/L, 319 U/L, 320 U/L, 321 U/L, 322 U/L, 323 U/L, 324 U/L, 325 U/L, 330 U/L, 340 U/L, 350 U/L, 400 U/L, and 500 U/L).

IIaiii. Aspartate Aminotransferase

In some embodiments, a patient is determined to exhibit cholestasis or one or more symptoms thereof and is administered an anti-cholestatic agent when the patient exhibits an AST level, as measured in a LFT, that is greater than the norm.

In some embodiments, a is determined to exhibit cholestasis or one or more symptoms thereof and is administered an anti-cholestatic agent when the patient's AST level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

IIaiv. Alanine Aminotransferase

In some embodiments, a patient is determined to exhibit cholestasis or one or more symptoms thereof and is administered an anti-cholestatic agent when the patient exhibits an ALT level, as measured in a LFT, that is greater than the norm.

In some embodiments, a patient is determined to exhibit cholestasis or one or more symptoms thereof and is administered an anti-cholestatic agent when the patient's ALT level is greater than 50 U/L (e.g., 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

Recommended Clinical Parameters for Determining that a Patient Exhibits Hyperbilirubinemia or a Symptom Thereof

### Bilirubin Test

In some embodiments, a patient is determined to exhibit hyperbilirubinemia or one or more symptoms thereof and is administered an anti-cholestatic agent when the patient exhibits a bilirubin level, as measured in a blood test (e.g., a bilirubin test), that is greater than the norm.

In some embodiments, a patient is determined to exhibit hyperbilirubinemia or one or more symptoms thereof and is administered an anti-cholestatic agent when the patient's total bilirubin level is greater than 1.2 mg/dL (e.g., 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, a patient is determined to exhibit hyperbilirubinemia or one or more symptoms thereof and is administered an anti-cholestatic agent when the patient's direct bilirubin level is greater than 0.2 mg/dL (e.g., 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, the patient is determined to exhibit hyperbilirubinemia or one or more symptoms thereof and is administered an anti-cholestatic agent when the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Resamirigene Bilparvovec-Emergent Hepatobiliary Disorders and Ursodiol as a Prophylaxis for Cholestatic Syndromes

The objective of this study was to examine the potential adverse effects of resamirigene bilparvovec in human patients having X-linked myotubular myopathy (XLMTM) and less than or equal to 5 years old for a period of up to five years post-dosing.

### Materials and Methods

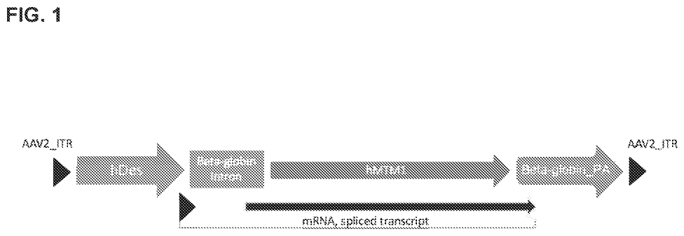

Thirty patients were enrolled on study. Twenty-three patients with having XLMTM and less than or equal to 5 years old were administered resamirigene bilparvovec (FIG. 1) at doses of 1.0×10¹⁴ vg/kg (n=6) or 3.0×10¹⁴ vg/kg (n=17). Seven controls were untreated.

Patients were monitored daily for general health status; detailed clinical observations, functional improvements, and Treatment-Emergent Adverse Events (TEAEs), for up to 27.9 months.

A summary of the groups and duration of monitorization are presented in Table 2.

TABLE 2
Duration on Study, by Dose Level of Resamirigene Bilparvovec

| | | 1.0 × 10¹⁴ vg/kg | 3.0 × 10¹⁴ vg/kg | |
|---|---|---|---|---|
| | | Resamirigene | Resamirigene | |
| | Controls | Bilparvovec | Bilparvovec | All Dosed |
| | (n = 7) | (n = 6) | (n = 17) | (n = 23) |
| Duration on Study, mean | 11.1 (2.50) | 27.9 (1.11) | 10.1 (1.4) | 14.7 (1.98) |
| months (standard error) | [5.7, 24.1] | [24.9, 31.0] | [3.4, 21.2] | [3.4, 31.0] |
| [range] | | | | |

### Results

All 23 subjects dosed in the study experienced TEAEs, defined as adverse effects (AEs) that occurred after administration of resamirigene bilparvovec. AEs reported by untreated controls during the study window were included as comparisons. Table 3 presents the most frequent TEAEs that were reported in ≥2 subjects. TEAEs occurring at a severity of Grade ≥3 in ≥2 subjects included hyperbilirubinemia/blood bilirubin increase (n=4), cholestasis (n=2), alanine aminotransferase (ALT) increase (n=2), aspartate aminotransferase (AST) increase (n=2), and gamma-glutamyl transferase (GGT) increase (n=2). Grade 1 TEAEs were defined as mild; asymptomatic or mild symptoms; clinical or diagnostic observations only; no intervention indicated. Grade 2 TEAEs were defined as moderate; minimal, local or noninvasive intervention indicated. Grade 3 TEAEs were defined as severe or medically significant but not immediately life threatening; hospitalization or prolongation of hospitalization indicated; disabling. Grade 4 TEAEs were defined as life-threatening consequences; urgent intervention indicated, while Grade 5 TEAEs were defined as death related to AE.

TABLE 3
Most Frequent TEAEs Reported in ≥2 Subjects, by Preferred Term

| | | | | 1.0 × 10¹⁴ vg/kg | | | 3.0 × 10¹⁴ vg/kg | | | | |
|---|---|---|---|---|---|---|---|---|---|---|---|
| | | | | Resamirigene | | | Resamirigene | | | | |
| | Controls | | | Bilparvovec | | | Bilparvovec | | All Dosed | | |
| | (n = 7) | | | (n = 6) | | | (n = 17) | | (n = 23) | | |
| | | Subjects | | | Subjects | | | Subjects | | Subjects | |
| Preferred Term | Events | n (%) | | Events | n (%) | | Events | n (%) | Events | n (%) | |
| Hyperbilirubinemia | 0 | 0 | (0) | 2 | 2 | (33.3) | 6 | 3 (17.6) | 8 | 5 | (21.7) |
| Transaminases | 0 | 0 | (0) | 3 | 3 | (35.0) | 4 | 3 (17.6) | 7 | 6 | (26.1) |
| increased | | | | | | | | | | | |
| Alanine | 1 | 1 | (14.3) | 1 | 1 | (16.7) | 3 | 3 (17.6) | 4 | 4 | (17.4) |
| aminotransferase | | | | | | | | | | | |
| increase | | | | | | | | | | | |
| Aspartate | 1 | 1 | (14.3) | 1 | 1 | (16.7) | 3 | 3 (17.6) | 4 | 4 | (17.4) |
| aminotransferase | | | | | | | | | | | |
| increased | | | | | | | | | | | |
| Gamma-glutamyl | 2 | 2 | (28.6) | 2 | 2 | (33.3) | 2 | 2 (11.8) | 4 | 4 | (17.4) |
| transferase | | | | | | | | | | | |
| increased | | | | | | | | | | | |
| Blood bilirubin | 1 | 1 | (14.3) | 0 | 0 | (0) | 3 | 3 (17.6) | 3 | 3 | (13.0) |
| increased | | | | | | | | | | | |
| Liver function test | 0 | 0 | (0) | 1 | 1 | (16.7) | 2 | 2 (11.8) | 3 | 3 | (13.0) |
| abnormal | | | | | | | | | | | |
| Cholestasis | 0 | 0 | (0) | 0 | 0 | (0) | 2 | 2 (11.8) | 2 | 2 | (8.7) |

A summary of TEAEs considered possibly or probably related to resamirigene bilparvovec by Investigators participating in the study is provided in Table 4.

Among others, frequently reported related TEAEs in subjects treated at the 1 0.0×10¹⁴ vg/kg dose level included elevated aminotransferases (including ALT increase, AST increase, transaminases increased, liver function test abnormal, and hepatic function abnormal) in 5 subjects (83.3%) and hyperbilirubinemia (including blood bilirubin increase) occurred in 2 subjects (33.3%). At the 1 0.0×10¹⁴ vg/kg dose level, several TEAEs were reported. Among others, GGT increased (Grade 3) in one subject, transaminases increased (Grade 4) in 1 subject, and hyperbilirubinemia occurred (Grade 3) in 1 subject.

Among others, frequently reported related TEAEs in subjects treated at the 3.0×10¹⁴ vg/kg dose level included elevated aminotransferases (including ALT increased, AST increased, transaminases increased, liver function test abnormal, and hepatic enzyme increased) in 8 subjects (47.1%) and hyperbilirubinemia (including blood bilirubin increased) occurred in 3 subjects (17.6%). At the 3.0×10¹⁴ vg/kg dose level, several TEAEs were reported. Among others, Grade 4 TEAEs included hyperbilirubinemia in one subject; hyperbilirubinemia in one subject; AST increased in one subject, ALT increased in one subject, GGT increased in one subject, and an abnormal liver function test (Grade 4) was observed in one subject.

TABLE 4
TEAEs Considered at Least Possibly Related to Resamirigene Bilparvovec
Reported in ≥2 Subjects, by System Organ Class and Preferred Term

| | 1.0 × 10¹⁴ vg/kg | | 3.0 × 10¹⁴ vg/kg | | | |
|---|---|---|---|---|---|---|
| | Resamirigene | | Resamirigene | | | |
| | Bilparvovec | | Bilparvovec | | All Dosed | |
| System Organ | (n = 6) | | (n = 17) | | (n = 23) | |
| Class Preferred | | Subjects | | Subjects | | Subjects |
| Term | Events | n (%) | Events | n (%) | Events | n (%) |

Hepatobiliary disorders

| | | | | | | | | |
|---|---|---|---|---|---|---|---|---|
| Hyperbilirubinemia | 2 | 2 | (33.3) | 5 | 2 (11.8) | 7 | 4 | (17.4) |
| Cholestasis | 0 | 0 | (0) | 2 | 2 (11.8) | 2 | 2 | (8.7) |

Investigations

| | | | | | | | | |
|---|---|---|---|---|---|---|---|---|
| Transaminases | 3 | 3 | (50.0) | 4 | 3 (17.6) | 7 | 6 | (26.1) |
| increased | | | | | | | | |
| Alanine | 1 | 1 | (16.7) | 3 | 3 (17.6) | 4 | 4 | (17.4) |
| aminotransferase | | | | | | | | |
| increased | | | | | | | | |
| Aspartate | 1 | 1 | (16.7) | 3 | 3 (17.6) | 4 | 4 | (17.4) |
| aminotransferase | | | | | | | | |
| increased | | | | | | | | |
| Gamma-glutamyl | 1 | 1 | (16.7) | 2 | 2 (11.8) | 3 | 3 | (13.0) |
| transferase | | | | | | | | |
| increased | | | | | | | | |
| Liver function test | 1 | 1 | (16.7) | 2 | 2 (11.8) | 3 | 3 | (13.0) |
| abnormal | | | | | | | | |
| Blood bilirubin | 0 | 0 | (0) | 2 | 2 (11.8) | 2 | 2 | (8.7) |

A summary of all treatment-emergent SAEs occurring in dosed subjects is presented in Table 5.

Thirty severe AEs (SAEs) in 9 subjects (1 subject at the 1.0×10¹⁴ vg/kg dose level and 8 subjects at the 3.0×10¹⁴ vg/kg dose level) were assessed as at least possibly related to resamirigene bilparvovec. Twenty-five treatment-emergent SAEs in 10 subjects were assessed as not related to resamirigene bilparvovec by the Investigator. The majority included respiratory issues that are known complications of underlying XLMTM disease, including respiratory type infections (e.g., pneumonia or respiratory tract infection; 10 events in 6 subjects).

Following review of all available clinical safety data of subjects exposed to resamirigene bilparvovec during participation in the study, events of hyperbilirubinemia and cholestasis, and elevations in AST, and ALT, were determined to be identified risks associated with resamirigene bilparvovec.

TABLE 5
Treatment-Emergent SAEs in Study, by System Organ Class and Preferred Term

| | | | 1.0 × 10¹⁴ vg/kg | | 3.0 × 10¹⁴ vg/kg | | | |
|---|---|---|---|---|---|---|---|---|
| | | | Resamirigene | | Resamirigene | | | |
| | Controls | | Bilparvovec | | Bilparvovec | | All Dosed | |
| System Organ | (n = 7) | | (n = 6) | | (n = 17) | | (n = 23) | |
| Class Preferred | | Subjects | | Subjects | | Subjects | | Subjects |
| Term | Events | n (%) | Events | n (%) | Events | n (%) | Events | n (%) |

Hepatobiliary disorders

| | | | | | | | | | |
|---|---|---|---|---|---|---|---|---|---|
| Hyperbilirubinemia | 0 | 0 (0) | 0 | 0 (0) | 4 | 2 | (11.8) | 4 | 2 (8.7) |
| Cholestasis | 0 | 0 (0) | 0 | 0 (0) | 1 | 1 | (5.9) | 1 | 1 (4.3) |

Eight subjects administered resamirigene bilparvovec at the 3.0×10¹⁴ vg/kg dose level experienced 26 SAEs considered at least possibly related to resamirigene bilparvovec. Most of these 26 SAEs were thought to be related to severe cholestatic hepatic dysfunction. All of these resamirigene bilparvovec-related SAEs are described briefly below.

Five subjects administered resamirigene bilparvovec at the 3.0×10¹⁴ vg/kg dose level experienced cases involving serious hepatobiliary events. An SAE of cholestasis was reported in 1 subject, which resolved and is briefly described below.

A 6.8-year-old subject (115-9001) experienced an SAE of Grade 3 cholestasis which required hospitalization. The subject's medical history included cholestasis and elevated total bilirubin. Approximately 5 weeks after dosing, the subject had elevated direct bilirubin levels (Grade 2) which was treated with ursodiol and resolved 6 weeks later. Approximately 11 weeks after dosing, the subject experienced cholestasis and decision was made to hospitalize the subject 9 days after cholestasis was reported. The cholestasis resolved after approximately 12 weeks. Cholestasis was considered by the Investigator to be possibly related to resamirigene bilparvovec infusion. The subject's baseline history of cholestasis supports underlying disease as causal factor. It was notable that apart from slight jaundice, the patient did not show any other signs of illness.

### CONCLUSION

At the 1.0×10¹⁴ vg/kg dose level, hepatobiliary SAEs were not observed. At the 1.0×10¹⁴ vg/kg dose level, 2 of 6 subjects reported nonserious events of hyperbilirubinemia.

At the 3.0×10¹⁴ vg/kg dose level, SAEs representing significant hepatobiliary dysfunction were observed in 4 subjects, one which led to death. More particularly, in 3 subjects reporting significantly elevated bilirubin levels, the cholestatic liver dysfunction led to fatal AEs of sepsis in 2 subjects and putatively led to fatal gastrointestinal hemorrhage in a third subject. Each of the 3 subjects had a history of hyperbilirubinemia. A fourth subject reporting an SAE of cholestasis had a pretreatment history of cholestasis, and other than slight jaundice, no clinically significant changes to hepatobiliary function were reported. SAEs of hyperbilirubinemia and cholestasis were reported at the 3.0×10¹⁴ vg/kg dose level and are considered identified risks associated with the use of resamirigene bilparvovec.

Investigations into the nature of liver dysfunction in patients with XLMTM both before and after resamirigene bilparvovec administration suggest intrahepatic cholestasis as a likely central feature.

Hyperbilirubinemia and cholestasis are reported in the natural history of the disease, though its pathophysiology is poorly understood (e.g., see Amburgey, Kimberly, et al. “A natural history study of X-linked myotubular myopathy.” Neurology 89.13 (2017): 1355-1364; Herman, Gail E., et al. “Medical complications in long-term survivors with X-linked myotubular myopathy.” J. Pediatr. 134.2 (1999): 206-214). In our natural history study, 35 subjects had evaluable hepatic laboratory data (including 1 subject who was ultimately a screen fail; mean duration on study for remaining 34 subjects of 13 months [range, 0.5-32.9]), and the majority had at least 1 value above the upper limit of normal (ULN) range (ALT level abnormal in 85.7% of patients ( 30/35), AST level abnormal in 68.6% of patients ( 24/35), GGT level abnormal in 54.3% of patients ( 19/35), total bilirubin abnormal in 31.4% of patients ( 11/35), and direct bilirubin abnormal in 28.6% of patients ( 10/35). Some subjects receiving resamirigene bilparvovec at 1.0×10¹⁴ vg/kg and 3.0×10¹⁴ vg/kg in this study have been noted to have hyperbilirubinemia at baseline or medical histories consistent with cholestasis and have not reported hepatic SAEs following dosing. While underlying XLMTM disease may contribute to events of cholestasis and hyperbilirubinemia in this population (e.g., see Herman, Gail E., et al. “Medical complications in long-term survivors with X-linked myotubular myopathy.” J. Pediatr. 134.2 (1999): 206-214), resamirigene bilparvovec is thought to contribute to events of cholestasis and hyperbilirubinemia observed to date. Therefore, cholestasis and hyperbilirubinemia are identified risks of resamirigene bilparvovec and as described herein, this invention established routine monitoring assessments regarding hepatobiliary assessments to monitor for hepatobiliary changes.

Cholestasis and hyperbilirubinemia are considered identified risks associated with resamirigene bilparvovec. Therefore, for subjects receiving resamirigene bilparvovec, ursodiol is recommended as prophylaxis for possible cholestatic syndromes. Ursodiol (ursodeoxycholic acid) is an enterally administered hydrophilic bile acid that can decrease the hydrophobic bile acid content within bile. As hydrophilic bile acids are generally non-toxic to hepatocytes, while hydrophobic bile acids can be toxic to these same cells in direct contact, ursodiol has been used to treat severe cholestatic syndromes, such as progressive intrafamilial intrahepatic cholestasis (e.g., see Balistreri. “Bile acid therapy in pediatric hepatobiliary disease: the role of ursodeoxycholic acid.” J. Pediatr. Gastroenterol. Nutr. 24.5 (1997): 573-589; Strauss, et al. “Management of hyperbilirubinemia and prevention of kernicterus in 20 patients with Crigler-Najjar disease.” Eur J Pediatr 165.5 (2006): 306-319; Suskind, et al. “A child with Kabuki syndrome and primary sclerosing cholangitis successfully treated with ursodiol and cholestryamine.” J. Pediatr. Gastroenterol. Nutr. 43.4 (2006): 542-544). This procedure has demonstrated the ability to reduce the toxic effects of elevated serum bile acid levels in certain other intrahepatic cholestatic disorders. For example, transient hyperbilirubinemia has been observed following resamirigene bilparvovec treatment and in some cases has been responsive to treatment with ursodiol.

Additional measures such as nasobiliary drainage (NBD) may be considered in the event of progressive hyperbilirubinemia that is refractory to ursodiol.

### Example 2. Post Resamirigene Bilparvovec-Dose Bilirubin Laboratory Trends

The objective of this longitudinal study was to examine the level of total and direct bilirubin values in human patients having XLMTM and less than or equal to 5 years old for 48 weeks after resamirigene bilparvovec dosing.

Materials and Methods are described in Example 1.

### Results

35 subjects had at least 1 evaluable measurement of total and direct bilirubin.

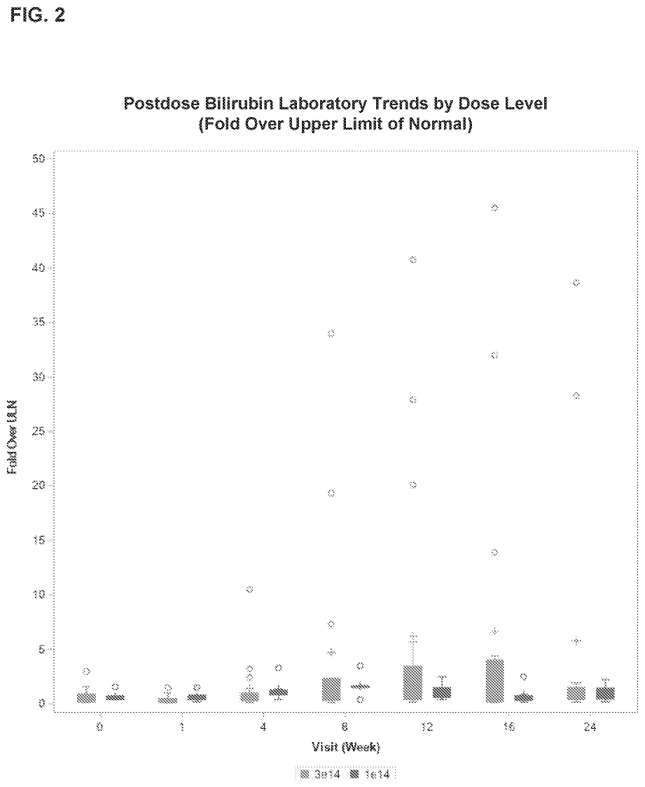

FIG. 2 is a box plot of the number of observations of patients with a total or direct bilirubin level with a fold difference over the ULN, attributed to the 1.0×10¹⁴ vg/kg or 3.0×10¹⁴ vg/kg dose level of resamirigene bilparvovec in this study. Regarding total bilirubin, 11 subjects (31.4%) had at least one result >ULN; 5 subjects (14.3%) had at least 1 result ≥2×ULN; 3 subjects (8.6%) had at least 1 value ≥3×ULN; and 2 subjects (5.7%) had at least 1 value ≥5×ULN. Among the 11 subjects with at least 1 elevated total bilirubin level, 8 (73%) also demonstrated normal values at other timepoints.

Regarding direct bilirubin, 10 subjects (28.6%) had at least 1 result >ULN; 5 subjects (14.3%) had at least 1 result ≥2×ULN; 5 subjects (14.3%) had at least one value ≥3×ULN; and 2 subjects (5.7%) had at least one value ≥5×ULN. Among the 10 subjects with at least 1 elevated direct bilirubin level, 7 (70%) also demonstrated normal values at other timepoints.

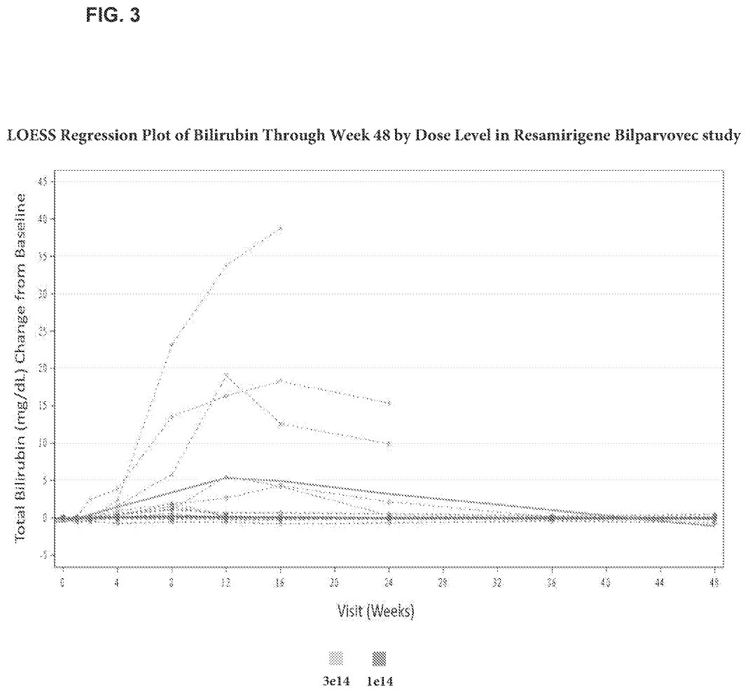

FIG. 3 is a LOESS regression plot of the same total bilirubin data through week 48.

### CONCLUSION

In summary, following resamirigene bilparvovec administration, elevation in the total hyperbilirubinemia was observed in patients having XLMTM. The absolute elevations from baseline were generally higher in the dose level of 3.0×10¹⁴ vg/kg, as compared to the 1.0×10¹⁴ vg/kg dose level.

### Example 3. Safety and Efficacy of One-Time Gene Replacement Therapy for X-Linked Myotubular Myopathy, Resamirigene Bilparvovec (ASPIRO): A Phase 1/2/3, Multinational, Randomized, Open-Label Trial

This example describes ASPIRO (NCT03199469), a Phase 1/2/3 randomized, open-label study of the safety and efficacy of resamirigene bilparvovec, a single-dose gene replacement therapy for patients with XLMTM, which is a rare, life-threatening congenital myopathy caused by mutations in the MTM1 gene resulting in profound muscle weakness, and early death.

### Introduction

Resamirigene bilparvovec is an AAV8 vector designed to deliver full-length human MTM1 complementary DNA (cDNA) to skeletal muscle under the control of the muscle-specific desmin promoter and enhancer. In mouse and dog models of XLMTM, a single administration of AAV vectors expressing the murine or canine version of the MTM1 cDNA, respectively, led to reversal of the disease phenotype and persistence of treatment effect. The ASPIRO clinical trial (NCT03199469) evaluated the safety and efficacy of a single infusion of resamirigene bilparvovec at two dose levels in children with XLMTM.

### Materials and Methods

In brief, we report the open-label ASPIRO randomized trial (NCT03199469), in which participants were enrolled and received a single intravenous dose of AAV vector delivering human MTM1, resamirigene bilparvovec. As of January 2021, six participants received 1×10¹⁴ vg/kg and 17 received 3×10¹⁴ vg/kg, which were compared with 15 untreated (control) participants. In a subsequent phase of the trial, seven participants received 1×10¹⁴ vg/kg and 17 received 3×10¹⁴ vg/kg, which were compared with 14 untreated (control) participants. Efficacy was assessed as a change from baseline to Week 48 in hours of daily ventilator support, maximal inspiratory pressure (MIP), Children's Hospital of Philadelphia Infant Test of Neuromuscular Disorders (CHOP INTEND) motor function score, and motor development assessed in each participant via a Motor Developmental Milestones assessment (10 developmental items based primarily on the Bayley Scales of Infant and Toddler Development III (Bayley III), assessing typical development, such as sitting, standing and walking unsupported).

Further materials and methods are detailed in the sections below.

### I. Study Population

ASPIRO is an ongoing two-part, multinational, randomized, open-label trial initiated in 2017 in which patients were randomized to receive a one-time intravenous administration of resamirigene bilparvovec or to delayed-treatment control. Part 1 was a safety and dose-escalation assessment. Part 2 (ongoing) is a confirmatory phase using the dose for further study identified from Part 1.

Before ASPIRO, 34 patients with XLMTM were enrolled in INCEPTUS, a prospective run-in study (NCT02704273), which informed the choice of clinically relevant endpoints to assess treatment efficacy in ASPIRO. Males <4 years of age with a genetically confirmed diagnosis of XLMTM and receiving mechanical ventilator support (invasive or noninvasive) were enrolled in INCEPTUS and followed for up to 33 months.

In ASPIRO, participants were boys <5 years of age (mean age: 20.4 months [range 9.5, 49.7]) with genetically confirmed XLMTM who required ventilator support and had no clinically significant underlying liver disease (>5× of the ULN of alanine aminotransferase or aspartate aminotransferase, or hepatic peliosis by imaging). Motor development, a secondary efficacy endpoint, was assessed in 23 resamirigene bilparvovec treated participants (n=6, lower-dose of 1×10¹⁴ vg/kg; n=17, higher-dose of 3×10¹⁴ vg/kg) and compared with 15 untreated controls (including 12 participants from INCEPTUS, NCT02704273). Specifically, untreated control participants (n=15) comprise those enrolled in INCEPTUS who did not transition to ASPIRO (n=12) and those enrolled directly in ASPIRO with or without participating in INCEPTUS (n=3) but not yet treated as of the data cutoff on Jan. 29, 2021. In a subsequent phase of the trial, one additional control participant (participant 40) was treated with the lower-dose of 1×10¹⁴ vg/kg.

Ia. Randomization and Treatment

In Part 1, participants were enrolled in one of two dose cohorts: cohort 1 (‘lower dose’) received a resamirigene bilparvovec dose of 1×10¹⁴ vector genomes (vg) per kilogram (kg) of body weight and cohort 2 (‘higher dose’) received 3×10¹⁴ vg/kg.

The first (sentinel) participant in each cohort received resamirigene bilparvovec; the absence of safety concerns 4 weeks after infusion permitted three additional participants to be randomly assigned (2:1) to immediately receive treatment at the same dose, or to a delayed-treatment control to eventually receive treatment at the dose selected for Part 2. Cohort 1 was expanded to include three additional treated participants by recommendation of the data and safety monitoring committee, for a total of six participants receiving this lower dose. Cohort 2 was then initiated, and, after expanding to include an additional five participants, ten participants in Part 1 received the higher dose.

Part 2 was initiated to confirm what appeared to be the maximal tolerated dose of 3×10¹⁴ vg/kg identified in Part 1. Based on power analysis with 80% at a 0.05 level of significance detecting a difference of at least 13 hours in ventilation requirement reduction, ten participants were enrolled, and age-matched dyads randomized (1:1) to immediate treatment or to delayed-treatment control. As of January 2021, seven participants have been dosed at the higher dose in Part 2. In total, 17 participants have received the higher dose to date.

### II. Vector

Resamirigene bilparvovec is a non-replicating recombinant AAV8 vector expressing a non-codon-optimized human MTM1 cDNA under the control of the muscle-specific human desmin promoter. The MTM1 expression cassette was built by cloning a synthetic DNA sequence complementary to the coding portion (nucleotides 43-1864) of the wild-type human MTM1 transcript (NCBI Ref. Seq NM_000252.3) downstream of the 1.05 kb human desmin enhancer/promoter region. The second intron and polyadenylation sequence of the human p-globin gene (HBB) were inserted upstream and downstream respectively of the MTM1 synthetic cDNA to mediate RNA processing. The expression cassette was flanked by AAV2 inverted terminal repeats (ITRs). The vector was produced in an AAV8 capsid by two-plasmid transfection in HEK293 cells in suspension culture in bioreactors a full GMP process.

### III. Procedures

Resamirigene bilparvovec was administered as a single dose by intravenous infusion. Participants received prednisolone (1 mg/kg) beginning 1 day before treatment to mitigate potential T-cell-mediated hepatic inflammation, which had been observed in previous trials of gene therapy with AAV vectors. The first three participants received this dose daily for 4 weeks, followed by a 4-week taper. This period was extended to 8 weeks with an 8-week taper in the remaining participants, in response to increases in aminotransferases in one participant and troponin I in another observed 7 weeks after treatment.

### IV. Assessments

The primary efficacy outcome was the change in hours of daily ventilator support from baseline through Week 48, with ventilator independence defined when participant reported 0 hours/day on ventilator. Participant responses to gradual modification of ventilator settings and eventual ventilator weaning were overseen by local pulmonologists who monitored measures of gas exchange (oxygen saturation, transcutaneous carbon dioxide levels, and serum bicarbonate levels). A normal polysomnogram, weight gain, developmental progress, and reassuring clinical examination were prerequisites to discontinue ventilation. Measurements of MIP were obtained at individual study sites and sent to a central reader to evaluate respiratory muscle strength.

Motor skills were assessed at individual study sites using the CHOP INTEND (scores range from 0 to 64 points, with higher scores indicating better motor function; a 4-point increment is considered clinically significant), and for selected major gross motor milestones using relevant items from the Bayley III, CHOP INTEND, and Motor Function Measure scale (MFM-32).

Open muscle-biopsy specimens were obtained at baseline from the left gastrocnemius, post-treatment Week 24 from the right gastrocnemius, and Week 48 from the vastus lateralis, and processed for histopathological analysis. Assessment of vector biodistribution was performed by quantitative polymerase chain reaction (qPCR) and of myotubularin RNA and protein expression by RNA sequencing and western blot, respectively. Immunologic assessments included measurement of anti-AAV8 neutralizing antibodies and anti-myotubularin antibodies in accordance with study protocol, as well as immunohistochemical stains for inflammatory markers in muscle-biopsy specimens.

AEs were recorded from the time of obtaining informed consent and coded using the Medical Dictionary for Regulatory Activities (MedDRA®), version 20.0. SAEs were defined according to International Conference on Harmonisation criteria.

IVa. Determination of Maximal Inspiratory Pressure (MIP)

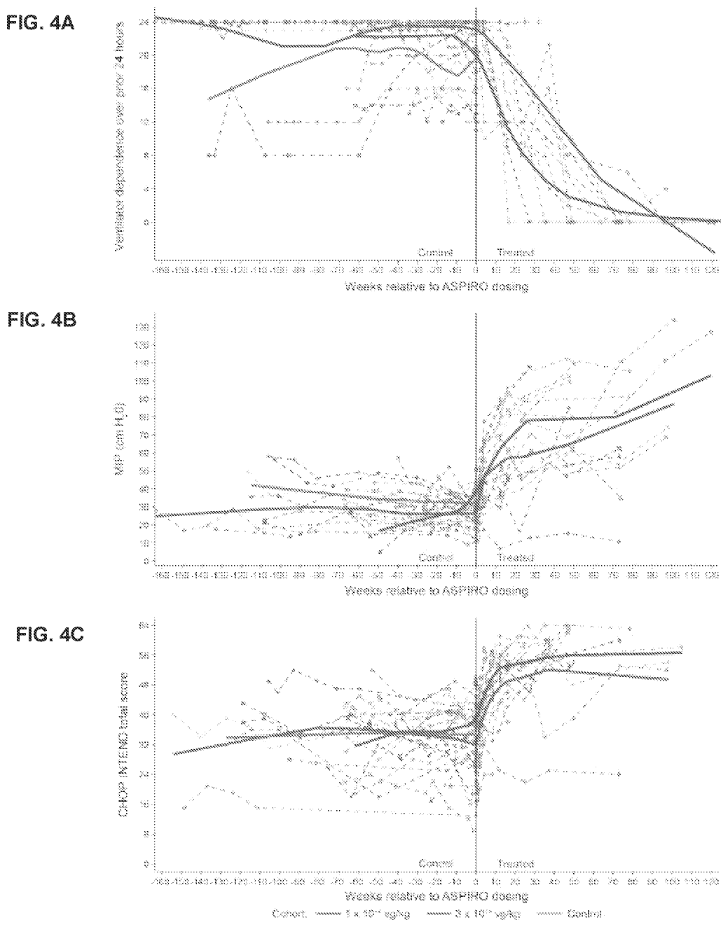

For patients requiring invasive ventilatory support, MIP was assessed by temporary occlusion of the airway with a one-way valve attached to the cuffed tracheostomy. For the few subjects requiring BiPAP, or who were successfully weaned from invasive ventilatory support, the valve apparatus was attached to a tightfitting facemask placed over the child's nose and mouth. Thus, the flow of air was occluded in one direction (e.g., during inspiration while assessing the MIP) while non-occluded in the other direction, allowing patients to exhale to residual volume and thereby maximize inspiratory pressure generation. While a child makes an inspiratory effort during the occlusion, a pressure is generated by the muscles of inspiration, which can be measured by a pressure transducer attached to the valve apparatus. To ensure consistency and in alignment with international guidelines, occlusions were maintained for at least eight breaths and at least five sets of occlusions were performed. MIP was determined electronically as being the largest negative pressure generated during an occlusion. All pressure trace electronic data were uploaded to an online portal and read by an expert respiratory physiologist (FIGS. 4A-4B).

IVb. Determination of Myotubularin Expression

Expression of MTM1 in muscle biopsies was analyzed by semi-quantitative Western blotting. Total protein extracts obtained from patient's biopsies with a Fastprep tissue lyser were quantified by a Bradford assay and analyzed by SDS-PAGE (15 μg of total protein/well). Protein extracted from muscle biopsies of healthy individuals and MTM1 knockout mice were used as positive and negative controls, respectively, on each gel. Recombinant SUMO-MTM1 fusion protein was spiked in healthy muscle extracts as an internal standard calibrator at a concentration of 0.01, 0.033, 0.11, 0.37, 1.22, 4.05, 13.53, and 45.05 ng/lane, respectively. Due to its larger size (˜83 kDa), SUMO-MTM1 was easily distinguished from endogenous MTM1 (˜70 kDa) on SDS-PAGE gel scans. For Western blotting, proteins were electro transferred to nitrocellulose membranes and transfer monitored by REVERT (Li-COR) total protein staining. After de-staining and an overnight blocking step at 5±3° C., MTM1 and glyceraldehyde 3-phosphate dehydrogenase (GAPDH), used for loading normalization, were detected by simultaneous incubation to a goat anti-human MTM1 (ABNOVA, Cat #: ABNOVAPAB6061) and a mouse anti-human GAPDH primary antibodies (Millipore, Cat #: MAB374), respectively (3 hours at room temperature), and to secondary antibodies conjugated to infrared fluorescent dyes (donkey anti-goat IRDye8000W, LiCor, Cat #: 926-32214 and goat anti-mouse IRDye680, LiCor, Cat #: 926-68070) (1 hr at room temperature). Signal was captured and analyzed with the Odyssey system (LiCor). For the study samples, MTM1 expression is reported as fold expression over the healthy control sample by REVERT normalization.

IVc. Determination of Vector Copy Number

Resamirigene bilparvovec vector DNA was quantified in the genomic DNA extracted from muscle biopsies by TaqMan qPCR (Applied Biosystems, Cat #: 4440040) as vector genomes per diploid genomes (Vector Copy Number, VCN). For the qPCR reaction, 600 nM each of the forward (MTM1-3F: 5′-CCCCAACTTCACCTTCCAG-3′; SEQ ID NO: 16) and reverse primers (MTM1-3R: 5′-ATTAGCCACACCAGCCAC-3′; SEQ ID NO: 17) and 300 nM of the MTM1-3P probe (5′-6-FAM-TGCCCCATG/ZEN/TGCAAACTCACTTC-3′IBFQ-3′; SEQ ID NO: 18) were used in 50 μL reaction volume. The thermocycle conditions were: 50° C. for 2 min, 95° C. for 10 min, and then 40 cycles of 95° C., 15 seconds, and 60° C., 30 sec. Linearized pAAVAud-Des-hMTM1 plasmid was used as a standard for quantification.

IVd. Determination of Anti-AAV8 Neutralizing Antibodies

Anti-AAV8 neutralizing antibody (Nab) titers in the patient's sera were determined by a cell-based assay that measures antibody-mediated entry inhibition of an AAV8 vector expressing a luciferase reporter gene (AAV8-luc) in 293T cells. Briefly, 30,000 293T cells were seeded into 96-well plates and transduced with a 1:1 mixture of the AAV8-luc vector at a multiplicity of infection (MOI) of 10,000 and serial dilutions of patient's sera. Cells were incubated with the mixture for 1 hr, followed by addition of Etoposide (20 μM) and then incubated overnight. Cells were then lysed in a buffer containing the luciferase substrate (Steady-Glo, Promega), and luminescence was read using a 96-well plate reader. A pre-tested serum negative for anti-AAV8 neutralizing activity is used as a Negative Control (NC). Signals from the test sample wells are divided by the average signal from the NC wells for normalization. A sample is considered positive for the anti-AAV8 neutralizing antibody if its normalized signal is below the predetermined ratio, i.e., the cut point (0.78). The anti-AAV8 NAb titer of a serum sample is calculated by linear interpolation of the two normalized signal values from two of the dilutions that flank the predetermined cut point of 0.78 and their corresponding dilution factors.

IVe. Determination of Anti-MTM1 Antibody Titer

The anti-MTM1 antibody titer was analyzed in patient's sera by an electrochemiluminescence assay using the Meso Scale Discovery (MSD) platform (Meso Scale Diagnostics, Rockville MD) following a tiered approach in which samples are first screened and then confirmed for signal specificity by competition with unlabeled MTM1 (Confirmatory Buffer). The anti-MTM1 antibody titer was measured in all confirmed positive samples. For the anti-MTM1 antibody assay, Streptavidin-impregnated MSD plates were blocked with the MSD wash buffer containing 5% BSA for approximately two hours at room temperature. Samples and controls were diluted appropriately in Dilution Buffer or Confirmatory Buffer (2.22 μg/mL MTM-1 in Dilution Buffer). 100 μL of Master Mix solution containing biotinylated-MTM1 and Ruthenylated-MTM1 was added to the appropriate wells on a polypropylene (PP) plate. Fifty microliters of the diluted samples or controls was added to the wells of the PP plate with the labeled MTM1 Master Mix. The plate was sealed and incubated for approximately two hours at room temperature to allow bridging of the two labeled species of MTM1 by the anti-MTM1 antibody in the sample, if present.

Meanwhile, the Streptavidin-MSD plate was blocked and washed using MSD wash buffer. 50 μL of sample or control in Master Mix from the PP plate was added to the Streptavidin-MSD plate for capturing the anti-MTM1 antibodies via the biotinylated MTM1. The plate was sealed and incubated for approximately one hour at room temperature. After incubation, the Streptavidin-MSD plate was washed using MSD wash buffer. 150 μL of 2×MSD Read buffer T without surfactant was added to each well, and the plate was read using an MSD Sector Imager (S600) plate reader. The presence of anti-MTM1 antibody was determined by comparing the signal to a statistically derived threshold, the assay cut point.

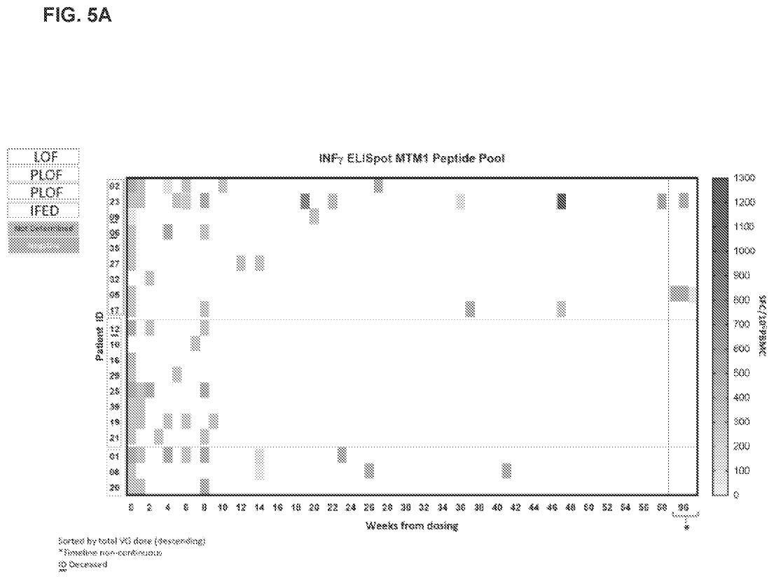

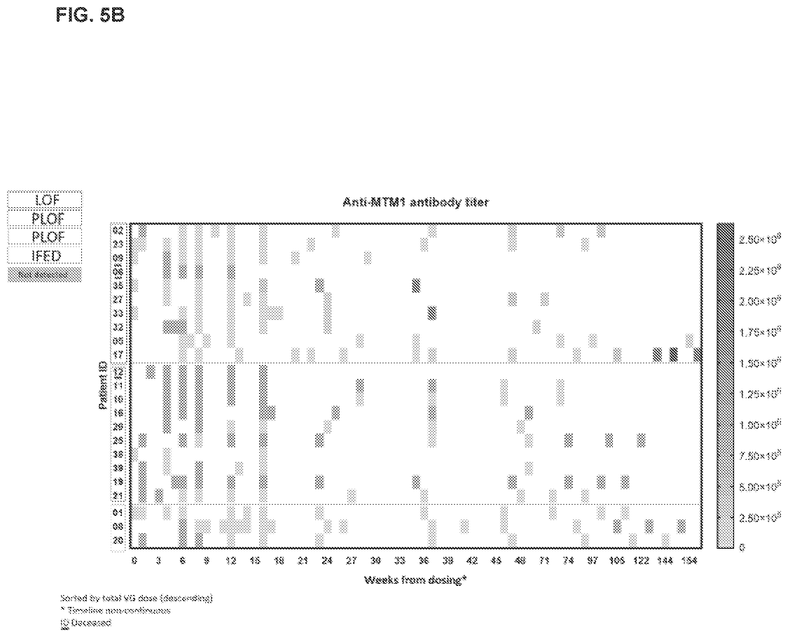

For the determination of anti-MTM1 antibody titer in the confirmed positive samples (FIG. 5B), each sample was serially diluted, and the set of dilutions was tested in a manner essentially similar to that of the screening assay. Two-fold serial dilutions from each sample was prepared in 10% human serum pool (NC) in dilution buffer and assayed in duplicate, until the signal fell below the cut point. The dilution factor above which the normalized signal fell below the titration cut point (1.32 Sample/NC signal, Titration Cut Point) for the first time was multiplied by the dilution factor to determine the final titer value.

IVf. RNA Sequencing Analysis

The level of vector-derived MTM1 mRNA expression was measured by RNA sequencing. Total RNA was extracted from frozen muscle biopsies, and quality and quantity assessed by Nanodrop (Thermo Fisher Scientific, Waltham, MA), Qubit (Thermo Fisher), and TapeStation (Agilent, Santa Clara, CA) before sequencing library preparation. ERCC (External RNA Control Consortium, National Institute of Standards and Technology, Gaithersburg, MD). RNA spike-ins were used according to manufacturer's instructions. Sequencing libraries were prepared using the standard Illumina strand-specific protocol with poly-A selection and unique dual index barcodes for sample multiplexing. Samples were multiplexed and sequenced together across two lanes of an Illumina (San Diego, CA) HiSeq 4000 (2×150 bp) for a total of ten lanes of raw data. Sequencing reads were trimmed of adapter sequences and aligned to the human genome supplemented with the transgene sequence. Quality control and analysis of the RNA-seq data were performed with an internally developed bioinformatic pipeline. Read count normalization, downstream analyses, and plotting were performed using R version 3.5.1.

As outlined in Table 6, each allele was classified according to likely null mutations leading to little or no stable protein versus mutations where muscle might express stable intact or internally deleted proteins (e.g., possibly with residual activity). Loss of function (LOF) alleles included all predicted genetic null mutations. Partial loss of function (PLOF) variants included inframe single exon duplications, three base pair inframe deletions, inframe insertions, small inframe indels and missense variants. Inframe exonic deletions (IFED) include larger deletions predicted to encode stable but internally deleted proteins that might be missing entire functional domains and antigenic epitopes.

TABLE 6
Genotype Data and Potential Mutation Impact

| | | | | | | | | |
|---|---|---|---|---|---|---|---|---|
| | | | Predicted | | | ClinVar | | |
| | Genomic location | cDNA change | protein change | Molecular | | Variation | dbSNP | HGMD |
| Patient No. | (hg19) | (NM_000252.2) | (NP_000243.1) | consequence | Mutation impact* | ID | ID | ID |

Treated ASPIRO Patients
Dose: 1 × 10¹⁴ vg/kg

| | | | | | | | | |
|---|---|---|---|---|---|---|---|---|
| 08 | chr23: | c.232- | p.Asp78_Lys114del | Splicing | IFED | n/a | n/a | n/a |
| | 149783046T > G | 16T > G | | | | | | |
| 20 | chr23: | c.(444 + | p.(Pro149_Pro226del) | Deletion | IFED | n/a | n/a | CG073869 |
| | (?_149807416)_(149809891_?)del | 1_445 − | | exons 7-8 | | | | |
| | | 1)_(678 + | | | | | | |
| | | 1_679 − | | | | | | |
| | | 1)del | | | | | | |
| 21 | chr23: | c.197C > T | p.(Thr66Ile) | Missense | PLOF | n/a | n/a | n/a |
| | 149767116C > T | | | | | | | |
| 19 | chr23: | c.1262G > T | p.(Arg421Leu) | Missense | PLOF | 599006 | n/a | CM1513749 |
| | 149828138G > T | | | | | | | |
| 17 | chr23: | c.1558C > T | p.(Arg520*) | Nonsense | LOF | 158950 | rs587783805 | CM001733 |
| | 149831996C > T | | | | | | | |
| 05 | chr23: | c.1109C > G | p.(Ser370*) | Nonsense | LOF | n/a | n/a | n/a |
| | 149826349C > G | | | | | | | |

Dose: 3 × 10¹⁴ vg/kg

| | | | | | | | | |
|---|---|---|---|---|---|---|---|---|
| 01 | chr23: | c.342 + 1G > A | p.? | Splicing | IFED | 435903 | rs1557413092 | CS1724128 |
| | 149783173G > A | | | | | | | |
| 25 | chr23: | c.1228G > C | p.(Glu410Gln) | Missense | PLOF | n/a | n/a | n/a |
| | 149826468G > C | | | | | | | |
| 12 | chr23: | c.1262G > A | p.(Arg421Gln) | Missense | PLOF | 158914 | rs587783772 | CM970999 |
| | 149828138G > A | | | | | | | |
| 06 | chr23: | c.1262G > A | p.(Arg421Gln) | Missense | PLOF | 158914 | rs587783772 | CM970999 |
| | 149828138G > A | | | | | | | |
| 16 | chr23: | c.1490C > A | p.(Ser497Tyr) | Missense | PLOF | 158945 | rs587783800 | CM1814203 |
| | 149831928C > A | | | | | | | |
| 10 | chr23: | c.1505T > A | p.(Ile502Lys) | Missense | PLOF | 158963 | n/a | CM194892 |
| | 149831943T > A | | | | | | | |
| 11 | chr23: | c.1505T > A | p.(Ile502Lys) | Missense | PLOF | 158963 | n/a | CM194892 |
| | 149831943T > A | | | | | | | |
| 29 | chr23: | c.688T > C | p.(Trp230Arg) | Missense | PLOF | 92677 | rs398123274 | CM050296 |
| | 149814165T > C | | | | | | | |
| 23 | chr23: | c.1178dupT | p.(Leu393Phefs*3) | Frameshift | LOF | 435902 | n/a | n/a |
| | 149826418dupT | | | | | | | |
| 02 | chr23: | c.949_950dupA | p.(Met317Asnfs*15) | Frameshift | LOF | 211538 | rs797045722 | n/a |
| | 149818270_149818271dupA | | | | | | | |
| 09 | chr23: | c.(?_- | p.(0) | Gene deletion | LOF | n/a | n/a | n/a |
| | (?_149737047)_(149841616_?)del | 76)_(*1548_?)del | | | | | | |
| 33 | chr23: | c.814delT | p.(Ser272Leufs*12) | Frameshift | LOF | n/a | n/a | n/a |
| | 149814291delT | | | | | | | |
| 27 | chr23: | c.70C > T | p.(Arg24*) | Nonsense | LOF | 92678 | rs398123275 | CM970981 |
| | 149764968C > T | | | | | | | |
| 35 | chr23: | c.757C > T | p.(Arg253*) | Nonsense | LOF | 159001 | rs587783854 | CM990873 |
| | 149814234C > T | | | | | | | |
| 32 | chr23: | c.1456C > T | p.(Arg486*) | Nonsense | LOF | 158938 | rs587783795 | CM990881 |
| | 149828946C > T | | | | | | | |
| 38 | chr23: | c.(1467 + | p.? | Deletion | LOF | n/a | n/a | n/a |
| | (?_149831905)_(149841616_?)del | 1_1468 − | | exons 14-15 | | | | |
| | | 1)_(*1548_?)del | | | | | | |
| 39 | chr23: | c.614C > T | p.(Pro205Leu) | Missense | PLOF | 158987 | rs587783841 | CM960998 |
| | 149809827C > T | | | | | | | |

Untreated ASPIRO-INCEPTUS Control Patients

| | | | | | | | | |
|---|---|---|---|---|---|---|---|---|
| 26 | chr23: | c.(1053 + | p.(Leu352_Ser420del) | Deletion | IFED | n/a | n/a | n/a |
| | (?_149826294)_(149826500_?)del | 1_1054 − | | exon 11 | | | | |
| | | 1)_(1260 + | | | | | | |
| | | 1_1261 − | | | | | | |
| | | 1)del | | | | | | |
| 31 | chr23: | c.(444+ | p.(Pro149_Gln176del) | Deletion | IFED | n/a | n/a | n/a |
| | (?_149807406- | 1_445 − | | exon 7 | | | | |
| | 149807509_?)del | 1)_(528 + | | | | | | |
| | | 1_529 − | | | | | | |
| | | 1)del | | | | | | |
| 03 | chr23: | c.1537_1564delinsAACTGGA | p.(Phe513_Leu522delinsAsnTrpIle) | Indel | PLOF | 92674 | rs398123271 | n/a |
| | 149831975_149832002delinsAACTGGA | | | | | | | |
| 07 | chr23: | c.721C > T | p.(Arg241Cys) | Missense | PLOF | 11059 | rs132630305 | CM970993 |
| | 149814198C > T | | | | | | | |
| 30 | chr23: | c.1261-10A > G | p.Ser420Arg421insPheIleGln | Splicing | PLOF | 11058 | rs397518445 | CS971813 |
| | 149828127A > G | | | | | | | |
| 04 | chr23: | c.(?_-76)_(231 + | p.(0) | Deletion | LOF | n/a | n/a | n/a |
| | (?_149737047)_(149767150_?)del | 1_232 − | | exons 1-4 | | | | |
| | | 1)del | | | | | | |
| 13 | chr23: | c.70C > T | p.(Arg24*) | Nonsense | LOF | 92678 | rs398123275 | CM970981 |
| | 149764968C > T | | | | | | | |
| 14 | chr23: | c.915delA | p.(Glu305Aspfs*5) | Frameshift | LOF | n/a | n/a | n/a |
| | 149818236_149818236delA | | | | | | | |
| 15 | chr23: | c.1611C > A | p.(Tyr537*) | Nonsense | LOF | 280453 | rs886041657 | n/a |
| | 149832049C > A | | | | | | | |
| 18 | chr23: | c.1228G > T | p.(Glu410*) | Nonsense | LOF | n/a | n/a | n/a |
| | 149826468G > T | | | | | | | |
| 22 | chr23: | c.109C > T | p.(Arg37*) | Nonsense | LOF | 158895 | rs587783753 | CM970982 |
| | 149765007C > T | | | | | | | |
| 24 | chr23: | c. 109C > T | p.(Arg37*) | Nonsense | LOF | 158895 | rs587783753 | CM970982 |
| | 149765007C > T | | | | | | | |
| 28 | chr23: | c.141_144delAGAA | p.(Glu48Leufs*24) | Frameshift | LOF | 26096 | rs587783791 | CM970981 |
| | 149767060-149767063delAGAA | | | | | | | |
| 36 | chr23: | c.1132G > A | p.(Gly378Arg) | Missense | PLOF | 158897 | rs587783755 | CM961000 |
| | 149826372G > A | | | | | | | |
| 40 | chr23: | c. 1420C > T | p.(Arg474*) | Nonsense | LOF | 158935 | rs587783792 | CM971002 |
| | 149828910C > T | | | | | | | |

*MTM1 mutation status and predicted impact was derived from clinical testing as described in Graham et al. Arch Dis Child 2019 Sep. 4; 10.1136/archdischild-2019-31791.
dbSNP ID: Single Nucleotide Polymorphism Database;
HGMD ID—The Human Gene Mutation Database

### V. Statistical Methods

Results are reported as of the data cut-off Jan. 29, 2021. The change from baseline over time in daily ventilation hours, CHOP INTEND score, and MIP were calculated and compared among the lower- and higher-dose groups and control group with a repeated-measures analysis of variance model, with participant as a random effect and week and treatment-by-week interaction as fixed effects. Change from baseline was reported as least squares mean (LSM) and standard error (SE). The odds of reaching a milestone in the treated group as compared with the control group were estimated as the relative risk and associated 95% confidence intervals [CI], with continuity correction used to avoid dividing by zero. Time to ventilator independence and attainment of motor milestones were analyzed with Kaplan-Meier and Wilcoxon tests. A 95% confidence interval was calculated for median time-to-event analyses. SAS v9.4 was used for all analyses.

### Results

### Participants

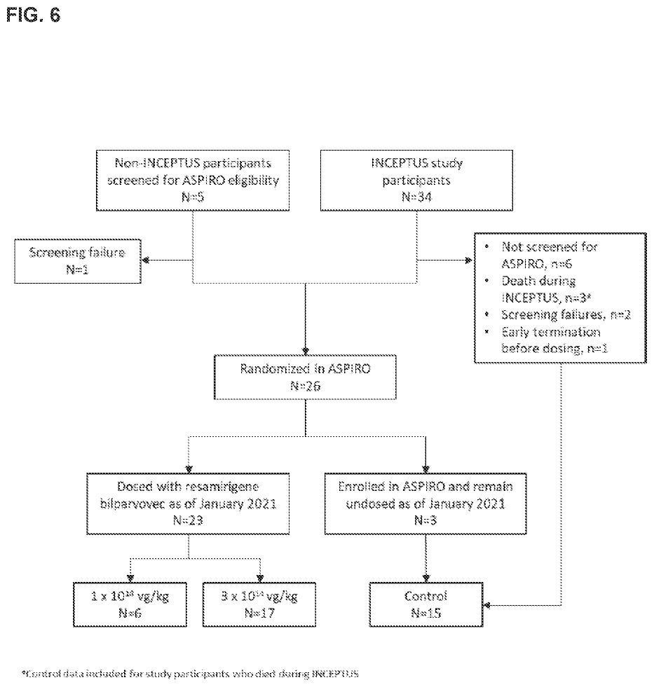

As of January 2021, 23 participants had received resamirigene bilparvovec: six at the lower dose and 17 at the higher dose (FIG. 6). No control participants (n=15) had received resamirigene bilparvovec as of January 2021. In a subsequent phase of the trial, one additional control participant (participant 40) had received resamirigene bilparvovec at the lower dose.

Participant characteristics at baseline were similar in the treated and control groups (see Table 7, below). The mean age was 20.4 months (range: 9.5, 49.7) at treatment administration in the lower-dose cohort, 39.4 months (range: 6.8, 72.7) in the higher-dose cohort, and 19.6 months (range: 5.9, 39.3) at enrollment among control participants. In all three groups, invasive ventilation was used for a mean of 22 hours per day at baseline, and mean MIP values were four standard deviations below the lower limit of the normal range for children of similar age, 80 cmH₂O. The mean baseline CHOP INTEND scores were approximately 50% lower than expected in healthy children 3 to 6 months of age. Only three participants (one from each group) were able to sit unassisted for 30 seconds at baseline. Genotypic data are provided in Table 6.

### Respiratory Function

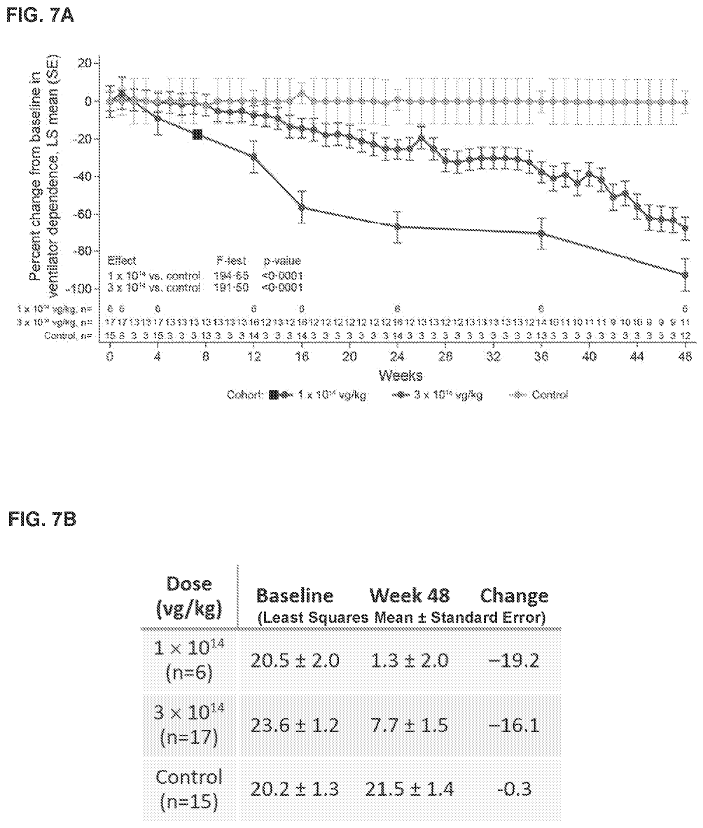

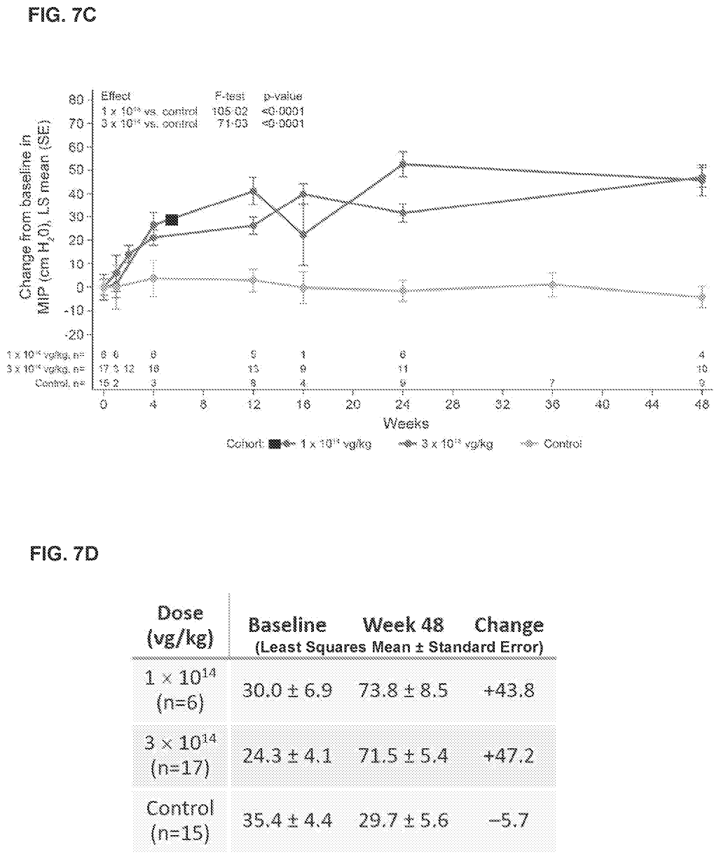

Daily hours of ventilator support decreased significantly from baseline to Week 48 among both dose groups but not among controls (P<0.0001, FIGS. 7A-7B). Support was decreased from LSM (±SE) 20.5±2.0 hours to 1.3±2.0 hours (LSM change analyzed using repeated-measures analysis of variance model, −19.2 hours) in the lower-dose cohort, from 23.6±1.2 hours to 7.7±1.5 hours (LSM change −15.9 hours) in the higher-dose cohort, and from 20.2±1.3 hours to 21.5±1.4 hours (LSM change −0.3 hours) among controls. There was a significantly (P<0.0001) greater percent decrease from baseline in daily hours of ventilator dependence in treated vs control participants (FIGS. 7A-7B). Ventilator independence was achieved by fifteen treated participants (all six lower dose and nine higher dose) between 16 and 72 weeks after treatment; however, one lower-dose participant subsequently required intermittent ventilatory support in part due to underlying scoliosis, a frequent comorbidity of XLMTM. Among treated participants with ventilator independence, the median time to the event was 50.7 weeks (95% Cl, 41.9 to 71.1). No control patients achieved reductions of ventilator independence.

MIP, a measure of respiratory muscle strength, increased significantly among both dose groups compared with controls from baseline to Week 48 (P<0.0001, FIGS. 7C-7D). MIP increased from LSM (±SE) 30.0±6.9 cmH₂O to 73.8±8.5 cmH₂O (LSM change 43.8 cmH₂O) in the lower-dose cohort and from 24.3±4.1 cmH₂O to 71.5±5.4 cmH₂O (LSM change 47.2 cmH₂O) in the higher-dose cohort and decreased from 35.4±4.4 cmH₂O to 29.7±5.6 cmH₂O (LSM change −5.7 cmH₂O) among control participants.

### Motor Function

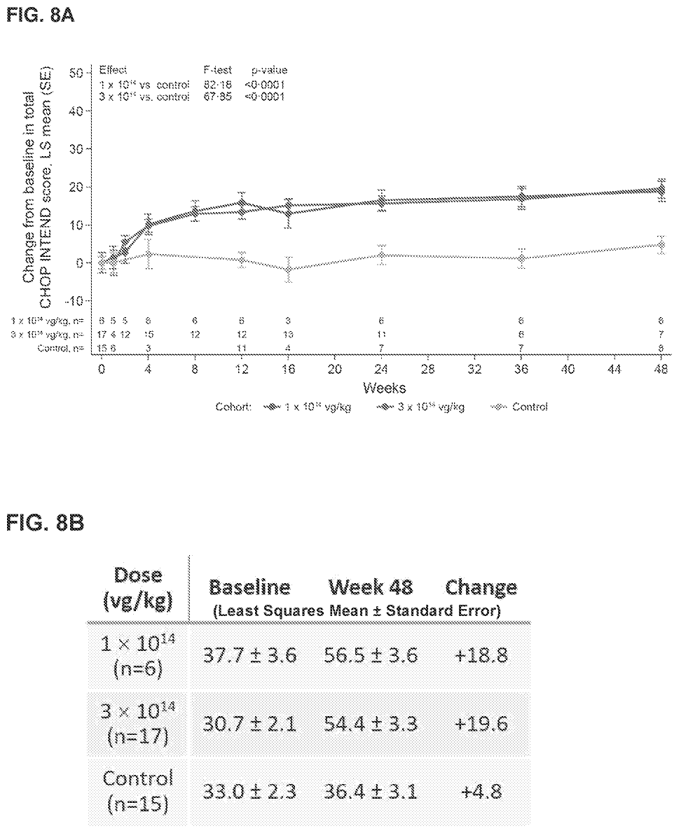

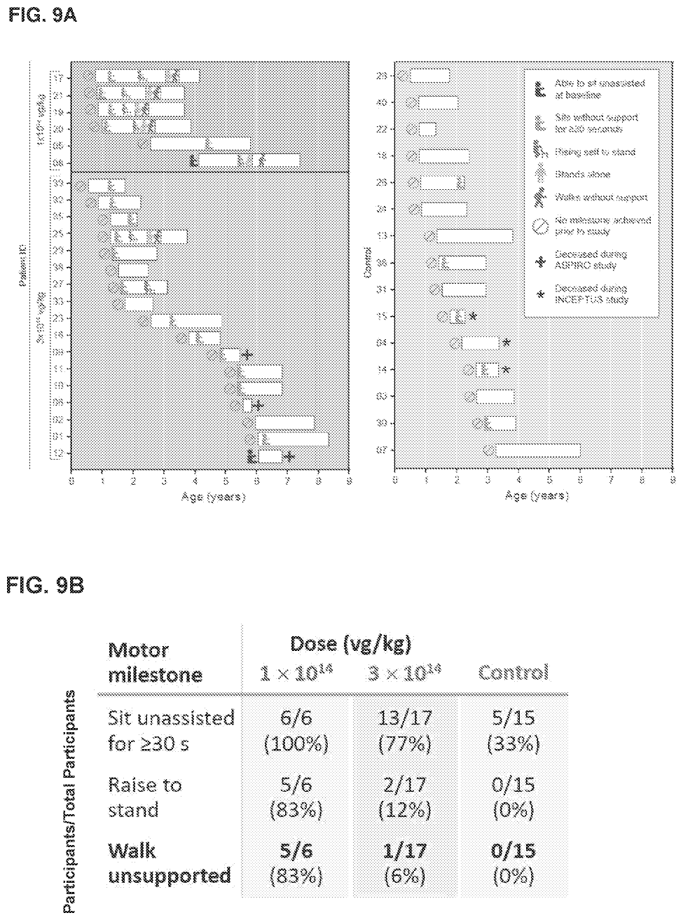

Among participants receiving either dose, CHOP INTEND scores increased significantly from baseline to Week 48 compared with control (P<0.0001, FIGS. 8A-8B and 4C). Change in LSM (±SE, analyzed using repeated-measures analysis of variance model) were 18.8 (from 37.7±3.6 to 56.5±3.6) in the lower-dose cohort, 19.6 (from 30.7±2.1 to 54.4±3.3) in the higher-dose cohort, and 4.8 (from 33.0±2.3 to 36.4±3.1) among control. Noticeable improvements occurred as early as 4 weeks after treatment; 78% ( 18/23) of treated participants achieved a clinically meaningful 4-point or greater increase in CHOP scores by Week 4. A higher percentage of treated participants than control participants attained essential motor milestones between baseline and last observation (Table 8, below). At the time of data cutoff, all six lower-dose participants (100%) and 13/17 (77%) higher-dose participants were able to sit unassisted for at least 30 seconds, compared with 5/15 (33%) control participants. Five (83%) lower-dose participants could pull themselves to stand, compared with 2/17 (12%) higher-dose participants and no control participants. Five of six (83%) lower-dose participants and 1/17 (6%) higher-dose participants could walk unsupported beginning at a mean 21.1 months (range: 16.4-29.8) post dosing compared with no control participants. Motor function outcomes in individual participants are shown in FIGS. 9A-9B.

### Muscle Biopsy Findings

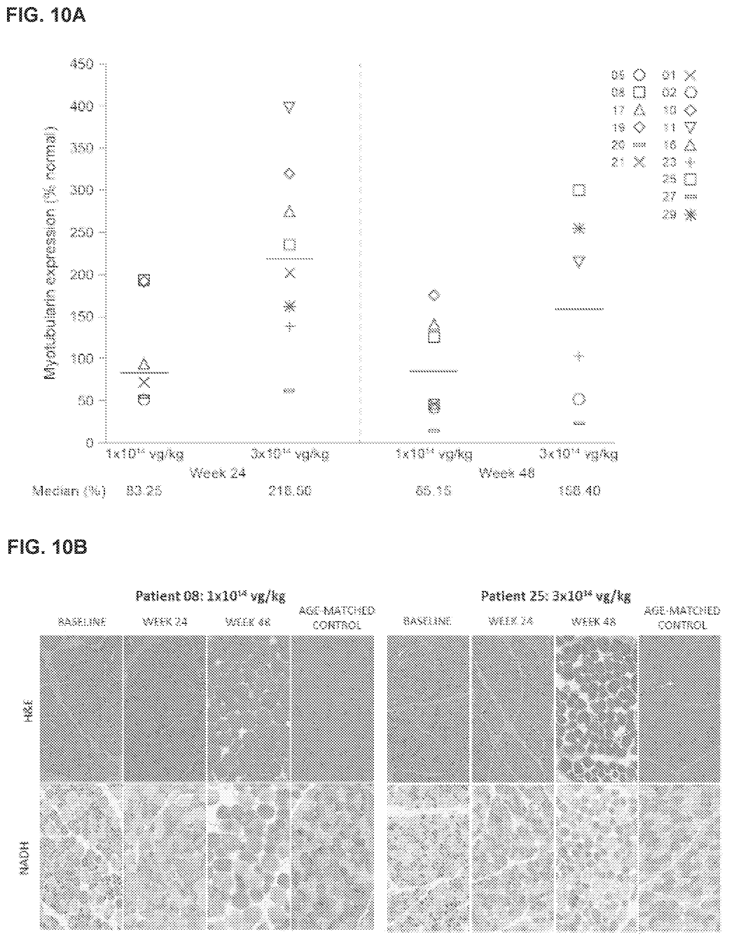

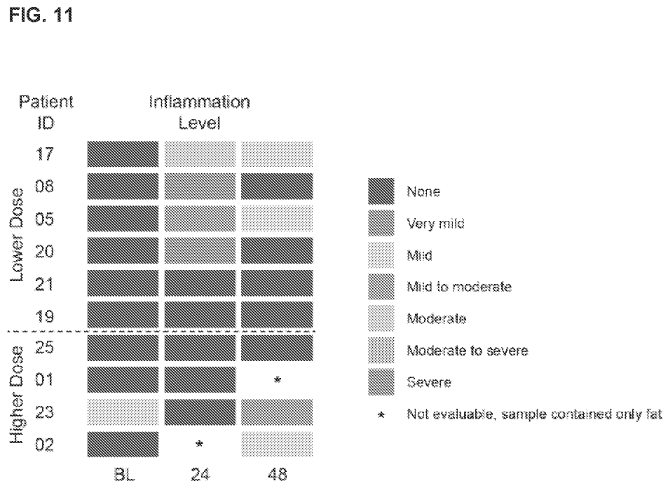

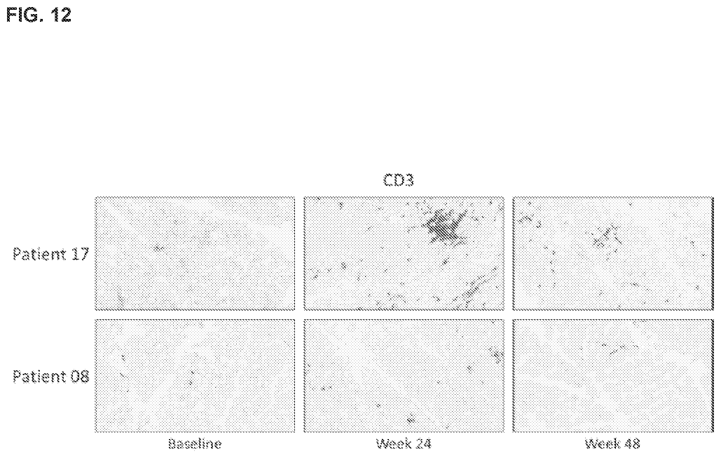

Dose-dependent changes in VCN and expression of mRNA and MTM1 protein were observed at 24 and 48 weeks after treatment (FIGS. 10A-B). Pretreatment muscle-biopsy findings in all participants were characteristic of XLMTM, including myofiber hypotrophy, central aggregates of organelles, and increased proportions of fibers with internally placed nuclei. Muscle biopsy specimens obtained 24 weeks after treatment in the lower-dose cohort showed improvements in organelle (mitochondria) organization with minimal effect on myofiber size or in the percentage of internally nucleated fibers. Muscle biopsies 48 weeks after treatment continued to show appropriate organelle localization and similar levels of internal nucleation, but myofiber size had increased relative to baseline. Greater increases in fiber size were observed in some patients in the higher-dose cohort by Week 24 compared with the lower-dose cohort, suggesting a more rapid reduction in histopathologic abnormalities in some patients at the higher dose. Post-treatment biopsy specimens from four participants contained variable degrees of inflammation (FIGS. 11-12) and mixed lymphocytic infiltrate; the degree found in a given participant did not correlate well with evidence of muscle damage on clinical evaluation or laboratory testing (e.g., some patients with elevated creatine phosphokinase at the time of biopsy displayed no lymphocytic infiltrates, and some patients with lymphocytic infiltrates showed no elevations in creatine phosphokinase) (FIG. 5A).

### Survival and Safety

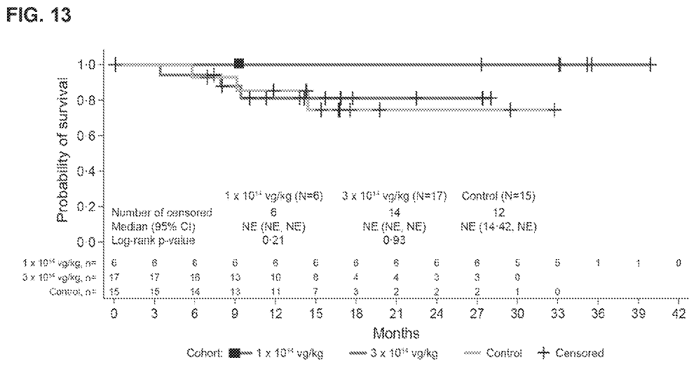

Kaplan-Meier survival analysis for the first phase of the trial is shown in FIG. 13. During this phase, there were no deaths in the lower-dose cohort, three deaths in the higher-dose cohort, and three deaths in the control cohort (shock secondary to hepatic hemorrhage presumed to be related to peliosis; aspiration pneumonia with acute respiratory failure; and acute on chronic bronchopneumonia with evidence of cardiac dysfunction due to longstanding pulmonary disease). Among the three participants who received the higher dose and died, the suspected underlying cause of death was severe cholestatic liver injury with decompensated liver disease. The immediate causes of death as reported by investigators were: (1) sepsis (participant 06); (2) hepatopathy, severe immune dysfunction, and pseudomonal sepsis (participant 09); and (3) circulatory collapse due to gastrointestinal bleed (participant 12). All three participants had ongoing cholestatic hepatobiliary SAEs with decompensated liver disease at the time of death. At the time of dosing, these three participants were ages 4.8 years (17.3 kg), 5.6 years (15.8 kg), and 6.1 years (25.8 kg). Beginning 3-4 weeks after treatment, all showed dramatic increases in direct and total bilirubin values (eventually peaking at 28-92-fold ULN and 34-54-fold ULN, respectively) which was followed by increases in ALT, AST, and GGT values (peaking at 7-22-fold ULN, 7-23-fold-ULN, and 4-11-fold-ULN, respectively). All then experienced progression to severe decompensated liver disease, characterized by ascites, extensive liver fibrosis, and poor hepatic synthetic function. The three participants had evidence of pre-existing (e.g., pre-treatment) cholestasis but met the trial eligibility criteria of no clinically significant liver disease, as defined by >5×ULN ALT or AST or imaging evidence of hepatic peliosis, at the time of enrollment. The three fatal events were considered to be clinically related to severe, cholestatic, decompensated liver disease in the setting of a high total dose of AAV8; these participants were among the heaviest participants and thus received among the highest total doses of resamirigene bilparvovec (4.8×10¹⁵-7.7×10¹⁵ total vgIn the first phase of the trial, similar SAEs were not observed in the lower-dose cohort or among lighter participants in the higher-dose cohort despite more than half of ASPIRO participants having evidence of pre-existing hepatobiliary disease, including intermittent aminotransferase elevation, hyperbilirubinemia, and/or historical cholestasis or jaundice. While one participant weighing 9.4 kg in the higher-dose cohort reported cholestatic hepatitis nearly one year from dosing, the event was not complicated by progression to fibrotic, decompensated liver disease. No participants who received the lower dose have experienced hepatobiliary SAEs despite four of the six participants having evidence of pre-existing hepatobiliary disease, three of whom had medical histories consistent with cholestasis and/or documented laboratory hyperbilirubinemia prior to dosing. In the higher-dose cohort, apart from the three deceased participants, three other participants have experienced hepatobiliary SAEs, including cholestasis, hepatitis, and transaminitis. Participant 01, dosed at 6.8 years of age, weighed approximately 21 kg at dosing and had peak direct and total bilirubin values of 10×ULN and 32×ULN over a period of 10-17 weeks after receiving resamirigene bilparvovec, before his bilirubin values decreased, and the SAE resolved without hepatic sequelae. Participants 39 and 38 received the higher dose at 0.6 and 1.5 years of age, respectively, and demonstrated at 1 week and 23 weeks after treatment abrupt elevations in ALT (19× and 20×ULN) and AST values (23× and 6×ULN) with normal bilirubin values before resolving. Subsequently, just over one year after treatment, Participant 38 developed recurrent elevation in ALT and AST of similar magnitude along with an elevation in direct and total bilirubin. Diagnostic liver biopsy was consistent with intrahepatic cholestasis, and serum bile acid levels were elevated. At last report, ALT and total bilirubin levels had normalized, while AST and direct bilirubin had trended toward normal but remained slightly elevated. In all study participants with liver-related SAEs, serological, cellular, complement, cytokine, routine laboratory and histopathological assessments do not suggest that the SAEs were driven by immune responses, though there are insufficient data to draw definitive conclusions.

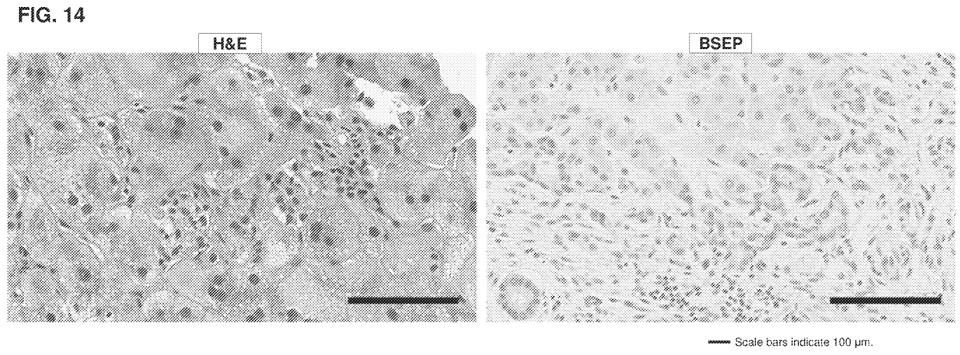

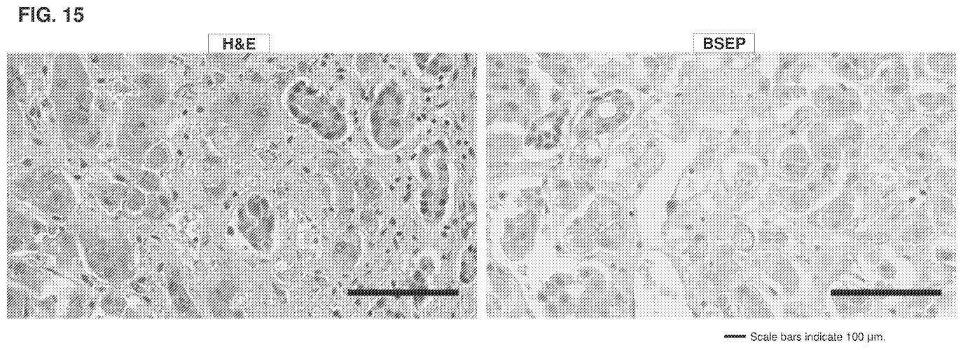

In a subsequent phase of the trial, 24 participants had received resamirigene bilparvovec: seven at the lower dose and 17 at the higher dose. There was one death in the lower-dose cohort (participant 40), three deaths in the higher-dose cohort (described above), and three deaths in the control cohort. Like the three higher-dose deaths, participant 40 exhibited evidence of cholestasis pre-dating dosing and showed an increase in liver function tests within 1-4 weeks after dosing. The participant experienced progression to severe decompensated liver disease, characterized by ascites, extensive liver fibrosis, and poor hepatic synthetic function. The immediate cause of death was reported to be sepsis. Histopathological assessments at autopsy of all four patients, as well as biopsies taken during life in subject 12, are consistent with a severe and rapidly progressive cholestatic liver failure that included giant cells and intracellular and intracanalicular bile collections, and there is indication that a key bile transport protein, BSEP, was decreased in expression in the livers of these patients for at least the first few months following treatment. Liver biopsy of participant 12 taken prior to the fatal serious adverse events on day 85 post dose showed hepatocyte degeneration and giant cell formation, intracellular and extracellular bile collections, bile duct proliferation, and minimal inflammation consistent with a response to hepatocyte damage (FIG. 14). Liver biopsy of participant 06 taken post-mortem from an autopsy sample showed hepatocyte degeneration, necrosis, giant cell formation, intracellular and extracellular bile collections, bile ductular proliferation and severe fibrosis (FIG. 15). A summary of the four deceased participants is found in Table 9. Of the seven participants in the lower-dose cohort, one participant had no liver laboratory abnormalities reported as adverse events or serious adverse events, six participants had liver-related adverse events reported, one participant had non-fatal liver-related serious adverse events reported, and one participant had fatal hepatobiliary serious adverse events. Of the 17 participants in the higher-dose cohort, four participants had no liver laboratory abnormalities reported as adverse events or serious adverse events, 13 participants had liver-related adverse events reported, four participants had non-fatal liver-related serious adverse events reported, and three participants had fatal hepatobiliary serious adverse events. A summary of the number of liver-related adverse events for all 24 treated participants is found in Table 10.

Among all treated participants, the most common adverse events reported were respiratory tract infections, increases in creatine phosphokinase, and pyrexia. Troponin I was elevated in two higher-dose participants, one lower-dose participant, and no controls. One participant from each dose cohort (Patient 23 and Patient 05), both with predicted null MTM1 mutations expected to produce no myotubularin protein but neither of whom demonstrated among the highest anti-myotubularin antibody titers observed on the study, were considered to have probable cases of myocarditis, and investigators elected to treat both participants with pulsed methylprednisolone and sirolimus with mycophenolate mofetil and intravenous immunoglobulin additionally given to one participant. In both participants, myocarditis was reported to have resolved (within approximately 4 months and 10 months, each) and serial echocardiograms continued to show normal myocardial function, although one participant continued on sirolimus past resolution. Adverse events of transient, asymptomatic thrombocytopenia within the first two weeks of dosing and considered at least possibly treatment-related were reported in six patients who received the higher dose (five with mild intensity, one with moderate intensity). In five patients, resolution was reported without treatment, while the sixth patient received a single dose of methylprednisolone prior to resolution. Flow cytometry analyses of samples taken during the first month dosing demonstrated non-specific binding to glycoproteins (GpIIb/IIIa, GpIb/IX, GpIV, HLA Class I, GpIa/IIa) in different platelet populations.

SAEs reported in ≥1 participant are summarized in Table 11, below. In the lower-dose cohort, 19 SAEs were reported among four participants. Four of these events, all occurring in participant 05, were deemed possibly or probably related to treatment and were thought to be associated with suspected clinical myocarditis. In the higher-dose cohort, 45 SAEs occurred among 12 participants. Of these, 29 occurring in nine participants were deemed possibly or probably related to treatment. In the control cohort, 41 SAEs were observed among 13 participants, including numerous respiratory infections and illnesses not observed in treated participants (Table 11, below).

Detectable anti-AAV8 neutralizing antibodies developed in all treated participants. Following dosing, response to stimulation with a myotubularin peptide pool in the IFNg-ELISpot assay was documented in five participants (01, 02, 05, 08, and 23), all with predicted LOF or IFED mutations (FIG. 13A). Four participants had only negative assay responses following dosing, and 14 participants had no evaluable post-dose data. Two participants with reactivity (23 and 05) had reported SAEs of myocarditis, and both were treated with immunosuppression, including with T-cell modulating therapies under the assumption of an anti-transgene cytotoxic T-cell reaction. In the remaining three participants with documented reactivity, the signal appeared transient and subsided without treatment. Detectable anti-MTM1 antibodies developed after treatment in 19 of 23 treated participants (FIGS. 10A-B and 13B), and the presence of antibodies was not associated with differences in clinical outcomes or findings on muscle biopsy.

TABLE 7
Demographic and Clinical Characteristics at Baseline

| | 1 × 10¹⁴ vg/kg | | 3 × 10¹⁴ vg/kg | | Control | |
|---|---|---|---|---|---|---|
| | N = 6 | | N = 17 | | (N = 15) | |
| Characteristic | n (%) | | n (%) | | n (%) | |
| Mean (range) age at dosing - | 20.4 | (9.5, 49.7) | 39.4 | (6.8, 72.7) | 19.6 | (5.9, 39.3) |
| months* | | | | | | |
| Mean (range) weight - kg | 11.7 | (8.1, 21.5) | 15.3 | (6.9, 25.8) | 11.2 | (6.7, 14.7) |

Race - no. (%)

| White | 6 | (100.0) | 11 | (64.7) | 10 | (66.7) |
|---|---|---|---|---|---|---|
| Asian | 0 | | 1 | (5.9) | 2 | (13.3) |
| Black Or African American | 0 | | 4 | (23.5) | 0 | |
| Not Reported | 0 | | 1 | (5.9) | 3 | (20.0) |
| Mean (range) age at genetic | 3.3 | (0.4, 6.5) | 6.5 | (−2.7, 31.6) | 4.2 | (−2.0, 25.5) |

diagnosis - months

Functional classification of MTM1 variants by predicted
consequence on protein function^{†} - no. (%)

| Loss of function | 1 | (16.7) | 8 | (47.1) | 9 | (60.0) |
|---|---|---|---|---|---|---|
| Partial loss of function | 3 | (50.0) | 8 | (47.1) | 4 | (26.7) |
| Inframe exonic deletion | 2 | (33.3) | 1 | (5.9) | 2 | (13.3) |
| Variant of Unknown | 0 | | 0 | | 0 | |
| Significance | | | | | | |
| Missing | 0 | | 0 | | 0 | |
| Mean (range) total CHOP | 37.7 | (29.0, 45.0) | 30.7 | (9.0, 50.0) | 33.0 | (17.0, 45.0) |
| INTEND Score^{‡} | | | | | | |
| Number (%) of subjects with | 5 | (83.3) | 17 | (100.0) | 10 | (66.7) |
| invasive ventilation | | | | | | |
| Number (%) of subjects with | 1 | (16.7) | 0 | | 5 | (33.3) |
| noninvasive ventilation^{¶} | | | | | | |
| Invasive ventilator dependence, | 22.2 | (17.0, 24.0) | 23.6 | (20.5, 24.0) | 22.2 | (16.0, 24.0) |
| mean hours per day (range) | | | | | | |
| Non-invasive ventilator | 12.0 | (12.0, 12.0) | NA | | 16.1 | (12.0, 24.0) |
| dependence, mean hours per | | | | | | |
| day (range) ^{¶} | | | | | | |
| Mean (range) MIP - cmH₂O | 30.0 | (14.1, 44.1) | 24.3 | (10.4, 44.6) | 35.4 | (20.1, 50.9) |
| Mean (range) study duration - | 36.7 | (33.4, 40.4) | 16.1 | (3.4, 29.8) | 16.1 | (5.7, 32.7) |

months

Sitting unassisted for 30 seconds - no. (%)

| | | | | | | |
|---|---|---|---|---|---|---|
| Achieved | 1 | (16.7) | 1 | (5.9) | 1 | (6.7) |
| Not Achieved | 5 | (83.3) | 15 | (88.2) | 9 | (60.0) |
| Unknown | 0 | (0.0) | 1 | (5.9) | 5 | (33.3) |

NA = not applicable.
*Age for untreated controls given as age of INCEPTUS informed consent.
^{†}See Table 6 for genotype data and potential mutation impact
^{‡}Scores range 0 to 64, with higher scores indicating better function.
^{¶}Includes bilevel positive airway pressure or continuous positive airway pressure.

TABLE 8
Achievement of Motor Milestones after Resamirigene Bilparvovec

| | | | | |
|---|---|---|---|---|
| | 1 × 10¹⁴ vg/kg | 3 × 10¹⁴ vg/kg | Controls | Relative Risk |
| | N = 6 | N = 17 | N = 15 | Treated vs. |
| Milestone | n (%) | n (%) | n (%) | Control |

Sitting without Support 30 s, n (%)

| | | | | | | | | |
|---|---|---|---|---|---|---|---|---|
| Achieved* | 6 | (100.0)^{#} | 13 | (76.5)^{#} | 5 | (33.3) | NE | (NE, NE)^{‡} |
| Not Achievedt | 0 | (0.0) | 4 | (23.5) | 10 | (66.7) | 2.53 | (1.05, 6.07)^{¶} |

Rises to stand, n (%)

| | | | | | | | | |
|---|---|---|---|---|---|---|---|---|
| Achieved* | 5 | (83.3) | 2 | (11.8) | 0 | (0.0) | 16.00 | (2.40, 106.7)^{‡} |
| Not Achievedt | 1 | (16.7) | 15 | (88.2) | 15 | (100.0) | 2.00 | (1.40, 2.86)^{¶} |

Stands alone, n (%)

| | | | | | | | | |
|---|---|---|---|---|---|---|---|---|
| Achieved* | 5 | (83.3) | 1 | (5.9) | 0 | (0.0) | 16.00 | (2.40, 106.7)^{‡} |
| Not Achievedt | 1 | (16.7) | 16 | (94.1) | 15 | (100.0) | 1.94 | (1.38, 2.72)^{¶} |

Walks without support, n(%)

| | | | | | | | | |
|---|---|---|---|---|---|---|---|---|
| Achieved* | 5 | (83.3) | 1 | (5.9) | 0 | (0.0) | 16.00 | (2.40, 106.7)^{‡} |
| Not Achievedt | 1 | (16.7) | 16 | (94.1) | 15 | (100.0) | 1.94 | (1.38, 2.72)^{¶} |

Age at enrollment or dosing, years

| | | | | |
|---|---|---|---|---|
| Q1, Q2, Q3 | 0.84, 0.94, 2.57 | 1.33, 2.60, 5.39 | 0.77, 1.42, 2.63 | NA |
| Range (Min, Max) | (0.79, 4.14) | (0.56, 6.06) | (0.49, 3.27) | |

Age at first assessment, years

| | | | | |
|---|---|---|---|---|
| Q1, Q2, Q3 | 0.83, 0.94, 2.57 | 1.27, 2.59, 5.30 | 0.77, 1.44, 2.63 | NA |
| Range (Min, Max) | (0.79, 4.14) | (0.48, 6.06) | (0.50, 3.27) | |

Age at last assessment, years

| | | | | |
|---|---|---|---|---|
| Q1, Q2, Q3 | 2.37, 2.97, 4.43 | 2.42, 4.56, 6.37 | 1.98, 2.77, 3.81 | NA |
| Range (Min, Max) | (2.29, 6.17) | (1.49, 7.92) | (1.00, 5.99) | |

NE = not estimable.
*Achieved milestones are based on Bayley-Ill assessments (Bayley Item 26: Sits Without Support: 30 seconds; Bayley Item 35: Raises Self to Standing Position; Bayley Item 40: Stands Alone; Bayley Item 42: Walks Alone).
^{†}In absence of achievement of the milestone in Bayley-III, MFM-32 (Item 9: Sitting on the Floor; Item 11:
Going from sitting to standing up; Item 25: Standing), and/or CHOP INTEND (Item 12: Head Control).
^{‡}Relative risk to 1 x 1014 vg/kg control
^{¶}Relative risk to 3 x 1014 vg/kg control
^{#}One patient in the 1 x 1014 vg/kg dose group and one patient in the 3 x 1014 vg/kg dose group could sit without support at baseline.

TABLE 9
Increased Liver Function Parameters and Signs of Liver
Disease After Dosing in Deceased Participants

| | Participant 06 | Participant 09 | Participant 12 | Participant 40 |
|---|---|---|---|---|
| Age at dosing | 5.6 | 4.8 | 6.1 | 2.5 |
| Evidence of cholestasis | Yes | Yes | Yes | Yes |
| pre-dating dosing? | | | | |
| Dose received | Higher dose | Higher dose | Higher dose | Lower dose |
| Weight (total dose, vg) | 15.8 kg (4.8 × | 17.3 kg (5.2 × | 25.8 kg (7.7 × | 14.7 kg (5.8 × |
| | 1015 vg) | 1015 vg) | 1015 vg) | 1015 vg) |
| Initial SAE onset after | 41 days | 132 days | 50 days | 38 days |
| dosing | | | | |
| Liver symptoms | Initial, acute increases in LFTs occurred 1-4 weeks after dosing | | | |
| | Peak liver laboratory ranges observed after dosing: | | | |
| | Direct bilirubin: 28-92 × ULN | | | |
| | Total bilirubin: 34-54 × ULN | | | |
| | ALT: 7-22 × ULN | | | |
| | AST: 3-23 × ULN | | | |
| | GGT: 4-11 × ULN | | | |
| | Severe decompensated liver disease/dysfunction characterized by ascites, | | | |
| | extensive fibrosis, poor synthetic function characterized by both | | | |
| | hepatocellular injury and intrahepatic cholestasis | | | |
| Immediate cause of | Sepsis | Pseudomonas | Circulatory | Sepsis |
| death | | sepsis | collapse due to | |
| | | Hepatopathy, | gastro-intestinal | |
| | | severe immune | bleeding | |
| | | dysfunction | | |

ALT = alanine aminotransferase
AST = aspartate aminotransferase
GGT = gamma-glutamyl transferase
LFTs = liver function test parameters
SAE = serious adverse event
ULN = upper limit of normal

TABLE 10
Number of Liver-Related Adverse Events in Treated Subjects

| | Lower-dose | Higher-dose |
|---|---|---|
| | 1 × 10¹⁴ vg/kg | 3 × 10¹⁴ vg/kg |
| 24 treated participants | N = 7 | N = 17 |
| No liver laboratory abnormalities | 1 | 4 |
| reported as AEs or SAEs | | |
| Liver-related AEs reported | 6 | 13 |
| Non-fatal liver-related SAEs reported | 1 | 4 |
| Fatal hepatobiliary serious adverse | 1 | 3 |
| SAEs | | |

AEs = adverse events
SAEs = serious adverse events

TABLE 11
Treatment-emergent Serious Adverse Events Occurring in ≥1 Participant

| | 1 × 10¹⁴ vg/kg | | | 3 × 10¹⁴ vg/kg | | | Controls | | |
|---|---|---|---|---|---|---|---|---|---|
| System Organ Class | (N = 6) | | | (N = 17) | | | (N = 15) | | |
| Preferred Term | Events | Participants | | Events | Participants | | Events | Participants | |
| Total events/Number of | 19 | 4 | (66.7%) | 45 | 12 | (70.6%) | 41 | 13 | (86.7%) |
| Patients Reporting ≥1 SAE | | | | | | | | | |
| Blood and lymphatic system | 0 | 0 | (0.0%) | 1 | 1 | (5.9%) | 0 | 0 | (0.0%) |
| disorders | | | | | | | | | |
| Thrombocytopenia* | 0 | 0 | (0.0%) | 1 | 1 | (5.9%) | 0 | 0 | (0.0%) |
| Cardiac disorders | 2 | 1 | (16.7%) | 2 | 2 | (11.8%) | 1 | 1 | (6.7%) |
| Myocarditis | 1 | 1 | (16.7%) | 1 | 1 | (5.9%) | 0 | 0 | (0.0%) |
| Atrial tachycardia | 1 | 1 | (16.7%) | 0 | 0 | (0.0%) | 0 | 0 | (0.0%) |
| Tachycardia | 0 | 0 | (0.0%) | 1 | 1 | (5.9%) | 0 | 0 | (0.0%) |
| Cardiopulmonary failure | 0 | 0 | (0.0%) | 0 | 0 | (0.0%) | 1 | 1 | (6.7%) |
| Congenital, familial and | 0 | 0 | (0.0%) | 1 | 1 | (5.9%) | 0 | 0 | (0.0%) |
| genetic disorders | | | | | | | | | |
| Combined | 0 | 0 | (0.0%) | 1 | 1 | (5.9%) | 0 | 0 | (0.0%) |
| immunodeficiency | | | | | | | | | |
| Gastrointestinal disorders | 0 | 0 | (0.0%) | 8 | 5 | (29.4%) | 0 | 0 | (0.0%) |
| Ascites | 0 | 0 | (0.0%) | 4 | 3 | (17.6%) | 0 | 0 | (0.0%) |
| Colitis | 0 | 0 | (0.0%) | 1 | 1 | (5.9%) | 0 | 0 | 0.0%) |
| Constipation | 0 | 0 | (0.0%) | 1 | 1 | (5.9%) | 0 | 0 | (0.0%) |
| Gastrointestinal | 0 | 0 | (0.0%) | 1 | 1 | (5.9%) | 0 | 0 | (0.0%) |
| haemorrhage | | | | | | | | | |
| Protein-losing | 0 | 0 | (0.0%) | 1 | 1 | (5.9%) | 0 | 0 | (0.0%) |
| gastroenteropathy | | | | | | | | | |
| General disorders and | 0 | 0 | (0.0%) | 3 | 3 | (17.6%) | 0 | 0 | (0.0%) |
| administration site | | | | | | | | | |
| conditions | | | | | | | | | |
| Complication associated | 0 | 0 | (0.0%) | 2 | 2 | (11.8%) | 0 | 0 | (0.0%) |
| with device | | | | | | | | | |
| Pyrexia | 0 | 0 | (0.0%) | 1 | 1 | (5.9%) | 0 | 0 | (0.0%) |
| Hepatobiliary disorders | 0 | 0 | (0.0%) | 8 | 5 | (29.4%) | 2 | 2 | (13.3%) |
| Hyperbilirubinaemia | 0 | 0 | (0.0%) | 5 | 2 | (11.8%) | 0 | 0 | (0.0%) |
| Cholestasis^{†} | 0 | 0 | (0.0%) | 1 | 1 | (5.9%) | 0 | 0 | (0.0%) |
| Hepatitis | 0 | 0 | (0.0%) | 1 | 1 | (5.9%) | 0 | 0 | (0.0%) |
| Liver disorder^{‡} | 0 | 0 | (0.0%) | 1 | 1 | (5.9%) | 0 | 0 | (0.0%) |
| Cholecystitis acute | 0 | 0 | (0.0%) | 0 | 0 | (0.0%) | 1 | 1 | (6.7%) |
| Hepatic haemorrhage | 0 | 0 | (0.0%) | 0 | 0 | (0.0%) | 1 | 1 | (6.7%) |
| Immune system disorders | 0 | 0 | (0.0%) | 1 | 1 | (5.9%) | 0 | 0 | (0.0%) |
| Immune system disorder | 0 | 0 | (0.0%) | 1 | 1 | (5.9%) | 0 | 0 | (0.0%) |
| Infections and infestations | 8 | 4 | (66.7%) | 11 | 6 | (35.3%) | 24 | 10 | (66.7%) |
| Tracheitis | 0 | 0 | (0.0%) | 3 | 2 | (11.8%) | 0 | 0 | (0.0%) |
| Viral upper respiratory tract | 2 | 1 | (16.7%) | 0 | 0 | (0.0%) | 1 | 1 | (6.7%) |
| infection | | | | | | | | | |
| Bacterial sepsis | 0 | 0 | (0.0%) | 1 | 1 | (5.9%) | 0 | 0 | (0.0%) |
| Gastroenteritis | 0 | 0 | (0.0%) | 1 | 1 | (5.9%) | 0 | 0 | (0.0%) |
| Metapneumovirus infection | 1 | 1 | (16.7%) | 0 | 0 | (0.0%) | 0 | 0 | (0.0%) |
| Pneumocystis jirovecii | 0 | 0 | (0.0%) | 1 | 1 | (5.9%) | 0 | 0 | (0.0%) |
| pneumonia | | | | | | | | | |
| Pneumonia parainfluenzae | 1 | 1 | (16.7%) | 0 | 0 | (0.0%) | 0 | 0 | (0.0%) |
| viral | | | | | | | | | |
| Pneumonia respiratory | 1 | 1 | (16.7%) | 0 | 0 | (0.0%) | 0 | 0 | (0.0%) |
| syncytial viral | | | | | | | | | |
| Pneumonia viral | 1 | 1 | (16.7%) | 0 | 0 | (0.0%) | 1 | 1 | (6.7%) |
| Pseudomonal sepsis | 0 | 0 | (0.0%) | 1 | 1 | (5.9%) | 0 | 0 | (0.0%) |
| Respiratory syncytial virus | 0 | 0 | (0.0%) | 1 | 1 | (5.9%) | 0 | 0 | (0.0%) |
| infection | | | | | | | | | |
| Respiratory tract infection | 1 | 1 | (16.7%) | 0 | 0 | (0.0%) | 0 | 0 | (0.0%) |
| Sepsis | 0 | 0 | (0.0%) | 1 | 1 | (5.9%) | 0 | 0 | (0.0%) |
| Serratia sepsis | 0 | 0 | (0.0%) | 1 | 1 | (5.9%) | 0 | 0 | (0.0%) |
| Upper respiratory tract | 1 | 1 | (16.7%) | 0 | 0 | (0.0%) | 0 | 0 | (0.0%) |
| infection | | | | | | | | | |
| Viral infection | 0 | 0 | (0.0%) | 1 | 1 | (5.9%) | 0 | 0 | (0.0%) |
| Adenovirus infection | 0 | 0 | (0.0%) | 0 | 0 | (0.0%) | 1 | 1 | (6.7%) |
| Bacterial tracheitis | 0 | 0 | (0.0%) | 0 | 0 | (0.0%) | 1 | 1 | (6.7%) |
| Lower respiratory tract | 0 | 0 | (0.0%) | 0 | 0 | (0.0%) | 8 | 3 | (20.0%) |
| infection | | | | | | | | | |
| Otitis media | 0 | 0 | (0.0%) | 0 | 0 | (0.0%) | 1 | 1 | (6.7%) |
| Parainfluenzae virus | 0 | 0 | (0.0%) | 0 | 0 | (0.0%) | 1 | 1 | (6.7%) |
| infection | | | | | | | | | |
| Pneumonia | 0 | 0 | (0.0%) | 0 | 0 | (0.0%) | 3 | 3 | (20.0%) |
| Pneumonia moraxella | 0 | 0 | (0.0%) | 0 | 0 | (0.0%) | 1 | 1 | (6.7%) |
| Respiratory tract infection | 0 | 0 | (0.0%) | 0 | 0 | (0.0%) | 2 | 2 | (13.3%) |
| viral | | | | | | | | | |
| Rhinovirus infection | 0 | 0 | (0.0%) | 0 | 0 | (0.0%) | 3 | 2 | (13.3%) |
| Viral tracheitis | 0 | 0 | (0.0%) | 0 | 0 | (0.0%) | 1 | 1 | (6.7%) |
| Injury, poisoning and | 0 | 0 | (0.0%) | 0 | 0 | (0.0%) | 1 | 1 | (6.7%) |
| procedural complications | | | | | | | | | |
| Femur fracture | 0 | 0 | (0.0%) | 0 | 0 | (0.0%) | 1 | 1 | (6.7%) |
| Investigations | 2 | 1 | (16.7%) | 1 | 1 | (5.9%) | 3 | 2 | (13.3%) |
| Blood creatine | 1 | 1 | (16.7%) | 0 | 0 | (0.0%) | 0 | 0 | (0.0%) |
| phosphokinase increased | | | | | | | | | |
| Transaminases increased | 0 | 0 | (0.0%) | 1 | 1 | (5.9%) | 0 | 0 | (0.0%) |
| Troponin I increased | 1 | 1 | (16.7%) | 0 | 0 | (0.0%) | 0 | 0 | (0.0%) |
| Human rhinovirus test | 0 | 0 | (0.0%) | 0 | 0 | (0.0%) | 1 | 1 | (6.7%) |
| positive | | | | | | | | | |
| Pseudomonas test positive | 0 | 0 | (0.0%) | 0 | 0 | (0.0%) | 1 | 1 | (6.7%) |
| Respiratory syncytial virus | 0 | 0 | (0.0%) | 0 | 0 | (0.0%) | 1 | 1 | (6.7%) |
| test positive | | | | | | | | | |
| Metabolism and nutrition | 1 | 1 | (16.7%) | 0 | 0 | (0.0%) | 0 | 0 | (0.0%) |
| disorders | | | | | | | | | |
| Dehydration | 1 | 1 | (16.7%) | 0 | 0 | (0.0%) | 0 | 0 | (0.0%) |
| Musculoskeletal and | 0 | 0 | (0.0%) | 1 | 1 | (5.9%) | 0 | 0 | (0.0%) |
| connective tissue disorders | | | | | | | | | |
| Joint swelling | 0 | 0 | (0.0%) | 1 | 1 | (5.9%) | 0 | 0 | (0.0%) |
| Neoplasms benign, | 0 | 0 | (0.0%) | 3 | 1 | (5.9%) | 0 | 0 | (0.0%) |
| malignant and unspecified | | | | | | | | | |
| (incl cysts and polyps) | | | | | | | | | |
| Cholesteatoma | 0 | 0 | (0.0%) | 3 | 1 | (5.9%) | 0 | 0 | (0.0%) |
| Nervous system disorders | 1 | 1 | (16.7%) | 1 | 1 | (5.9%) | 2 | 1 | (6.7%) |
| Seizure | 1 | 1 | 16.7%) | 1 | 1 | (5.9%) | 1 | 1 | (6.7%) |
| Haemorrhage intracranial | 0 | 0 | (0.0%) | 0 | 0 | (0.0%) | 1 | 1 | (6.7%) |
| Respiratory, thoracic and | 5 | 1 | 16.7%) | 2 | 2 | (11.8%) | 8 | 7 | (46.7%) |
| mediastinal disorders | | | | | | | | | |
| Hypoxia | 3 | 1 | 16.7%) | 0 | 0 | (0.0%) | 0 | 0 | (0.0%) |
| Acute respiratory distress | 0 | 0 | (0.0%) | 1 | 1 | (5.9%) | 0 | 0 | (0.0%) |
| syndrome | | | | | | | | | |
| Acute respiratory failure | 1 | 1 | (16.7%) | 0 | 0 | 0.0%) | 0 | 0 | (0.0%) |
| Chronic respiratory failure | 0 | 0 | (0.0%) | 1 | 1 | (5.9%) | 0 | 0 | (0.0%) |
| Respiratory failure | 1 | 1 | (16.7%) | 0 | 0 | (0.0%) | 0 | 0 | (0.0%) |
| Atelectasis | 0 | 0 | (0.0%) | 0 | 0 | (0.0%) | 3 | 2 | (13.3%) |
| Bronchial secretion | 0 | 0 | (0.0%) | 0 | 0 | (0.0%) | 1 | 1 | (6.7%) |
| retention | | | | | | | | | |
| Pneumonia aspiration | 0 | 0 | (0.0%) | 0 | 0 | (0.0%) | 2 | 2 | (13.3%) |
| Respiratory distress | 0 | 0 | (0.0%) | 0 | 0 | (0.0%) | 2 | 2 | (13.3%) |
| Vascular disorders | 0 | 0 | (0.0%) | 2 | 2 | (11.8%) | 0 | 0 | (0.0%) |
| Hypertension | 0 | 0 | (0.0%) | 1 | 1 | (5.9%) | 0 | 0 | (0.0%) |
| Withdrawal hypertension | 0 | 0 | (0.0%) | 1 | 1 | (5.9%) | 0 | 0 | (0.0%) |

*Platelet count <150 × 10⁹/L
^{†}The 3 patients with cholestasis in the higher dose group are the 3 patients who died.

### Discussion

We report here dramatic improvement in ventilatory status, motor function and attainment of major motor milestones in a clinical trial of resamirigene bilparvovec, a gene therapy treatment for XLMTM. This represents the first effective therapy for this disease and an important milestone towards successful systemic gene therapy for a congenital myopathy.

Based on existing natural history data, XLMTM patients receiving long-term ventilation are extremely unlikely to wean from the ventilator. In a study following 33 ventilated XLMTM individuals prospectively for one year, no patients achieved ventilator independence or had meaningful reduction in time utilizing the ventilator. The ventilator independence achieved by some treated participants thus represents an extremely rare occurrence in the natural course of the disease. These improvements occurred in children ranging in age from 10 months to 6.8 years, demonstrating that even patients who have been chronically and invasively ventilated for several years can attain ventilator independence. As respiratory comorbidities are the primary cause of death for children with XLMTM, ventilator independence is likely to improve survival and overall quality of life by reducing risk of aspiration pneumonia and hospitalization and dependence on caregivers.

While treated participants improved greatly on respiratory parameters, untreated patients did not improve spontaneously, as also observed in natural history studies. We caution against comparing changes in ventilator support between lower-dose and higher-dose participants, as the former had longer follow-up and the latter were weaned from ventilation more gradually using a more conservative algorithm (manuscript in preparation). Similarly, the marked improvements in MIP occurred more quickly in lower-dose than higher-dose participants. It is important to note that once patients achieved ventilator independence and reached MIP of 80 cmH₂O on two separate assessments, MIP testing was ceased to minimize testing burden, which is arduous and distressing for the child, family and investigators. Because all six lower-dose participants achieved ventilator independence (one participant did resume noninvasive ventilation due to underlying scoliosis), there were fewer MIP measurements in the lower-dose cohort.

Treated participants had rapid increases in CHOP INTEND scores from baseline, with some participants reaching the ceiling of the scale (64 points), in contrast to little or no improvement in these scores among untreated control participants or patients in natural history studies. A strong treatment effect was also observed in older participants with XLMTM who had longer disease duration and greater accumulated XLMTM-related medical comorbidities. These children have achieved and subsequently maintained critical motor milestones, which is unprecedented in XLMTM patients with such an extensive disease burden, including five of six lower-dose and one of seventeen higher-dose participants walking independently (one using braces) at last assessment.

Treated participants had improvements in muscle pathology that paralleled their clinical improvements. Of note, the elevated proportion of myofibers with internal nuclei, which is a defining pathologic feature of XLMTM, was not altered with treatment, despite the observed clinical improvements.

Further studies are needed to determine whether changes in nuclear internalization may occur after the window when post-treatment biopsy specimens were collected. Additionally, although exogenous MTM1 expression was quite variable, treated participants showed clinically significant improvements, including ventilator independence and independent ambulation. We speculate that as an enzymatic phosphatase, relatively low levels of transgene expression within myofibers may be sufficient for clear histopathologic and impactful functional improvement.

The deaths of three participants following higher-dose resamirigene bilparvovec administration and one participant following lower-dose resamarigene bilparvovec has prompted re-examination of hepatobiliary disease in the natural history of XLMTM and consideration of the potential interaction of AAV-mediated therapy in this setting. All four participants showed dramatic elevations in total and direct bilirubin levels above baseline beginning 3-4 weeks after treatment, progressing to severe decompensated liver dysfunction characterized by ascites, extensive liver fibrosis, and reduced hepatic synthetic function, which is thought to be causative for the fatal events. The three higher-dose deceased participants who experienced hepatobiliary SAEs characterized by dramatic hyperbilirubinemia had different mutation types (Table 6) but shared notable clinical characteristics: as older (and therefore heavier) participants, they received among the highest total vector genome doses (4.8×10¹⁵-7.7×10¹⁵ vg) and had clinical evidence potentially consistent with cholestasis pre-dating resamirigene bilparvovec dosing, including intermittent hyperbilirubinemia, cholestatic hepatitis, and hepatic ultrasounds showing increased echogenicity. Among the heavier half of higher-dose participants (all receiving >4.5×10¹⁵ vg), the three participants without evidence of previous cholestasis did not experience hepatobiliary SAEs. Among eight lighter participants who received <4.5×10¹⁵ vg, including three with prior histories consistent with cholestasis, only one (Participant 38) had a hepatobiliary SAE. In comparison to the participants with fatal liver outcomes, he had delayed onset of symptoms (in reference to timing of resamirigene bilparvovec administration), absence of fibrotic injury on two liver biopsies, and no progression to decompensated disease. The changes in this case thus may be more reflective of underlying XLMTM pathology. In the lower-dose cohort (maximum dose 8.1×10¹⁴ vg), one of the seven participants have reported post-treatment hepatobiliary SAEs.

When the ASPIRO study began, the primary liver disease known to associate with XLMTM was hepatic peliosis—a rare, well-described, life-threatening vascular condition characterized by multiple, randomly distributed, blood-filled cavities throughout the liver. In addition, a non-specific cholestatic tendency, reported primarily as jaundice, cholelithiasis, and pruritus, had also been described in a small number of XLMTM patients, but the nature, extent, and pathophysiology were not well characterized and, unlike peliosis, were not known to be associated with morbidity or mortality in this population. In INCEPTUS, 24% of participants had histories of hepatobiliary disease at enrollment and, during that study, 12 participants (35%) had at least one elevated total bilirubin, including two with levels >5×ULN.

In addition, five recently documented cases in untreated XLMTM patients had clinical, laboratory, and histopathologic findings associated with intrahepatic cholestasis, often characterized by recurring episodes including risk for subsequent decompensated liver failure, further illustrating cholestasis as a significant part of the natural history of XLMTM. Despite frequent hepatic laboratory abnormalities, XLMTM patients do not routinely undergo diagnostic liver biopsies due to the low frequency of symptomatic disease and the risk for life-threatening hemorrhage.

Prior to the deaths of three higher-dose participants, the decision was made to move forward with the higher dose (3×10¹⁴ vg/kg) in the confirmatory phase of ASPIRO. This decision was based on the absence of significant differences in respiratory and neuromuscular outcomes between the two dose cohorts, lack of dose-limiting toxicities at either dose level, and muscle biopsy assessments at Week 24 suggesting more rapid histopathological improvements, dose-dependent transduction, transcription, and protein expression with the higher dose. Following the deaths of the three higher-dose participants, the remaining participants in ASPIRO are receiving the lower dose (1×10¹⁴ vg/kg) and will undergo increased hepatic monitoring and prophylactic or reactive therapies known to benefit children with chronic or recurring cholestasis due to other causes.

Overall, the percentage of participants with SAEs was similar among the treated and control groups. Three control participants died of causes similar to what has been observed in the natural history of the disease. Given that control participants were younger on average than treated participants, it is possible that they had more severe disease (i.e., increased fitness may be associated with living to an older age). However, treated and control participants shared similar degrees of baseline ventilator dependence. With the exception of the deaths related to hepatobiliary disease, most adverse events in treated participants were consistent with those reported in patients who received other AAV-based gene-therapy products, including transient thrombocytopenia and troponin elevation. Given the flow cytometry results, the rapid kinetics of thrombocytopenia resolution, and the giant platelets in blood smears, compensation may have been biphasic, first from splenic pool and later by platelet production from bone marrow.

For this first-in-human clinical trial in a pediatric disease with very high mortality, an open-label trial design with a delayed-treatment control was deemed appropriate, because a double-blind, placebo-controlled trial would have required sham muscle biopsies, double-dummy drug administration, use of placebo prednisolone for up to 16 weeks, and an intense visit schedule, often requiring considerable travel. Most participants were relatively young and dependent on invasive ventilation, reflecting the great unmet medical need in XLMTM. Clinical studies in older XLMTM patients are needed to assess the potential impact of resamirigene bilparvovec on potential reversibility, particularly with muscle stiffness/joint contractures as well as diaphragm and respiratory muscle progression in this population. Resamirigene bilparvovec's mechanism of action is not expected to be age-dependent, and XLMTM is not typically characterized by progressive loss of muscle mass due to fibroadipose replacement. The two oldest participants were almost 7 years old at the time of dosing: one (6.1 years at dosing) died as a result of hepatobiliary SAEs and one (6.8 years at dosing and requiring 24 hours of invasive ventilation daily) acquired the ability to sit unassisted for 30 seconds and achieved ventilator independence. The risk:benefit balance associated with administration of 1×10¹⁴ vg/kg warrants further investigation. Nevertheless, data from the ASPIRO trial to date support the clinical efficacy, safety, and histopathologic improvements with resamirigene bilparvovec treatment, and highlight the potential for this therapy to provide transformative clinical improvements for this rare, severe and fatal pediatric neuromuscular disease.

### CONCLUSION

In XLMTM patients, a rapid increase in muscle strength and motor developmental milestone acquisition was observed subsequent to resamirigene bilparvovec treatment compared to untreated controls. Specifically, motor function improved significantly, including attainment of the ability to walk unassisted for some. Furthermore, we also observed that resamirigene bilparvovec significantly reduced ventilator dependence, resulting in ventilator independence for some. Taken together, these results demonstrate the improved muscle strength and attainment of motor milestones and respiratory function in boys <5 years with XLMTM administered single-dose resamirigene bilparvovec.

### Example 4. Treatment of X-Linked Myotubular Myopathy in Human Patients by Administration of a Pseudotyped AAV2/8 Vector Including a Nucleic Acid Sequence Encoding a Myotubularin 1 Gene Operably Linked to a Desmin Promotor and an Anti-Cholestatic Agent

Using the compositions and methods of the disclosure, a patient (e.g., five years old or younger) having XLMTM may be administered a pseudotyped AAV2/8 vector including a nucleic acid sequence encoding an Myotubularin 1 (MTM1) gene operably linked to a desmin promotor (e.g., resamirigene bilparvovec), for example in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less), and an anti-cholestatic agent (e.g., ursodiol), for example in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the viral vector comprising a transgene encoding MTM1 (e.g., resamirigene bilparvovec) to the patient, the patient exhibits a change from baseline in hours of mechanical ventilation support over time. For example the patient exhibits the change from baseline in hours of mechanical ventilation support over time by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient. Upon administering the viral vector comprising a transgene encoding MTM1 (e.g., resamirigene bilparvovec) to the patient, the patient displays a bilirubin level that is greater than 1 mg/dL (e.g., 2 mg/dL, 3 mg/dL, 4 mg/dL, or 5 mg/dL) in a bilirubin test by about 3 weeks after administration of the viral vector to the patient.

### Example 5. Treatment of X-Linked Myotubular Myopathy in Human Patients by Administration of Resamirigene Bilparvovec and an Anti-Cholestatic Agent

Using the compositions and methods of the disclosure, a patient (e.g., five years old or younger) having XLMTM may be administered resamirigene bilparvovec. For example, in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less), and an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering resamirigene bilparvovec to the patient, the patient achieves functionally independent sitting for at least 30 seconds. For example, the patient achieves the functionally independent sitting by about 24 weeks (e.g., by about 20 weeks, 16 weeks, 12 weeks, 8 weeks, or 4 weeks) after administration of resamirigene bilparvovec to the patient.

### Example 6. Treatment of X-Linked Myotubular Myopathy in Human Patients by Administration of a Pseudotyped AAV2/8 Vector Including a Nucleic Acid Sequence Encoding a Myotubularin 1 Gene Operably Linked to a Desmin Promotor and Ursodiol

Using the compositions and methods of the disclosure, a patient (e.g., five years old or younger) having XLMTM may be administered a pseudotyped AAV2/8 vector including a nucleic acid sequence encoding an MTM1 gene operably linked to a desmin promotor (e.g., resamirigene bilparvovec). For example, in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less), and ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a reduction in required mechanical ventilator support to about 16 hours or less per day. For example, the patient displays the reduction in required ventilator support by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 7. Treatment of X-Linked Myotubular Myopathy in Human Patients by Administration of Resamirigene Bilparvovec and Ursodiol

Using the compositions and methods of the disclosure, a patient (e.g., five years old or younger) having XLMTM may be administered resamirigene bilparvovec. For example, in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less), and ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering resamirigene bilparvovec to the patient, the patient displays a change from baseline on the CHOP INTEND. For example, the patient displays the change from baseline on the CHOP INTEND by about 24 weeks (e.g., by about 20 weeks, 16 weeks, 12 weeks, 8 weeks, or 4 weeks) after administration of resamirigene bilparvovec to the patient.

### Example 8. Treatment of Cholestasis or Hyperbilirubinemia in a Human Patient that has X-Linked Myotubular Myopathy by Administration of a Pseudotyped AAV2/8 Vector Including a Nucleic Acid Sequence Encoding a Myotubularin 1 Gene Operably Linked to a Desmin Promotor

Using the compositions and methods of the disclosure, a patient (e.g., five years old or younger) having XLMTM that has been previously administered an anti-cholestatic agent may be administered a pseudotyped AAV2/8 vector including a nucleic acid sequence encoding an MTM1 gene operably linked to a desmin promotor (e.g., resamirigene bilparvovec). For example, in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less), and ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a reduction in required mechanical ventilator support to about 16 hours or less per day. For example, the patient displays the reduction in required ventilator support by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 9. Treatment of X-Linked Myotubular Myopathy in Human Patients by Administration of a Pseudotyped AAV2/8 Vector Including a Nucleic Acid Sequence Encoding a Myotubularin 1 Gene Operably Linked to a Desmin Promotor

Using the compositions and methods of the disclosure, a patient having XLMTM may be administered a pseudotyped AAV2/8 vector including a nucleic acid sequence encoding an MTM1 gene operably linked to a desmin promotor (e.g., resamirigene bilparvovec) in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less). Afterwards, the patient may be monitored for the development of cholestasis and/or hyperbilirubinemia, and if the patient is determined to exhibit cholestasis or hyperbilirubinemia or one or more symptoms thereof, the patient is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a change from baseline in maximal inspiratory pressure. For example, the patient displays the change from baseline in maximal inspiratory pressure by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 10. Treatment of X-Linked Myotubular Myopathy in Human Patients by Administration of Resamirigene Bilparvovec

Using the compositions and methods of the disclosure, a patient having XLMTM may be administered resamirigene bilparvovec in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less). Afterwards, the patient may be monitored for the development of cholestasis and/or hyperbilirubinemia, and if the patient is determined to exhibit cholestasis or hyperbilirubinemia or one or more symptoms thereof, the patient is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering resamirigene bilparvovec to the patient, the patient displays a change from baseline in maximal inspiratory pressure. For example, the patient displays the change from baseline in maximal inspiratory pressure by about 24 weeks (e.g., by about 20 weeks, 16 weeks, 12 weeks, 8 weeks, or 4 weeks) after administration of resamirigene bilparvovec to the patient.

### Example 11. Treatment of X-Linked Myotubular Myopathy in Human Patients that are Five Years Old or Younger by Administration of a Pseudotyped AAV2/8 Vector Including a Nucleic Acid Sequence Encoding a Myotubularin 1 Gene Operably Linked to a Desmin Promotor

Using the compositions and methods of the disclosure, a patient having XLMTM that is less than about five years old may be administered a pseudotyped AAV2/8 vector including a nucleic acid sequence encoding an MTM1 gene operably linked to a desmin promotor (e.g., resamirigene bilparvovec). For example, in a dose of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less). Afterwards, the patient may be monitored for the development of cholestasis, and if the patient is determined to exhibit cholestasis or one or more symptoms thereof, the patient is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a change from baseline in quantitative analysis of myotubularin expression in a muscle biopsy. For example, the patient displays the change from baseline in quantitative analysis of myotubularin expression in a muscle biopsy by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient. Upon administering the pseudotyped AAV2/8 vector including a nucleic acid sequence encoding an MTM1 gene operably linked to a desmin promotor (e.g., resamirigene bilparvovec) to the patient, the patient displays a reduction of stiffness and/or joint contractures by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 12. Treatment of X-Linked Myotubular Myopathy in Human Patients that are Five Years Old or Younger by Administration of Resamirigene Bilparvovec

Using the compositions and methods of the disclosure, a patient having XLMTM that is less than about five years old may be administered resamirigene bilparvovec. For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less). Afterwards, the patient may be monitored for the development of cholestasis, and if the patient is determined to exhibit cholestasis or one or more symptoms thereof, the patient is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering resamirigene bilparvovec to the patient, the patient displays a change from baseline in quantitative analysis of myotubularin expression in a muscle biopsy. For example, the patient displays the change from baseline in quantitative analysis of myotubularin expression in a muscle biopsy by about 24 weeks (e.g., by about 20 weeks, 16 weeks, 12 weeks, 8 weeks, or 4 weeks) after administration of resamirigene bilparvovec to the patient. For example, the change from baseline in quantitative analysis of myotubularin expression in a muscle biopsy persists for at least 48 weeks after administration of the viral vector to the patient. Upon administering resamirigene bilparvovec to the patient, the patient displays diaphragm and/or respiratory muscle progression by about 24 weeks (e.g., by about 20 weeks, 16 weeks, 12 weeks, 8 weeks, or 4 weeks) after administration of resamirigene bilparvovec to the patient.

### Example 13. Treatment of X-Linked Myotubular Myopathy in Human Patients by Administration of a Pseudotyped AAV2/8 Vector Including a Nucleic Acid Sequence Encoding a Myotubularin 1 Gene Operably Linked to a Desmin Promotor and an Anti-Cholestatic Agent

Using the compositions and methods of the disclosure, a patient having XLMTM may be administered a pseudotyped AAV2/8 vector including a nucleic acid sequence encoding an MTM1 gene operably linked to a desmin promotor (e.g., resamirigene bilparvovec) in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less). Afterwards, it is determined that the patient exhibits cholestasis or hyperbilirubinemia or one or more symptoms thereof, and the patient is be administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor to the patient, the patient displays a change from baseline in maximal inspiratory pressure. For example, the patient displays the change from baseline in maximal inspiratory pressure by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 14. Treatment of X-Linked Myotubular Myopathy in Human Patients by Administration of Resamirigene Bilparvovec and an Anti-Cholestatic Agent

Using the compositions and methods of the disclosure, a patient having XLMTM may be administered resamirigene bilparvovec in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10² vg/kg, 1×10¹¹ vg/kg, 1×10¹⁰ vg/kg, 1×10⁹ vg/kg, 1×10⁸ vg/kg, or less). Afterwards, it is determined that the patient exhibits cholestasis or hyperbilirubinemia or one or more symptoms thereof, and the patient is be administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering resamirigene bilparvovec to the patient, the patient displays a change from baseline in maximal inspiratory pressure. For example, the patient displays the change from baseline in maximal inspiratory pressure by about 24 weeks (e.g., by about 20 weeks, 16 weeks, 12 weeks, 8 weeks, or 4 weeks) after administration of resamirigene bilparvovec to the patient.

### Example 15. Treatment of X-Linked Myotubular Myopathy in Human Patients that are Five Years Old or Younger by Administration of a Pseudotyped AAV2/8 Vector Including a Nucleic Acid Sequence Encoding a Myotubularin 1 Gene Operably Linked to a Desmin Promotor and an Anti-Cholestatic Agent

Using the compositions and methods of the disclosure, a patient having XLMTM that is less than about five years old may be administered a pseudotyped AAV2/8 vector including a nucleic acid sequence encoding an MTM1 gene operably linked to a desmin promotor (e.g., resamirigene bilparvovec). For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10² vg/kg, 1×10¹¹ vg/kg, 1×10¹⁰ vg/kg, 1×10⁹ vg/kg, 1×10⁸ vg/kg, or less). Afterwards, it is determined that the patient exhibits cholestasis or hyperbilirubinemia or one or more symptoms thereof, and the patient is be administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a change from baseline in quantitative analysis of myotubularin expression in a muscle biopsy. For example, the patient displays the change from baseline in quantitative analysis of myotubularin expression in a muscle biopsy by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient. For example, the change from baseline in quantitative analysis of myotubularin expression in a muscle biopsy persists for at least 48 weeks after administration of the viral vector to the patient.

### Example 16. Treatment of X-Linked Myotubular Myopathy in Human Patients that are Five Years Old or Younger by Administration of Resamirigene Bilparvovec and an Anti-Cholestatic Agent

Using the compositions and methods of the disclosure, a patient having XLMTM that is less than about five years old may be administered resamirigene bilparvovec. For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less). Afterwards, it is determined that the patient exhibits cholestasis or hyperbilirubinemia or one or more symptoms thereof, and the patient is be administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering resamirigene bilparvovec to the patient, the patient displays a change from baseline in quantitative analysis of myotubularin expression in a muscle biopsy. For example, the patient displays the change from baseline in quantitative analysis of myotubularin expression in a muscle biopsy by about 24 weeks (e.g., by about 20 weeks, 16 weeks, 12 weeks, 8 weeks, or 4 weeks) after administration of resamirigene bilparvovec to the patient. For example, the change from baseline in quantitative analysis of myotubularin expression in a muscle biopsy persists for at least 48 weeks after administration of the viral vector to the patient.

### Example 17. Treatment or Prevention of Cholestasis or Hyperbilirubinemia in a Human Patient that has X-Linked Myotubular Myopathy by Administration of an Anti-Cholestatic Agent

Using the compositions and methods of the disclosure, a patient having XLMTM that was previously administered a pseudotyped AAV2/8 vector including a nucleic acid sequence encoding an MTM1 gene operably linked to a desmin promotor (e.g., resamirigene bilparvovec) in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient exhibits a change from baseline in hours of mechanical ventilation support overtime. For example, the patient exhibits the change from baseline in hours of mechanical ventilation support over time by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 18. Treatment or Prevention of Cholestasis or Hyperbilirubinemia in a Human Patient that has X-Linked Myotubular Myopathy by Administration of an Anti-Cholestatic Agent

Using the compositions and methods of the disclosure, a patient having XLMTM that was previously administered resamirigene bilparvovec in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient achieves functionally independent sitting for at least 30 seconds. For example, the patient achieves the functionally independent sitting by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 19. Treatment or Prevention of Cholestasis or Hyperbilirubinemia in a Human Patient that has X-Linked Myotubular Myopathy by Administration of Ursodiol

Using the compositions and methods of the disclosure, a patient having XLMTM that was previously administered a pseudotyped AAV2/8 vector including a nucleic acid sequence encoding an MTM1 gene operably linked to a desmin promotor (e.g., resamirigene bilparvovec) in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a reduction in required mechanical ventilator support to about 16 hours or less per day. For example, the patient displays the reduction in required ventilator support by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 20. Treatment or Prevention of Cholestasis or Hyperbilirubinemia in a Human Patient that has X-Linked Myotubular Myopathy by Administration of Ursodiol

Using the compositions and methods of the disclosure, a patient having XLMTM that was previously administered resamirigene bilparvovec in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

### Example 21. Treatment or Prevention of Cholestasis or Hyperbilirubinemia in a Human Patient that is Five Years Old or Younger and Who has X-Linked Myotubular Myopathy by Administration of an Anti-Cholestatic Agent

Using the compositions and methods of the disclosure, a patient that is five years old or younger having XLMTM that was previously administered a pseudotyped AAV2/8 vector including a nucleic acid sequence encoding an MTM1 gene operably linked to a desmin promotor (e.g., resamirigene bilparvovec). For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient exhibits a change from baseline in hours of mechanical ventilation support overtime. For example, the patient exhibits the change from baseline in hours of mechanical ventilation support over time by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 22. Treatment or Prevention of Cholestasis or Hyperbilirubinemia in a Human that is Five Years Old or Younger and Who has X-Linked Myotubular Myopathy by Administration of an Anti-Cholestatic Agent

Using the compositions and methods of the disclosure, a patient that is five years old or younger having XLMTM that was previously administered resamirigene bilparvovec. For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered an anti-cholestatic agent (e.g., ursodiol). For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient achieves functionally independent sitting for at least 30 seconds. For example, the patient achieves the functionally independent sitting by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 23. Treatment or Prevention of Cholestasis or Hyperbilirubinemia in a Human Patient that is Five Years Old or Younger and Who has X-Linked Myotubular Myopathy by Administration of Ursodiol

Using the compositions and methods of the disclosure, a patient that is five years old or younger having XLMTM that was previously administered a pseudotyped AAV2/8 vector including a nucleic acid sequence encoding an MTM1 gene operably linked to a desmin promotor (e.g., resamirigene bilparvovec). For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less) is administered ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

Upon administering the pseudotyped AAV2/8 vector including a nucleic acid sequence encoding an MTM1 gene operably linked to a desmin promotor (e.g., resamirigene bilparvovec) to the patient, the patient displays a reduction in required mechanical ventilator support to about 16 hours or less per day. For example, the patient displays the reduction in required ventilator support by about 24 weeks (e.g., by about 20 weeks, 16 weeks, 12 weeks, 8 weeks, or 4 weeks) after administration of the pseudotyped AAV2/8 vector including a nucleic acid sequence encoding an MTM1 gene operably linked to a desmin promotor (e.g., resamirigene bilparvovec) to the patient.

### Example 24. Treatment or Prevention of Cholestasis or Hyperbilirubinemia in a Human Patient that is Five Years Old or Younger and Who has X-Linked Myotubular Myopathy by Administration of Ursodiol

Using the compositions and methods of the disclosure, a patient that is five years old or younger having XLMTM that was previously administered resamirigene bilparvovec. For example, in a dose less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less) is administered ursodiol. For example, in one or doses of from about 5 mg/kg/dose to about 20 mg/kg/dose and by way of a unit dosage form comprising 250 mg or 500 mg and/or from 5 mg/kg/day to about 40 mg/kg/day.

### OTHER EMBODIMENTS

In addition to the sections outlined above, the compositions and methods of the present disclosure are also captured in the following enumerated embodiments:

[1] A method of treating XLMTM in a human patient in need thereof, the method comprising administering to the patient (i) a therapeutically effective amount of a transgene encoding MTM1 and (ii) an anti-cholestatic agent, wherein the anti-cholestatic agent is administered to the patient in one or more (e.g., one or more, two or more, three or more, four or more, five or more, six or more, seven or more, eight or more, nine or more, or ten or more) doses that commence within about six weeks (e.g., about six weeks before or about six weeks after) of administration of the transgene to the patient.

[2] A method of reducing stiffness and/or joint contractures in a human patient diagnosed as having XLMTM, the method comprising administering to the patient (i) a therapeutically effective amount of a transgene encoding MTM1 and (ii) an anti-cholestatic agent, wherein the anti-cholestatic agent is administered to the patient in one or more doses that commence within about six weeks of administration of the viral vector to the patient.

[3] A method of increasing diaphragm and/or respiratory muscle progression in a human patient diagnosed as having XLMTM, the method comprising administering to the patient (i) a therapeutically effective amount of a transgene encoding MTM1 and (ii) an anti-cholestatic agent, wherein the anti-cholestatic agent is administered to the patient in one or more doses that commence within about six weeks of administration of the viral vector to the patient.

[4] The method of any one of embodiments 1-3, wherein the transgene encoding MTM1 is administered to the patient by transduction with a viral vector comprising a transgene encoding MTM1.

[5] The method of any one of embodiments 1-4, wherein the anti-cholestatic agent is administered to the patient in one or more (e.g., one or more, two or more, three or more, four or more, five or more, six or more, seven or more, eight or more, nine or more, or ten or more) doses that commence within about five weeks (e.g., about five weeks before or about five weeks after) of administration of the transgene or viral vector to the patient.

[6] The method of embodiment 5, wherein the anti-cholestatic agent is administered to the patient in one or more (e.g., one or more, two or more, three or more, four or more, five or more, six or more, seven or more, eight or more, nine or more, or ten or more) doses that commence within about four weeks (e.g., about four weeks before or about four weeks after) of administration of the transgene or viral vector to the patient.

[7] The method of embodiment 5, wherein the anti-cholestatic agent is administered to the patient in one or more (e.g., one or more, two or more, three or more, four or more, five or more, six or more, seven or more, eight or more, nine or more, or ten or more) doses that commence within about three weeks (e.g., about three weeks before or about three weeks after) of administration of the transgene or viral vector to the patient.

[8] The method of embodiment 5, wherein the anti-cholestatic agent is administered to the patient in one or more (e.g., one or more, two or more, three or more, four or more, five or more, six or more, seven or more, eight or more, nine or more, or ten or more) doses that commence within about two weeks (e.g., about two weeks before or about two weeks after) of administration of the transgene or viral vector to the patient.

[9] The method of embodiment 5, wherein the anti-cholestatic agent is administered to the patient in one or more (e.g., one or more, two or more, three or more, four or more, five or more, six or more, seven or more, eight or more, nine or more, or ten or more) doses that commence within about one week (e.g., about one week before or about one week after, about six days before or about six days after, about five days before or about five days after, about four days before or about four days after, about three days before or about three days after, about two days before or about two days after, or about one day before or about one day after) of administration of the transgene or viral vector to the patient.

[10] The method of embodiment 5, wherein the anti-cholestatic agent is administered to the patient in one or more (e.g., one or more, two or more, three or more, four or more, five or more, six or more, seven or more, eight or more, nine or more, or ten or more) doses that commence on the same day (e.g., 24^{th} hour, on the 23^{rd} hour, on the 22^{nd} hour, on the 21^{st} hour, on the 20^{th} hour, on the 19^{th} hour, on the 18^{th} hour, on the 17^{th} hour, on the 16^{th} hour, on the 15^{th} hour, on the 14^{th} hour, on the 13^{th} hour, on the 12^{th} hour, on the 11^{th} hour, on the 10^{th} hour, on the 9^{th} hour, on the 8^{th} hour, on the 7^{th} hour, on the 6^{th} hour, on the 5^{th} hour, on the 4^{th} hour, on the 3^{rd} hour, on the 2^{nd} hour, on the 1^{st} hour, on the 60^{th} minute, on the 59^{th} minute, on the 58^{th} minute, on the 57^{th} minute, on the 56^{th} minute, on the 55^{th} minute, on the 50^{th} minute, on the 40^{th} minute, on the 30^{th} minute, on the 20^{th} minute, on the 10^{th} minute, or on the same minute) as administration of the transgene or viral vector to the patient.

[11] A method of treating XLMTM in a human patient in need thereof and who has been previously administered an anti-cholestatic agent, the method comprising administering to the patient a therapeutically effective amount of a transgene encoding MTM1.

[12] A method of reducing stiffness and/or joint contractures in a human patient diagnosed as having XLMTM and who has been previously administered an anti-cholestatic agent, the method comprising administering to the patient a therapeutically effective amount of a transgene encoding MTM1.

[13] A method of increasing diaphragm and/or respiratory muscle progression in a human patient diagnosed as having XLMTM and who has been previously administered an anti-cholestatic agent, the method comprising administering to the patient a therapeutically effective amount of a transgene encoding MTM1.

[14] The method of any one of embodiments 11-13, wherein the transgene encoding MTM1 is administered to the patient by transduction with a viral vector comprising a transgene encoding MTM1.

[15] The method of any one of embodiments 4-10 or 14, wherein the viral vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less).

[16] The method of embodiment 15, wherein the viral vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[17] The method of embodiment 16, wherein the viral vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[18] The method of embodiment 16, wherein the viral vector is administered to the patient in an amount of less than about 1.5×10¹⁴ vg/kg (e.g., in an amount of less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[19] The method of embodiment 16, wherein the viral vector is administered to the patient in an amount of less than about 1.4×10¹⁴ vg/kg (e.g., in an amount of less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[20] The method of any one of embodiments 4-10 or 14, wherein the viral vector is administered to the patient in an amount of from about 3×10¹³ vg/kg to about 2.3×10¹⁴ vg/kg (e.g., 3×10¹³ vg/kg to about 2.3×10¹⁴ vg/kg, 3.1×10¹³ vg/kg to about 2.3×10¹⁴ vg/kg, 3.2×10¹³ vg/kg to about 2.3×10¹⁴ vg/kg, 3.3×10¹³ vg/kg to about 2.3×10¹⁴ vg/kg, 3.4×10¹³ vg/kg to about 2.3×10¹⁴ vg/kg, 3.5×10¹³ vg/kg to about 2.3×10¹⁴ vg/kg, 3.6×10¹³ vg/kg to about 2.3×10¹⁴ vg/kg, 3.7×10¹³ vg/kg to about 2.3×10¹⁴ vg/kg, 3.8×10¹³ vg/kg to about 2.3×10¹⁴ vg/kg, 3.9×10¹³ vg/kg to about 2.3×10¹⁴ vg/kg, 4×10¹³ vg/kg to about 2.3×10¹⁴ vg/kg, 4.1×10¹³ vg/kg to about 2.3×10¹⁴ vg/kg, 4.2×10¹³ vg/kg to about 2.3×10¹⁴ vg/kg, 4.3×10¹³ vg/kg to about 2.3×10¹⁴ vg/kg, 4.4×10¹³ vg/kg to about 2.3×10¹⁴ vg/kg, 4.5×10¹³ vg/kg to about 2.3×10¹⁴ vg/kg, 4.6×10¹³ vg/kg to about 2.3×10¹⁴ vg/kg, 4.7×10¹³ vg/kg to about 2.3×10¹⁴ vg/kg, 4.8×10¹³ vg/kg to about 2.3×10¹⁴ vg/kg, 4.9×10¹³ vg/kg to about 2.3×10¹⁴ vg/kg, 5×10¹³ vg/kg to about 2.3×10¹⁴ vg/kg, 5.1×10¹³ vg/kg to about 2.3×10¹⁴ vg/kg, 5.2×10¹³ vg/kg to about 2.3×10¹⁴ vg/kg, 5.3×10¹³ vg/kg to about 2.3×10¹⁴ vg/kg, 5.4×10¹³ vg/kg to about 2.3×10¹⁴ vg/kg, 5.5×10¹³ vg/kg to about 2.3×10¹⁴ vg/kg, 5.6×10¹³ vg/kg to about 2.3×10¹⁴ vg/kg, 5.7×10¹³ vg/kg to about 2.3×10¹⁴ vg/kg, 5.8×10¹³ vg/kg to about 2.3×10¹⁴ vg/kg, 5.9×10¹³ vg/kg to about 2.3×10¹⁴ vg/kg, 6×10¹³ vg/kg to about 2.3×10¹⁴ vg/kg, 6.1×10¹³ vg/kg to about 2.3×10¹⁴ vg/kg, 6.2×10¹³ vg/kg to about 2.3×10¹⁴ vg/kg, 6.3×10¹³ vg/kg to about 2.3×10¹⁴ vg/kg, 6.4×10¹³ vg/kg to about 2.3×10¹⁴ vg/kg, 6.5×10¹³ vg/kg to about 2.3×10¹⁴ vg/kg, 6.6×10¹³ vg/kg to about 2.3×10¹⁴ vg/kg, 6.7×10¹³ vg/kg to about 2.3×10¹⁴ vg/kg, 6.8×10¹³ vg/kg to about 2.3×10¹⁴ vg/kg, 6.9×10¹³ vg/kg to about 2.3×10¹⁴ vg/kg, 7×10¹³ vg/kg to about 2.3×10¹⁴ vg/kg, 7.1×10¹³ vg/kg to about 2.3×10¹⁴ vg/kg, 7.2×10¹³ vg/kg to about 2.3×10¹⁴ vg/kg, 7.3×10¹³ vg/kg to about 2.3×10¹⁴ vg/kg, 7.4×10¹³ vg/kg to about 2.3×10¹⁴ vg/kg, 7.5×10¹³ vg/kg to about 2.3×10¹⁴ vg/kg, 7.6×10¹³ vg/kg to about 2.3×10¹⁴ vg/kg, 7.7×10¹³ vg/kg to about 2.3×10¹⁴ vg/kg, 7.8×10¹³ vg/kg to about 2.3×10¹⁴ vg/kg, 7.9×10¹³ vg/kg to about 2.3×10¹⁴ vg/kg, 8×10¹³ vg/kg to about 2.3×10¹⁴ vg/kg, 8.1×10¹³ vg/kg to about 2.3×10¹⁴ vg/kg, 8.2×10¹³ vg/kg to about 2.3×10¹⁴ vg/kg, 8.3×10¹³ vg/kg to about 2.3×10¹⁴ vg/kg, 8.4×10¹³ vg/kg to about 2.3×10¹⁴ vg/kg, 8.5×10¹³ vg/kg to about 2.3×10¹⁴ vg/kg, 8.6×10¹³ vg/kg to about 2.3×10¹⁴ vg/kg, 8.7×10¹³ vg/kg to about 2.3×10¹⁴ vg/kg, 8.8×10¹³ vg/kg to about 2.3×10¹⁴ vg/kg, 8.9×10¹³ vg/kg to about 2.3×10¹⁴ vg/kg, 9×10¹³ vg/kg to about 2.3×10¹⁴ vg/kg, 9.1×10¹³ vg/kg to about 2.3×10¹⁴ vg/kg, 9.2×10¹³ vg/kg to about 2.3×10¹⁴ vg/kg, 9.3×10¹³ vg/kg to about 2.3×10¹⁴ vg/kg, 9.4×10¹³ vg/kg to about 2.3×10¹⁴ vg/kg, 9.5×10¹³ vg/kg to about 2.3×10¹⁴ vg/kg, 9.6×10¹³ vg/kg to about 2.3×10¹⁴ vg/kg, 9.7×10¹³ vg/kg to about 2.3×10¹⁴ vg/kg, 9.8×10¹³ vg/kg to about 2.3×10¹⁴ vg/kg, 9.9×10¹³ vg/kg to about 2.3×10¹⁴ vg/kg, 1×10¹⁴ vg/kg to about 2.3×10¹⁴ vg/kg, 1.1×10¹⁴ vg/kg to about 2.3×10¹⁴ vg/kg, 1.2×10¹⁴ vg/kg to about 2.3×10¹⁴ vg/kg, 1.3×10¹⁴ vg/kg to about 2.3×10¹⁴ vg/kg, 1.4×10¹⁴ vg/kg to about 2.3×10¹⁴ vg/kg, 1.5×10¹⁴ vg/kg to about 2.3×10¹⁴ vg/kg, 1.6×10¹⁴ vg/kg to about 2.3×10¹⁴ vg/kg, 1.7×10¹⁴ vg/kg to about 2.3×10¹⁴ vg/kg, 1.8×10¹⁴ vg/kg to about 2.3×10¹⁴ vg/kg, 1.9×10¹⁴ vg/kg to about 2.3×10¹⁴ vg/kg, 2×10¹⁴ vg/kg to about 2.3×10¹⁴ vg/kg, 2.1×10¹⁴ vg/kg to about 2.3×10¹⁴ vg/kg, or 2.2×10¹⁴ vg/kg to about 2.3×10¹⁴ vg/kg).

[21] The method of embodiment 20, wherein the viral vector is administered to the patient in an amount of from about 8×10¹³ vg/kg to about 1.8×10⁴ vg/kg (e.g., 8×10¹³ vg/kg to about 1.8×10¹⁴ vg/kg, 8.1×10¹³ vg/kg to about 1.8×10¹⁴ vg/kg, 8.2×10¹³ vg/kg to about 1.8×10¹⁴ vg/kg, 8.3×10¹³ vg/kg to about 1.8×10¹⁴ vg/kg, 8.4×10¹³ vg/kg to about 1.8×10¹⁴ vg/kg, 8.5×10¹³ vg/kg to about 1.8×10¹⁴ vg/kg, 8.6×10¹³ vg/kg to about 1.8×10¹⁴ vg/kg, 8.7×10¹³ vg/kg to about 1.8×10¹⁴ vg/kg, 8.8×10¹³ vg/kg to about 1.8×10¹⁴ vg/kg, 8.9×10¹³ vg/kg to about 1.8×10¹⁴ vg/kg, 9×10¹³ vg/kg to about 1.8×10¹⁴ vg/kg, 9.1×10¹³ vg/kg to about 1.8×10¹⁴ vg/kg, 9.2×10¹³ vg/kg to about 1.8×10¹⁴ vg/kg, 9.3×10¹³ vg/kg to about 1.8×10¹⁴ vg/kg, 9.4×10¹³ vg/kg to about 1.8×10¹⁴ vg/kg, 9.5×10¹³ vg/kg to about 1.8×10¹⁴ vg/kg, 9.6×10¹³ vg/kg to about 1.8×10¹⁴ vg/kg, 9.7×10¹³ vg/kg to about 1.8×10¹⁴ vg/kg, 9.8×10¹³ vg/kg to about 1.8×10¹⁴ vg/kg, 9.9×10¹³ vg/kg to about 1.8×10¹⁴ vg/kg, 1×10¹⁴ vg/kg to about 1.8×10¹⁴ vg/kg, 1.1×10⁴ vg/kg to about 1.8×10¹⁴ vg/kg, 1.2×10¹⁴ vg/kg to about 1.8×10¹⁴ vg/kg, 1.3×10¹⁴ vg/kg to about 1.8×10¹⁴ vg/kg, 1.4×10¹⁴ vg/kg to about 1.8×10¹⁴ vg/kg, 1.5×10¹⁴ vg/kg to about 1.8×10¹⁴ vg/kg, 1.6×10¹⁴ vg/kg to about 1.8×10¹⁴ vg/kg, or 1.7×10¹⁴ vg/kg to about 1.8×10¹⁴ vg/kg).

[22] The method of embodiment 20, wherein the viral vector is administered to the patient in an amount of from about 1×10¹⁴ vg/kg to about 1.6×10¹⁴ vg/kg (e.g., 1×10¹⁴ vg/kg to about 1.6×10¹⁴ vg/kg, 1.1×10⁴ vg/kg to about 1.6×10¹⁴ vg/kg, 1.2×10¹⁴ vg/kg to about 1.6×10¹⁴ vg/kg, 1.3×10¹⁴ vg/kg to about 1.6×10¹⁴ vg/kg, 1.4×10¹⁴ vg/kg to about 1.6×10¹⁴ vg/kg, 1.5×10¹⁴ vg/kg to about 1.6×10¹⁴ vg/kg, 1.6×10¹⁴ vg/kg to about 1.6×10¹⁴ vg/kg, or 1.7×10¹⁴ vg/kg to about 1.6×10¹⁴ vg/kg).

[23] The method of embodiment 20, wherein the viral vector is administered to the patient in an amount of 1.1×10⁴ vg/kg to about 1.5×10¹⁴ vg/kg (e.g., 1.1×10⁴ vg/kg to about 1.5×10¹⁴ vg/kg, 1.2×10¹⁴ vg/kg to about 1.5×10¹⁴ vg/kg, 1.3×10¹⁴ vg/kg to about 1.5×10¹⁴ vg/kg, or 1.4×10¹⁴ vg/kg to about 1.5×10¹⁴ vg/kg).

[24] The method of embodiment 20, wherein the viral vector is administered to the patient in an amount of from about 1.2×10¹⁴ vg/kg to about 1.4×10¹⁴ vg/kg (e.g., 1.2×10¹⁴ vg/kg to about 1.4×10¹⁴ vg/kg, or 1.3×10¹⁴ vg/kg to about 1.4×10¹⁴ vg/kg).

[25] The method of any one of embodiments 4-10 or 14-24, wherein the viral vector is administered to the patient in an amount of about 1.3×10¹⁴ vg/kg.

[26] The method of any one of embodiments 1-25, wherein the patient is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[27] The method of embodiment 26, wherein the patient is four years old or younger (e.g., 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[28] The method of embodiment 27, wherein the patient is three years old or younger (e.g., 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[29] The method of embodiment 27, wherein the patient is two years old or younger (e.g., 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[30] The method of embodiment 27, wherein the patient is one year old or younger (e.g., 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[31] The method of embodiment 27, wherein the patient is six months old or younger (e.g., 6 months old or younger, 5 months old or younger, 4 months old or younger, 3 months old or younger, 2 months old or younger, or 1 month old or younger) at the time of administration of the transgene or viral vector.

[32] The method of any one of embodiments 1-25, wherein the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

[33] The method of any one of embodiments 1-32, the method further comprising monitoring the patient for development of cholestasis, hyperbilirubinemia, or one or more symptoms thereof.

[34] The method of embodiment 33, wherein the patient is monitored for the development of cholestasis, hyperbilirubinemia, or one or more symptoms thereof by evaluating a parameter in a blood sample obtained from the patient, wherein a finding that the parameter is above a reference level identifies the patient as having cholestasis, hyperbilirubinemia, or one or more symptoms thereof.

[35] The method of embodiment 34, wherein the parameter comprises the level of a serum bile acid in the blood sample.

[36] The method of embodiment 35, wherein the serum bile acid is cholic acid, chenodeoxycholic acid, deoxycholic acid, or ursodeoxycholic acid.

[37] The method of embodiment 34, wherein the parameter comprises one or more results of a liver function test.

[38] The method of embodiment 37, wherein the parameter comprises the level of aspartate aminotransferase or alanine aminotransferase in the blood sample.

[39] A method of treating XLMTM in a human patient in need thereof, the method comprising:
(a) administering to the patient a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

[40] A method of reducing stiffness and/or joint contractures in a human patient diagnosed as having XLMTM, the method comprising:
(a) administering to the patient a transgene encoding MTM1 in an amount of less than about 3×10¹⁴ vg/kg,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

[41] A method of increasing diaphragm and/or respiratory muscle progression in a human patient diagnosed as having XLMTM, the method comprising:
a) administering to the patient a transgene encoding MTM1 in an amount of less than about 3×10¹⁴ vg/kg,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

[42] The method of any one of embodiments 39-41, wherein the transgene encoding MTM1 is administered to the patient by transduction with a viral vector comprising a transgene encoding MTM1.

[43] A method of treating XLMTM in a human patient in need thereof, the method comprising:
(a) administering to the patient a transgene encoding MTM1,
(b) determining that the patient exhibits cholestasis, hyperbilirubinemia, or one or more symptoms thereof, and
(c) administering to the patient an anti-cholestatic agent.

[44] A method of reducing stiffness and/or joint contractures in a human patient diagnosed as having XLMTM, the method comprising:
(a) administering to the patient a transgene encoding MTM1 in an amount of less than about 3×10¹⁴ vg/kg,
(b) determining that the patient exhibits cholestasis, hyperbilirubinemia, or one or more symptoms thereof, and
(c) administering to the patient an anti-cholestatic agent.

[45] A method of increasing diaphragm and/or respiratory muscle progression in a human patient diagnosed as having XLMTM, the method comprising:
(a) administering to the patient a transgene encoding MTM1 in an amount of less than about 3×10¹⁴ vg/kg,
(b) determining that the patient exhibits cholestasis, hyperbilirubinemia, or one or more symptoms thereof, and
(c) administering to the patient an anti-cholestatic agent.

[46] The method of any one of embodiments 42 or 44-45, wherein the transgene encoding MTM1 is administered to the patient by transduction with a viral vector comprising a transgene encoding MTM1.

[47] The method of embodiment 42 or 46, wherein the viral vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10³ vg/kg, 1×10¹² vg/kg, 1×10¹¹ vg/kg, 1×10¹⁰ vg/kg, 1×10⁹ vg/kg, 1×10⁸ vg/kg, or less).

[48] The method of embodiment 47, wherein the viral vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[49] The method of embodiment 47, wherein the viral vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[50] The method of embodiment 47, wherein the viral vector is administered to the patient in an amount of less than about 1.5×10¹⁴ vg/kg (e.g., in an amount of less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[51] The method of embodiment 47, wherein the viral vector is administered to the patient in an amount of less than about 1.4×10¹⁴ vg/kg (e.g., in an amount of less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[52] The method of embodiment 42 or 46, wherein the viral vector is administered to the patient in an amount of from about 3×10¹³ vg/kg to about 2.3×10¹⁴ vg/kg (e.g., 3×10¹³ vg/kg to about 2.3×10¹⁴ vg/kg, 3.1×10¹³ vg/kg to about 2.3×10¹⁴ vg/kg, 3.2×10¹³ vg/kg to about 2.3×10¹⁴ vg/kg, 3.3×10¹³ vg/kg to about 2.3×10¹⁴ vg/kg, 3.4×10¹³ vg/kg to about 2.3×10¹⁴ vg/kg, 3.5×10¹³ vg/kg to about 2.3×10¹⁴ vg/kg, 3.6×10¹³ vg/kg to about 2.3×10¹⁴ vg/kg, 3.7×10¹³ vg/kg to about 2.3×10¹⁴ vg/kg, 3.8×10¹³ vg/kg to about 2.3×10¹⁴ vg/kg, 3.9×10¹³ vg/kg to about 2.3×10¹⁴ vg/kg, 4×10¹³ vg/kg to about 2.3×10¹⁴ vg/kg, 4.1×10¹³ vg/kg to about 2.3×10¹⁴ vg/kg, 4.2×10¹³ vg/kg to about 2.3×10¹⁴ vg/kg, 4.3×10¹³ vg/kg to about 2.3×10¹⁴ vg/kg, 4.4×10¹³ vg/kg to about 2.3×10¹⁴ vg/kg, 4.5×10¹³ vg/kg to about 2.3×10¹⁴ vg/kg, 4.6×10¹³ vg/kg to about 2.3×10¹⁴ vg/kg, 4.7×10¹³ vg/kg to about 2.3×10¹⁴ vg/kg, 4.8×10¹³ vg/kg to about 2.3×10¹⁴ vg/kg, 4.9×10¹³ vg/kg to about 2.3×10¹⁴ vg/kg, 5×10¹³ vg/kg to about 2.3×10¹⁴ vg/kg, 5.1×10¹³ vg/kg to about 2.3×10¹⁴ vg/kg, 5.2×10¹³ vg/kg to about 2.3×10¹⁴ vg/kg, 5.3×10¹³ vg/kg to about 2.3×10¹⁴ vg/kg, 5.4×10¹³ vg/kg to about 2.3×10¹⁴ vg/kg, 5.5×10¹³ vg/kg to about 2.3×10¹⁴ vg/kg, 5.6×10¹³ vg/kg to about 2.3×10¹⁴ vg/kg, 5.7×10¹³ vg/kg to about 2.3×10¹⁴ vg/kg, 5.8×10¹³ vg/kg to about 2.3×10¹⁴ vg/kg, 5.9×10¹³ vg/kg to about 2.3×10¹⁴ vg/kg, 6×10¹³ vg/kg to about 2.3×10¹⁴ vg/kg, 6.1×10¹³ vg/kg to about 2.3×10¹⁴ vg/kg, 6.2×10¹³ vg/kg to about 2.3×10¹⁴ vg/kg, 6.3×10¹³ vg/kg to about 2.3×10¹⁴ vg/kg, 6.4×10¹³ vg/kg to about 2.3×10¹⁴ vg/kg, 6.5×10¹³ vg/kg to about 2.3×10¹⁴ vg/kg, 6.6×10¹³ vg/kg to about 2.3×10¹⁴ vg/kg, 6.7×10¹³ vg/kg to about 2.3×10¹⁴ vg/kg, 6.8×10¹³ vg/kg to about 2.3×10¹⁴ vg/kg, 6.9×10¹³ vg/kg to about 2.3×10¹⁴ vg/kg, 7×10¹³ vg/kg to about 2.3×10¹⁴ vg/kg, 7.1×10¹³ vg/kg to about 2.3×10¹⁴ vg/kg, 7.2×10¹³ vg/kg to about 2.3×10¹⁴ vg/kg, 7.3×10¹³ vg/kg to about 2.3×10¹⁴ vg/kg, 7.4×10¹³ vg/kg to about 2.3×10¹⁴ vg/kg, 7.5×10¹³ vg/kg to about 2.3×10¹⁴ vg/kg, 7.6×10¹³ vg/kg to about 2.3×10¹⁴ vg/kg, 7.7×10¹³ vg/kg to about 2.3×10¹⁴ vg/kg, 7.8×10¹³ vg/kg to about 2.3×10¹⁴ vg/kg, 7.9×10¹³ vg/kg to about 2.3×10¹⁴ vg/kg, 8×10¹³ vg/kg to about 2.3×10¹⁴ vg/kg, 8.1×10¹³ vg/kg to about 2.3×10¹⁴ vg/kg, 8.2×10¹³ vg/kg to about 2.3×10¹⁴ vg/kg, 8.3×10¹³ vg/kg to about 2.3×10¹⁴ vg/kg, 8.4×10¹³ vg/kg to about 2.3×10¹⁴ vg/kg, 8.5×10¹³ vg/kg to about 2.3×10¹⁴ vg/kg, 8.6×10¹³ vg/kg to about 2.3×10¹⁴ vg/kg, 8.7×10¹³ vg/kg to about 2.3×10¹⁴ vg/kg, 8.8×10¹³ vg/kg to about 2.3×10¹⁴ vg/kg, 8.9×10¹³ vg/kg to about 2.3×10¹⁴ vg/kg, 9×10¹³ vg/kg to about 2.3×10¹⁴ vg/kg, 9.1×10¹³ vg/kg to about 2.3×10¹⁴ vg/kg, 9.2×10¹³ vg/kg to about 2.3×10¹⁴ vg/kg, 9.3×10¹³ vg/kg to about 2.3×10¹⁴ vg/kg, 9.4×10¹³ vg/kg to about 2.3×10¹⁴ vg/kg, 9.5×10¹³ vg/kg to about 2.3×10¹⁴ vg/kg, 9.6×10¹³ vg/kg to about 2.3×10¹⁴ vg/kg, 9.7×10¹³ vg/kg to about 2.3×10¹⁴ vg/kg, 9.8×10¹³ vg/kg to about 2.3×10¹⁴ vg/kg, 9.9×10¹³ vg/kg to about 2.3×10¹⁴ vg/kg, 1×10¹⁴ vg/kg to about 2.3×10¹⁴ vg/kg, 1.1×10⁴ vg/kg to about 2.3×10¹⁴ vg/kg, 1.2×10¹⁴ vg/kg to about 2.3×10¹⁴ vg/kg, 1.3×10¹⁴ vg/kg to about 2.3×10¹⁴ vg/kg, 1.4×10¹⁴ vg/kg to about 2.3×10¹⁴ vg/kg, 1.5×10¹⁴ vg/kg to about 2.3×10¹⁴ vg/kg, 1.6×10¹⁴ vg/kg to about 2.3×10¹⁴ vg/kg, 1.7×10¹⁴ vg/kg to about 2.3×10¹⁴ vg/kg, 1.8×10¹⁴ vg/kg to about 2.3×10¹⁴ vg/kg, 1.9×10¹⁴ vg/kg to about 2.3×10¹⁴ vg/kg, 2×10¹⁴ vg/kg to about 2.3×10¹⁴ vg/kg, 2.1×10¹⁴ vg/kg to about 2.3×10¹⁴ vg/kg, or 2.2×10¹⁴ vg/kg to about 2.3×10¹⁴ vg/kg).

[53] The method of embodiment 52, wherein the viral vector is administered to the patient in an amount of from about 8×10¹³ vg/kg to about 1.8×10¹⁴ vg/kg (e.g., 8×10¹³ vg/kg to about 1.8×10¹⁴ vg/kg, 8.1×10¹³ vg/kg to about 1.8×10¹⁴ vg/kg, 8.2×10¹³ vg/kg to about 1.8×10¹⁴ vg/kg, 8.3×10¹³ vg/kg to about 1.8×10¹⁴ vg/kg, 8.4×10¹³ vg/kg to about 1.8×10¹⁴ vg/kg, 8.5×10¹³ vg/kg to about 1.8×10¹⁴ vg/kg, 8.6×10¹³ vg/kg to about 1.8×10¹⁴ vg/kg, 8.7×10¹³ vg/kg to about 1.8×10¹⁴ vg/kg, 8.8×10¹³ vg/kg to about 1.8×10¹⁴ vg/kg, 8.9×10¹³ vg/kg to about 1.8×10¹⁴ vg/kg, 9×10¹³ vg/kg to about 1.8×10¹⁴ vg/kg, 9.1×10¹³ vg/kg to about 1.8×10¹⁴ vg/kg, 9.2×10¹³ vg/kg to about 1.8×10¹⁴ vg/kg, 9.3×10¹³ vg/kg to about 1.8×10¹⁴ vg/kg, 9.4×10¹³ vg/kg to about 1.8×10¹⁴ vg/kg, 9.5×10¹³ vg/kg to about 1.8×10¹⁴ vg/kg, 9.6×10¹³ vg/kg to about 1.8×10¹⁴ vg/kg, 9.7×10¹³ vg/kg to about 1.8×10¹⁴ vg/kg, 9.8×10¹³ vg/kg to about 1.8×10¹⁴ vg/kg, 9.9×10¹³ vg/kg to about 1.8×10¹⁴ vg/kg, 1×10¹⁴ vg/kg to about 1.8×10¹⁴ vg/kg, 1.1×10¹⁴ vg/kg to about 1.8×10¹⁴ vg/kg, 1.2×10¹⁴ vg/kg to about 1.8×10¹⁴ vg/kg, 1.3×10¹⁴ vg/kg to about 1.8×10¹⁴ vg/kg, 1.4×10¹⁴ vg/kg to about 1.8×10¹⁴ vg/kg, 1.5×10¹⁴ vg/kg to about 1.8×10¹⁴ vg/kg, 1.6×10¹⁴ vg/kg to about 1.8×10¹⁴ vg/kg, or 1.7×10¹⁴ vg/kg to about 1.8×10¹⁴ vg/kg).

[54] The method of embodiment 52, wherein the viral vector is administered to the patient in an amount of from about 1×10¹⁴ vg/kg to about 1.6×10¹⁴ vg/kg (e.g., 1×10¹⁴ vg/kg to about 1.6×10¹⁴ vg/kg, 1.1×10⁴ vg/kg to about 1.6×10¹⁴ vg/kg, 1.2×10¹⁴ vg/kg to about 1.6×10¹⁴ vg/kg, 1.3×10¹⁴ vg/kg to about 1.6×10¹⁴ vg/kg, 1.4×10¹⁴ vg/kg to about 1.6×10¹⁴ vg/kg, 1.5×10¹⁴ vg/kg to about 1.6×10¹⁴ vg/kg, 1.6×10¹⁴ vg/kg to about 1.6×10¹⁴ vg/kg, or 1.7×10¹⁴ vg/kg to about 1.6×10¹⁴ vg/kg).

[55] The method of embodiment 52, wherein the viral vector is administered to the patient in an amount of 1.1×10⁴ vg/kg to about 1.5×10¹⁴ vg/kg (e.g., 1.1×10⁴ vg/kg to about 1.5×10¹⁴ vg/kg, 1.2×10¹⁴ vg/kg to about 1.5×10¹⁴ vg/kg, 1.3×10¹⁴ vg/kg to about 1.5×10¹⁴ vg/kg, or 1.4×10¹⁴ vg/kg to about 1.5×10¹⁴ vg/kg).

[56] The method of embodiment 52, wherein the viral vector is administered to the patient in an amount of from about 1.2×10¹⁴ vg/kg to about 1.4×10¹⁴ vg/kg (e.g., 1.2×10¹⁴ vg/kg to about 1.4×10¹⁴ vg/kg, or 1.3×10¹⁴ vg/kg to about 1.4×10¹⁴ vg/kg).

[57] The method of any one of embodiments 42 or 46-56, wherein the viral vector is administered to the patient in an amount of about 1.3×10¹⁴ vg/kg.

[58] The method of any one of embodiments 39-57, wherein the patient is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[59] The method of embodiment 58, wherein the patient is four years old or younger (e.g., 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[60] The method of embodiment 59, wherein the patient is three years old or younger (e.g., 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[61] The method of embodiment 59, wherein the patient is two years old or younger (e.g., 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[62] The method of embodiment 59, wherein the patient is one year old or younger (e.g., 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[63] The method of embodiment 59, wherein the patient is six months old or younger (e.g., 6 months old or younger, 5 months old or younger, 4 months old or younger, 3 months old or younger, 2 months old or younger, or 1 month old or younger) at the time of administration of the transgene or viral vector.

[64] The method of any one of embodiments 39-57, wherein the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

[65] A method of treating XLMTM in a human patient in need thereof that is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), the method comprising:
(a) administering to the patient a therapeutically effective amount of a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

[66] A method of reducing stiffness and/or joint contractures in a human patient diagnosed as having XLMTM, the method comprising:
(a) administering to the patient a therapeutically effective amount of a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

[67] A method of increasing diaphragm and/or respiratory muscle progression in a human patient diagnosed as having XLMTM, the method comprising:
(a) administering to the patient a therapeutically effective amount of a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

[68] The method of any one of embodiments 65-67, wherein the transgene encoding MTM1 is administered to the patient by transduction with a viral vector comprising a transgene encoding MTM1 comprising a transgene encoding MTM1.

[69] A method of treating XLMTM in a human patient in need thereof that is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger), the method comprising:
(a) administering to the patient a therapeutically effective amount of a transgene encoding MTM1,
(b) determining that the patient exhibits cholestasis, hyperbilirubinemia, or one or more symptoms thereof, and
(c) administering to the patient an anti-cholestatic agent.

[70] A method of reducing stiffness and/or joint contractures in a human patient diagnosed as having XLMTM, the method comprising:
(a) administering to the patient a therapeutically effective amount of a transgene encoding MTM1,
(b) determining that the patient exhibits cholestasis, hyperbilirubinemia, or one or more symptoms thereof, and
(c) administering to the patient an anti-cholestatic agent.

[71] A method of increasing diaphragm and/or respiratory muscle progression in a human patient diagnosed as having XLMTM, the method comprising:
(a) administering to the patient a therapeutically effective amount of a transgene encoding MTM1,
(b) determining that the patient exhibits cholestasis, hyperbilirubinemia, or one or more symptoms thereof, and
(c) administering to the patient an anti-cholestatic agent.

[72] The method of any one of embodiments 69-71, wherein the transgene encoding MTM1 is administered to the patient by transduction with a viral vector comprising a transgene encoding MTM1 comprising a transgene encoding MTM1.

[73] The method of any one of embodiments 65-72, wherein the patient is four years old or younger (e.g., 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[74] The method of embodiment 73, wherein the patient is three years old or younger (e.g., 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[75] The method of embodiment 73, wherein the patient is two years old or younger (e.g., 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[76] The method of embodiment 73, wherein the patient is one year old or younger (e.g., 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[77] The method of embodiment 73, wherein the patient is six months old or younger (e.g., 6 months old or younger, 5 months old or younger, 4 months old or younger, 3 months old or younger, 2 months old or younger, or 1 month old or younger) at the time of administration of the transgene or viral vector.

[78] The method of any one of embodiments 65-72, wherein the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

[79] The method of embodiment 68 or 72-78, wherein the viral vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[80] The method of embodiment 79, wherein the viral vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[81] The method of embodiment 80, wherein the viral vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[82] The method of embodiment 80, wherein the viral vector is administered to the patient in an amount of less than about 1.5×10¹⁴ vg/kg (e.g., in an amount of less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[83] The method of embodiment 80, wherein the viral vector is administered to the patient in an amount of less than about 1.4×10¹⁴ vg/kg (e.g., in an amount of less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[84] The method of any one of embodiments 68 or 72-78, wherein the viral vector is administered to the patient in an amount of from about 3×10¹³ vg/kg to about 2.3×10¹⁴ vg/kg (e.g., 3×10¹³ vg/kg to about 2.3×10¹⁴ vg/kg, 3.1×10¹³ vg/kg to about 2.3×10¹⁴ vg/kg, 3.2×10¹³ vg/kg to about 2.3×10¹⁴ vg/kg, 3.3×10¹³ vg/kg to about 2.3×10¹⁴ vg/kg, 3.4×10¹³ vg/kg to about 2.3×10¹⁴ vg/kg, 3.5×10¹³ vg/kg to about 2.3×10¹⁴ vg/kg, 3.6×10¹³ vg/kg to about 2.3×10¹⁴ vg/kg, 3.7×10¹³ vg/kg to about 2.3×10¹⁴ vg/kg, 3.8×10¹³ vg/kg to about 2.3×10¹⁴ vg/kg, 3.9×10¹³ vg/kg to about 2.3×10¹⁴ vg/kg, 4×10¹³ vg/kg to about 2.3×10¹⁴ vg/kg, 4.1×10¹³ vg/kg to about 2.3×10¹⁴ vg/kg, 4.2×10¹³ vg/kg to about 2.3×10¹⁴ vg/kg, 4.3×10¹³ vg/kg to about 2.3×10¹⁴ vg/kg, 4.4×10¹³ vg/kg to about 2.3×10¹⁴ vg/kg, 4.5×10¹³ vg/kg to about 2.3×10¹⁴ vg/kg, 4.6×10¹³ vg/kg to about 2.3×10¹⁴ vg/kg, 4.7×10¹³ vg/kg to about 2.3×10¹⁴ vg/kg, 4.8×10¹³ vg/kg to about 2.3×10¹⁴ vg/kg, 4.9×10¹³ vg/kg to about 2.3×10¹⁴ vg/kg, 5×10¹³ vg/kg to about 2.3×10¹⁴ vg/kg, 5.1×10¹³ vg/kg to about 2.3×10¹⁴ vg/kg, 5.2×10¹³ vg/kg to about 2.3×10¹⁴ vg/kg, 5.3×10¹³ vg/kg to about 2.3×10¹⁴ vg/kg, 5.4×10¹³ vg/kg to about 2.3×10¹⁴ vg/kg, 5.5×10¹³ vg/kg to about 2.3×10¹⁴ vg/kg, 5.6×10¹³ vg/kg to about 2.3×10¹⁴ vg/kg, 5.7×10¹³ vg/kg to about 2.3×10¹⁴ vg/kg, 5.8×10¹³ vg/kg to about 2.3×10¹⁴ vg/kg, 5.9×10¹³ vg/kg to about 2.3×10¹⁴ vg/kg, 6×10¹³ vg/kg to about 2.3×10¹⁴ vg/kg, 6.1×10¹³ vg/kg to about 2.3×10¹⁴ vg/kg, 6.2×10¹³ vg/kg to about 2.3×10¹⁴ vg/kg, 6.3×10¹³ vg/kg to about 2.3×10¹⁴ vg/kg, 6.4×10¹³ vg/kg to about 2.3×10¹⁴ vg/kg, 6.5×10¹³ vg/kg to about 2.3×10¹⁴ vg/kg, 6.6×10¹³ vg/kg to about 2.3×10¹⁴ vg/kg, 6.7×10¹³ vg/kg to about 2.3×10¹⁴ vg/kg, 6.8×10¹³ vg/kg to about 2.3×10¹⁴ vg/kg, 6.9×10¹³ vg/kg to about 2.3×10¹⁴ vg/kg, 7×10¹³ vg/kg to about 2.3×10¹⁴ vg/kg, 7.1×10¹³ vg/kg to about 2.3×10¹⁴ vg/kg, 7.2×10¹³ vg/kg to about 2.3×10¹⁴ vg/kg, 7.3×10¹³ vg/kg to about 2.3×10¹⁴ vg/kg, 7.4×10¹³ vg/kg to about 2.3×10¹⁴ vg/kg, 7.5×10¹³ vg/kg to about 2.3×10¹⁴ vg/kg, 7.6×10¹³ vg/kg to about 2.3×10¹⁴ vg/kg, 7.7×10¹³ vg/kg to about 2.3×10¹⁴ vg/kg, 7.8×10¹³ vg/kg to about 2.3×10¹⁴ vg/kg, 7.9×10¹³ vg/kg to about 2.3×10¹⁴ vg/kg, 8×10¹³ vg/kg to about 2.3×10¹⁴ vg/kg, 8.1×10¹³ vg/kg to about 2.3×10¹⁴ vg/kg, 8.2×10¹³ vg/kg to about 2.3×10¹⁴ vg/kg, 8.3×10¹³ vg/kg to about 2.3×10¹⁴ vg/kg, 8.4×10¹³ vg/kg to about 2.3×10¹⁴ vg/kg, 8.5×10¹³ vg/kg to about 2.3×10¹⁴ vg/kg, 8.6×10¹³ vg/kg to about 2.3×10¹⁴ vg/kg, 8.7×10¹³ vg/kg to about 2.3×10¹⁴ vg/kg, 8.8×10¹³ vg/kg to about 2.3×10¹⁴ vg/kg, 8.9×10¹³ vg/kg to about 2.3×10¹⁴ vg/kg, 9×10¹³ vg/kg to about 2.3×10¹⁴ vg/kg, 9.1×10¹³ vg/kg to about 2.3×10¹⁴ vg/kg, 9.2×10¹³ vg/kg to about 2.3×10¹⁴ vg/kg, 9.3×10¹³ vg/kg to about 2.3×10¹⁴ vg/kg, 9.4×10¹³ vg/kg to about 2.3×10¹⁴ vg/kg, 9.5×10¹³ vg/kg to about 2.3×10¹⁴ vg/kg, 9.6×10¹³ vg/kg to about 2.3×10¹⁴ vg/kg, 9.7×10¹³ vg/kg to about 2.3×10¹⁴ vg/kg, 9.8×10¹³ vg/kg to about 2.3×10¹⁴ vg/kg, 9.9×10¹³ vg/kg to about 2.3×10¹⁴ vg/kg, 1×10¹⁴ vg/kg to about 2.3×10¹⁴ vg/kg, 1.1×10¹⁴ vg/kg to about 2.3×10¹⁴ vg/kg, 1.2×10¹⁴ vg/kg to about 2.3×10¹⁴ vg/kg, 1.3×10¹⁴ vg/kg to about 2.3×10¹⁴ vg/kg, 1.4×10¹⁴ vg/kg to about 2.3×10¹⁴ vg/kg, 1.5×10¹⁴ vg/kg to about 2.3×10¹⁴ vg/kg, 1.6×10¹⁴ vg/kg to about 2.3×10¹⁴ vg/kg, 1.7×10¹⁴ vg/kg to about 2.3×10¹⁴ vg/kg, 1.8×10¹⁴ vg/kg to about 2.3×10¹⁴ vg/kg, 1.9×10¹⁴ vg/kg to about 2.3×10¹⁴ vg/kg, 2×10¹⁴ vg/kg to about 2.3×10¹⁴ vg/kg, 2.1×10¹⁴ vg/kg to about 2.3×10¹⁴ vg/kg, or 2.2×10¹⁴ vg/kg to about 2.3×10¹⁴ vg/kg).

[85] The method of embodiment 84, wherein the viral vector is administered to the patient in an amount of from about 8×10¹³ vg/kg to about 1.8×10¹⁴ vg/kg (e.g., 8×10¹³ vg/kg to about 1.8×10¹⁴ vg/kg, 8.1×10¹³ vg/kg to about 1.8×10¹⁴ vg/kg, 8.2×10¹³ vg/kg to about 1.8×10¹⁴ vg/kg, 8.3×10¹³ vg/kg to about 1.8×10¹⁴ vg/kg, 8.4×10¹³ vg/kg to about 1.8×10¹⁴ vg/kg, 8.5×10¹³ vg/kg to about 1.8×10¹⁴ vg/kg, 8.6×10¹³ vg/kg to about 1.8×10¹⁴ vg/kg, 8.7×10¹³ vg/kg to about 1.8×10¹⁴ vg/kg, 8.8×10¹³ vg/kg to about 1.8×10¹⁴ vg/kg, 8.9×10¹³ vg/kg to about 1.8×10¹⁴ vg/kg, 9×10¹³ vg/kg to about 1.8×10¹⁴ vg/kg, 9.1×10¹³ vg/kg to about 1.8×10¹⁴ vg/kg, 9.2×10¹³ vg/kg to about 1.8×10¹⁴ vg/kg, 9.3×10¹³ vg/kg to about 1.8×10¹⁴ vg/kg, 9.4×10¹³ vg/kg to about 1.8×10¹⁴ vg/kg, 9.5×10¹³ vg/kg to about 1.8×10¹⁴ vg/kg, 9.6×10¹³ vg/kg to about 1.8×10¹⁴ vg/kg, 9.7×10¹³ vg/kg to about 1.8×10¹⁴ vg/kg, 9.8×10¹³ vg/kg to about 1.8×10¹⁴ vg/kg, 9.9×10¹³ vg/kg to about 1.8×10¹⁴ vg/kg, 1×10¹⁴ vg/kg to about 1.8×10¹⁴ vg/kg, 1.1×10¹⁴ vg/kg to about 1.8×10¹⁴ vg/kg, 1.2×10¹⁴ vg/kg to about 1.8×10¹⁴ vg/kg, 1.3×10¹⁴ vg/kg to about 1.8×10¹⁴ vg/kg, 1.4×10¹⁴ vg/kg to about 1.8×10¹⁴ vg/kg, 1.5×10¹⁴ vg/kg to about 1.8×10¹⁴ vg/kg, 1.6×10¹⁴ vg/kg to about 1.8×10¹⁴ vg/kg, or 1.7×10¹⁴ vg/kg to about 1.8×10¹⁴ vg/kg).

[86] The method of embodiment 84, wherein the viral vector is administered to the patient in an amount of from about 1×10¹⁴ vg/kg to about 1.6×10¹⁴ vg/kg (e.g., 1×10¹⁴ vg/kg to about 1.6×10¹⁴ vg/kg, 1.1×10⁴ vg/kg to about 1.6×10¹⁴ vg/kg, 1.2×10¹⁴ vg/kg to about 1.6×10¹⁴ vg/kg, 1.3×10¹⁴ vg/kg to about 1.6×10¹⁴ vg/kg, 1.4×10¹⁴ vg/kg to about 1.6×10¹⁴ vg/kg, 1.5×10¹⁴ vg/kg to about 1.6×10¹⁴ vg/kg, 1.6×10¹⁴ vg/kg to about 1.6×10¹⁴ vg/kg, or 1.7×10¹⁴ vg/kg to about 1.6×10¹⁴ vg/kg).

[87] The method of embodiment 84, wherein the viral vector is administered to the patient in an amount of 1.1×10⁴ vg/kg to about 1.5×10¹⁴ vg/kg (e.g., 1.1×10⁴ vg/kg to about 1.5×10¹⁴ vg/kg, 1.2×10¹⁴ vg/kg to about 1.5×10¹⁴ vg/kg, 1.3×10¹⁴ vg/kg to about 1.5×10¹⁴ vg/kg, or 1.4×10¹⁴ vg/kg to about 1.5×10¹⁴ vg/kg).

[88] The method of embodiment 84, wherein the viral vector is administered to the patient in an amount of from about 1.2×10¹⁴ vg/kg to about 1.4×10¹⁴ vg/kg (e.g., 1.2×10¹⁴ vg/kg to about 1.4×10¹⁴ vg/kg, or 1.3×10¹⁴ vg/kg to about 1.4×10¹⁴ vg/kg).

[89] The method of any one of embodiments 68 or 72-84, wherein the viral vector is administered to the patient in an amount of about 1.3×10¹⁴ vg/kg.

[90] A method of treating or preventing cholestasis or hyperbilirubinemia in a human patient that has XLMTM and who has been previously administered a transgene encoding MTM1, the method comprising administering to the patient an anti-cholestatic agent.

[91] A method of treating or preventing cholestasis or hyperbilirubinemia in a human patient that has XLMTM and who has been previously administered a viral vector comprising a transgene encoding MTM1 in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less), the method comprising administering to the patient an anti-cholestatic agent.

[92] The method of embodiment 91, wherein the viral vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[93] The method of embodiment 92, wherein the viral vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[94] The method of embodiment 92, wherein the viral vector is administered to the patient in an amount of less than about 1.5×10¹⁴ vg/kg (e.g., in an amount of less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[95] The method of embodiment 92, wherein the viral vector is administered to the patient in an amount of less than about 1.4×10¹⁴ vg/kg (e.g., in an amount of less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[96] The method of embodiment 91, wherein the viral vector is administered to the patient in an amount of from about 3×10¹³ vg/kg to about 2.3×10¹⁴ vg/kg (e.g., 3×10¹³ vg/kg to about 2.3×10¹⁴ vg/kg, 3.1×10¹³ vg/kg to about 2.3×10¹⁴ vg/kg, 3.2×10¹³ vg/kg to about 2.3×10¹⁴ vg/kg, 3.3×10¹³ vg/kg to about 2.3×10¹⁴ vg/kg, 3.4×10¹³ vg/kg to about 2.3×10¹⁴ vg/kg, 3.5×10¹³ vg/kg to about 2.3×10¹⁴ vg/kg, 3.6×10¹³ vg/kg to about 2.3×10¹⁴ vg/kg, 3.7×10¹³ vg/kg to about 2.3×10¹⁴ vg/kg, 3.8×10¹³ vg/kg to about 2.3×10¹⁴ vg/kg, 3.9×10¹³ vg/kg to about 2.3×10¹⁴ vg/kg, 4×10¹³ vg/kg to about 2.3×10¹⁴ vg/kg, 4.1×10¹³ vg/kg to about 2.3×10¹⁴ vg/kg, 4.2×10¹³ vg/kg to about 2.3×10¹⁴ vg/kg, 4.3×10¹³ vg/kg to about 2.3×10¹⁴ vg/kg, 4.4×10¹³ vg/kg to about 2.3×10¹⁴ vg/kg, 4.5×10¹³ vg/kg to about 2.3×10¹⁴ vg/kg, 4.6×10¹³ vg/kg to about 2.3×10¹⁴ vg/kg, 4.7×10¹³ vg/kg to about 2.3×10¹⁴ vg/kg, 4.8×10¹³ vg/kg to about 2.3×10¹⁴ vg/kg, 4.9×10¹³ vg/kg to about 2.3×10¹⁴ vg/kg, 5×10¹³ vg/kg to about 2.3×10¹⁴ vg/kg, 5.1×10¹³ vg/kg to about 2.3×10¹⁴ vg/kg, 5.2×10¹³ vg/kg to about 2.3×10¹⁴ vg/kg, 5.3×10¹³ vg/kg to about 2.3×10¹⁴ vg/kg, 5.4×10¹³ vg/kg to about 2.3×10¹⁴ vg/kg, 5.5×10¹³ vg/kg to about 2.3×10¹⁴ vg/kg, 5.6×10¹³ vg/kg to about 2.3×10¹⁴ vg/kg, 5.7×10¹³ vg/kg to about 2.3×10¹⁴ vg/kg, 5.8×10¹³ vg/kg to about 2.3×10¹⁴ vg/kg, 5.9×10¹³ vg/kg to about 2.3×10¹⁴ vg/kg, 6×10¹³ vg/kg to about 2.3×10¹⁴ vg/kg, 6.1×10¹³ vg/kg to about 2.3×10¹⁴ vg/kg, 6.2×10¹³ vg/kg to about 2.3×10¹⁴ vg/kg, 6.3×10¹³ vg/kg to about 2.3×10¹⁴ vg/kg, 6.4×10¹³ vg/kg to about 2.3×10¹⁴ vg/kg, 6.5×10¹³ vg/kg to about 2.3×10¹⁴ vg/kg, 6.6×10¹³ vg/kg to about 2.3×10¹⁴ vg/kg, 6.7×10¹³ vg/kg to about 2.3×10¹⁴ vg/kg, 6.8×10¹³ vg/kg to about 2.3×10¹⁴ vg/kg, 6.9×10¹³ vg/kg to about 2.3×10¹⁴ vg/kg, 7×10¹³ vg/kg to about 2.3×10¹⁴ vg/kg, 7.1×10¹³ vg/kg to about 2.3×10¹⁴ vg/kg, 7.2×10¹³ vg/kg to about 2.3×10¹⁴ vg/kg, 7.3×10¹³ vg/kg to about 2.3×10¹⁴ vg/kg, 7.4×10¹³ vg/kg to about 2.3×10¹⁴ vg/kg, 7.5×10¹³ vg/kg to about 2.3×10¹⁴ vg/kg, 7.6×10¹³ vg/kg to about 2.3×10¹⁴ vg/kg, 7.7×10¹³ vg/kg to about 2.3×10¹⁴ vg/kg, 7.8×10¹³ vg/kg to about 2.3×10¹⁴ vg/kg, 7.9×10¹³ vg/kg to about 2.3×10¹⁴ vg/kg, 8×10¹³ vg/kg to about 2.3×10¹⁴ vg/kg, 8.1×10¹³ vg/kg to about 2.3×10¹⁴ vg/kg, 8.2×10¹³ vg/kg to about 2.3×10¹⁴ vg/kg, 8.3×10¹³ vg/kg to about 2.3×10¹⁴ vg/kg, 8.4×10¹³ vg/kg to about 2.3×10¹⁴ vg/kg, 8.5×10¹³ vg/kg to about 2.3×10¹⁴ vg/kg, 8.6×10¹³ vg/kg to about 2.3×10¹⁴ vg/kg, 8.7×10¹³ vg/kg to about 2.3×10¹⁴ vg/kg, 8.8×10¹³ vg/kg to about 2.3×10¹⁴ vg/kg, 8.9×10¹³ vg/kg to about 2.3×10¹⁴ vg/kg, 9×10¹³ vg/kg to about 2.3×10¹⁴ vg/kg, 9.1×10¹³ vg/kg to about 2.3×10¹⁴ vg/kg, 9.2×10¹³ vg/kg to about 2.3×10¹⁴ vg/kg, 9.3×10¹³ vg/kg to about 2.3×10¹⁴ vg/kg, 9.4×10¹³ vg/kg to about 2.3×10¹⁴ vg/kg, 9.5×10¹³ vg/kg to about 2.3×10¹⁴ vg/kg, 9.6×10¹³ vg/kg to about 2.3×10¹⁴ vg/kg, 9.7×10¹³ vg/kg to about 2.3×10¹⁴ vg/kg, 9.8×10¹³ vg/kg to about 2.3×10¹⁴ vg/kg, 9.9×10¹³ vg/kg to about 2.3×10¹⁴ vg/kg, 1×10¹⁴ vg/kg to about 2.3×10¹⁴ vg/kg, 1.1×10¹⁴ vg/kg to about 2.3×10¹⁴ vg/kg, 1.2×10¹⁴ vg/kg to about 2.3×10¹⁴ vg/kg, 1.3×10¹⁴ vg/kg to about 2.3×10¹⁴ vg/kg, 1.4×10¹⁴ vg/kg to about 2.3×10¹⁴ vg/kg, 1.5×10¹⁴ vg/kg to about 2.3×10¹⁴ vg/kg, 1.6×10¹⁴ vg/kg to about 2.3×10¹⁴ vg/kg, 1.7×10¹⁴ vg/kg to about 2.3×10¹⁴ vg/kg, 1.8×10¹⁴ vg/kg to about 2.3×10¹⁴ vg/kg, 1.9×10¹⁴ vg/kg to about 2.3×10¹⁴ vg/kg, 2×10¹⁴ vg/kg to about 2.3×10¹⁴ vg/kg, 2.1×10¹⁴ vg/kg to about 2.3×10¹⁴ vg/kg, or 2.2×10¹⁴ vg/kg to about 2.3×10¹⁴ vg/kg).

[97] The method of embodiment 96, wherein the viral vector is administered to the patient in an amount of from about 8×10¹³ vg/kg to about 1.8×10¹⁴ vg/kg (e.g., 8×10¹³ vg/kg to about 1.8×10¹⁴ vg/kg, 8.1×10¹³ vg/kg to about 1.8×10¹⁴ vg/kg, 8.2×10¹³ vg/kg to about 1.8×10¹⁴ vg/kg, 8.3×10¹³ vg/kg to about 1.8×10¹⁴ vg/kg, 8.4×10¹³ vg/kg to about 1.8×10¹⁴ vg/kg, 8.5×10¹³ vg/kg to about 1.8×10¹⁴ vg/kg, 8.6×10¹³ vg/kg to about 1.8×10¹⁴ vg/kg, 8.7×10¹³ vg/kg to about 1.8×10¹⁴ vg/kg, 8.8×10¹³ vg/kg to about 1.8×10¹⁴ vg/kg, 8.9×10¹³ vg/kg to about 1.8×10¹⁴ vg/kg, 9×10¹³ vg/kg to about 1.8×10¹⁴ vg/kg, 9.1×10¹³ vg/kg to about 1.8×10¹⁴ vg/kg, 9.2×10¹³ vg/kg to about 1.8×10¹⁴ vg/kg, 9.3×10¹³ vg/kg to about 1.8×10¹⁴ vg/kg, 9.4×10¹³ vg/kg to about 1.8×10¹⁴ vg/kg, 9.5×10¹³ vg/kg to about 1.8×10¹⁴ vg/kg, 9.6×10¹³ vg/kg to about 1.8×10¹⁴ vg/kg, 9.7×10¹³ vg/kg to about 1.8×10¹⁴ vg/kg, 9.8×10¹³ vg/kg to about 1.8×10¹⁴ vg/kg, 9.9×10¹³ vg/kg to about 1.8×10¹⁴ vg/kg, 1×10¹⁴ vg/kg to about 1.8×10¹⁴ vg/kg, 1.1×10¹⁴ vg/kg to about 1.8×10¹⁴ vg/kg, 1.2×10¹⁴ vg/kg to about 1.8×10¹⁴ vg/kg, 1.3×10¹⁴ vg/kg to about 1.8×10¹⁴ vg/kg, 1.4×10¹⁴ vg/kg to about 1.8×10¹⁴ vg/kg, 1.5×10¹⁴ vg/kg to about 1.8×10¹⁴ vg/kg, 1.6×10¹⁴ vg/kg to about 1.8×10¹⁴ vg/kg, or 1.7×10¹⁴ vg/kg to about 1.8×10¹⁴ vg/kg).

[98] The method of embodiment 96, wherein the viral vector is administered to the patient in an amount of from about 1×10¹⁴ vg/kg to about 1.6×10¹⁴ vg/kg (e.g., 1×10¹⁴ vg/kg to about 1.6×10¹⁴ vg/kg, 1.1×10¹⁴ vg/kg to about 1.6×10¹⁴ vg/kg, 1.2×10¹⁴ vg/kg to about 1.6×10¹⁴ vg/kg, 1.3×10¹⁴ vg/kg to about 1.6×10¹⁴ vg/kg, 1.4×10¹⁴ vg/kg to about 1.6×10¹⁴ vg/kg, 1.5×10¹⁴ vg/kg to about 1.6×10¹⁴ vg/kg, 1.6×10¹⁴ vg/kg to about 1.6×10¹⁴ vg/kg, or 1.7×10¹⁴ vg/kg to about 1.6×10¹⁴ vg/kg).

[99] The method of embodiment 96, wherein the viral vector is administered to the patient in an amount of 1.1×10¹⁴ vg/kg to about 1.5×10¹⁴ vg/kg (e.g., 1.1×10¹⁴ vg/kg to about 1.5×10¹⁴ vg/kg, 1.2×10¹⁴ vg/kg to about 1.5×10¹⁴ vg/kg, 1.3×10¹⁴ vg/kg to about 1.5×10¹⁴ vg/kg, or 1.4×10¹⁴ vg/kg to about 1.5×10¹⁴ vg/kg).

[100] The method of embodiment 96, wherein the viral vector is administered to the patient in an amount of from about 1.2×10¹⁴ vg/kg to about 1.4×10¹⁴ vg/kg (e.g., 1.2×10¹⁴ vg/kg to about 1.4×10¹⁴ vg/kg, or 1.3×10¹⁴ vg/kg to about 1.4×10¹⁴ vg/kg).

[101] The method of any one of embodiments 91-100, wherein the viral vector is administered to the patient in an amount of about 1.3×10¹⁴ vg/kg.

[102] The method of any one of embodiments 91-101, wherein the patient is five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[103] The method of embodiment 102, wherein the patient is four years old or younger (e.g., 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[104] The method of embodiment 103, wherein the patient is three years old or younger (e.g., 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[105] The method of embodiment 103, wherein the patient is two years old or younger (e.g., 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[106] The method of embodiment 103, wherein the patient is one year old or younger (e.g., 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[107] The method of embodiment 103, wherein the patient is six months old or younger (e.g., 6 months old or younger, 5 months old or younger, 4 months old or younger, 3 months old or younger, 2 months old or younger, or 1 month old or younger) at the time of administration of the transgene or viral vector.

[108] The method of any one of embodiments 90-101, wherein the patient is from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

[109] A method of treating or preventing cholestasis or hyperbilirubinemia in a human patient that has XLMTM, has been previously administered a transgene encoding MTM1, and that was five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene, the method comprising administering to the patient an anti-cholestatic agent.

[110] A method of treating or preventing cholestasis or hyperbilirubinemia in a human patient that has XLMTM, has been previously administered a viral vector comprising a transgene encoding MTM1, and that was five years old or younger (e.g., 5 years old or younger, 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the viral vector, the method comprising administering to the patient an anti-cholestatic agent.

[111] The method of embodiment 109 or 110, wherein the patient was four years old or younger (e.g., 4 years old or younger, 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[112] The method of embodiment 111, wherein the patient is three years old or younger (e.g., 3 years old or younger, 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[113] The method of embodiment 111, wherein the patient is two years old or younger (e.g., 2 years old or younger, 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[114] The method of embodiment 111, wherein the patient is one year old or younger (e.g., 1 year old or younger, 12 months old or younger, 11 months old or younger, 10 months old or younger, 9 months old or younger, 8 months old or younger, 7 months old or younger, 6 months old or younger, 5 months old or younger, 4 months old or younger, 3 months old or younger, 2 months old or younger, or 1 month old or younger) at the time of administration of the transgene or viral vector.

[115] The method of embodiment 111, wherein the patient is six months old or younger (e.g., 6 months old or younger, 5 months old or younger, 4 months old or younger, 3 months old or younger, 2 months old or younger, or 1 month old or younger) at the time of administration of the transgene or viral vector.

[116] The method of embodiment 109 or 110, wherein the patient was from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

[117] The method of any one of embodiments 110-116, wherein the viral vector is administered to the patient in an amount of less than about 3×10¹⁴ vg/kg (e.g., in an amount of less than about 3×10¹⁴ vg/kg, 2.9×10¹⁴ vg/kg, 2.8×10¹⁴ vg/kg, 2.7×10¹⁴ vg/kg, 2.6×10¹⁴ vg/kg, 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[118] The method of embodiment 117, wherein the viral vector is administered to the patient in an amount of less than about 2.5×10¹⁴ vg/kg (e.g., in an amount of less than about 2.5×10¹⁴ vg/kg, 2.4×10¹⁴ vg/kg, 2.3×10¹⁴ vg/kg, 2.2×10¹⁴ vg/kg, 2.1×10¹⁴ vg/kg, 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[119] The method of embodiment 118, wherein the viral vector is administered to the patient in an amount of less than about 2×10¹⁴ vg/kg (e.g., in an amount of less than about 2×10¹⁴ vg/kg, 1.9×10¹⁴ vg/kg, 1.8×10¹⁴ vg/kg, 1.7×10¹⁴ vg/kg, 1.6×10¹⁴ vg/kg, 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[120] The method of embodiment 118, wherein the viral vector is administered to the patient in an amount of less than about 1.5×10¹⁴ vg/kg (e.g., in an amount of less than about 1.5×10¹⁴ vg/kg, 1.4×10¹⁴ vg/kg, 1.3×10¹⁴ vg/kg, 1.2×10¹⁴ vg/kg, 1.1×10¹⁴ vg/kg, 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[121] The method of embodiment 118, wherein the viral vector is administered to the patient in an amount of less than about 1.4×10¹⁴ vg/kg (e.g., in an amount of less than about 1×10¹⁴ vg/kg, 1×10¹⁴ vg/kg, 1×10¹³ vg/kg, 1×10¹² vg/kg, 1×10¹¹ vg/kg, 1×10¹⁰ vg/kg, 1×10⁹ vg/kg, 1×10⁸ vg/kg, or less).

[122] The method of any one of embodiments 110-116, wherein the viral vector is administered to the patient in an amount of from about 3×10¹³ vg/kg to about 2.3×10¹⁴ vg/kg (e.g., 3×10¹³ vg/kg to about 2.3×10¹⁴ vg/kg, 3.1×10¹³ vg/kg to about 2.3×10¹⁴ vg/kg, 3.2×10¹³ vg/kg to about 2.3×10¹⁴ vg/kg, 3.3×10¹³ vg/kg to about 2.3×10¹⁴ vg/kg, 3.4×10¹³ vg/kg to about 2.3×10¹⁴ vg/kg, 3.5×10¹³ vg/kg to about 2.3×10¹⁴ vg/kg, 3.6×10¹³ vg/kg to about 2.3×10¹⁴ vg/kg, 3.7×10¹³ vg/kg to about 2.3×10¹⁴ vg/kg, 3.8×10¹³ vg/kg to about 2.3×10¹⁴ vg/kg, 3.9×10¹³ vg/kg to about 2.3×10¹⁴ vg/kg, 4×10¹³ vg/kg to about 2.3×10¹⁴ vg/kg, 4.1×10¹³ vg/kg to about 2.3×10¹⁴ vg/kg, 4.2×10¹³ vg/kg to about 2.3×10¹⁴ vg/kg, 4.3×10¹³ vg/kg to about 2.3×10¹⁴ vg/kg, 4.4×10¹³ vg/kg to about 2.3×10¹⁴ vg/kg, 4.5×10¹³ vg/kg to about 2.3×10¹⁴ vg/kg, 4.6×10¹³ vg/kg to about 2.3×10¹⁴ vg/kg, 4.7×10¹³ vg/kg to about 2.3×10¹⁴ vg/kg, 4.8×10¹³ vg/kg to about 2.3×10¹⁴ vg/kg, 4.9×10¹³ vg/kg to about 2.3×10¹⁴ vg/kg, 5×10¹³ vg/kg to about 2.3×10¹⁴ vg/kg, 5.1×10¹³ vg/kg to about 2.3×10¹⁴ vg/kg, 5.2×10¹³ vg/kg to about 2.3×10¹⁴ vg/kg, 5.3×10¹³ vg/kg to about 2.3×10¹⁴ vg/kg, 5.4×10¹³ vg/kg to about 2.3×10¹⁴ vg/kg, 5.5×10¹³ vg/kg to about 2.3×10¹⁴ vg/kg, 5.6×10¹³ vg/kg to about 2.3×10¹⁴ vg/kg, 5.7×10¹³ vg/kg to about 2.3×10¹⁴ vg/kg, 5.8×10¹³ vg/kg to about 2.3×10¹⁴ vg/kg, 5.9×10¹³ vg/kg to about 2.3×10¹⁴ vg/kg, 6×10¹³ vg/kg to about 2.3×10¹⁴ vg/kg, 6.1×10¹³ vg/kg to about 2.3×10¹⁴ vg/kg, 6.2×10¹³ vg/kg to about 2.3×10¹⁴ vg/kg, 6.3×10¹³ vg/kg to about 2.3×10¹⁴ vg/kg, 6.4×10¹³ vg/kg to about 2.3×10¹⁴ vg/kg, 6.5×10¹³ vg/kg to about 2.3×10¹⁴ vg/kg, 6.6×10¹³ vg/kg to about 2.3×10¹⁴ vg/kg, 6.7×10¹³ vg/kg to about 2.3×10¹⁴ vg/kg, 6.8×10¹³ vg/kg to about 2.3×10¹⁴ vg/kg, 6.9×10¹³ vg/kg to about 2.3×10¹⁴ vg/kg, 7×10¹³ vg/kg to about 2.3×10¹⁴ vg/kg, 7.1×10¹³ vg/kg to about 2.3×10¹⁴ vg/kg, 7.2×10¹³ vg/kg to about 2.3×10¹⁴ vg/kg, 7.3×10¹³ vg/kg to about 2.3×10¹⁴ vg/kg, 7.4×10¹³ vg/kg to about 2.3×10¹⁴ vg/kg, 7.5×10¹³ vg/kg to about 2.3×10¹⁴ vg/kg, 7.6×10¹³ vg/kg to about 2.3×10¹⁴ vg/kg, 7.7×10¹³ vg/kg to about 2.3×10¹⁴ vg/kg, 7.8×10¹³ vg/kg to about 2.3×10¹⁴ vg/kg, 7.9×10¹³ vg/kg to about 2.3×10¹⁴ vg/kg, 8×10¹³ vg/kg to about 2.3×10¹⁴ vg/kg, 8.1×10¹³ vg/kg to about 2.3×10¹⁴ vg/kg, 8.2×10¹³ vg/kg to about 2.3×10¹⁴ vg/kg, 8.3×10¹³ vg/kg to about 2.3×10¹⁴ vg/kg, 8.4×10¹³ vg/kg to about 2.3×10¹⁴ vg/kg, 8.5×10¹³ vg/kg to about 2.3×10¹⁴ vg/kg, 8.6×10¹³ vg/kg to about 2.3×10¹⁴ vg/kg, 8.7×10¹³ vg/kg to about 2.3×10¹⁴ vg/kg, 8.8×10¹³ vg/kg to about 2.3×10¹⁴ vg/kg, 8.9×10¹³ vg/kg to about 2.3×10¹⁴ vg/kg, 9×10¹³ vg/kg to about 2.3×10¹⁴ vg/kg, 9.1×10¹³ vg/kg to about 2.3×10¹⁴ vg/kg, 9.2×10¹³ vg/kg to about 2.3×10¹⁴ vg/kg, 9.3×10¹³ vg/kg to about 2.3×10¹⁴ vg/kg, 9.4×10¹³ vg/kg to about 2.3×10¹⁴ vg/kg, 9.5×10¹³ vg/kg to about 2.3×10¹⁴ vg/kg, 9.6×10¹³ vg/kg to about 2.3×10¹⁴ vg/kg, 9.7×10¹³ vg/kg to about 2.3×10¹⁴ vg/kg, 9.8×10¹³ vg/kg to about 2.3×10¹⁴ vg/kg, 9.9×10¹³ vg/kg to about 2.3×10¹⁴ vg/kg, 1×10¹⁴ vg/kg to about 2.3×10¹⁴ vg/kg, 1.1×10¹⁴ vg/kg to about 2.3×10¹⁴ vg/kg, 1.2×10¹⁴ vg/kg to about 2.3×10¹⁴ vg/kg, 1.3×10¹⁴ vg/kg to about 2.3×10¹⁴ vg/kg, 1.4×10¹⁴ vg/kg to about 2.3×10¹⁴ vg/kg, 1.5×10¹⁴ vg/kg to about 2.3×10¹⁴ vg/kg, 1.6×10¹⁴ vg/kg to about 2.3×10¹⁴ vg/kg, 1.7×10¹⁴ vg/kg to about 2.3×10¹⁴ vg/kg, 1.8×10¹⁴ vg/kg to about 2.3×10¹⁴ vg/kg, 1.9×10¹⁴ vg/kg to about 2.3×10¹⁴ vg/kg, 2×10¹⁴ vg/kg to about 2.3×10¹⁴ vg/kg, 2.1×10¹⁴ vg/kg to about 2.3×10¹⁴ vg/kg, or 2.2×10¹⁴ vg/kg to about 2.3×10¹⁴ vg/kg).

[123] The method of embodiment 122, wherein the viral vector is administered to the patient in an amount of from about 8×10¹³ vg/kg to about 1.8×10¹⁴ vg/kg (e.g., 8×10¹³ vg/kg to about 1.8×10¹⁴ vg/kg, 8.1×10¹³ vg/kg to about 1.8×10¹⁴ vg/kg, 8.2×10¹³ vg/kg to about 1.8×10¹⁴ vg/kg, 8.3×10¹³ vg/kg to about 1.8×10¹⁴ vg/kg, 8.4×10¹³ vg/kg to about 1.8×10¹⁴ vg/kg, 8.5×10¹³ vg/kg to about 1.8×10¹⁴ vg/kg, 8.6×10¹³ vg/kg to about 1.8×10¹⁴ vg/kg, 8.7×10¹³ vg/kg to about 1.8×10¹⁴ vg/kg, 8.8×10¹³ vg/kg to about 1.8×10¹⁴ vg/kg, 8.9×10¹³ vg/kg to about 1.8×10¹⁴ vg/kg, 9×10¹³ vg/kg to about 1.8×10¹⁴ vg/kg, 9.1×10¹³ vg/kg to about 1.8×10¹⁴ vg/kg, 9.2×10¹³ vg/kg to about 1.8×10¹⁴ vg/kg, 9.3×10¹³ vg/kg to about 1.8×10¹⁴ vg/kg, 9.4×10¹³ vg/kg to about 1.8×10¹⁴ vg/kg, 9.5×10¹³ vg/kg to about 1.8×10¹⁴ vg/kg, 9.6×10¹³ vg/kg to about 1.8×10¹⁴ vg/kg, 9.7×10¹³ vg/kg to about 1.8×10¹⁴ vg/kg, 9.8×10¹³ vg/kg to about 1.8×10¹⁴ vg/kg, 9.9×10¹³ vg/kg to about 1.8×10¹⁴ vg/kg, 1×10¹⁴ vg/kg to about 1.8×10¹⁴ vg/kg, 1.1×10¹⁴ vg/kg to about 1.8×10¹⁴ vg/kg, 1.2×10¹⁴ vg/kg to about 1.8×10¹⁴ vg/kg, 1.3×10¹⁴ vg/kg to about 1.8×10¹⁴ vg/kg, 1.4×10¹⁴ vg/kg to about 1.8×10¹⁴ vg/kg, 1.5×10¹⁴ vg/kg to about 1.8×10¹⁴ vg/kg, 1.6×10¹⁴ vg/kg to about 1.8×10¹⁴ vg/kg, or 1.7×10¹⁴ vg/kg to about 1.8×10¹⁴ vg/kg).

[124] The method of embodiment 122, wherein the viral vector is administered to the patient in an amount of from about 1×10¹⁴ vg/kg to about 1.6×10¹⁴ vg/kg (e.g., 1×10¹⁴ vg/kg to about 1.6×10¹⁴ vg/kg, 1.1×10¹⁴ vg/kg to about 1.6×10¹⁴ vg/kg, 1.2×10¹⁴ vg/kg to about 1.6×10¹⁴ vg/kg, 1.3×10¹⁴ vg/kg to about 1.6×10¹⁴ vg/kg, 1.4×10¹⁴ vg/kg to about 1.6×10¹⁴ vg/kg, 1.5×10¹⁴ vg/kg to about 1.6×10¹⁴ vg/kg, 1.6×10¹⁴ vg/kg to about 1.6×10¹⁴ vg/kg, or 1.7×10¹⁴ vg/kg to about 1.6×10¹⁴ vg/kg).

[125] The method of embodiment 122, wherein the viral vector is administered to the patient in an amount of 1.1×10¹⁴ vg/kg to about 1.5×10¹⁴ vg/kg (e.g., 1.1×10¹⁴ vg/kg to about 1.5×10¹⁴ vg/kg, 1.2×10¹⁴ vg/kg to about 1.5×10¹⁴ vg/kg, 1.3×10¹⁴ vg/kg to about 1.5×10¹⁴ vg/kg, or 1.4×10¹⁴ vg/kg to about 1.5×10¹⁴ vg/kg).

[126] The method of embodiment 122, wherein the viral vector is administered to the patient in an amount of from about 1.2×10¹⁴ vg/kg to about 1.4×10¹⁴ vg/kg (e.g., 1.2×10¹⁴ vg/kg to about 1.4×10¹⁴ vg/kg, or 1.3×10¹⁴ vg/kg to about 1.4×10¹⁴ vg/kg).

[127] The method of any one of embodiments 110-126, wherein the viral vector is administered to the patient in an amount of about 1.3×10¹⁴ vg/kg.

[128] The method of any one of embodiments 1-108 and 117-127, wherein the transgene or viral vector is administered to the patient in a single dose comprising the amount.

[129] The method of any one of embodiments 1-108 and 117-127, wherein the transgene or viral vector is administered to the patient in two or more (e.g., two or more, three or more, four or more, five or more, six or more, seven or more, eight or more, nine or more, or ten or more) doses that, together, comprise the amount.

[130] The method of any one of embodiments 1-108 and 117-127, wherein the transgene or viral vector is administered to the patient in two or more (e.g., two or more, three or more, four or more, five or more, six or more, seven or more, eight or more, nine or more, or ten or more) doses that each, individually, comprise the amount.

[131] The method of embodiment 129 or 130, wherein the two or more (e.g., two or more, three or more, four or more, five or more, six or more, seven or more, eight or more, nine or more, or ten or more) doses are separated from one another by one year or more (e.g., one year or more, two years or more, three years or more, four years or more, or five years or more).

[132] The method of embodiment 129 and 130, wherein the two or more doses are administered to the patient within about 12 months (e.g., within about 12 months, within about 11 months, within about 10 months, within about 9 months, within about 8 months, within about 7 months, within about 6 months, within about 5 months, within about 4 months, within about 3 months, within about 2 months, or within about 1 month) of one another.

[133] The method of any one of embodiments 4-10, 14-38, 42, 46-64, 68, 72-89, 91-108, or 110-132, wherein the viral vector is selected from the group consisting of adeno-associated virus (AAV), adenovirus, lentivirus, retrovirus, poxvirus, baculovirus, herpes simplex virus, vaccinia virus, and a synthetic virus.

[134] The method of embodiment 133, wherein the viral vector is an AAV.

[135] The method of embodiment 134, wherein the AAV is an AAV1, AAV2, AAV3, AAV4, AAV5, AAV6, AAV7, AAV8, AAV9, AAVrh10, or AAVrh74 serotype.

[136] The method of embodiment 134, wherein the viral vector is a pseudotyped AAV.

[137] The method of embodiment 136, wherein the pseudotyped AAV is AAV2/8 or AAV2/9.

[138] The method of embodiment 137, wherein the pseudotyped AAV is AAV2/8.

[139] The method of any one of embodiments 1-138, wherein the transgene encoding MTM1 is operably linked to a muscle specific promoter.

[140] The method of embodiment 139, wherein the muscle specific promotor is a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3.

[141] The method of embodiment 140, wherein the muscle specific promoter is a desmin promoter.

[142] The method of any one of embodiments 4-10, 14-38, 42, 46-64, 68, 72-89, 91-108, or 110-141, wherein the viral vector is resamirigene bilparvovec.

[143] The method of any one of embodiments 4-10, 14-38, 42, 46-64, 68, 72-89, 91-108, or 110-142, wherein the viral vector is administered to the patient by way of intravenous, intramuscular, intradermal, or subcutaneous administration.

[144] The method of any one of embodiments 1-143, wherein the anti-cholestatic agent is selected from the group consisting of a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor.

[145] The method of embodiment 144, wherein the FXR ligand is obeticholic acid.

[146] The method of embodiment 144, wherein the FXR ligand is cilofexor.

[147] The method of embodiment 144, wherein the FXR ligand is tropifextor.

[148] The method of embodiment 144, wherein the FXR ligand is tretinoin.

[149] The method of embodiment 144, wherein the FXR ligand is EDP-305.

[150] The method of embodiment 144, wherein the FGF-19 mimetic is aldafermin.

[151] The method of embodiment 144, wherein the TGR5 agonist is INT-777.

[152] The method of embodiment 144, wherein the TGR5 agonist is INT-767.

[153] The method of embodiment 144, wherein the PPAR agonist is bezafibrate.

[154] The method of embodiment 144, wherein the PPAR agonist is seladelpar.

[155] The method of embodiment 144, wherein the PPAR agonist is elafibrinor.

[156] The method of embodiment 144, wherein the PPAR-alpha agonist is fenofibrate.

[157] The method of embodiment 144, wherein the PPAR-delta agonist is seladelpar.

[158] The method of embodiment 144, wherein the dual PPAR-alpha and PPAR-delta agonist is elafibranor.

[159] The method of embodiment 144, wherein the ASBT inhibitor is odevixibat.

[160] The method of embodiment 144, wherein the ASBT inhibitor is maralixibat.

[161] The method of embodiment 144, wherein the ASBT inhibitor is linerixibat.

[162] The method of embodiment 144, wherein the immunomodulatory drug is rituximab.

[163] The method of embodiment 144, wherein the immunomodulatory drug is abatacept.

[164] The method of embodiment 144, wherein the immunomodulatory drug is ustekinumab.

[165] The method of embodiment 144, wherein the immunomodulatory drug is infliximab.

[166] The method of embodiment 144, wherein the immunomodulatory drug is baricitinib.

[167] The method of embodiment 144, wherein the immunomodulatory drug is FFP-104.

[168] The method of embodiment 144, wherein the antifibrotic therapy is a vitamin D receptor agonist.

[169] The method of embodiment 144, wherein the antifibrotic therapy is simtuzumab.

[170] The method of embodiment 144, wherein the NOX inhibitor is setanaxib.

[171] The method of embodiment 144, wherein the bile acid is ursodeoxycholic acid (e.g., ursodiol) or a pharmaceutically acceptable salt thereof.

[172] The method of embodiment 144, wherein the bile acid is nor-ursodeoxycholic acid or a pharmaceutically acceptable salt thereof.

[173] The method of embodiment 171, wherein the bile acid is ursodiol.

[174] The method of embodiment 144, 171, or 173, wherein the bile acid is administered to the patient in a single dose.

[175] The method of embodiment 144, 171, or 173, wherein the bile acid is administered to the patient in a plurality of doses.

[176] The method of embodiment 174 or 175, wherein the bile acid is administered to the patient in an amount of from about 5 mg/kg/dose to about 20 mg/kg/dose (e.g., 5 mg/kg/dose to about 20 mg/kg/dose, 6 mg/kg/dose to about 20 mg/kg/dose, 7 mg/kg/dose to about 20 mg/kg/dose, 8 mg/kg/dose to about 20 mg/kg/dose, 9 mg/kg/dose to about 20 mg/kg/dose, 10 mg/kg/dose to about 20 mg/kg/dose, 11 mg/kg/dose to about 20 mg/kg/dose, 12 mg/kg/dose to about 20 mg/kg/dose, 13 mg/kg/dose to about 20 mg/kg/dose, 14 mg/kg/dose to about 20 mg/kg/dose, 15 mg/kg/dose to about 20 mg/kg/dose, 16 mg/kg/dose to about 20 mg/kg/dose, 17 mg/kg/dose to about 20 mg/kg/dose, 18 mg/kg/dose to about 20 mg/kg/dose, or 19 mg/kg/dose to about 20 mg/kg/dose).

[177] The method of embodiment 176, wherein the bile acid is administered to the patient in an amount of from about 6 mg/kg/dose to about 19 mg/kg/dose (e.g., 6 mg/kg/dose to about 19 mg/kg/dose, 7 mg/kg/dose to about 19 mg/kg/dose, 8 mg/kg/dose to about 19 mg/kg/dose, 9 mg/kg/dose to about 19 mg/kg/dose, 10 mg/kg/dose to about 19 mg/kg/dose, 11 mg/kg/dose to about 19 mg/kg/dose, 12 mg/kg/dose to about 19 mg/kg/dose, 13 mg/kg/dose to about 19 mg/kg/dose, 14 mg/kg/dose to about 19 mg/kg/dose, 15 mg/kg/dose to about 19 mg/kg/dose, 16 mg/kg/dose to about 19 mg/kg/dose, 17 mg/kg/dose to about 19 mg/kg/dose, or 18 mg/kg/dose to about 19 mg/kg/dose).

[178] The method of embodiment 176, wherein the bile acid is administered to the patient in an amount of from about 7 mg/kg/dose to about 18 mg/kg/dose (e.g., 7 mg/kg/dose to about 18 mg/kg/dose, 8 mg/kg/dose to about 18 mg/kg/dose, 9 mg/kg/dose to about 18 mg/kg/dose, 10 mg/kg/dose to about 18 mg/kg/dose, 11 mg/kg/dose to about 18 mg/kg/dose, 12 mg/kg/dose to about 18 mg/kg/dose, 13 mg/kg/dose to about 18 mg/kg/dose, 14 mg/kg/dose to about 18 mg/kg/dose, 15 mg/kg/dose to about 18 mg/kg/dose, 16 mg/kg/dose to about 18 mg/kg/dose, or 17 mg/kg/dose to about 18 mg/kg/dose).

[179] The method of embodiment 176, wherein the bile acid is administered to the patient in an amount of from about 8 mg/kg/dose to about 17 mg/kg/dose (e.g., 8 mg/kg/dose to about 17 mg/kg/dose, 9 mg/kg/dose to about 17 mg/kg/dose, 10 mg/kg/dose to about 17 mg/kg/dose, 11 mg/kg/dose to about 17 mg/kg/dose, 12 mg/kg/dose to about 17 mg/kg/dose, 13 mg/kg/dose to about 17 mg/kg/dose, 14 mg/kg/dose to about 17 mg/kg/dose, 15 mg/kg/dose to about 17 mg/kg/dose, or 16 mg/kg/dose to about 17 mg/kg/dose).

[180] The method of embodiment 176, wherein the bile acid is administered to the patient in an amount of from about 10 mg/kg/dose to about 15 mg/kg/dose (e.g., 10 mg/kg/dose to about 15 mg/kg/dose, 11 mg/kg/dose to about 15 mg/kg/dose, 12 mg/kg/dose to about 15 mg/kg/dose, 13 mg/kg/dose to about 15 mg/kg/dose, or 14 mg/kg/dose to about 15 mg/kg/dose).

[181] The method of embodiment 176, wherein the bile acid is administered to the patient in an amount of from about 12 mg/kg/dose to about 13 mg/kg/dose.

[182] The method of embodiment 176, wherein the bile acid is administered to the patient in an amount of from about 5 mg/kg/dose to about 11 mg/kg/dose (e.g., 5 mg/kg/dose to about 11 mg/kg/dose, 6 mg/kg/dose to about 11 mg/kg/dose, 7 mg/kg/dose to about 11 mg/kg/dose, 8 mg/kg/dose to about 11 mg/kg/dose, 9 mg/kg/dose to about 11 mg/kg/dose, or 10 mg/kg/dose to about 11 mg/kg/dose).

[183] The method of embodiment 182, wherein the bile acid is administered to the patient in an amount of from about 6 mg/kg/dose to about 10 mg/kg/dose (e.g., 6 mg/kg/dose to about 10 mg/kg/dose, 7 mg/kg/dose to about 10 mg/kg/dose, 8 mg/kg/dose to about 10 mg/kg/dose, or 9 mg/kg/dose to about 10 mg/kg/dose).

[184] The method of embodiment 182, wherein the bile acid is administered to the patient in an amount of from about 7 mg/kg/dose to about 9 mg/kg/dose (e.g., 7 mg/kg/dose to about 7 mg/kg/dose or 8 mg/kg/dose to about 9 mg/kg/dose).

[185] The method of embodiment 182, wherein the bile acid is administered to the patient in one or more (e.g., one or more, two or more, three or more, four or more, five or more, six or more, seven or more, eight or more, nine or more, or ten or more) doses per day, week, or month.

[186] The method of embodiment 185, wherein the bile acid is administered to the patient in one or more (e.g., one or more, two or more, three or more, four or more, five or more, six or more, seven or more, eight or more, nine or more, or ten or more) doses per day.

[187] The method of embodiment 186, wherein the bile acid is administered to the patient in one dose per day.

[188] The method of embodiment 186, wherein the bile acid is administered to the patient in two doses per day.

[189] The method of embodiment 186, wherein the bile acid is administered to the patient in three doses per day.

[190] The method of embodiment 186, wherein the bile acid is administered to the patient in four doses per day.

[191] The method of embodiment 186, wherein the bile acid is administered to the patient in five doses per day.

[192] The method of any one of embodiments 144-191, wherein the bile acid is administered to the patient in an amount of from about 5 mg/kg/day to about 40 mg/kg/day (e.g., 5 mg/kg/day to about 40 mg/kg/day, 5.1 mg/kg/day to about 40 mg/kg/day, 5 mg/kg/day to about 40 mg/kg/day, 5.1 mg/kg/day to about 40 mg/kg/day, 5.2 mg/kg/day to about 40 mg/kg/day, 5.3 mg/kg/day to about 40 mg/kg/day, 5.4 mg/kg/day to about 40 mg/kg/day, 5.5 mg/kg/day to about 40 mg/kg/day, 6 mg/kg/day to about 40 mg/kg/day, 6.5 mg/kg/day to about 40 mg/kg/day, 7 mg/kg/day to about 40 mg/kg/day, 8 mg/kg/day to about 40 mg/kg/day, 9 mg/kg/day to about 40 mg/kg/day, 10 mg/kg/day to about 40 mg/kg/day, 11 mg/kg/day to about 40 mg/kg/day, 12 mg/kg/day to about 40 mg/kg/day, 13 mg/kg/day to about 40 mg/kg/day, 14 mg/kg/day to about 40 mg/kg/day, 15 mg/kg/day to about 40 mg/kg/day, 16 mg/kg/day to about 40 mg/kg/day, 17 mg/kg/day to about 40 mg/kg/day, 18 mg/kg/day to about 40 mg/kg/day, 19 mg/kg/day to about 40 mg/kg/day, 20 mg/kg/day to about 40 mg/kg/day, 25 mg/kg/day to about 40 mg/kg/day, 30 mg/kg/day to about 40 mg/kg/day, 35 mg/kg/day to about 40 mg/kg/day, or 40 mg/kg/day to about 40 mg/kg/day).

[193] The method of embodiment 192, wherein the bile acid is administered to the patient in an amount of from about 6 mg/kg/day to about 39 mg/kg/day (e.g., 6 mg/kg/day to about 39 mg/kg/day, 6.5 mg/kg/day to about 39 mg/kg/day, 7 mg/kg/day to about 39 mg/kg/day, 8 mg/kg/day to about 39 mg/kg/day, 9 mg/kg/day to about 39 mg/kg/day, 10 mg/kg/day to about 39 mg/kg/day, 11 mg/kg/day to about 39 mg/kg/day, 12 mg/kg/day to about 39 mg/kg/day, 13 mg/kg/day to about 39 mg/kg/day, 14 mg/kg/day to about 39 mg/kg/day, 15 mg/kg/day to about 39 mg/kg/day, 16 mg/kg/day to about 39 mg/kg/day, 17 mg/kg/day to about 39 mg/kg/day, 18 mg/kg/day to about 39 mg/kg/day, 19 mg/kg/day to about 39 mg/kg/day, 20 mg/kg/day to about 39 mg/kg/day, 25 mg/kg/day to about 39 mg/kg/day, 30 mg/kg/day to about 39 mg/kg/day, or 35 mg/kg/day to about 39 mg/kg/day).

[194] The method of embodiment 192, wherein the bile acid is administered to the patient in an amount of from about 8 mg/kg/day to about 37 mg/kg/day (e.g., 8 mg/kg/day to about 37 mg/kg/day, 9 mg/kg/day to about 37 mg/kg/day, 10 mg/kg/day to about 37 mg/kg/day, 11 mg/kg/day to about 37 mg/kg/day, 12 mg/kg/day to about 37 mg/kg/day, 13 mg/kg/day to about 37 mg/kg/day, 14 mg/kg/day to about 37 mg/kg/day, 15 mg/kg/day to about 37 mg/kg/day, 16 mg/kg/day to about 37 mg/kg/day, 17 mg/kg/day to about 37 mg/kg/day, 18 mg/kg/day to about 37 mg/kg/day, 19 mg/kg/day to about 37 mg/kg/day, 20 mg/kg/day to about 37 mg/kg/day, 25 mg/kg/day to about 37 mg/kg/day, 30 mg/kg/day to about 37 mg/kg/day, or 35 mg/kg/day to about 37 mg/kg/day).

[195] The method of embodiment 192, wherein the bile acid is administered to the patient in an amount of from about 13 mg/kg/day to about 32 mg/kg/day (e.g., 13 mg/kg/day to about 32 mg/kg/day, 14 mg/kg/day to about 32 mg/kg/day, 15 mg/kg/day to about 32 mg/kg/day, 16 mg/kg/day to about 32 mg/kg/day, 17 mg/kg/day to about 32 mg/kg/day, 18 mg/kg/day to about 32 mg/kg/day, 19 mg/kg/day to about 32 mg/kg/day, 20 mg/kg/day to about 32 mg/kg/day, 25 mg/kg/day to about 32 mg/kg/day, or 30 mg/kg/day to about 32 mg/kg/day).

[196] The method of embodiment 192, wherein the bile acid is administered to the patient in an amount of from about 20 mg/kg/day to about 25 mg/kg/day (e.g., 20 mg/kg/day to about 25 mg/kg/day, 21 mg/kg/day to about 25 mg/kg/day, 22 mg/kg/day to about 25 mg/kg/day, 23 mg/kg/day to about 25 mg/kg/day, or 24 mg/kg/day to about 25 mg/kg/day).

[197] The method of embodiment 192, wherein the bile acid is administered to the patient in an amount of from about 17 mg/kg/day to about 23 mg/kg/day (e.g., 17 mg/kg/day to about 23 mg/kg/day, 18 mg/kg/day to about 23 mg/kg/day, 19 mg/kg/day to about 23 mg/kg/day, 20 mg/kg/day to about 23 mg/kg/day, 21 mg/kg/day to about 23 mg/kg/day, or 22 mg/kg/day to about 23 mg/kg/day).

[198] The method of embodiment 192, wherein the bile acid is administered to the patient in an amount of from about 18 mg/kg/day to about 22 mg/kg/day (e.g., 18 mg/kg/day to about 22 mg/kg/day, 19 mg/kg/day to about 22 mg/kg/day, 20 mg/kg/day to about 22 mg/kg/day, or 21 mg/kg/day to about 22 mg/kg/day).

[199] The method of embodiment 192, wherein the bile acid is administered to the patient in an amount of from about 19 mg/kg/day to about 21 mg/kg/day (e.g., 19 mg/kg/day to about 21 mg/kg/day or 20 mg/kg/day to about 21 mg/kg/day).

[200] The method of any one of embodiments 144-199, wherein the bile acid is administered to the patient in an amount of 20 mg/kg/day.

[201] The method of any one of embodiments 144-200, wherein the bile acid is administered to the patient by way of a unit dosage form comprising 250 mg of the bile acid.

[202] The method of any one of embodiments 144-200, wherein the bile acid is administered to the patient by way of a unit dosage form comprising 500 mg of the bile acid.

[203] The method of any one of embodiments 144-202, wherein the bile is administered to the patient by way of enteral administration.

[204] The method of any one of embodiments 1-203, wherein the patient does not have a history of cholestasis or hyperbilirubinemia.

[205] The method of embodiment 204, wherein the patient does not have a history of any underlying liver disease.

[206] The method of any one of embodiments 1-205, wherein the patient was born at greater than or equal to 35 weeks of gestational age and is or was from term age (e.g., adjusted term age) to about 5 years old (e.g., 1 day old to about 5 years old, 2 days old to about 5 years old, 3 days old to about 5 years old, 4 days old to about 5 years old, 5 days old to about 5 years old, 6 days old to about 5 years old, 7 days old to about 5 years old, 8 days old to about 5 years old, 9 days old to about 5 years old, 10 days old to about 5 years old, 11 days old to about 5 years old, 12 days old to about 5 years old, 13 days old to about 5 years old, 14 days old to about 5 years old, 15 days old to about 5 years old, 16 days old to about 5 years old, 17 days old to about 5 years old, 18 days old to about 5 years old, 19 days old to about 5 years old, 20 days old to about 5 years old, 25 days old to about 5 years old, one month old to about 5 years old, two months old to about 5 years old, 3 months old to about 5 years old, 4 months old to about 5 years old, 5 months old to about 5 years old, 6 months old to about 5 years old, 1 year old to about 5 years old, 2 years old to about 5 years old, 3 years old to about 5 years old, and 4 years old to about 5 years old) at the time of administration of the transgene or viral vector.

[207] The method of any one of embodiments 1-206, wherein the patient is male.

[208] The method of any one of embodiments 1-207, wherein the patient requires mechanical ventilatory support.

[209] The method of embodiment 208, wherein mechanical ventilatory support comprises invasive mechanical ventilatory support.

[210] The method of embodiment 208, wherein mechanical ventilatory support comprises noninvasive mechanical ventilatory support.

[211] The method of any one of embodiments 1-210, wherein upon administering the transgene or the viral vector to the patient, the patient exhibits a change from baseline in hours of mechanical ventilation support over time.

[212] The method of embodiment 211, wherein the patient exhibits the change from baseline in hours of mechanical ventilation support over time by about 24 weeks after administration of the transgene or viral vector to the patient.

[213] The method of embodiment 212, wherein the patient displays a change from baseline in hours of mechanical ventilation support over time by about 20 weeks, 16 weeks, 12 weeks, 8 weeks, or 4 weeks after administration of the viral vector to the patient.

[214] The method of any one of embodiments 1-212, wherein upon administering the transgene or the viral vector to the patient the patient achieves the functionally independent sitting by about 24 weeks after administration of the transgene or viral vector to the patient.

[215] The method of embodiment 214, wherein upon administering the transgene or the viral vector to the patient the patient achieves a functionally independent sitting by about 20 weeks, 16 weeks, 12 weeks, 8 weeks, or 4 weeks after administration of the viral vector to the patient.

[216] The method of any one of embodiments 1-215, wherein upon administering the transgene or the viral vector to the patient, the patient displays a reduction in required mechanical ventilator support to about 16 hours or less per day.

[217] The method of embodiment 216, wherein the patient displays the reduction in required mechanical ventilator support by about 24 weeks after administration of the transgene or viral vector to the patient.

[218] The method of embodiment 217, wherein upon administering the transgene or the viral vector to the patient, the patient displays a reduction in required mechanical ventilator support by about 20 weeks, 16 weeks, 12 weeks, 8 weeks, or 4 weeks after administration of the viral vector to the patient.

[219] The method of any one of embodiments 1-218, wherein upon administering the transgene or the viral vector to the patient, the patient displays a change from baseline on the CHOP INTEND.

[220] The method of embodiment 219, wherein the patient displays the change from baseline on the CHOP INTEND by about 24 weeks after administration of the transgene or viral vector to the patient.

[221] The method of embodiment 220, wherein upon administering the transgene or the viral vector to the patient, the patient displays a change from baseline on the CHOP INTEND by about 20 weeks, 16 weeks, 12 weeks, 8 weeks, or 4 weeks after administration of the viral vector to the patient.

[222] The method of any one of embodiments 1-221, wherein upon administering the transgene or the viral vector to the patient, the patient displays a change from baseline in maximal inspiratory pressure (MIP).

[223] The method of embodiment 222, wherein the patient displays the change from baseline in MIP by about 24 weeks after administration of the transgene or viral vector to the patient.

[224] The method of embodiment 223, wherein upon administering the transgene or the viral vector to the patient, the patient displays a change from baseline in MIP by about 20 weeks, 16 weeks, 12 weeks, 8 weeks, or 4 weeks after administration of the viral vector to the patient.

[225] The method of any one of embodiments 1-224, wherein upon administering the transgene or the viral vector to the patient, the patient displays a change from baseline in quantitative analysis of myotubularin expression in a muscle biopsy.

[226] The method of embodiment 225, wherein the patient displays the change from baseline in quantitative analysis of myotubularin expression in a muscle biopsy by about 24 weeks after administration of the transgene or viral vector to the patient.

[227] The method of embodiment 226, wherein upon administering the transgene or the viral vector to the patient, the patient displays a change from baseline in quantitative analysis of myotubularin expression in a muscle biopsy by about 20 weeks, 16 weeks, 12 weeks, 8 weeks, or 4 weeks after administration of the viral vector to the patient.

[228] The method of embodiment 226 or 227 wherein the change from baseline in quantitative analysis of myotubularin expression in a muscle biopsy persists for at least 48 weeks after administration of the viral vector to the patient.

[229] The method of any one of embodiments 1-228, wherein upon administering the transgene or the viral vector to the patient, the patient displays a reduction of stiffness and/or joint contractures, optionally wherein the patient displays the reduction of stiffness and/or joint contractures by about 24 weeks after administration of the viral vector to the patient, optionally wherein the patient displays the reduction of stiffness and/or joint contractures by about 20 weeks, 16 weeks, 12 weeks, 8 weeks, or 4 weeks after administration of the viral vector to the patient.

[230] The method of any one of embodiments 1-229, wherein upon administering the transgene or the viral vector to the patient, the patient displays diaphragm and/or respiratory muscle progression, optionally wherein the patient displays the diaphragm and/or respiratory muscle progression by about 24 weeks after administration of the viral vector to the patient, optionally wherein the patient displays the diaphragm and/or respiratory muscle progression by about 20 weeks, 16 weeks, 12 weeks, 8 weeks, or 4 weeks after administration of the viral vector to the patient.

[231] The method of any one of embodiments 33-226, wherein the patient is determined to exhibit cholestasis or one or more symptoms thereof by a finding that the patient exhibits a serum total bile acids level that is greater than 14 μmol/L (e.g., greater than 14 μmol/L, 15 μmol/L, 16 μmol/L, 17 μmol/L, 18 μmol/L, 19 μmol/L, 20 μmol/L, 21 μmol/L, 22 μmol/L, 23 μmol/L, 24 μmol/L, 25 μmol/L, 26 μmol/L, 27 μmol/L, 28 μmol/L, 29 μmol/L, 30 μmol/L, 31 μmol/L, 32 μmol/L, 33 μmol/L, 34 μmol/L, 35 μmol/L, 36 μmol/L, 37 μmol/L, 38 μmol/L, 39 μmol/L, 40 μmol/L, 41 μmol/L, 42 μmol/L, 43 μmol/L, 44 μmol/L, 45 μmol/L, 46 μmol/L, 47 μmol/L, 48 μmol/L, 49 μmol/L, 50 μmol/L, 51 μmol/L, 52 μmol/L, 53 μmol/L, 54 μmol/L, 55 μmol/L, 56 μmol/L, 57 μmol/L, 58 μmol/L, 59 μmol/L, 60 μmol/L, 61 μmol/L, 62 μmol/L, 63 μmol/L, 64 μmol/L, 65 μmol/L, 66 μmol/L, 67 μmol/L, 68 μmol/L, 69 μmol/L, 70 μmol/L, 71 μmol/L, 72 μmol/L, 73 μmol/L, 74 μmol/L, 75 μmol/L, 76 μmol/L, 77 μmol/L, 78 μmol/L, 79 μmol/L, 80 μmol/L, 81 μmol/L, 82 μmol/L, 83 μmol/L, 84 μmol/L, 85 μmol/L, 86 μmol/L, 87 μmol/L, 88 μmol/L, 89 μmol/L, 90 μmol/L, 91 μmol/L, 92 μmol/L, 93 μmol/L, 94 μmol/L, 95 μmol/L, 96 μmol/L, 97 μmol/L, 98 μmol/L, 99 μmol/L, or 100 μmol/L).

[232] The method of any one of embodiments 33-231, wherein the patient is determined to exhibit cholestasis or one or more symptoms thereof by a finding that the patient exhibits one or more parameters in a blood test that is increased or decreased relative to a reference level.

[233] The method of embodiment 232, wherein the blood test is a liver function test.

[234] The method of embodiment 232 or 233, wherein the one or more parameters comprises the level of gamma-glutamyl transferase, alkaline phosphatase, aspartate aminotransferase, and/or alanine aminotransferase.

[235] The method of any one of embodiments 33-234, wherein the patient is determined to exhibit hyperbilirubinemia or one or more symptoms thereof by a finding that the patient exhibits a bilirubin level that is greater than 1 mg/dL in a bilirubin test (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL).

[236] The method of embodiment 235 wherein the bilirubin level that is greater than 1 mg/dL in a bilirubin test occurs by about 3 weeks after administration of the transgene or the viral vector to the patient.

[237] The method of embodiment 235 or 236, wherein the bilirubin level comprises a direct bilirubin level or a total bilirubin level.

[238] The method of any one of embodiments 33-237, wherein the patient is determined to exhibit cholestasis, hyperbilirubinemia, or one or more symptoms thereof by a finding that the patient exhibits a parameter in blood test that is increased relative to a reference level.

[239] The method of embodiment 238, wherein the parameter comprises the level of a serum bile acid.

[240] The method of embodiment 239, wherein the serum bile acid is cholic acid, chenodeoxycholic acid, deoxycholic acid, or ursodeoxycholic acid.

[241] The method of embodiment 238, wherein the blood test is a liver function test.

[242] The method of embodiment 241, wherein the parameter comprises the level of aspartate aminotransferase or alanine aminotransferase.

[243] A kit comprising a transgene encoding MTM1 and a package insert, wherein the package insert instructs a user of the kit to administer the transgene to a patient having XLMTM in accordance with the method of any one of embodiments 1-3, 11-13, 39-41, 43-45, 65-67, and 69-71.

[244] A kit comprising a viral vector comprising a transgene encoding MTM1 and a package insert, wherein the package insert instructs a user of the kit to administer the viral vector to a patient having XLMTM in accordance with the method of any one of embodiments 1-89 and 128-243.

[245] A kit comprising an anti-cholestatic agent and a package insert, wherein the package insert instructs a user of the kit to administer the anti-cholestatic agent to a patient to treat or prevent cholestasis or hyperbilirubinemia in accordance with the method of any one of embodiments 90-127.

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating X-linked myotubular myopathy (XLMTM) in a human patient in need thereof, the method comprising administering to the patient (i) a therapeutically effective amount of a viral vector comprising a transgene encoding myotubularin 1 (MTM1) and (ii) an anti-cholestatic agent, wherein the anti-cholestatic agent is administered to the patient in one or more doses that commence within about six weeks of administration of the viral vector to the patient.

2. A method of reducing stiffness and/or joint contractures in a human patient diagnosed as having XLMTM, the method comprising administering to the patient (i) a therapeutically effective amount of a viral vector comprising a transgene encoding MTM1 and (ii) an anti-cholestatic agent, wherein the anti-cholestatic agent is administered to the patient in one or more doses that commence within about six weeks of administration of the viral vector to the patient.

3. A method of increasing diaphragm and/or respiratory muscle progression in a human patient diagnosed as having XLMTM, the method comprising administering to the patient (i) a therapeutically effective amount of a viral vector comprising a transgene encoding MTM1 and (ii) an anti-cholestatic agent, wherein the anti-cholestatic agent is administered to the patient in one or more doses that commence within about six weeks of administration of the viral vector to the patient.

4. The method of any one of claims 1-3, wherein the anti-cholestatic agent is administered to the patient in one or more doses that commence within about five weeks of administration of the viral vector to the patient, optionally wherein the anti-cholestatic agent is administered to the patient in one or more doses that commence within about four weeks, within about three weeks, within about two weeks, or within about one week of administration of the viral vector to the patient.

5. The method of claim 4, wherein the anti-cholestatic agent is administered to the patient in one or more doses that commence on the same day as administration of the viral vector to the patient.

6. A method of treating XLMTM in a human patient in need thereof and who has been previously administered an anti-cholestatic agent, the method comprising administering to the patient a therapeutically effective amount of a viral vector comprising a transgene encoding MTM1.

7. A method of reducing stiffness and/or joint contractures in a human patient diagnosed as having XLMTM and who has been previously administered an anti-cholestatic agent, the method comprising administering to the patient a therapeutically effective amount of a viral vector comprising a transgene encoding MTM1.

8. A method of increasing diaphragm and/or respiratory muscle progression in a human patient diagnosed as having XLMTM and who has been previously administered an anti-cholestatic agent, the method comprising administering to the patient a therapeutically effective amount of a viral vector comprising a transgene encoding MTM1.

9. The method of any one of claims 1-8, wherein the viral vector is administered to the patient in an amount of less than about 3×10¹⁴ vg/kg.

10. The method of claim 9, wherein the viral vector is administered to the patient in an amount of less than about 2.5×10¹⁴ vg/kg, optionally wherein the viral vector is administered to the patient in an amount of less than about 2×10¹⁴ vg/kg, less than about 1.5×10¹⁴ vg/kg, or less than about 1.4×10¹⁴ vg/kg.

11. The method of any one of claims 1-10, wherein the viral vector is administered to the patient in an amount of from about 3×10¹³ vg/kg to about 2.3×10¹⁴ vg/kg, optionally wherein the viral vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg.

12. The method of any one of claims 1-11, wherein the viral vector is administered to the patient in an amount of about 1.3×10¹⁴ vg/kg.

13. The method of any one of claims 1-12, wherein the patient is five years old or younger at the time of administration of the viral vector.

14. The method of claim 13, wherein the patient is four years old or younger at the time of administration of the viral vector, optionally wherein the patient is three years old or younger, two years old or younger, one year old or younger, or six months old or younger.

15. The method of any one of claims 1-12, wherein the patient is from about 1 month old to about 5 years old at the time of administration of the viral vector.

16. The method of any one of claims 1-15, the method further comprising monitoring the patient for development of cholestasis, hyperbilirubinemia, or one or more symptoms thereof.

17. The method of claim 16, wherein the patient is monitored for the development of cholestasis, hyperbilirubinemia, or one or more symptoms thereof by evaluating a parameter in a blood sample obtained from the patient, wherein a finding that the parameter is above a reference level identifies the patient as having cholestasis, hyperbilirubinemia, or one or more symptoms thereof.

18. The method of claim 17, wherein the parameter comprises the level of a serum bile acid in the blood sample.

19. The method of claim 18, wherein the serum bile acid is cholic acid, chenodeoxycholic acid, deoxycholic acid, or ursodeoxycholic acid.

20. The method of claim 17, wherein the parameter comprises one or more results of a liver function test.

21. The method of claim 20, wherein the parameter comprises the level of aspartate aminotransferase or alanine aminotransferase in the blood sample.

22. A method of treating XLMTM in a human patient in need thereof, the method comprising:
(a) administering to the patient a viral vector comprising a transgene encoding MTM1 in an amount of less than about 3×10¹⁴ vg/kg,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

23. A method of reducing stiffness and/or joint contractures in a human patient diagnosed as having XLMTM, the method comprising:
(a) administering to the patient a viral vector comprising a transgene encoding MTM1 in an amount of less than about 3×10¹⁴ vg/kg,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

24. A method of increasing diaphragm and/or respiratory muscle progression in a human patient diagnosed as having XLMTM, the method comprising:
a) administering to the patient a viral vector comprising a transgene encoding MTM1 in an amount of less than about 3×10¹⁴ vg/kg,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

25. A method of treating XLMTM in a human patient in need thereof, the method comprising:
(a) administering to the patient a viral vector comprising a transgene encoding MTM1 in an amount of less than about 3×10¹⁴ vg/kg,
(b) determining that the patient exhibits cholestasis, hyperbilirubinemia, or one or more symptoms thereof, and
(c) administering to the patient an anti-cholestatic agent.

26. A method of reducing stiffness and/or joint contractures in a human patient diagnosed as having XLMTM, the method comprising:
(a) administering to the patient a viral vector comprising a transgene encoding MTM1 in an amount of less than about 3×10¹⁴ vg/kg,
(b) determining that the patient exhibits cholestasis, hyperbilirubinemia, or one or more symptoms thereof, and
(c) administering to the patient an anti-cholestatic agent.

27. A method of increasing diaphragm and/or respiratory muscle progression in a human patient diagnosed as having XLMTM, the method comprising:
(a) administering to the patient a viral vector comprising a transgene encoding MTM1 in an amount of less than about 3×10¹⁴ vg/kg,
(b) determining that the patient exhibits cholestasis, hyperbilirubinemia, or one or more symptoms thereof, and
(c) administering to the patient an anti-cholestatic agent.

28. The method of any one of claims 22-27, wherein the viral vector is administered to the patient in an amount of less than about 2.5×10¹⁴ vg/kg, optionally wherein the viral vector is administered to the patient in an amount of less than about 2×10¹⁴ vg/kg, less than about 1.5×10¹⁴ vg/kg, or less than about 1.4×10¹⁴ vg/kg.

29. The method of any one of claims 22-27, wherein the viral vector is administered to the patient in an amount of from about 3×10¹³ vg/kg to about 2.3×10¹⁴ vg/kg, optionally wherein the viral vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg.

30. The method of any one of claims 22-29, wherein the viral vector is administered to the patient in an amount of about 1.3×10¹⁴ vg/kg.

31. The method of any one of claims 22-30, wherein the patient is five years old or younger at the time of administration of the viral vector.

32. The method of claim 24, wherein the patient is four years old or younger at the time of administration of the viral vector, optionally wherein the patient is three years old or younger, two years old or younger, one year old or younger, or six months old or younger.

33. The method of any one of claims 22-30, wherein the patient is from about 1 month old to about 5 years old at the time of administration of the viral vector.

34. A method of treating XLMTM in a human patient in need thereof that is five years old or younger, the method comprising:
(a) administering to the patient a therapeutically effective amount of a viral vector comprising a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

35. A method of reducing stiffness and/or joint contractures in a human patient diagnosed as having XLMTM, the method comprising:
(a) administering to the patient a therapeutically effective amount of a viral vector comprising a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

36. A method of increasing diaphragm and/or respiratory muscle progression in a human patient diagnosed as having XLMTM, the method comprising:
(a) administering to the patient a therapeutically effective amount of a viral vector comprising a transgene encoding MTM1,
(b) monitoring the patient for development of cholestasis, hyperbilirubinemia, or one or more symptoms thereof, and, if the patient exhibits cholestasis, hyperbilirubinemia, or one or more symptoms thereof,
(c) administering to the patient an anti-cholestatic agent.

37. A method of treating XLMTM in a human patient in need thereof that is five years old or younger, the method comprising:
(a) administering to the patient a therapeutically effective amount of a viral vector comprising a transgene encoding MTM1,
(b) determining that the patient exhibits cholestasis, hyperbilirubinemia, or one or more symptoms thereof, and
(c) administering to the patient an anti-cholestatic agent.

38. A method of reducing stiffness and/or joint contractures in a human patient diagnosed as having XLMTM, the method comprising:
(a) administering to the patient a therapeutically effective amount of a viral vector comprising a transgene encoding MTM1,
(b) determining that the patient exhibits cholestasis, hyperbilirubinemia, or one or more symptoms thereof, and
(c) administering to the patient an anti-cholestatic agent.

39. A method of increasing diaphragm and/or respiratory muscle progression in a human patient diagnosed as having XLMTM, the method comprising:
(a) administering to the patient a therapeutically effective amount of a viral vector comprising a transgene encoding MTM1,
(b) determining that the patient exhibits cholestasis, hyperbilirubinemia, or one or more symptoms thereof, and
(c) administering to the patient an anti-cholestatic agent.

40. The method of any one of claims 34-39, wherein the patient is four years old or younger at the time of administration of the viral vector, optionally wherein the patient is three years old or younger, two years old or younger, one year old or younger, or six months old or younger.

41. The method of any one of claims 34-39, wherein the patient is from about 1 month old to about 5 years old at the time of administration of the viral vector.

42. The method of any one of claims 34-41, wherein the viral vector is administered to the patient in an amount of less than about 3×10¹⁴ vg/kg.

43. The method of claim 42, wherein the viral vector is administered to the patient in an amount of less than about 2.5×10¹⁴ vg/kg, optionally wherein the viral vector is administered to the patient in an amount of less than about 2×10¹⁴ vg/kg, less than about 1.5×10¹⁴ vg/kg, or less than about 1.4×10¹⁴ vg/kg.

44. The method of any one of claims 34-41, wherein the viral vector is administered to the patient in an amount of from about 3×10¹³ vg/kg to about 2.3×10¹⁴ vg/kg, optionally wherein the viral vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg.

45. The method of any one of claims 34-44, wherein the viral vector is administered to the patient in an amount of about 1.3×10¹⁴ vg/kg.

46. A method of treating or preventing cholestasis or hyperbilirubinemia in a human patient that has XLMTM and who has been previously administered a viral vector comprising a transgene encoding MTM1 in an amount of less than about 3×10¹⁴ vg/kg, the method comprising administering to the patient an anti-cholestatic agent.

47. The method of claim 46, wherein the viral vector is administered to the patient in an amount of less than about 2.5×10¹⁴ vg/kg, optionally wherein the viral vector is administered to the patient in an amount of less than about 2×10¹⁴ vg/kg, less than about 1.5×10¹⁴ vg/kg, or less than about 1.4×10¹⁴ vg/kg.

48. The method of claim 46, wherein the viral vector is administered to the patient in an amount of from about 3×10¹³ vg/kg to about 2.3×10¹⁴ vg/kg, optionally wherein the viral vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg.

49. The method of any one of claims 46-48, wherein the viral vector is administered to the patient in an amount of about 1.3×10¹⁴ vg/kg.

50. The method of any one of claims 46-49, wherein the patient is five years old or younger at the time of administration of the viral vector.

51. The method of claim 50, wherein the patient is four years old or younger at the time of administration of the viral vector, optionally wherein the patient is three years old or younger, two years old or younger, one year old or younger, or six months old or younger.

52. The method of any one of claims 46-49, wherein the patient is from about 1 month old to about 5 years old at the time of administration of the viral vector.

53. A method of treating or preventing cholestasis or hyperbilirubinemia in a human patient that has XLMTM, has been previously administered a viral vector comprising a transgene encoding MTM1, and that was five years old or younger at the time of administration of the viral vector, the method comprising administering to the patient an anti-cholestatic agent.

54. The method of claim 53, wherein the patient was four years old or younger at the time of administration of the viral vector, optionally wherein the patient was three years old or younger, two years old or younger, one year old or younger, or six months old or younger.

55. The method of claim 53, wherein the patient was from about 1 month old to about 5 years old at the time of administration of the viral vector.

56. The method of any one of claims 53-55, wherein the viral vector is administered to the patient in an amount of less than about 3×10¹⁴ vg/kg.

57. The method of claim 56, wherein the viral vector is administered to the patient in an amount of less than about 2.5×10¹⁴ vg/kg, optionally wherein the viral vector is administered to the patient in an amount of less than about 2×10¹⁴ vg/kg, less than about 1.5×10¹⁴ vg/kg, or less than about 1.4×10¹⁴ vg/kg.

58. The method of any one of claims 53-55, wherein the viral vector is administered to the patient in an amount of from about 3×10¹³ vg/kg to about 2.3×10¹⁴ vg/kg, optionally wherein the viral vector is administered to the patient in an amount of from about 8×10¹³ vg/kg to about 1.8×10¹⁴ vg/kg, from about 1×10¹⁴ vg/kg to about 1.6×10¹⁴ vg/kg, from about 1.1×10¹⁴ vg/kg to about 1.5×10¹⁴ vg/kg, or from about 1.2×10¹⁴ vg/kg to about 1.4×10¹⁴ vg/kg.

59. The method of any one of claims 53-58, wherein the viral vector is administered to the patient in an amount of about 1.3×10¹⁴ vg/kg.

60. The method of any one of claims 1-52 and 56-59, wherein the viral vector is administered to the patient in a single dose comprising the amount.

61. The method of any one of claims 1-52 and 56-59, wherein the viral vector is administered to the patient in two or more doses that, together, comprise the amount.

62. The method of any one of claims 1-52 and 56-59, wherein the viral vector is administered to the patient in two or more doses that each, individually, comprise the amount.

63. The method of claim 61 or 62, wherein the two or more doses are separated from one another by one year or more.

64. The method of claim 61 or 62, wherein the two or more doses are administered to the patient within about 12 months of one another.

65. The method of any one of claims 1-64, wherein the viral vector is selected from the group consisting of adeno-associated virus (AAV), adenovirus, lentivirus, retrovirus, poxvirus, baculovirus, herpes simplex virus, vaccinia virus, and a synthetic virus.

66. The method of claim 65, wherein the viral vector is an AAV.

67. The method of claim 66, wherein the AAV is an AAV1, AAV2, AAV3, AAV4, AAV5, AAV6, AAV7, AAV8, AAV9, AAVrh10, or AAVrh74 serotype.

68. The method of claim 66, wherein the viral vector is a pseudotyped AAV.

69. The method of claim 68, wherein the pseudotyped AAV is AAV2/8 or AAV2/9, optionally wherein the pseudotyped AAV is AAV2/8.

70. The method of any one of claims 1-69, wherein the transgene encoding MTM1 is operably linked to a muscle specific promoter.

71. The method of claim 70, wherein the muscle specific promotor is a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3.

72. The method of claim 71, wherein the muscle specific promoter is a desmin promoter.

73. The method of any one of claims 1-72, wherein the viral vector is resamirigene bilparvovec.

74. The method of any one of claims 1-73, wherein the viral vector is administered to the patient by way of intravenous, intramuscular, intradermal, or subcutaneous administration.

75. The method of any one of claims 1-74, wherein the anti-cholestatic agent is selected from the group consisting of a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent bile acid transporter (ASBT) inhibitor, an immunomodulatory drug, an antifibrotic therapy, and a nicotinamide adenine dinucleotide phosphate oxidase (NOX) inhibitor.

76. The method of claim 75, wherein:
(i) the FXR ligand is obeticholic acid, cilofexor, tropifexor, tretinoin, or EDP-305;
(ii) the FGF-19 mimetic is aldafermin;
(iii) the TGR5 agonist is INT-777 or INT-767;
(iv) the PPAR agonist is bezafibrate, seladelpar, or elafibrinor;
(v) the PPAR-alpha agonist is fenofibrate;
(vi) the PPAR-delta agonist is seladelpar;
(vii) the dual PPAR-alpha and PPAR-delta agonist is elafibranor;
(viii) the ASBT inhibitor is odevixibat, maralixibat, or linerixibat;
(ix) the immunomodulatory drug is rituximab, abatacept, ustekinumab, infliximab, baricitinib, or FFP-104;
(x) the antifibrotic therapy is a vitamin D receptor agonist or simtuzumab; and/or
(xi) the NOX inhibitor is setanaxib.

77. The method of claim 75, wherein the bile acid is ursodeoxycholic acid, nor-ursodeoxycholic acid, or a pharmaceutically acceptable salt thereof.

78. The method of claim 77, wherein the bile acid is ursodiol.

79. The method of any one of claims 75, 77, and 78, wherein the bile acid is administered to the patient in a single dose.

80. The method of any one of claims 75, 77, and 78, wherein the bile acid is administered to the patient in a plurality of doses.

81. The method of claim 79 or 80, wherein the bile acid is administered to the patient in an amount of from about 5 mg/kg/dose to about 20 mg/kg/dose, optionally wherein the bile acid is administered to the patient in an amount of from about 6 mg/kg/dose to about 19 mg/kg/dose, from about 7 mg/kg/dose to about 18 mg/kg/dose, from about 8 mg/kg/dose to about 17 mg/kg/dose, from about 10 mg/kg/dose to about 15 mg/kg/dose, or from about 12 mg/kg/dose to about 13 mg/kg/dose.

82. The method of claim 81, wherein the bile acid is administered to the patient in an amount of from about 5 mg/kg/dose to about 11 mg/kg/dose, optionally wherein the bile acid is administered to the patient in an amount of from about 6 mg/kg/dose to about 10 mg/kg/dose, or from about 7 mg/kg/dose to about 9 mg/kg/dose.

83. The method of claim 82, wherein the bile acid is administered to the patient in one or more doses per day, week, or month.

84. The method of claim 83, wherein the bile acid is administered to the patient in one or more doses per day, optionally wherein the bile acid is administered to the patient in one dose per day, in two doses per day, three doses per day, four doses per day, or five doses per day.

85. The method of claim 84, wherein the bile acid is administered to the patient in one dose per day.

86. The method of any one of claims 75-85, wherein the bile acid is administered to the patient in an amount of from about 5 mg/kg/day to about 40 mg/kg/day, optionally wherein (i) the bile acid is administered to the patient in an amount of from about 6 mg/kg/day to about 39 mg/kg/day, from about 8 mg/kg/day to about 37 mg/kg/day, from about 13 mg/kg/day to about 32 mg/kg/day, or from about 20 mg/kg/day to about 25 mg/kg/day, or (ii) the bile acid is administered to the patient in an amount of from about 17 mg/kg/day to about 23 mg/kg/day, from about 18 mg/kg/day to about 22 mg/kg/day, or from about 19 mg/kg/day to about 21 mg/kg/day.

87. The method of any one of claims 75-86, wherein the bile acid is administered to the patient in an amount of 20 mg/kg/day.

88. The method of any one of claims 75-87, wherein the bile acid is administered to the patient by way of a unit dosage form comprising 250 mg of the bile acid.

89. The method of any one of claims 75-87, wherein the bile acid is administered to the patient by way of a unit dosage form comprising 500 mg of the bile acid.

90. The method of any one of claims 75-89, wherein the bile is administered to the patient by way of enteral administration.

91. The method of any one of claims 1-90, wherein the patient does not have a history of cholestasis or hyperbilirubinemia.

92. The method of claim 91, wherein the patient does not have a history of any underlying liver disease.

93. The method of any one of claims 1-92, wherein the patient was born at greater than or equal to 35 weeks of gestational age and is or was from term age to about 5 years old at the time of administration of the viral vector.

94. The method of any one of claims 1-93, wherein the patient is male.

95. The method of any one of claims 1-94, wherein the patient requires mechanical ventilatory support, optionally wherein mechanical ventilatory support comprises invasive mechanical ventilatory support and noninvasive mechanical ventilatory support.

96. The method of any one of claims 1-95, wherein upon administering the viral vector to the patient, the patient exhibits a change from baseline in hours of mechanical ventilation support over time, optionally wherein the patient exhibits the change from baseline in hours of mechanical ventilation support overtime by about 24 weeks after administration of the viral vector to the patient, optionally wherein the patient displays the change from baseline in hours of mechanical ventilation support over time by about 20 weeks, 16 weeks, 12 weeks, 8 weeks, or 4 weeks after administration of the viral vector to the patient.

97. The method of any one of claims 1-96, wherein upon administering the viral vector to the patient, the patient achieves functionally independent sitting for at least 30 seconds, optionally wherein the patient achieves the functionally independent sitting by about 24 weeks after administration of the viral vector to the patient, optionally wherein the patient displays the functionally independent sitting for at least 30 seconds by about 20 weeks, 16 weeks, 12 weeks, 8 weeks, or 4 weeks after administration of the viral vector to the patient.

98. The method of any one of claims 1-97, wherein upon administering the viral vector to the patient, the patient displays a reduction in required mechanical ventilator support to about 16 hours or less per day, optionally wherein the patient displays the reduction in required mechanical ventilator support by about 24 weeks after administration of the viral vector to the patient, optionally wherein the patient displays the reduction in required mechanical ventilator support by about 20 weeks, 16 weeks, 12 weeks, 8 weeks, or 4 weeks after administration of the viral vector to the patient.

99. The method of any one of claims 1-98, wherein upon administering the viral vector to the patient, the patient displays a change from baseline on the Children's Hospital of Philadelphia Infant Test of Neuromuscular Disorders (CHOP INTEND), optionally wherein the patient displays the change from baseline on the CHOP INTEND by about 24 weeks after administration of the viral vector to the patient, optionally wherein the patient displays the change from baseline on the CHOP INTEND by about 20 weeks, 16 weeks, 12 weeks, 8 weeks, or 4 weeks after administration of the viral vector to the patient.

100. The method of any one of claims 1-99, wherein upon administering the viral vector to the patient, the patient displays a change from baseline in maximal inspiratory pressure (MIP), optionally wherein the patient displays the change from baseline in MIP by about 24 weeks after administration of the viral vector to the patient, optionally wherein the patient displays the change from baseline in MIP by about 20 weeks, 16 weeks, 12 weeks, 8 weeks, or 4 weeks after administration of the viral vector to the patient.

101. The method of any one of claims 1-100, wherein upon administering the viral vector to the patient, the patient displays a change from baseline in quantitative analysis of myotubularin expression in a muscle biopsy, optionally wherein the patient displays the change from baseline in quantitative analysis of myotubularin expression in a muscle biopsy by about 24 weeks after administration of the viral vector to the patient, optionally wherein the patient displays the change from baseline in quantitative analysis of myotubularin expression in a muscle biopsy by about 20 weeks, 16 weeks, 12 weeks, 8 weeks, or 4 weeks after administration of the viral vector to the patient.

102. The method of claim 101, wherein the change from baseline in quantitative analysis of myotubularin expression in a muscle biopsy persists for at least 48 weeks after administration of the viral vector to the patient.

103. The method of any one of claims 1-102, wherein upon administering the viral vector to the patient, the patient displays a reduction of stiffness and/or joint contractures, optionally wherein the patient displays the reduction of stiffness and/or joint contractures by about 24 weeks after administration of the viral vector to the patient, optionally wherein the patient displays the reduction of stiffness and/or joint contractures by about 20 weeks, 16 weeks, 12 weeks, 8 weeks, or 4 weeks after administration of the viral vector to the patient.

104. The method of any one of claims 1-103, wherein upon administering the viral vector to the patient, the patient displays diaphragm and/or respiratory muscle progression, optionally wherein the patient displays the diaphragm and/or respiratory muscle progression by about 24 weeks after administration of the viral vector to the patient, optionally wherein the patient displays the diaphragm and/or respiratory muscle progression by about 20 weeks, 16 weeks, 12 weeks, 8 weeks, or 4 weeks after administration of the viral vector to the patient.

105. The method of any one of claims 16-104, wherein the patient is determined to exhibit cholestasis or one or more symptoms thereof by a finding that the patient exhibits a serum total bile acids level that is greater than 14 μmol/L.

106. The method of any one of claims 16-105, wherein the patient is determined to exhibit cholestasis or one or more symptoms thereof by a finding that the patient exhibits one or more parameters in a blood test that is increased or decreased relative to a reference level.

107. The method of claim 106, wherein the blood test is a liver function test.

108. The method of claim 106 or 107, wherein the one or more parameters comprises the level of gamma-glutamyl transferase, alkaline phosphatase, aspartate aminotransferase, and/or alanine aminotransferase.

109. The method of any one of claims 16-108, wherein the patient is determined to exhibit hyperbilirubinemia or one or more symptoms thereof by a finding that the patient exhibits a bilirubin level that is greater than 1 mg/dL in a bilirubin test.

110. The method of claim 109, wherein upon administering the viral vector to the patient, the patient displays a bilirubin level that is greater than 1 mg/dL in a bilirubin test by about 3 weeks after administration of the viral vector to the patient.

111. The method of claim 109 or 110, wherein the bilirubin level comprises a direct bilirubin level or a total bilirubin level.

112. The method of any one of claims 16-111, wherein the patient is determined to exhibit cholestasis, hyperbilirubinemia, or one or more symptoms thereof by a finding that the patient exhibits a parameter in blood test that is increased relative to a reference level.

113. The method of claim 112, wherein the parameter comprises the level of a serum bile acid.

114. The method of claim 113, wherein the serum bile acid is cholic acid, chenodeoxycholic acid, deoxycholic acid, or ursodeoxycholic acid.

115. The method of claim 112, wherein the blood test is a liver function test.

116. The method of claim 115, wherein the parameter comprises the level of aspartate aminotransferase or alanine aminotransferase.

117. A kit comprising a viral vector comprising a transgene encoding MTM1 and a package insert, wherein the package insert instructs a user of the kit to administer the viral vector to a patient having XLMTM in accordance with the method of any one of claims 1-52 and 60-116.

118. A kit comprising an anti-cholestatic agent and a package insert, wherein the package insert instructs a user of the kit to administer the anti-cholestatic agent to a patient to treat or prevent cholestasis or hyperbilirubinemia in accordance with the method of any one of claims 53-116.
